SEQUENCE LISTING (16 sequences)

SEQ ID NO: 1 - protein, 3392 aa, Dengue virus serotype 1
MNNQRKKTGRPSFNMLKRARNRVSTGSQLAKRFSKGLLSGQGPMKLVMAFIAFLRFLAIPPTAGILARWSSFKKNGAIKVLRGFKKEISSMLNIMNRRKKSVTMLLMLLPTALAFHLTTRGGEPHMIVSKQERGKSLLFKTSAGINMCTLIAMDLGELCEDTMTYKCPRITEAEPDDVDCWCNATDTWVTYGTCSQTGEHRRDKRSVALAPHVGLGLETRTETWMSSEGAWKQIQKVETWALRHPGFTVTALFLAHAIGTSITQKGIIFILLMLVTPSMAMRCVGIGNRDFVEGLSGATWVDVVLEHGSCVTTMAKDKPTLDIELLKTEVTNPAVLRKLCIEAKISNTTTDSRCPTQGEATLVEEQDSNFVCRRTFVDRGWGNGCGLFGKGSLLTCAKFKCVTKLEGKIVQYENLKYSVIVTVHTGDQHQVGNETTEHGTIATITPQAPTSEIQLTDYGALTLDCSPRTGLDFNEMVLLTMKEKSWLVHKQWFLDLPLPWTSGASTSQETWNRQDLLVTFKTAHAKKQEVVVLGSQEGAMHTALTGATEIQTSGTTTIFAGHLKCRLKMDKLTLKGMSYVMCTGSFKLEKEVAETQHGTVLVQVKYEGTDAPCKIPFSAQDEKGVTQNGRLITANPIVTDKEKPVNIETEPPFGESYIVVGAGEKALKLSWFKKGSSIGKMFEATARGARRMAILGDTAWDFGSIGGVFTSVGKLVHQVFGAAYGVLFSGVSWTMKIGIGILLTWLGLNSRSTSLSMTCIAVGMVTLYLGVMVQADSGCVINWKGRELKCGSGIFVTNEVHTWTEQYKFQADSPKRLSAAIGKAWEEGVCGIRSATRLENIMWKQISNELNHILLENDMKFTVVVGDANGILAQGKKMIRPQPMEHRYSWKSWGKAKIIGADIQNSTFIIDGPDTPECPDDQRAWNIWEVEDYGFGIFTTNIWLKLRDSYTQMCDHRLMSAAIKDSKAVHADMGYWIESEKNETWKLARASFIEVKTCIWPKSHTLWSNGVLESEMIIPKIYGGPISQHNYRPGYFTQTAGPWHLGKLELDFDLCEGTTVVVDEHCGNRGPSLRTTTVTGKIIHEWCCRSCTLPPLRFRGEDGCWYGMEIRPVKEKEENLVRSMVSAGSGEVDSFSLGILCVSIMIEEVMRSRWSRKMLMTGTLAVFLLLIMGQLTWNDLIRLCIMVGANASDRMGMGTTYLALMATFKMRPMFAVGLLFRRLTSREVLLLTIGLSLVASVELPNSLEELGDGLAMGIMMLKLLTEFQPHQLWTTLLSLTFIKTTLSLDYAWRTMAMVLSIVSLFPLCLSTTSQKTTWLPVLLGSFGCKPLTMFLITENKIWGRKSWPLNEGIMAIGIVSILLSSLLKNDVPLAGPLIAGGMLIACYVISGSSADLSLEKAAEVSWEEEAEHSGTSHNILVEVQDDGTMKIKDEERDDTLTILLKATLLAVSGVYPMSIPATLFVWYFWQKKKQRSGVLWDTPSPPEVERAVLDDGIYRILQRGLLGRSQVGVGVFQDGVFHTMWHVTRGAVLMYQGKRLEPSWASVKKDLISYGGGWRFQGSWNTGEEVQVIAVEPGKNPKNVQTTPGTFKTPEGEVGAIALDFKPGTSGSPIVNREGKIVGLYGNGVVTTSGTYVSAIAQAKTSQEGPLPEIEEEVFKKRNLTIMDLHPGSGKTRRYLPAIVREAIKRKLRTLILAPTRVVASEMAEALKGMPIRYQTTAVKSEHTGREIVDLMCHATFTMRLLSPVRVPNYNMIIMDEAHFTDPASIAARGYISTRVGMGEAAAIFMTATPPGSVEAFPQSNAVIQDEERDIPERSWNSGYDWITDFPGKTVWFVPSIKSGNDIANCLRKNGKRVIQLSRKTFDTEYQKTRNNDWDYVVTTDISEMGANFRADRVIDPRRCLKPVILKDGPERVILAGPMPVTVASAAQRRGRIGRNQNKEGDQYVYMGQPLNNDEDHAHWTEAKMLLDNINTPEGIIPALFEPEREKSAAIDGEYRLRGEARKTFVELMRRGDLPVWLSYKVASEGFQYSDRRWCFDGERNNQVLEENMDVEIWTKEGERKKLRPRWLDARTYSDPLALREFKEFAAGRRSVSGDLILEIGKLPQHLTLRAQNALDNLVMLHNSEQGGKAYRHAMEELPDTIETLMLLALIAVLTGGVTLFFLSGKGLGKTSIGLLCVMASSALLWMANVEPHWIAASIILEFFLMVLLIPEPDRQRTPQDNQLAYVVIGLLFMILTVAANEMGLLETTKKDLGIGYVAAENQHHATMLDVDLRPASAWTLYAVATTVITPMMRHTIENTTANISLTAIANQAAILMGLDKGWPISKMDIGVPLLALGCYSQVNPLTLTAAVLMLVAHYAIIGPGLQAKATREAQKRTAAGIMKNPTVDGIVAIDLDPVVYDAKFEKQLGQIMLLILCTSQILLMRTTWALCESITLATGPLTTLWEGSPGKFWNTTIAVSMANIFRGSYLAGAGLAFSLMKSLGGGRRGTGAQGETLGEKWKRQLNQLSKSEFNTYKRSGIMEVDRSEAKEGLKRGETTKHAVSRGTAKLRWFVERNLVKPEGKVIDLGCGRGGWSYYCAGLKKVTEVKGYTKGGPGHEEPIPMATYGWNLVKLYSGKDVFFTPPEKCDTLLCDIGESSPNPTIEEGRTLRVLKMVEPWLRGNQFCIKILNPYMPSVVETLEQMQRKHGGMLVRNPLSRNSTHEMYWVSCGTGNIVSAVNMTSRMLLNRFTMTHRKPTYERDVDLGAGTRHVAVEPEVANLDIIGQRIENIKNEHKSTWYYDEDNPYKTWAYHGSYEVKPSGSASSMVNGVVRLLTKPWDVIPMVTQIAMTDTTPFGQQRVFKEKVDTRTPRAKRGTAQIMEVTAKWLWGFLSRNKKPRICTREEFTRKVRSNAAIGAVFVDENQWNSAKEAVEDERFWDLVHRERELHKQGKCATCVYNMMGKREKKLGEFGKAKGSRAIWYMWLGARFLEFEALGFMNEDHWFSRENSFSGVEGEGLHKLGYILRDISKIPGGNMYADDTAGWDTRITEDDLQNEAKITDIMEPEHALLATSIFKLTYQNKVVRVQRPAKNGTVMDVISRRDQRGSGQVGTYGLNTFTNMEVQLIRQMESEGIFLPSELETPNLAERVLNWLEKHGTERLKRMAISGDDCVVKPVDDRFATALIALNDMGKVRKDIPQWEPSKGWNDWQQVPFCSHHFHQLIMKDGREIVVPCRNQDELVGRARVSQGAGWSLRETACLGKSYAQMWQLMYFHRRDLRLAANAICSAVPVDWVPTSRTTWSIHAHHQWMTTEDMLSVWNRVWIEENPWMEDKTHVSSWEEVPYLGKREDQWCGSLIGLTARATWATNIQVAINQVRRLIGNENYLDYMTSMKRFKNESDPEGALW

SEQ ID NO: 2 - protein, 3391 aa, Dengue virus serotype 2
MNNQRKKAKNTPFNMLKRERNRVSTVQQLTKRFSLGMLQGRGPLKLFMALVAFLRFLTIPPTAGILKRWGTIKKSKAINVLRGFRKEIGRMLNILNRRRRSAGMIIMLIPTVMAFHLTTRNGEPHMIVSRQEKGKSLLFKTEDGVNMCTLMAMDLGELCEDTITYKCPLLRQNEPEDIDCWCNSTSTWVTYGTCTTMGEHRREKRSVALVPHVGMGLETRTETWMSSEGAWKHVQRIETWILRHPGFTMMAAILAYTIGTTHFQRALIFILLTAVTPSMTMRCIGMSNRDFVEGVSGGSWVDIVLEHGSCVTTMAKNKPTLDFELIKTEAKQPATLRKYCIEAKLTNTTTESRCPTQGEPSLNEEQDKRFVCKHSMVDRGWGNGCGLFGKGGIVTCAMFRCKKNMEGKVVQPENLEYTIVITPHSGEEHAVGNDTGKHGKEIKITPQSSITEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLNWFKKGSSIGQMFETTMRGAKRMAILGDTAWDFGSLGGVFTSIGKALHQVFGAIYGAAFSGVSWTMKILIGVIITWIGMNSRSTSLSVTLVLVGIVTLYLGVMVQADSGCVVSWKNKELKCGSGIFITDNVHTWTEQYKFQPESPSKLASAIQKAHEEGICGIRSVTRLENLMWKQITPELNHILSENEVKLTIMTGDIKGIMQAGKRSLRPQPTELKYSWKTWGKAKMLSTESHNQTFLIDGPETAECPNTNRAWNSLEVEDYGFGVFTTNIWLKLKEKQDVFCDSKLMSAAIKDNRAVHADMGYWIESALNDTWKIEKASFIEVKNCHWPKSHTLWSNGVLESEMIIPKNLAGPVSQHNYRPGYHTQITGPWHLGKLEMDFDFCDGTTVVVTEDCGNRGPSLRTTTASGKLITEWCCRSCTLPPLRYRGEDGCWYGMEIRPLKEKEENLVNSLVTAGHGQVDNFSLGVLGMALFLEEMLRTRVGTKHAILLVAVSFVTLITGNMSFRDLGRVMVMVGATMTDDIGMGVTYLALLAAFKVRPTFAAGLLLRKLTSKELMMTTIGIVLLSQSTIPETILELTDALALGMMVLKMVRNMEKYQLAVTIMAILCVPNAVILQNAWKVSCTILAVVSVSPLLLTSSQQKTDWIPLALTIKGLNPTAIFLTTLSRTSKKRSWPLNEAIMAVGMVSILASSLLKNDIPMTGPLVAGGLLTVCYVLTGRSADLELERAADVKWEDQAEISGSSPILSITISEDGSMSIKNEEEEQTLTILIRTGLLVISGLFPVSIPITAAAWYLWEVKKQRAGVLWDVPSPPPMGKAELEDGAYRIKQKGILGYSQIGAGVYKEGTFHTMWHVTRGAVLMHKGKRIEPSWADVKKDLISYGGGWKLEGEWKEGEEVQVLALEPGKNPRAVQTKPGLFKTNAGTIGAVSLDFSPGTSGSPIIDKKGKVVGLYGNGVVTRSGAYVSAIAQTEKSIEDNPEIEDDIFRKRRLTIMDLHPGAGKTKRYLPAIVREAIKRGLRTLILAPTRVVAAEMEEALRGLPIRYQTPAIRAEHTGREIVDLMCHATFTMRLLSPVRVPNYNLIIMDEAHFTDPASIAARGYISTRVEMGEAAGIFMTATPPGSRDPFPQSNAPIIDEEREIPERSWNSGHEWVTDFKGKTVWFVPSIKAGNDIAACLRKNGKKVIQLSRKTFDSEYVKTRTNDWDFVVTTDISEMGANFKAERVIDPRRCMKPVILTDGEERVILAGPMPVTHSSAAQRRGRIGRNPKNENDQYIYMGEPLENDEDCAHWKEAKMLLDNINTPEGIIPSMFEPEREKVDAIDGEYRLRGEARKTFVDLMRRGDLPVWLAYRVAAEGINYADRRWCFDGVKNNQILEENVEVEIWTKEGERKKLKPRWLDARIYSDPLALKEFKEFAAGRKSLTLNLITEMGRLPTFMTQKARDALDNLAVLHTAEAGGRAYNHALSELPETLETLLLLTLLATVTGGIFLFLMSGRGIGKMTLGMCCIITASILLWYAQIQPHWIAASIILEFFLIVLLIPEPEKQRTPQDNQLTYVVIAILTVVAATMANEMGFLEKTKKDLGLGSIATQQPESNILDIDLRPASAWTLYAVATTFVTPMLRHSIENSSVNVSLTAIANQATVLMGLGKGWPLSKMDIGVPLLAIGCYSQVNPITLTAALFLLVAHYAIIGPGLQAKATREAQKRAAAGIMKNPTVDGITVIDLDPIPYDPKFEKQLGQVMLLVLCVTQVLMMRTTWALCEALTLATGPISTLWEGNPGRFWNTTIAVSMANIFRGSYLAGAGLLFSIMKNTTNTRRGTGNIGETLGEKWKSRLNALGKSEFQIYKKSGIQEVDRTLAKEGIKRGETDHHAVSRGSAKLRWFVERNMVTPEGKVVDLGCGRGGWSYYCGGLKNVREVKGLTKGGPGHEEPIPMSTYGWNLVRLQSGVDVFFIPPEKCDTLLCDIGESSPNPTVEAGRTLRVLNLVENWLNNNTQFCIKVLNPYMPSVIEKMEALQRKYGGALVRNPLSRNSTHEMYWVSNASGNIVSSVNMISRMLINRFTMRYKKATYEPDVDLGSGTRNIGIESEIPNLDIIGKRIEKIKQEHETSWHYDQDHPYKTWAYHGSYETKQTGSASSMVNGVVRLLTKPWDVVPMVTQMAMTDTTPFGQQRVFKEKVDTRTQEPKEGTKKLMKITAEWLWKELGKKKTPRMCTREEFTRKVRSNAALGAIFTDENKWKSAREAVEDSRFWELVDKERNLHLEGKCETCVYNMMGKREKKLGEFGKAKGSRAIWYMWLGARFLEFEALGFLNEDHWFSRENSLSGVEGEGLHKLGYILRDVSKKEGGAMYADDTAGWDTRITLEDLKNEEMVTNHMEGEHKKLAEAIFKLTYQNKVVRVQRPTPRGTVMDIISRRDQRGSGQVGTYGLNTFTNMEAQLIRQMEGEGVFKSIQHLTITEEIAVQNWLARVGRERLSRMAISGDDCVVKPLDDRFASALTALNDMGKIRKDIQQWEPSRGWNDWTQVPFCSHHFHELIMKDGRVLVVPCRNQDELIGRARISQGAGWSLRETACLGKSYAQMWSLMYFHRRDLRLAANAICSAVPSHWVPTSRTTWSIHAKHEWMTTEDMLTVWNRVWIQENPWMEDKTPVESWEEIPYLGKREDQWCGSLIGLTSRATWAKNIQAAINQVRSLIGNEEYTDYMPSMKRFRREEEEAGVLW

SEQ ID NO: 3 - protein, 3390 aa, Dengue virus serotype 3
MNNQRKKTGKPSINMLKRVRNRVSTGSQLAKRFSKGLLNGQGPMKLVMAFIAFLRFLAIPPTAGVLARWGTFKKSGAIKVLKGFKKEISNMLSIINQRKKTSLCLMMILPAALAFHLTSRDGEPRMIVGKNERGKSLLFKTASGINMCTLIAMDLGEMCDDTVTYKCPHITEVEPEDIDCWCNLTSTWVTYGTCNQAGEHRRDKRSVALAPHVGMGLDTRTQTWMSAEGAWRQVEKVETWALRHPGFTILALFLAHYIGTSLTQKVVIFILLMLVTPSMTMRCVGVGNRDFVEGLSGATWVDVVLEHGGCVTTMAKNKPTLDIELQKTEATQLATLRKLCIEGKITNITTDSRCPTQGEAVLPEEQDQNYVCKHTYVDRGWGNGCGLFGKGSLVTCAKFQCLEPIEGKVVQYENLKYTVIITVHTGDQHQVGNETQGVTAEITPQASTTEAILPEYGTLGLECSPRTGLDFNEMILLTMKNKAWMVHRQWFFDLPLPWASGATTETPTWNRKELLVTFKNAHAKKQEVVVLGSQEGAMHTALTGATEIQNSGGTSIFAGHLKCRLKMDKLELKGMSYAMCTNTFVLKKEVSETQHGTILIKVEYKGEDAPCKIPFSTEDGQGKAHNGRLITANPVVTKKEEPVNIEAEPPFGESNIVIGIGDNALKINWYKKGSSIGKMFEATERGARRMAILGDTAWDFGSVGGVLNSLGKMVHQIFGSAYTALFSGVSWVMKIGIGVLLTWIGLNSKNTSMSFSCIAIGIITLYLGAVVQADMGCVINWKGKELKCGSGIFVTNEVHTWTEQYKFQADSPKRLATAIAGAWENGVCGIRSTTRMENLLWKQIANELNYILWENNIKLTVVVGDTLGVLEQGKRTLTPQPMELKYSWKTWGKAKIVTAETQNSSFIIDGPNTPECPSASRAWNVWEVEDYGFGVFTTNIWLKLREVYTQLCDHRLMSAAVKDERAVHADMGYWIESQKNGSWKLEKASLIEVKTCTWPKSHTLWTNGVLESDMIIPKSLAGPISQHNYRPGYHTQTAGPWHLGKLELDFNYCEGTTVVITESCGTRGPSLRTTTVSGKLIHEWCCRSCTLPPLRYMGEDGCWYGMEIRPISEKEENMVKSLVSAGSGKVDNFTMGVLCLAILFEEVLRGKFGKKHMIAGVFFTFVLLLSGQITWRDMAHTLIMIGSNASDRMGMGVTYLALIATFKIQPFLALGFFLRKLTSRENLLLGVGLAMATTLQLPEDIEQMANGVALGLMALKLITQFETYQLWTALVSLTCSNTIFTLTVAWRTATLILAGVSLLPVCQSSSMRKTDWLPMTVAAMGVPPLPLFIFSLKDTLKRRSWPLNEGVMAVGLVSILASSLLRNDVPMAGPLVAGGLLIACYVITGTSADLTVEKAPDVTWEEEAEQTGVSHNLMITVDDDGTMRIKDDETENILTVLLKTALLIVSGIFPYSIPATLLVWHTWQKQTQRSGVLWDVPSPPETQKAELEEGVYRIKQQGIFGKTQVGVGVQKEGVFHTMWHVTRGAVLTHNGKRLEPNWASVKKDLISYGGGWRLSAQWQKGEEVQVIAVEPGKNPKNFQTTPGTFQTTTGEIGAIALDFKPGTSGSPIINREGKVVGLYGNGVVTKNGGYVSGIAQTNAEPDGPTPELEEEMFKKRNLTIMDLHPGSGKTRKYLPAIVREAIKRRLRTLILAPTRVVAAEMEEALKGLPIRYQTTATKSEHTGREIVDLMCHATFTMRLLSPVRVPNYNLIIMDEAHFTDPASIAARGYISTRVGMGEAAAIFMTATPPGTADAFPQSNAPIQDEERDIPERSWNSGNEWITDFAGKTVWFVPSIKAGNDIANCLRKNGKKVIQLSRKTFDTEYQKTKLNDWDFVVTTDISEMGANFKADRVIDPRRCLKPVILTDGPERVILAGPMPVTAASAAQRRGRVGRNPQKENDQYIFTGQPLNNDEDHAHWTEAKMLLDNINTPEGIIPALFEPEREKSAAIDGEYRLKGESRKTFVELMRRGDLPVWLAHKVASEGIKYTDRKWCFDGQRNNQILEENMDVEIWTKEGEKKKLRPRWLDARTYSDPLALKEFKDFAAGRKSIALDLVTEIGRVPSHLAHRTRNALDNLVMLHTSEDGGRAYRHAVEELPETMETLLLLGLMILLTGGAMLFLISGKGIGKTSIGLICVIASSGMLWMAEVPLQWIASAIVLEFFMMVLLIPEPEKQRTPQDNQLAYVVIGILTLAATIAANEMGLLETTKRDLGMSKEPGVVSPTSYLDVDLHPASAWTLYAVATTVITPMLRHTIENSTANVSLAAIANQAVVLMGLDKGWPISKMDLGVPLLALGCYSQVNPLTLTAAVLLLITHYAIIGPGLQAKATREAQKRTAAGIMKNPTVDGIMTIDLDSVIFDSKFEKQLGQVMLLVLCAVQLLLMRTSWALCEALTLATGPITTLWEGSPGKFWNTTIAVSMANIFRGSYLAGAGLAFSIMKSVGTGKRGTGSQGETLGEKWKKKLNQLSRKEFDLYKKSGITEVDRTEAKEGLKRGETTHHAVSRGSAKLQWFVERNMVVPEGRVIDLGCGRGGWSYYCAGLKKVTEVRGYTKGGPGHEEPVPMSTYGWNIVKLMSGKDVFYLPPEKCDTLLCDIGESSPSPTVEESRTIRVLKMVEPWLKNNQFCIKVLNPYMPTVIEHLERLQRKHGGMLVRNPLSRNSTHEMYWISNGTGNIVSSVNMVSRLLLNRFTMTHRRPTIEKDVDLGAGTRHVNAEPETPNMDVIGERIKRIKEEHNSTWHYDDENPYKTWAYHGSYEVKATGSASSMINGVVKLLTKPWDVVPMVTQMAMTDTTPFGQQRVFKEKVDTRTPRPMPGTRKAMEITAEWLWRTLGRNKRPRLCTREEFTKKVRTNAAMGAVFTEENQWDSAKAAVEDEEFWKLVDRERELHKLGKCGSCVYNMMGKREKKLGEFGKAKGSRAIWYMWLGARYLEFEALGFLNEDHWFSRENSYSGVEGEGLHKLGYILRDISKIPGGAMYADDTAGWDTRITEDDLHNEEKIIQQMDPEHRQLANAIFKLTYQNKVVKVQRPTPTGTVMDIISRKDQRGSGQLGTYGLNTFTNMEAQLVRQMEGEGVLTKADLENPHLLEKKITQWLETKGVERLKRMAISGDDCVVKPIDDRFANALLALNDMGKVRKDIPQWQPSKGWHDWQQVPFCSHHFHELIMKDGRKLVVPCRPQDELIGRARISQGAGWSLRETACLGKAYAQMWSLMYFHRRDLRLASNAICSAVPVHWVPTSRTTWSIHAHHQWMTTEDMLTVWNRVWIEENPWMEDKTPVTTWENVPYLGKREDQWCGSLIGLTSRATWAQNIPTAIQQVRSLIGNEEFLDYMPSMKRFRKEEESEGAIW

SEQ ID NO: 4 - protein, 3387 aa, Dengue virus serotype 4
MNQRKKVVRPPFNMLKRERNRVSTPQGLVKRFSTGLFSGKGPLRMVLAFITFLRVLSIPPTAGILKRWGQLKKNKAIKILIGFRKEIGRMLNILNGRKRSTITLLCLIPTVMAFSLSTRDGEPLMIVAKHERGRPLLFKTTEGINKCTLIAMDLGEMCEDTVTYKCPLLVNTEPEDIDCWCNLTSTWVMYGTCTQSGERRREKRSVALTPHSGMGLETRAETWMSSEGAWKHAQRVESWILRNPGFALLAGFMAYMIGQTGIQRTVFFVLMMLVAPSYGMRCVGVGNRDFVEGVSGGAWVDLVLEHGGCVTTMAQGKPTLDFELTKTTAKEVALLRTYCIEASISNITTATRCPTQGEPYLKEEQDQQYICRRDVVDRGWGNGCGLFGKGGVVTCAKFSCSGKITGNLVQIENLEYTVVVTVHNGDTHAVGNDTSNHGVTAMITPRSPSVEVKLPDYGELTLDCEPRSGIDFNEMILMKMKKKTWLVHKQWFLDLPLPWTAGADTSEVHWNYKERMVTFKVPHAKRQDVTVLGSQEGAMHSALAGATEVDSGDGNHMFAGHLKCKVRMEKLRIKGMSYTMCSGKFSIDKEMAETQHGTTVVKVKYEGAGAPCKVPIEIRDVNKEKVVGRIISSTPLAENTNSVTNIELEPPFGDSYIVIGVGNSALTLHWFRKGSSIGKMFESTYRGAKRMAILGETAWDFGSVGGLFTSLGKAVHQVFGSVYTTMFGGVSWMIRILIGFLVLWIGTNSRNTSMAMTCIAVGGITLFLGFTVQADMGCVASWSGKELKCGSGIFVVDNVHTWTEQYKFQPESPARLASAILNAHKDGVCGIRSTTRLENVMWKQITNELNYVLWEGGHDLTVVAGDVKGVLTKGKRALTPPVSDLKYSWKTWGKAKIFTPEARNSTFLIDGPDTSECPNERRAWNSLEVEDYGFGMFTTNIWMKFREGSSEVCDHRLMSAAIKDQKAVHADMGYWIESSKNQTWQIEKASLIEVKTCLWPKTHTLWSNGVLESQMLIPKSYAGPFSQHNYRQGYATQTVGPWHLGKLEIDFGECPGTTVTIQEDCDHRGPSLRTTTASGKLVTQWCCRSCTMPPLRFLGEDGCWYGMEIRPLSEKEENMVKSQVTAGQGTSETFSMGLLCLTLFVEECLRRRVTRKHMILVVVITLCAIILGGLTWMDLLRALIMLGDTMSGRIGGQIHLAIMAVFKMSPGYVLGVFLRKLTSRETALMVIGMAMTTVLSIPHDLMELIDGISLGLILLKIVTQFDNTQVGTLALSLTFIRSTMPLVMAWRTIMAVLFVVTLIPLCRTSCLQKQSHWVEITALILGAQALPVYLMTLMKGASRRSWPLNEGIMAVGLVSLLGSALLKNDVPLAGPMVAGGLLLAAYVMSGSSADLSLEKAANVQWDEMADITGSSPIVEVKQDEDGSFSIRDVEETNMITLLVKLALITVSGLYPLAIPVTMTLWYMWQVKTQRSGALWDVPSPAATKKAALSEGVYRIMQRGLFGKTQVGVGIHMEGVFHTMWHVTRGSVICHETGRLEPSWADVRNDMISYGGGWRLGDKWDKEEDVQVLAIEPGKNPKHVQTKPGLFKTLTGEIGAVTLDFKPGTSGSPIINRKGKVIGLYGNGVVTKSGDYVSAITQAERIGEPDYEVDEDIFRKKRLTIMDLHPGAGKTKRILPSIVREALKRRLRTLILAPTRVVAAEMEEALRGLPIRYQTPAVKSEHTGREIVDLMCHATFTTRLLSSTRVPNYNLIVMDEAHFTDPSSVAARGYISTRVEMGEAAAIFMTATPPGATDPFPQSNSPIEDIEREIPERSWNTGFDWITDYQGKTVWFVPSIKAGNDIANCLRKSGKKVIQLSRKTFDTEYPKTKLTDWDFVVTTDISEMGANFRAGRVIDPRRCLKPVILPDGPERVILAGPIPVTPASAAQRRGRIGRNPAQEDDQYVFSGDPLKNDEDHAHWTEAKMLLDNIYTPEGIIPTLFGPEREKTQAIDGEFRLRGEQRKTFVELMRRGDLPVWLSYKVASAGISYEDREWCFTGERNNQILEENMEVEIWTREGEKKKLRPRWLDARVYADPMALKDFKEFASGRKSITLDILTEIASLPTYLSSRAKLALDNIVMLHTTERGGRAYQHALNELPESLETLMLVALLGAMTAGIFLFFMQGKGIGKLSMGLITIAVASGLLWVAEIQPQWIAASIILEFFLMVLLIPEPEKQRTPQDNQLIYVILTILTIIGLIAANEMGLIEKTKTDFGFYQVKTETTILDVDLRPASAWTLYAVATTILTPMLRHTIENTSANLSLAAIANQAAVLMGLGKGWPLHRMDLGVPLLAMGCYSQVNPTTLTASLVMLLVHYAIIGPGLQAKATREAQKRTAAGIMKNPTVDGITVIDLEPISYDPKFEKQLGQVMLLVLCAGQLLLMRTTWAFCEVLTLATGPILTLWEGNPGRFWNTTIAVSTANIFRGSYLAGAGLAFSLIKNAQTPRRGTGTTGETLGEKWKRQLNSLDRKEFEEYKRSGILEVDRTEAKSALKDGSKIKHAVSRGSSKIRWIVERGMVKPKGKVVDLGCGRGGWSYYMATLKNVTEVKGYTKGGPGHEEPIPMATYGWNLVKLHSGVDVFYKPTEQVDTLLCDIGESSSNPTIEEGRTLRVLKMVEPWLSSKPEFCIKVLNPYMPTVIEELEKLQRKHGGNLVRCPLSRNSTHEMYWVSGASGNIVSSVNTTSKMLLNRFTTRHRKPTYEKDVDLGAGTRSVSTETEKPDMTIIGRRLQRLQEEHKETWHYDQENPYRTWAYHGSYEAPSTGSASSMVNGVVKLLTKPWDVIPMVTQLAMTDTTPFGQQRVFKEKVDTRTPQPKPGTRMVMTTTANWLWALLGKKKNPRLCTREEFISKVRSNAAIGAVFQEEQGWTSASEAVNDSRFWELVDKERALHQEGKCESCVYNMMGKREKKLGEFGRAKGSRAIWYMWLGARFLEFEALGFLNEDHWFGRENSWSGVEGEGLHRLGYILEEIDKKDGDLMYADDTAGWDTRITEDDLQNEELITEQMAPHHKILAKAIFKLTYQNKVVKVLRPTPRGAVMDIISRKDQRGSGQVGTYGLNTFTNMEVQLIRQMEAEGVITQDDMQNPKGLKERVEKWLKECGVDRLKRMAISGDDCVVKPLDERFGTSLLFLNDMGKVRKDIPQWEPSKGWKNWQEVPFCSHHFHKIFMKDGRSLVVPCRNQDELIGRARISQGAGWSLRETACLGKAYAQMWSLMYFHRRDLRLASMAICSAVPTEWFPTSRTTWSIHAHHQWMTTEDMLKVWNRVWIEDNPNMTDKTPVHSWEDIPYLGKREDLWCGSLIGLSSRATWAKNIHTAITQVRNLIGKEEYVDYMPVMKRYSAPSESEGVL

SEQ ID NO: 5 - DNA, 10690 bp, Dengue virus serotype 1
acaagaacagtttcgaatcggaagcttgcttaacgtagttctaacagttttttattagagagcagatctctgatgaacaaccaacggaaaaagacgggtcgaccgtctttcaatatgctgaaacgcgcgagaaaccgcgtgtcaactggttcacagttggcgaagagattctcaaaaggattgctttcgggccaaggacccatgaaattggtgatggcttttatagcatttctaagatttctagccatacccccaacagcaggaattttggctagatggagctcattcaagaagaatggagcgatcaaagtgttacggggtttcaaaaaagagatctcaagcatgttgaatattatgaacaggaggaaaaaatctgtgaccatgctcctcatgctgctgcccacagccctggcgttccatttgactacacgagggggagagccacacatgatagttagtaagcaggaaagaggaaagtcactgttgtttaagacctctgcaggcatcaatatgtgcactctcattgcgatggatctgggagagttatgcgaggacacaatgacctacaaatgcccccggatcactgaggcggaaccagatgacgttgactgctggtgcaatgccacagacacatgggtgacctatgggacatgttctcaaaccggcgaacaccgacgagacaaacgttccgtggcactggccccacacgtgggacttggtctagaaacaagaaccgaaacatggatgtcctctgaaggtgcctggaaacaaatacaaaaagtggagacttgggctttgagacacccaggattcacggtgacagccctttttttagcacatgccataggaacatccattactcagaaagggatcattttcattttgctgatgctagtaacaccatcaatggccatgcgatgtgtgggaataggcaacagggacttcgttgaaggactgtcaggagcaacgtgggtggacgtagtattggagcatggaagctgcgtcaccaccatggcaaaagataaaccaacattggacattgaactcttgaagacggaggtcacaaaccctgccgtcttgcgcaaactgtgcattgaagctaaaatatcaaacaccaccaccgattcaaggtgtccaacacaaggagaggccacactggtggaagaacaagactcgaactttgtgtgtcgacgaacgtttgtggacagaggctggggtaatggctgcggactatttggaaaaggaagcctactgacgtgtgctaagttcaagtgtgtgacaaaactagaagggaagatagttcaatatgaaaacttaaaatattcagtgatagtcactgtccacactggggaccagcaccaggtgggaaacgagactacagaacatggaacaattgcaactataacacctcaagctcctacgtcggaaatacagctgactgactacggagccctcacattggactgctcacctagaacagggctggactttaatgagatggttctattgacaatgaaagaaaaatcatggcttgtccacaaacaatggtttctagacttaccactgccttggacttcaggagcttcaacatctcaagagacttggaacagacaagatttgctggtcacattcaagacagctcatgcaaagaaacaggaagtagtcgtactgggatcacaggaaggagcaatgcacactgcgttgactggggcgacagaaatccagacgtcaggaacgacaacgatctttgcaggacacctgaaatgcagactaaaaatggataaactgactttaaaagggatgtcatatgtaatgtgcacaggctcatttaagctagagaaggaagtggctgagacccagcatggaactgttctagtgcaggttaaatacgaaggaacagatgcaccatgcaagatccctttttcggcccaagatgagaaaggagtgacccagaatgggagattgataacagccaaccccatagtcactgacaaagaaaaaccagtcaacattgagacagaaccaccttttggtgagagctacatcgtggtaggggcaggtgaaaaagctttgaaactgagctggttcaagaaagggagcagcatagggaaaatgttcgaagcaactgcccgaggagcacgaaggatggctatcctgggagacaccgcatgggactttggctctataggaggagtgttcacatcagtgggaaaattggtacaccaggtttttggagccgcatatggggttctgttcagcggtgtttcttggaccatgaaaataggaatagggattctgctgacatggttgggattaaattcaaggagcacgtcactttcgatgacgtgcattgcggttggcatggtcacactgtacctaggagtcatggttcaagcggactcgggatgtgtaatcaactggaagggcagagaactcaaatgtggaagtggtatttttgtcactaatgaagtccacacttggacagagcaatacaaattccaggctgactccccaaaaagactgtcagcagccattgggaaggcatgggaagagggcgtgtgtggaattcgatcagccacgcgtcttgagaacatcatgtggaagcaaatatcaaatgaattgaaccacattctacttgaaaatgacatgaaattcacagtggttgtaggagatgctaatggaattttggcccaagggaaaaaaatgattaggccacaacccatggaacatagatactcatggaaaagctggggaaaagccaaaatcataggagcagacatacagaatagcaccttcatcatcgacggcccagatactccagaatgccccgatgaccaaagagcgtggaacatttgggaagttgaagactatgggtttggaattttcacgacaaacatatggctgaaattgcgcgactcctacacccaaatgtgtgaccaccggctaatgtccgctgccatcaaagacagcaaggcagtccatgctgacatggggtactggatagaaagtgaaaagaacgagacctggaagctagcgagagcctccttcatagaagtcaagacatgtatctggccaaaatcccacactctatggagtaatggagttttggaaagtgaaatgataatcccaaagatatatggaggaccaatatctcagcacaactacagaccagggtatttcacacaaacagcagggccatggcacctaggcaagttggaattggatttcgacttgtgtgaaggtaccacagttgttgtggatgaacattgtggaaatcgaggcccatctctcagaactacaacagtcacaggaaagataatccatgaatggtgttgcagatcctgcacgttaccccccttacgcttcagaggagaagacggatgttggtatggcatggaaatcagaccagttaaggagaaggaggaaaacctagttaggtcaatggtctctgcagggtcaggagaagtggacagtttttcactagggatactatgcgtatcaataatgattgaagaagtgatgagatccagatggagtagaaagatgctgatgactggaacattggctgttttcctccttcttataatgggacaactgacatggaatgacctgattaggttatgcatcatggttggagctaatgcttcagacaggatggggatgggaacaacgtacctggccttgatggctactttcaaaatgagaccaatgttcgctgttgggctactatttcgcagactaacatccagagaagttcttcttctcacgattggattaagcctggtggcatccgtggagttaccaaattctttggaggagctaggggatggacttgcaatgggtatcatgatgttaaaattattgactgaatttcagccacaccagttatggaccaccttactgtctttgacatttatcaaaacaactctttcattggattatgcatggaggacaatggctatggtgctgtcaatcgtatctctctttcctttatgtctgtctacgacctcccaaaaaacaacatggcttccggtgctgttaggatcttttggatgcaaaccattaaccatgtttcttataacagaaaacaaaatctggggaaggaaaagttggcccctcaatgaaggaattatggctattggaatagtcagcattctactaagttcactccttaaaaatgatgtgccgttggccggcccactaatagctggaggcatgctaatagcatgttatgtcatatccggaagctcagccgatttatcattggagaaagcggctgaggtatcctgggaagaagaagcagaacactctggtacctcacacaacatattagtagaggtccaagatgatggaactatgaaaataaaagatgaagagagggatgacacactcaccatactcctcaaagcaactctgctggcagtctcaggagtgtacccaatgtcaataccagcaactcttttcgtgtggtatttttggcagaaaaagaaacagagatcaggagtgttatgggacacacccagccctccggaagtggaaagagcagttcttgatgatggcatctatagaatcctgcaaagaggactgttgggcaggtcccaagtgggagtgggagttttccaggacggcgtgttccacacaatgtggcacgtcaccaggggagctgtccttatgtaccaagggaagaggttggaaccaagctgggccagtgtcaaaaaggacttgatctcatatggaggaggttggagatttcaaggatcatggaacacgggagaagaggtgcaggtgatagccgttgaaccaggaaaaaatcctaaaaatgtacagacaacgccgggtaccttcaagactcccgaaggcgaagttggagccatagctctagattttaaacccggcacatctggatctcccatcgtgaacagagagggaaaaatagtaggtctttatggaaatggagtggtgacaacaagtggaacctacgtcagtgccattgcccaagctaaaacatcacaagaagggcctctaccagagattgaggaggaggtgtttaagaaaaggaacttaacaataatggacctgcatccaggatcgggaaaaacaagaagatatcttccagccatagtccgtgaggccataaaaaggaaactgcgcacgttaatcttggctcccacaagagttgtcgcctctgaaatggcagaggcgctcaagggaatgccaataagatatcagacaacagcagtgaagagtgaacacacaggaagggagatagtagacctcatgtgccacgccacttttaccatgcgtctcttatctccagtgagagttcccaattacaacatgattatcatggatgaagcacattttaccgatccagccagcatagcggccagagggtacatctcaactcgagtgggtatgggtgaagcagctgcgatctttatgacagccactcccccaggatcggtggaggcctttccacaaagtaatgcagttatccaagatgaggaaagagacattcctgagagatcatggaactcaggctacgactggatcactgattttccaggtaaaacagtctggtttgttccaagcattaagtcaggaaatgacattgctaactgtttaagaaagaacggaaaacgggtgatccaattgagcagaaaaacctttgacactgagtaccagaaaacaagaaacaatgactgggactatgttgtcacaacagacatttccgaaatgggggcaaatttccgggccgacagggtaatagacccaaggcggtgcttgaaaccagtgatactaaaagatggtccagagcgtgtcattctagccggaccgatgccagtgactgtggccagtgctgcccagagaagaggaagaattggaaggaaccaaaacaaggaaggtgatcagtacgtttatatgggacagcctttaaataatgatgaggatcacgctcattggacagaagcaaaaatgctccttgacaatataaacactccagaagggatcattccagccctctttgagccagagagagaaaagagtgcagcaatagatggagagtacagactgcggggagaagcaaggaaaacgttcgtggagctcatgagaagaggagatctaccagtttggctatcctacaaagttgcctcagaaggtttccagtactccgacagaaggtggtgctttgacggggaaaggaacaaccaggtgttggaggagaacatggacgtggagatctggacaaaggaaggagaaaggaagaaattgcgaccccgctggttggatgccagaacatactctgatccactggcccttcgtgagttcaaagagtttgcagcaggaagaagaagtgtctcaggtgacctaatattggaaatagggaaacttccacaacatttgacgctaagagcccagaatgctctggataacttggtcatgttgcacaattccgaacaaggaggaaaagcctatagacatgctatggaggaactaccagacaccatagaaacattgatgctcctagctttgatagctgtgctgactggtggagtgacgctgttcttcctatcaggaaaaggtctagggaaaacatccattggcctgctctgtgtgatggcctcaagcgcactgttatggatggccaatgtggagccacattggatagcggcctccatcatactagagttctttctgatggtgctgctcattccagagccagacagacagcgcactccacaggacaaccagctagcatacgtggtgataggtttgttattcatgatactgacagtggcagccaatgagatgggattattggaaaccacaaagaaagacctggggattggctatgtagccgccgaaaaccaacaccatgccacgatgctggacgtagacctacgcccagcttcagcctggaccctctatgcagtagccacaacagttatcactcccatgatgagacacacaattgaaaacacaacagcaaacatttccctgacagccattgcaaaccaggcagctatattgatgggacttgacaagggatggccaatatcgaagatggacataggagttccacttctcgccttagggtgctattcccaggtgaacccattgacactgacagcggcggtgttgatgttagtggctcattatgccataattggaccaggactgcaagcaaaggccactagagaagctcaaaaaaggacagcggccggaataatgaaaaatccaaccgtagacgggattgttgcaatagacttggatcctgtggtttatgatgcaaaatttgaaaaacagttaggccaaataatgttactgatactttgtacatcacagattctcttgatgcggaccacatgggccctgtgtgaatccatcacactggctactggacccttgaccactctctgggagggatctccaggaaaattctggaataccacgatagcagtgtccatggcaaacatcttcaggggaagttatctagcaggagcaggtctggccttctcgttaatgaaatctttgggaggaggcaggagaggtacgggggctcaaggagaaacactgggagagaaatggaaaagacagttaaaccaactgagcaagtcagaattcaacacctacaaaaggagtgggattatggaggtggacagatccgaagccaaagagggattaaaaagaggagaaacaaccaaacatgcagtgtcgagaggaacagccaaactgaggtggtttgtggagaggaacctcgtgaaaccagaagggaaagtcatagacctcggttgtggaagaggtggctggtcatattattgtgctgggctgaagaaagtcactgaagtcaagggatacacaaaaggaggacctggacatgaggaacctatcccaatggcgacctatggatggaacctagtaaagctatactctggaaaagatgtattttttacaccacctgagaaatgtgatacccttctgtgtgatataggagagtcctctccgaatccaactatagaagaaggaagaacgttacgtgttctaaagatggtagaaccatggctcagaggaaaccaattctgcataaaaattctaaatccttacatgccaagtgtggtagaaactctggagcaaatgcaaagaaaacatggagggatgctagtgcgaaacccactctcaagaaattccacccatgaaatgtactgggtttcatgtggaacaggaaacattgtgtcggcagtgaacatgacatccagaatgttactgaatcgattcacaatgactcacaggaaaccaacatatgaaagagacgtggacttaggcgctggaacaagacatgtggcagtggaaccagaggtagccaacctagatattattggccagaggatagagaatataaaaaatgaacacaagtcaacatggtattatgatgaagacaatccatacaaaacatgggcctatcatggatcatatgaggtcaaaccatcaggatcagcctcatctatggtgaatggcgtggtgagattgctcacaaaaccatgggatgtcatccccatggtcacacaaatagccatgaccgataccacacccttcggacaacagagagtgtttaaagagaaagtagatacgcgcacgccgagagcaaaacgaggcacagcacaaattatggaggtgacagccaagtggctatggggtttcctttccagaaacaaaaaacccagaatctgcacaagagaggagttcacaagaaaggttaggtcaaacgcagcaataggagcagtgttcgttgatgaaaaccaatggaactcagcaaaagaagcagtggaagacgaaaggttttgggatctcgtgcacagagagagggaacttcataaacagggaaaatgtgccacgtgtgtctacaacatgatggggaagagagagaaaaaattgggagagtttggaaaggcaaaaggaagtcgtgcaatatggtacatgtggctgggagcacgctttctagagttcgaagcccttggtttcatgaatgaagatcactggttcagtagagagaattcattcagtggagtggaaggagaaggactgcacaaacttggatacatactcagagacatatcaaagatcccggggggaaacatgtatgcagatgacacagccggatgggacacaagaataacagaggatgatcttcagaatgaggccaaaatcactgacatcatggagcctgaacacgctctattagctacgtcaatttttaagctgacctaccaaaacaaggtggtaagggtgcaaagaccagcaaaaaatggaaccgtgatggatgttatatccagacgagaccagagaggaagtgggcaggtcggaacctatggcttaaatactttcaccaatatggaggtccaactaataagacaaatggagtctgaaggaatctttttacccagcgaattggaaacccccaatctagctgagagagttcttaactggttggaaaaacatggcactgaaaggctgaaaagaatggcaattagcggagatgattgtgtggtgaaaccagttgatgacaggtttgcaacagccttaatagctctgaatgacatgggaaaagtaagaaaagacataccgcagtgggaaccttcaaaaggatggaatgactggcagcaagtgcctttctgttcacaccatttccaccagctgatcatgaaggatgggagggaaatagtggtgccatgccgcaaccaagatgaacttgtgggcagggctagagtatcacaaggcgccggatggagcctgagagaaactgcttgcctaggcaagtcatatgcacaaatgtggcagctgatgtacttccacaggagagacctaagactagcggctaatgctatctgttcagctgtcccagttgactgggttccaaccagccgcacaacctggtcaatccatgctcaccatcaatggatgacaacagaagatatgttatcagtgtggaatagggtttggatagaggaaaacccatggatggaggataaaactcatgtgtccagctgggaagaagtcccatacttagggaaaagggaagatcaatggtgtggatccctgataggcttgacagcgagggccacttgggccactaacatacaagtggccataaaccaagtgagaaggctcatcggaaatgagaattatctagattacatgacatcaatgaagagattcaagaatgagagtgatcccgaaggggcactctggtaagtcaacacactcatgaaataaaggaaaatggaagatcaaataaagtaagaagtcaggccagattaagccatagtacggaaaaagctatgctgcctgtgagccccgtccaaggacgtaaaatgaagtcaggccgaaagccacggcttgagcaagccgtgctgcctgtggctccatcgtggggatgtaaaaacccgggaggctgcaacccatggaagctgtacgcatggggtagcagactagtggttagaggagacccctcccaagacacaacgcagcagcggggcccaacaccaggggaagctgtaccctggtggtaaggactagaggttagaggagaccccccgcacaacaacaaacagcatattgacgctgggagagaccagagatcctgctgtctctacagcatcattccaggcacagaacgccagaaaatggaatg

SEQ ID NO: 6 - DNA, 10723 bp, Dengue virus serotype 2
agttgttagtctacgtggaccgacaaagacagattctttgagggagctaagctcaacgtagttctaacagttttttaattagagagcagatctctgatgaataaccaacggaaaaaggcgaaaaacacgcctttcaatatgctgaaacgcgagagaaaccgcgtgtcgactgtgcaacagctgacaaagagattctcacttggaatgctgcagggacgaggaccattaaaactgttcatggccctggtggcgttccttcgtttcctaacaatcccaccaacagcagggatattgaagagatggggaacaattaaaaaatcaaaagctattaatgttttgagagggttcaggaaagagattggaaggatgctgaacatcttgaataggagacgcagatctgcaggcatgatcattatgctgattccaacagtgatggcgttccatttaaccacacgtaacggagaaccacacatgatcgtcagcagacaagagaaagggaaaagtcttctgtttaaaacagaggatggcgtgaacatgtgtaccctcatggccatggaccttggtgaattgtgtgaagacacaatcacgtacaagtgtccccttctcaggcagaatgagccagaagacatagactgttggtgcaactctacgtccacgtgggtaacttatgggacgtgtaccaccatgggagaacatagaagagaaaaaagatcagtggcactcgttccacatgtgggaatgggactggagacacgaactgaaacatggatgtcatcagaaggggcctggaaacatgtccagagaattgaaacttggatcttgagacatccaggcttcaccatgatggcagcaatcctggcatacaccataggaacgacacatttccaaagagccctgattttcatcttactgacagctgtcactccttcaatgacaatgcgttgcataggaatgtcaaatagagactttgtggaaggggtttcaggaggaagctgggttgacatagtcttagaacatggaagctgtgtgacgacgatggcaaaaaacaaaccaacattggattttgaactgataaaaacagaagccaaacagcctgccaccctaaggaagtactgtatagaggcaaagctaaccaacacaacaacagaatctcgctgcccaacacaaggggaacccagcctaaatgaagagcaggacaaaaggttcgtctgcaaacactccatggtagacagaggatggggaaatggatgtggactatttggaaagggaggcattgtgacctgtgctatgttcagatgcaaaaagaacatggaaggaaaagttgtgcaaccagaaaacttggaatacaccattgtgataacacctcactcaggggaagagcatgcagtcggaaatgacacaggaaaacatggcaaggaaatcaaaataacaccacagagttccatcacagaagcagaattgacaggttatggcactgtcacaatggagtgctctccaagaacgggcctcgacttcaatgagatggtgttgctgcagatggaaaataaagcttggctggtgcacaggcaatggttcctagacctgccgttaccatggttgcccggagcggacacacaagggtcaaattggatacagaaagagacattggtcactttcaaaaatccccatgcgaagaaacaggatgttgttgttttaggatcccaagaaggggccatgcacacagcacttacaggggccacagaaatccaaatgtcatcaggaaacttactcttcacaggacatctcaagtgcaggctgagaatggacaagctacagctcaaaggaatgtcatactctatgtgcacaggaaagtttaaagttgtgaaggaaatagcagaaacacaacatggaacaatagttatcagagtgcaatatgaaggggacggctctccatgcaagatcccttttgagataatggatttggaaaaaagacatgtcttaggtcgcctgattacagtcaacccaattgtgacagaaaaagatagcccagtcaacatagaagcagaacctccattcggagacagctacatcatcataggagtagagccgggacaactgaagctcaactggtttaagaaaggaagttctatcggccaaatgtttgagacaacaatgaggggggcgaagagaatggccattttaggtgacacagcctgggattttggatccttgggaggagtgtttacatctataggaaaggctctccaccaagtctttggagcaatctatggagctgccttcagtggggtttcatggactatgaaaatcctcataggagtcattatcacatggataggaatgaattcacgcagcacctcactgtctgtgacactagtattggtgggaattgtgacactgtatttgggagtcatggtgcaggccgatagtggttgcgttgtgagctggaaaaacaaagaactgaaatgtggcagtgggattttcatcacagacaacgtgcacacatggacagaacaatacaagttccaaccagaatccccttcaaaactagcttcagctatccagaaagcccatgaagagggcatttgtggaatccgctcagtaacaagactggagaatctgatgtggaaacaaataacaccagaattgaatcacattctatcagaaaatgaggtgaagttaactattatgacaggagacatcaaaggaatcatgcaggcaggaaaacgatctctgcggcctcagcccactgagctgaagtattcatggaaaacatggggcaaagcaaaaatgctctctacagagtctcataaccagacctttctcattgatggccccgaaacagcagaatgccccaacacaaatagagcttggaattcgttggaagttgaagactatggctttggagtattcaccaccaatatatggctaaaattgaaagaaaaacaggatgtattctgcgactcaaaactcatgtcagcggccataaaagacaacagagccgtccatgccgatatgggttattggatagaaagtgcactcaatgacacatggaagatagagaaagcctctttcattgaagttaaaaactgccactggccaaaatcacacaccctctggagcaatggagtgctagaaagtgagatgataattccaaagaatctcgctggaccagtgtctcaacacaactatagaccaggctaccatacacaaataacaggaccatggcatctaggtaagcttgagatggactttgatttctgtgatggaacaacagtggtagtgactgaggactgcggaaatagaggaccctctttgagaacaaccactgcctctggaaaactcataacagaatggtgctgccgatcttgcacattaccaccgctaagatacagaggtgaggatgggtgctggtacgggatggaaatcagaccattgaaggagaaagaagagaatttggtcaactccttggtcacagctggacatgggcaggtcgacaacttttcactaggagtcttgggaatggcattgttcctggaggaaatgcttaggacccgagtaggaacgaaacatgcaatactactagttgcagtttcttttgtgacattgatcacagggaacatgtcctttagagacctgggaagagtgatggttatggtaggcgccactatgacggatgacataggtatgggcgtgacttatcttgccctactagcagccttcaaagtcagaccaacttttgcagctggactactcttgagaaagctgacctccaaggaattgatgatgactactataggaattgtactcctctcccagagcaccataccagagaccattcttgagttgactgatgcgttagccttaggcatgatggtcctcaaaatggtgagaaatatggaaaagtatcaattggcagtgactatcatggctatcttgtgcgtcccaaacgcagtgatattacaaaacgcatggaaagtgagttgcacaatattggcagtggtgtccgtttccccactgctcttaacatcctcacagcaaaaaacagattggataccattagcattgacgatcaaaggtctcaatccaacagctatttttctaacaaccctctcaagaaccagcaagaaaaggagctggccattaaatgaggctatcatggcagtcgggatggtgagcattttagccagttctctcctaaaaaatgatattcccatgacaggaccattagtggctggagggctcctcactgtgtgctacgtgctcactggacgatcggccgatttggaactggagagagcagccgatgtcaaatgggaagaccaggcagagatatcaggaagcagtccaatcctgtcaataacaatatcagaagatggtagcatgtcgataaaaaatgaagaggaagaacaaacactgaccatactcattagaacaggattgctggtgatctcaggactttttcctgtatcaataccaatcacggcagcagcatggtacctgtgggaagtgaagaaacaacgggccggagtattgtgggatgttccttcacccccacccatgggaaaggctgaactggaagatggagcctatagaattaagcaaaaagggattcttggatattcccagatcggagccggagtttacaaagaaggaacattccatacaatgtggcatgtcacacgtggcgctgttctaatgcataaaggaaagaggattgaaccatcatgggcggacgtcaagaaagacctaatatcatatggaggaggctggaagttagaaggagaatggaaggaaggagaagaagtccaggtattggcactggagcctggaaaaaatccaagagccgtccaaacgaaacctggtcttttcaaaaccaacgccggaacaataggtgctgtatctctggacttttctcctggaacgtcaggatctccaattatcgacaaaaaaggaaaagttgtgggtctttatggtaatggtgttgttacaaggagtggagcatatgtgagtgctatagcccagactgaaaaaagcattgaagacaacccagagatcgaagatgacattttccgaaagagaagactgaccatcatggacctccacccaggagcgggaaagacgaagagataccttccggccatagtcagagaagctataaaacggggtttgagaacattaatcttggcccccactagagttgtggcagctgaaatggaggaagcccttagaggacttccaataagataccagaccccagccatcagagctgagcacaccgggcgggagattgtggacctaatgtgtcatgccacatttaccatgaggctgctatcaccagttagagtgccaaactacaacctgattatcatggacgaagcccatttcacagacccagcaagtatagcagctagaggatacatctcaactcgagtggagatgggtgaggcagctgggatttttatgacagccactcccccgggaagcagagacccatttcctcagagcaatgcaccaatcatagatgaagaaagagaaatccctgaacgttcgtggaattccggacatgaatgggtcacggattttaaagggaagactgtttggttcgttccaagtataaaagcaggaaatgatatagcagcttgcctgaggaaaaatggaaagaaagtgatacaactcagtaggaagacctttgattctgagtatgtcaagactagaaccaatgattgggacttcgtggttacaactgacatttcagaaatgggtgccaatttcaaggctgagagggttatagaccccagacgctgcatgaaaccagtcatactaacagatggtgaagagcgggtgattctggcaggacctatgccagtgacccactctagtgcagcacaaagaagagggagaataggaagaaatccaaaaaatgagaatgaccagtacatatacatgggggaacctctggaaaatgatgaagactgtgcacactggaaagaagctaaaatgctcctagataacatcaacacgccagaaggaatcattcctagcatgttcgaaccagagcgtgaaaaggtggatgccattgatggcgaataccgcttgagaggagaagcaaggaaaacctttgtagacttaatgagaagaggagacctaccagtctggttggcctacagagtggcagctgaaggcatcaactacgcagacagaaggtggtgttttgatggagtcaagaacaaccaaatcctagaagaaaacgtggaagttgaaatctggacaaaagaaggggaaaggaagaaattgaaacccagatggttggatgctaggatctattctgacccactggcgctaaaagaatttaaggaatttgcagccggaagaaagtctctgaccctgaacctaatcacagaaatgggtaggctcccaaccttcatgactcagaaggcaagagacgcactggacaacttagcagtgctgcacacggctgaggcaggtggaagggcgtacaaccatgctctcagtgaactgccggagaccctggagacattgcttttactgacacttctggctacagtcacgggagggatctttttattcttgatgagcggaaggggcatagggaagatgaccctgggaatgtgctgcataatcacggctagcatcctcctatggtacgcacaaatacagccacactggatagcagcttcaataatactggagttttttctcatagttttgcttattccagaacctgaaaaacagagaacaccccaagacaaccaactgacctacgttgtcatagccatcctcacagtggtggccgcaaccatggcaaacgagatgggtttcctagaaaaaacgaagaaagatctcggattgggaagcattgcaacccagcaacccgagagcaacatcctggacatagatctacgtcctgcatcagcatggacgctgtatgccgtggccacaacatttgttacaccaatgttgagacatagcattgaaaattcctcagtgaatgtgtccctaacagctatagccaaccaagccacagtgttaatgggtctcgggaaaggatggccattgtcaaagatggacatcggagttccccttctcgccattggatgctactcacaagtcaaccccataactctcacagcagctcttttcttattggtagcacattatgccatcatagggccaggactccaagcaaaagcaaccagagaagctcagaaaagagcagcggcgggcatcatgaaaaacccaactgtcgatggaataacagtgattgacctagatccaataccttatgatccaaagtttgaaaagcagttgggacaagtaatgctcctagtcctctgcgtgactcaagtattgatgatgaggactacatgggctctgtgtgaggctttaaccttagctaccgggcccatctccacattgtgggaaggaaatccagggaggttttggaacactaccattgcggtgtcaatggctaacatttttagagggagttacttggccggagctggacttctcttttctattatgaagaacacaaccaacacaagaaggggaactggcaacataggagagacgcttggagagaaatggaaaagccgattgaacgcattgggaaaaagtgaattccagatctacaagaaaagtggaatccaggaagtggatagaaccttagcaaaagaaggcattaaaagaggagaaacggaccatcacgctgtgtcgcgaggctcagcaaaactgagatggttcgttgagagaaacatggtcacaccagaagggaaagtagtggacctcggttgtggcagaggaggctggtcatactattgtggaggactaaagaatgtaagagaagtcaaaggcctaacaaaaggaggaccaggacacgaagaacccatccccatgtcaacatatgggtggaatctagtgcgtcttcaaagtggagttgacgttttcttcatcccgccagaaaagtgtgacacattattgtgtgacataggggagtcatcaccaaatcccacagtggaagcaggacgaacactcagagtccttaacttagtagaaaattggttgaacaacaacactcaattttgcataaaggttctcaacccatatatgccctcagtcatagaaaaaatggaagcactacaaaggaaatatggaggagccttagtgaggaatccactctcacgaaactccacacatgagatgtactgggtatccaatgcttccgggaacatagtgtcatcagtgaacatgatttcaaggatgttgatcaacagatttacaatgagatacaagaaagccacttacgagccggatgttgacctcggaagcggaacccgtaacatcgggattgaaagtgagataccaaacctagatataattgggaaaagaatagaaaaaataaagcaagagcatgaaacatcatggcactatgaccaagaccacccatacaaaacgtgggcataccatggtagctatgaaacaaaacagactggatcagcatcatccatggtcaacggagtggtcaggctgctgacaaaaccttgggacgtcgtccccatggtgacacagatggcaatgacagacacgactccatttggacaacagcgcgtttttaaagagaaagtggacacgagaacccaagaaccgaaagaaggcacgaagaaactaatgaaaataacagcagagtggctttggaaagaattagggaagaaaaagacacccaggatgtgcaccagagaagaattcacaagaaaggtgagaagcaatgcagccttgggggccatattcactgatgagaacaagtggaagtcggcacgtgaggctgttgaagatagtaggttttgggagctggttgacaaggaaaggaatctccatcttgaaggaaagtgtgaaacatgtgtgtacaacatgatgggaaaaagagagaagaagctaggggaattcggcaaggcaaaaggcagcagagccatatggtacatgtggcttggagcacgcttcttagagtttgaagccctaggattcttaaatgaagatcactggttctccagagagaactccctgagtggagtggaaggagaagggctgcacaagctaggttacattctaagagacgtgagcaagaaagagggaggagcaatgtatgccgatgacaccgcaggatgggatacaagaatcacactagaagacctaaaaaatgaagaaatggtaacaaaccacatggaaggagaacacaagaaactagccgaggccattttcaaactaacgtaccaaaacaaggtggtgcgtgtgcaaagaccaacaccaagaggcacagtaatggacatcatatcgagaagagaccaaagaggtagtggacaagttggcacctatggactcaatactttcaccaatatggaagcccaactaatcagacagatggagggagaaggagtctttaaaagcattcagcacctaacaatcacagaagaaatcgctgtgcaaaactggttagcaagagtggggcgcgaaaggttatcaagaatggccatcagtggagatgattgtgttgtgaaacctttagatgacaggttcgcaagcgctttaacagctctaaatgacatgggaaagattaggaaagacatacaacaatgggaaccttcaagaggatggaatgattggacacaagtgcccttctgttcacaccatttccatgagttaatcatgaaagacggtcgcgtactcgttgttccatgtagaaaccaagatgaactgattggcagagcccgaatctcccaaggagcagggtggtctttgcgggagacggcctgtttggggaagtcttacgcccaaatgtggagcttgatgtacttccacagacgcgacctcaggctggcggcaaatgctatttgctcggcagtaccatcacattgggttccaacaagtcgaacaacctggtccatacatgctaaacatgaatggatgacaacggaagacatgctgacagtctggaacagggtgtggattcaagaaaacccatggatggaagacaaaactccagtggaatcatgggaggaaatcccatacttggggaaaagagaagaccaatggtgcggctcattgattgggttaacaagcagggccacctgggcaaagaacatccaagcagcaataaatcaagttagatcccttataggcaatgaagaatacacagattacatgccatccatgaaaagattcagaagagaagaggaagaagcaggagttctgtggtagaaagcaaaactaacatgaaacaaggctagaagtcaggtcggattaagccatagtacggaaaaaactatgctacctgtgagccccgtccaaggacgttaaaagaagtcaggccatcataaatgccatagcttgagtaaactatgcagcctgtagctccacctgagaaggtgtaaaaaatccgggaggccacaaaccatggaagctgtacgcatggcgtagtggactagcggttagaggagacccctcccttacaaatcgcagcaacaatgggggcccaaggcgagatgaagctgtagtctcgctggaaggactagaggttagaggagacccccccgaaacaaaaaacagcatattgacgctgggaaagaccagagatcctgctgtctcctcagcatcattccaggcacagaacgccagaaaatggaatggtgctgttgaatcaacaggttct

SEQ ID NO: 7 - DNA, 10707 bp, Dengue virus serotype 3
agttgttagtctacgtggaccgacaagaacagtttcgactcggaagcttgcttaacgtagtgctgacagttttttattagagagcagatctctgatgaacaaccaacggaagaagacgggaaaaccgtctatcaatatgctgaaacgcgtgagaaaccgtgtgtcaactggatcacagttggcgaagagattctcaaaaggactgctgaacggccagggaccaatgaaattggttatggcgttcatagctttcctcagatttctagccattccaccaacagcaggagtcttggctagatggggaaccttcaagaagtcgggggccattaaggtcctgaaaggcttcaagaaggagatctcaaacatgctgagcataatcaaccaacggaaaaagacatcgctctgtctcatgatgatattgccagcagcacttgctttccacttgacttcacgagatggagagccgcgcatgattgtggggaagaatgaaagaggtaaatccctactttttaagacagcctctggaatcaacatgtgcacactcatagccatggatttgggagagatgtgtgatgacacggtcacttacaaatgcccccacattaccgaagtggaacctgaagacattgactgctggtgcaaccttacatcaacatgggtgacttatggaacgtgcaatcaagctggagagcatagacgcgacaagagatcagtggcgttagctccccatgtcggcatgggactggacacacgcacccaaacctggatgtcggctgaaggagcttggagacaagtcgagaaggtagagacatgggcccttaggcacccagggttcaccatactagccctatttctcgcccattacataggcacttccctgacccagaaggtggttattttcatattattaatgctggtcaccccatccatgacaatgagatgtgtgggagtaggaaacagagattttgtggaagggctatcaggagctacgtgggttgacgtggtgctcgagcacggggggtgtgtgactaccatggctaagaacaagcccacgctggatatagagcttcagaagaccgaggccacccaactggcgaccctaaggaagctatgcattgaggggaaaattaccaacataacaactgactcaagatgtcctacccaaggggaagcggttttgcctgaggagcaggaccagaactacgtgtgtaagcatacatacgtagacagaggttgggggaacggttgtggtttgtttggcaaaggaagcttggtaacatgtgcgaaatttcaatgcctggaaccaatagagggaaaagtggtgcaatatgagaacctcaaatacaccgtcatcattacagtgcacacaggagaccaacaccaggtgggaaatgaaacgcaaggagtcacggctgagataacacctcaggcatcaaccactgaagccatcttgcctgaatatggaacccttgggctagaatgctcaccacggacaggtttggatttcaatgaaatgatcttactaacaatgaagaacaaagcatggatggtacatagacaatggttctttgacctacctctaccatgggcatcaggagctacaacagaaacaccaacctggaacaggaaggagcttcttgtgacattcaaaaacgcacatgcgaaaaaacaagaagtagttgtccttggatcgcaagagggagcaatgcataccgcactgacaggagctacagaaatccaaaactcaggaggcacaagcattttcgcggggcacttaaaatgtagacttaagatggacaaattggaactcaaggggatgagctatgcaatgtgcacgaatacctttgtgttgaagaaagaagtctcagaaacgcagcacgggacaatactcattaaggttgagtacaaaggggaagatgcaccttgcaagattcccttttccacagaggatggacaagggaaagctcataatggcagactgatcacagccaaccctgtggtgactaagaaggaggagcctgtcaatattgaggctgaacctccttttggggaaagcaatatagtaattggaattggagacaacgccttgaaaatcaactggtacaagaaggggagctcgattgggaagatgttcgaggccactgaaaggggtgcaaggcgcatggccatcttgggagacacagcttgggactttggatcagtgggtggtgttctgaactcattaggcaaaatggtgcaccaaatatttggaagtgcttatacagccctgttcagtggagtctcttgggtgatgaaaattggaataggtgtcctcttgacttggatagggttgaattcaaaaaacacatccatgtcattttcatgcattgcgataggaatcattacactctatctgggagctgtggtacaagctgacatggggtgtgtcataaactggaagggcaaagaactcaaatgtggaagcggaattttcgtcaccaatgaggtccatacctggacagagcaatacaaattccaagcagactccccaaaaagattggcaacagccattgcaggcgcctgggagaatggagtgtgtggaattaggtcaacaaccagaatggagaatctcttgtggaagcaaatagccaatgaactgaactacatattatgggaaaacaatatcaaattaacggtagttgtgggcgatacacttggggtcttagagcaagggaaaagaacactaacaccacaacccatggagctaaaatactcatggaaaacgtggggaaaggcaaaaatagtgacagctgaaacacaaaattcctctttcataatagacgggccaaacacaccggagtgtccaagtgcctcaagagcatggaatgtgtgggaggtggaagattacgggttcggagtcttcacaaccaacatatggctgaaactccgagaggtctacacccaactatgtgaccataggctaatgtcggcagctgtcaaggatgagagggccgtgcatgccgacatgggctactggatagaaagccaaaagaatggaagttggaagctagaaaaagcatccctcatagaggtaaaaacctgcacatggccaaaatcacacactctctggactaatggtgtgctagagagtgacatgatcatcccaaagagtctagctggtcctatctcacaacacaactacaggcccgggtaccacacccaaacggcaggaccctggcacttaggaaaattggagctggacttcaactactgtgaaggaacaacagttgtcatcacagaaagctgtgggacaagaggcccatcattgagaacaacaacagtgtcagggaagttgatacacgaatggtgttgccgctcgtgcacacttccccccctgcgatacatgggagaagacggctgctggtatggcatggaaatcagacccatcagtgagaaagaagagaacatggtaaagtctttagtctcagcgggaagtggaaaggtggacaacttcacaatgggtgtcttgtgtttggcaatcctctttgaagaggtgttgagaggaaaatttgggaagaaacacatgattgcaggggttttctttacgtttgtgctccttctctcagggcaaataacatggagagacatggcgcacacactaataatgatcgggtccaacgcctctgacaggatgggaatgggcgtcacctacctagctctaattgcaacatttaaaatccagccattcttggctttgggatttttcctaagaaagctgacatctagagaaaatttattgttaggagttgggttggccatggcaacaacgttacaactgccagaggacattgaacaaatggcaaatggagtcgctctggggctcatggctcttaaactgataacacaatttgaaacataccaattgtggacggcattagtctccttaacgtgttcaaacacaatttttacgttgactgttgcctggagaacagccactctgattttggccggagtttcgcttttaccagtgtgccagtcttcaagcatgaggaaaacagattggctcccaatgacagtggcagctatgggagttccaccccttccactttttatttttagcttgaaagacacactcaaaaggagaagctggccactgaatgaaggggtgatggctgttgggcttgtgagcattctggccagttctctccttagaaatgatgtgcccatggctggaccattagtggccgggggcttgctgatagcgtgctacgtcataactggcacgtcagcggacctcactgtagaaaaagccccagatgtaacatgggaggaagaggctgagcagacaggagtgtcccacaacttaatgatcacagttgatgatgatggaacaatgagaataaaagatgatgagactgagaacatcctaacagtgcttttaaaaacagcattactaatagtatcaggcatttttccatactccatacccgcaacattgttggtctggcacacttggcaaaaacaaacccaaagatccggcgttttatgggacgtacccagccccccagagacacagaaagcagaactggaagaaggggtttataggatcaaacagcaaggaatttttgggaaaacccaagtaggggttggagtacagaaagaaggagtcttccacaccatgtggcacgtcacaagaggggcagtgttgacacataatgggaaaagactggaaccaaactgggctagtgtgaaaaaagatctgatttcatatggaggaggatggagactgagcgcacaatggcaaaagggggaggaggtgcaggttattgccgtagagccagggaagaacccaaagaactttcaaaccacgccaggcactttccagactactacaggggaaataggagcaattgcactggatttcaagcctggaacttcaggatctcctatcataaatagagagggaaaggtagtgggactgtatggcaatggagtggttacaaagaatggtggctatgtcagcggaatagcgcaaacaaatgcagaaccagatggaccgacaccagagttggaagaagagatgttcaaaaagcgaaacctgaccataatggatcttcatcctgggtcaggaaagacacggaaataccttccagctattgtcagagaggcaatcaagagacgtttaagaaccttaattttggcaccgacaagggtggttgcagctgagatggaagaagcattgaaagggctcccaataaggtaccaaacaacagcaacaaaatctgaacacacaggaagagagattgttgatctaatgtgccacgcaacgttcacaatgcgtttgctgtcaccagttagggttccaaattacaacttgataataatggatgaggcccatttcacagacccagccagtatagcggctagagggtacatatcaactcgtgttggaatgggagaggcagccgcaatcttcatgacagcaacaccccctggaacagctgatgcctttcctcagagcaacgctccaattcaagatgaagaaagggacataccagaacgctcatggaattcaggcaatgaatggattaccgacttcgctgggaaaacggtgtggtttgtccctagcattaaagccggaaatgacatagcaaactgcttgcgaaaaaacgggaaaaaagtcattcaacttagtaggaagacttttgacacagaatatcagaagactaaactgaatgattgggactttgtggtgacaactgacatttcagaaatgggggccaatttcaaagcagatagagtgatcgacccaagaagatgtctcaaaccagtgatcttgacagatggaccagagcgggtgatcctggccggaccaatgccagtcaccgcggcgagtgctgcgcaaaggagagggagagttggcaggaacccacaaaaagagaatgaccagtacatattcacgggccagcctctcaacaatgatgaagaccatgctcactggacagaagcaaaaatgctgctggacaacatcaacacaccagaagggattataccagctctctttgaaccagaaagggagaagtcagccgccatagacggtgagtatcgcctgaagggtgagtccaggaagactttcgtggaactcatgaggaggggtgaccttccagtttggttagcccataaagtagcatcagaaggaatcaaatacacagatagaaaatggtgctttgatgggcaacgcaataatcaaattttagaggagaacatggatgtggaaatttggacaaaggaaggagaaaagaaaaaattgagacctaggtggcttgatgcccgcacttattcagatccattggcactcaaggaattcaaggactttgcggctggcagaaagtcaatcgcccttgatcttgtgacagaaataggaagagtgccttcacatctagcccacagaacaagaaacgctctggacaatctggtgatgctgcatacgtcagaagatggcggtagggcttacaggcatgcggtggaggaactaccagaaacaatggaaacactcctactcttgggactaatgatcttgttgacaggtggagcaatgcttttcttgatatcaggtaaagggattggaaagacttcaataggactcatttgtgtaatcgcttccagcggcatgttgtggatggccgaagttccactccaatggatcgcgtcggctatagtcctggagttttttatgatggtgttgctcataccagaaccagaaaagcagagaaccccccaagacaaccaactcgcatatgtcgtgataggcatacttacattggctgcaacaatagcagccaatgaaatgggactgctggaaaccacaaagagagacttaggaatgtctaaggagccaggtgttgtttctccaaccagctatttggatgtggacttgcacccagcatcagcctggacattgtacgccgtggccactacagtaataacaccaatgttaagacataccatagagaattctacagcaaatgtgtccctggcagctatagccaaccaggcagtggtcctgatgggtttggacaaaggatggccaatatcaaaaatggacttaggcgtgccactactggcactgggttgctattcacaagtgaacccactgactctaactgcggcagtacttttgctaatcacacattatgctatcataggtccaggattgcaagcaaaagccacccgtgaagctcagaaaaggacagctgctggaataatgaagaatccaacagtggatgggataatgacaatagacctagattctgtaatatttgattcaaaatttgaaaaacaactgggacaggttatgctcctggttttgtgcgcagtccaactcttgctaatgagaacatcatgggccttgtgtgaagctttaactctagctacaggaccaataacaacactctgggaaggatcacctggtaagttctggaacaccacgatagctgtttccatggcgaacatttttagagggagctatttagcaggagctgggcttgctttttctattatgaaatcagttggaacaggaaaaagaggaacaggctcacaaggtgaaactttaggagaaaaatggaaaaagaaattaaatcaattatcccggaaagagtttgacctttacaagaaatctggaatcactgaagtggatagaacagaagccaaagaagggttgaaaagaggagagacaacacatcatgccgtgtcccgaggtagcgcaaaacttcaatggtttgtggaaagaaacatggtcgttcccgaaggaagagtcatagacttgggctgtggaagaggaggctggtcatattactgtgcaggactgaaaaaagtcacagaagtgcgaggatacacaaaaggcggtccaggacacgaagaaccagtacctatgtctacatatggatggaacatagttaagttaatgagcggaaaggatgtgttctatctcccacctgaaaagtgtgataccctgttgtgtgacattggagaatcttcaccaagcccaacagtggaagagagcagaactataagagttttgaagatggttgaaccatggctaaaaaacaaccagttttgcattaaagttttgaacccttacatgccaactgtgattgagcacctagaaagactacaaaggaaacatggaggaatgcttgtgagaaatccactttcacgaaactccacgcacgaaatgtactggatatctaatggcacaggtaacattgtctcttcagtcaacatggtgtctagattgctactgaacaggttcacgatgacacacaggagacccaccatagagaaagatgtggatttaggagcaggaactcgacatgttaatgcggaaccagaaacacccaacatggatgtcattggggaaagaataaaaaggatcaaggaggagcataattcaacatggcactatgatgacgaaaacccctacaaaacgtgggcttaccatggatcctatgaagtcaaagccacaggctcagcctcctccatgataaatggagtcgtgaaactcctcaccaaaccatgggatgtggtgcccatggtgacacagatggcaatgacagacacaactccatttggccagcagagagtctttaaagagaaagtggacaccaggacgcccaggcccatgccagggacaagaaaggctatggagatcacagcggagtggctctggagaaccctgggaaggaacaaaagacccagattatgcacaagggaagagtttacaaaaaaggtcagaactaacgcagccatgggcgccgttttcacagaggagaaccaatgggacagtgcgaaagctgctgttgaggatgaagaattttggaaacttgtggacagagaacgtgaactccacaaattgggcaaatgtggaagctgcgtttataacatgatgggcaagagagagaaaaaacttggagagtttggcaaagcaaaaggcagtagagctatatggtacatgtggttgggagccaggtaccttgagttcgaagcccttggattcttaaatgaagaccactggttctcgcgtgaaaactcttacagtggagtagaaggagaaggactgcacaagctaggctacatattaagggacatttccaagatacccggaggagccatgtatgctgatgacacagctggttgggacacaagaataacagaagatgacctgcacaatgaggaaaagatcatacagcaaatggaccctgaacacaggcagttagcgaacgctatattcaagctcacataccaaaacaaagtggtcaaagttcaacgaccgactccaacgggcacggtaatggatattatatctaggaaagaccaaaggggcagtggacaactgggaacttatggcctgaatacattcaccaacatggaagcccagttagtcagacaaatggaaggagaaggtgtgctgacaaaggcagacctcgagaaccctcatctgctagagaagaaaatcacacaatggttggaaaccaaaggagtggagaggttaaaaagaatggccattagcggggatgattgcgtggtgaaaccaatcgatgacaggttcgctaatgccctgcttgctttgaacgatatgggaaaggttcggaaagacatacctcaatggcagccatcaaagggatggcatgattggcaacaggttcctttctgctcccaccactttcatgaattgatcatgaaagatggaagaaagttggtggttccctgcagaccccaggacgaactaataggaagagcaagaatctctcaaggagcgggatggagccttagagaaactgcatgtctggggaaagcctacgcccaaatgtggagtctcatgtattttcacagaagagatctcagattagcatccaacgccatatgttcagcagtaccagtccactgggttcccacaagtagaacgacatggtctattcatgctcaccatcagtggatgactacagaagacatgcttactgtttggaacagggtgtggatagaggaaaatccatggatggaagacaaaactccagttacaacttgggaaaatgttccatatctaggaaagagagaagaccaatggtgtggatcacttattggtctcacttccagagcaacctgggcccagaacatacccacagcaattcaacaggtgagaagccttataggcaatgaagagttcctggactacatgccttcaatgaagagattcaggaaggaagaggagtcggagggagccatttggtaaacgtaggaagtggaaaagaggctaactgtcaggccaccttaagccacagtacggaagaagctgtgctgcctgtgagccccgtccaaggacgttaaaagaagaagtcaggccccaaagccacggtttgagcaaaccgtgctgcctgtagctccgtcgtggggacgtaaaacctgggaggctgcaaactgtggaagctgtacgcacggtgtagcagactagcggttagaggagacccctcccatgacacaacgcagcagcggggcccgagcactgagggaagctgtacctccttgcaaaggactagaggttagaggagaccccccgcaaataaaaacagcatattgacgctgggagagaccagagatcctgctgtctcctcagcatcattccaggcacagaacgccagaaaatggaatggtgctgttgaatcaacaggttct

SEQ ID NO: 8 - DNA, 10649 bp, Dengue virus serotype 4
agttgttagtctgtgtggaccgacaaggacagttccaaatcggaagcttgcttaacacagttctaacagtttgtttgaatagagagcagatctctggaaaaatgaaccaacgaaaaaaggtggttagaccacctttcaatatgctgaaacgcgagagaaaccgcgtatcaacccctcaagggttggtgaagagattctcaaccggacttttttctgggaaaggacccttacggatggtgctagcattcatcacgtttttgcgagtcctttccatcccaccaacagcagggattctgaagagatggggacagttgaagaaaaataaggccatcaagatactgattggattcaggaaggagataggccgcatgctgaacatcttgaacgggagaaaaaggtcaacgataacattgctgtgcttgattcccaccgtaatggcgttttccctcagcacaagagatggcgaacccctcatgatagtggcaaaacatgaaagggggagacctctcttgtttaagacaacagaggggatcaacaaatgcactctcattgccatggacttgggtgaaatgtgtgaggacactgtcacgtataaatgccccctactggtcaataccgaacctgaagacattgattgctggtgcaacctcacgtctacctgggtcatgtatgggacatgcacccagagcggagaacggagacgagagaagcgctcagtagctttaacaccacattcaggaatgggattggaaacaagagctgagacatggatgtcatcggaaggggcttggaagcatgctcagagagtagagagctggatactcagaaacccaggattcgcgctcttggcaggatttatggcttatatgattgggcaaacaggaatccagcgaactgtcttctttgtcctaatgatgctggtcgccccatcctacggaatgcgatgcgtaggagtaggaaacagagactttgtggaaggagtctcaggtggagcatgggtcgacctggtgctagaacatggaggatgcgtcacaaccatggcccagggaaaaccaaccttggattttgaactgactaagacaacagccaaggaagtggctctgttaagaacctattgcattgaagcctcaatatcaaacataactacggcaacaagatgtccaacgcaaggagagccttatctgaaagaggaacaggaccaacagtacatttgccggagagatgtggtagacagagggtggggcaatggctgtggcttgtttggaaaaggaggagttgtgacatgtgcgaagttttcatgttcggggaagataacaggcaatttggtccaaattgagaaccttgaatacacagtggttgtaacagtccacaatggagacacccatgcagtaggaaatgacacatccaatcatggagttacagccatgataactcccaggtcaccatcggtggaagtcaaattgccggactatggagaactaacactcgattgtgaacccaggtctggaattgactttaatgagatgattctgatgaaaatgaaaaagaaaacatggctcgtgcataagcaatggtttttggatctgcctcttccatggacagcaggagcagacacatcagaggttcactggaattacaaagagagaatggtgacatttaaggttcctcatgccaagagacaggatgtgacagtgctgggatctcaggaaggagccatgcattctgccctcgctggagccacagaagtggactccggtgatggaaatcacatgtttgcaggacatcttaagtgcaaagtccgtatggagaaattgagaatcaagggaatgtcatacacgatgtgttcaggaaagttttcaattgacaaagagatggcagaaacacagcatgggacaacagtggtgaaagtcaagtatgaaggtgctggagctccgtgtaaagtccccatagagataagagatgtaaacaaggaaaaagtggttgggcgtatcatctcatccacccctttggctgagaataccaacagtgtaaccaacatagaattagaacccccctttggggacagctacatagtgataggtgttggaaacagcgcattaacactccattggttcaggaaagggagttccattggcaagatgtttgagtccacatacagaggtgcaaaacgaatggccattctaggtgaaacagcttgggattttggttccgttggtggactgttcacatcattgggaaaggctgtgcaccaggtttttggaagtgtgtatacaaccatgtttggaggagtctcatggatgattagaatcctaattgggttcttagtgttgtggattggcacgaactcgaggaacacttcaatggctatgacgtgcatagctgttggaggaatcactctgtttctgggcttcacagttcaagcagacatgggttgtgtggcgtcatggagtgggaaagaattgaagtgtggaagcggaatttttgtggttgacaacgtgcacacttggacagaacagtacaaatttcaaccagagtccccagcgagactagcgtctgcaatattaaatgcccacaaagatggggtctgtggaattagatcaaccacgaggctggaaaatgtcatgtggaagcaaataaccaacgagctaaactatgttctctgggaaggaggacatgacctcactgtagtggctggggatgtgaagggggtgttgaccaaaggcaagagagcactcacacccccagtgagtgatctgaaatattcatggaagacatggggaaaagcaaaaatcttcaccccagaagcaagaaatagcacatttttaatagacggaccagacacctctgaatgccccaatgaacgaagagcatggaactctcttgaggtggaagactatggatttggcatgttcacgaccaacatatggatgaaattccgagaaggaagttcagaagtgtgtgaccacaggttaatgtcagctgcaattaaagatcagaaagctgtgcatgctgacatgggttattggatagagagctcaaaaaaccagacctggcagatagagaaagcatctcttattgaagtgaaaacatgtctgtggcccaagacccacacactgtggagcaatggagtgctggaaagccagatgctcattccaaaatcatatgcgggccctttttcacagcacaattaccgccagggctatgccacgcaaaccgtgggcccatggcacttaggcaaattagagatagactttggagaatgccccggaacaacagtcacaattcaggaggattgtgaccatagaggcccatctttgaggaccaccactgcatctggaaaactagtcacgcaatggtgctgccgctcctgcacgatgcctcccttaaggttcttgggagaagatgggtgctggtatgggatggagattaggcccttgagtgaaaaagaagagaacatggtcaaatcacaggtgacggccggacagggcacatcagaaactttttctatgggtctgttgtgcctgaccttgtttgtggaagaatgcttgaggagaagagtcactaggaaacacatgatattagttgtggtgatcactctttgtgctatcatcctgggaggcctcacatggatggacttactacgagccctcatcatgttgggggacactatgtctggtagaataggaggacagatccacctagccatcatggcagtgttcaagatgtcaccaggatacgtgctgggtgtgtttttaaggaaactcacttcaagagagacagcactaatggtaataggaatggccatgacaacggtgctttcaattccacatgaccttatggaactcattgatggaatatcactgggactaattttgctaaaaatagtaacacagtttgacaacacccaagtgggaaccttagctctttccttgactttcataagatcaacaatgccattggtcatggcttggaggaccattatggctgtgttgtttgtggtcacactcattcctttgtgcaggacaagctgtcttcaaaaacagtctcattgggtagaaataacagcactcatcctaggagcccaagctctgccagtgtacctaatgactcttatgaaaggagcctcaagaagatcttggcctcttaacgagggcataatggctgtgggtttggttagtctcttaggaagcgctcttttaaagaatgatgtccctttagctggcccaatggtggcaggaggcttacttctggcggcttacgtgatgagtggtagctcagcagatctgtcactagagaaggccgccaacgtgcagtgggatgaaatggcagacataacaggctcaagcccaatcgtagaagtgaagcaggatgaagatggctctttctccatacgggacgtcgaggaaaccaatatgataacccttttggtgaaactggcactgataacagtgtcaggtctctaccccttggcaattccagtcacaatgaccttatggtacatgtggcaagtgaaaacacaaagatcaggagccctgtgggacgtcccctcacccgctgccactaaaaaagccgcactgtctgaaggagtgtacaggatcatgcaaagagggttattcgggaaaactcaggttggagtagggatacacatggaaggtgtatttcacacaatgtggcatgtaacaagaggatcagtgatctgccacgagactgggagattggagccatcttgggctgacgtcaggaatgacatgatatcatacggtgggggatggaggcttggagacaaatgggacaaagaagaagacgttcaggtcctcgccatagaaccaggaaaaaatcctaaacatgtccaaacgaaacctggccttttcaagaccctaactggagaaattggagcagtaacattagatttcaaacccggaacgtctggttctcccatcatcaacaggaaaggaaaagtcatcggactctatggaaatggagtagttaccaaatcaggtgattacgtcagtgccataacgcaagccgaaagaattggagagccagattatgaagtggatgaggacatttttcgaaagaaaagattaactataatggacttacaccccggagctggaaagacaaaaagaattcttccatcaatagtgagagaagccttaaaaaggaggctacgaactttgattttagctcccacgagagtggtggcggccgagatggaagaggccctacgtggactgccaatccgttatcagaccccagctgtgaaatcagaacacacaggaagagagattgtagacctcatgtgtcatgcaaccttcacaacaagacttttgtcatcaaccagggttccaaattacaaccttatagtgatggatgaagcacatttcaccgatccttctagtgtcgcggctagaggatacatctcgaccagggtggaaatgggagaggcagcagccatcttcatgaccgcaacccctcccggagcgacagatccctttccccagagcaacagcccaatagaagacatcgagagggaaattccggaaaggtcatggaacacagggttcgactggataacagactaccaagggaaaactgtgtggtttgttcccagcataaaagctggaaatgacattgcaaattgtttgagaaagtcgggaaagaaagttatccagttgagtaggaaaacctttgatacagagtatccaaaaacgaaactcacggactgggactttgtggtcactacagacatatctgaaatgggggccaattttagagccgggagagtgatagaccctagaagatgcctcaagccagttatcctaccagatgggccagagagagtcattttagcaggtcctattccagtgactccagcaagcgctgctcagagaagagggcgaataggaaggaacccagcacaagaagacgaccaatacgttttctccggagacccactaaaaaatgatgaagatcatgcccactggacagaagcaaagatgctgcttgacaatatctacaccccagaagggatcattccaacattgtttggtccggaaagggaaaaaacccaagccattgatggagagtttcgcctcagaggggaacaaaggaagacttttgtggaattaatgaggagaggagaccttccggtgtggctgagctataaggtagcttctgctggcatttcttacgaagatcgggaatggtgcttcacaggggaaagaaataaccaaattttagaagaaaacatggaggttgaaatttggactagagagggagaaaagaaaaagctaaggccaagatggttagatgcacgtgtatacgctgaccccatggctttgaaggatttcaaggagtttgccagtggaaggaagagtataactctcgacatcctaacagagattgccagtttgccaacttacctttcctctagggccaagctcgcccttgataacatagtcatgctccacacaacagaaagaggagggagggcctatcaacacgccctgaacgaacttccggagtcactggaaacactcatgcttgtagctttactaggtgctatgacagcaggcatcttcctgtttttcatgcaagggaaaggaatagggaaattgtcaatgggtttgataaccattgcggtggctagtggcttgctctgggtagcagaaattcaaccccagtggatagcggcctcaatcatactagagttttttctcatggtactgttgataccggaaccagaaaaacaaaggaccccacaagacaatcaattgatctacgtcatattgaccattctcaccatcattggtctaatagcagccaacgagatggggctgattgaaaaaacaaaaacggattttgggttttaccaggtaaaaacagaaaccaccatcctcgatgtggacttgagaccagcttcagcatggacgctctatgcagtagccaccacaattctgactcccatgctgagacacaccatagaaaacacgtcggccaacctatctctagcagccattgccaaccaggcagccgtcctaatggggcttggaaaaggatggccgctccacagaatggacctcggtgtgccgctgttagcaatgggatgctattctcaagtgaacccaacaaccttgacagcatccttagtcatgcttttagtccattatgcaataataggcccaggattgcaggcaaaagccacaagagaggcccagaaaaggacagctgctgggatcatgaaaaatcccacagtggacgggataacagtaatagatctagaaccaatatcctatgacccaaaatttgaaaagcaattagggcaggtcatgctactagtcttgtgtgctggacaactactcttgatgagaacaacatgggctttctgtgaagtcttgactttggccacaggaccaatcttgaccttgtgggagggcaacccgggaaggttttggaacacgaccatagccgtatccaccgccaacattttcaggggaagttacttggcgggagctggactggctttttcactcataaagaatgcacaaacccctaggaggggaactgggaccacaggagagacactgggagagaagtggaagagacagctaaactcattagacagaaaagagtttgaagagtataaaagaagtggaatactagaagtggacaggactgaagccaagtctgccctgaaagatgggtctaaaatcaagcatgcagtatcaagagggtccagtaagatcagatggattgttgagagagggatggtaaagccaaaagggaaagttgtagatcttggctgtgggagaggaggatggtcttattacatggcgacactcaagaacgtgactgaagtgaaagggtatacaaaaggaggtccaggacatgaagaaccgattcccatggctacttatggttggaatttggtcaaactccattcaggggttgacgtgttctacaaacccacagagcaagtggacaccctgctctgtgatattggggagtcatcttctaatccaacaatagaggaaggaagaacattaagagttttgaagatggtggagccatggctctcttcaaaacctgaattctgcatcaaagtccttaacccctacatgccaacagtcatagaagagctggagaaactgcagagaaaacatggtgggaaccttgtcagatgcccgctgtccaggaactccacccatgagatgtattgggtgtcaggagcgtcgggaaacattgtgagctctgtgaacacaacatcaaagatgttgttgaacaggttcacaacaaggcataggaaacccacttatgagaaggacgtagatcttggggcaggaacgagaagtgtctccactgaaacagaaaaaccagacatgacaatcattgggagaaggcttcagcgattgcaagaagagcacaaagaaacctggcattatgatcaggaaaacccatacagaacctgggcgtatcatggaagctatgaagctccttcgacaggctctgcatcctccatggtgaacggggtggtaaaactgctaacaaaaccctgggatgtgattccaatggtgactcagttagccatgacagatacaaccccttttgggcaacaaagagtgttcaaagagaaggtggataccagaacaccacaaccaaaacccggtacacgaatggttatgaccacgacagccaattggctgtgggccctccttggaaagaagaaaaatcccagactgtgcacaagggaagagttcatctcaaaagttagatcaaacgcagccataggcgcagtctttcaggaagaacagggatggacatcagccagtgaagctgtgaatgacagccggttttgggaactggttgacaaagaaagggccctacaccaggaagggaaatgtgaatcgtgtgtctataacatgatgggaaaacgtgagaaaaagttaggagagtttggcagagccaagggaagccgagcaatctggtacatgtggctgggagcgcggtttctggaatttgaagccctgggttttttgaatgaagatcactggtttggcagagaaaattcatggagtggagtggaaggggaaggtctgcacagattgggatatatcctggaggagatagacaagaaggatggagacctaatgtatgctgatgacacagcaggctgggacacaagaatcactgaggatgaccttcaaaatgaggaactgatcacggaacagatggctccccaccacaagatcctagccaaagccattttcaaactaacctatcaaaacaaagtggtgaaagtcctcagacccacaccgcggggagcggtgatggatatcatatccaggaaagaccaaagaggtagtggacaagttggaacatatggtttgaacacattcaccaacatggaagttcaactcatccgccaaatggaagctgaaggagtcatcacacaagatgacatgcagaacccaaaagggttgaaagaaagagttgagaaatggctgaaagagtgtggtgtcgacaggttaaagaggatggcaatcagtggagacgattgcgtggtgaagcccctagatgagaggtttggcacttccctcctcttcttgaacgacatgggaaaggtgaggaaagacattccgcagtgggaaccatctaagggatggaaaaactggcaagaggttcctttttgctcccaccactttcacaagatctttatgaaggatggccgctcactagttgttccatgtagaaaccaggatgaactgatagggagagccagaatctcgcagggagctggatggagcttaagagaaacagcctgcctgggcaaagcttacgcccagatgtggtcgcttatgtacttccacagaagggatctgcgtttagcctccatggccatatgctcagcagttccaacggaatggtttccaacaagcagaacaacatggtcaatccacgctcatcaccagtggatgaccactgaagatatgctcaaagtgtggaacagagtgtggatagaagacaaccctaatatgactgacaagactccagtccattcgtgggaagatataccttacctagggaaaagagaggatttgtggtgtggatccctgattggactttcttccagagccacctgggcgaagaacattcatacggccataacccaggtcaggaacctgatcggaaaagaggaatacgtggattacatgccagtaatgaaaagatacagtgctccttcagagagtgaaggagttctgtaattaccaacaacaaacaccaaaggctattgaagtcaggccacttgtgccacggtttgagcaaaccgtgctgcctgtagctccgccaataatgggaggcgtaataatccccagggaggccatgcgccacggaagctgtacgcgtggcatattggactagcggttagaggagacccctcccatcactgataaaacgcagcaaaagggggcccgaagccaggaggaagctgtactcctggtggaaggactagaggttagaggagacccccccaacacaaaaacagcatattgacgctgggaaagaccagagatcctgctgtctctgcaacatcaatccaggcacagagcgccgcaagatggattggtgttgttgatccaacaggttct

SEQ ID NO: 9 - protein, 3423 aa, Zika virus
MKNPKKKSGGFRIVNMLKRGVARVSPFGGLKRLPAGLLLGHGPIRMVLAILAFLRFTAIKPSLGLINRWGSVGKKEAMEIIKKFKKDLAAMLRIINARKEKKRRGADTSVGIVGLLLTTAMAAEVTRRGSAYYMYLDRNDAGEAISFPTTLGMNKCYIQIMDLGHTCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNAKNGSISLMCLALGGVLIFLSTAVSADVGCSVDFSKKETRCGTGVFVYNDVEAWRDRYKYHPDSPRRLAAAVKQAWEDGICGISSVSRMENIMWRSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTAGSTDHMDHFSLGVLVILLMVQEGLKKRMTTKIIISTSMAVLVAMILGGFSMSDLAKLAILMGATFAEMNTGGDVAHLALIAAFKVRPALLVSFIFRANWTPRESMLLALASCLLQTAISALEGDLMVLINGFALAWLAIRAMVVPRTDNITLAILAALTPLARGTLLVAWRAGLATCGGFMLLSLKGKGSVKKNLPFVMALGLTAVRLVDPINVVGLLLLTRSGKRSWPPSEVLTAVGLICALAGGFAKADIEMAGPMAAVGLLIVSYVVSGKSVDMYIERAGDITWEKDAEVTGNSPRLDVALDESGDFSLVEDDGPPMREIILKVVLMTICGMNPIAIPFAAGAWYVYVKTGKRSGALWDVPAPKEVKKGETTDGVYRVMTRRLLGSTQVGVGVMQEGVFHTMWHVTKGSALRSGEGRLDPYWGDVKQDLVSYCGPWKLDAAWDGHSEVQLLAVPPGERARNIQTLPGIFKTKDGDIGAVALDYPAGTSGSPILDKCGRVIGLYGNGVVIKNGSYVSAITQGRREEETPVECFEPSMLKKKQLTVLDLHPGAGKTRRVLPEIVREAIKTRLRTVILAPTRVVAAEMEEALRGLPVRYMTTAVNVTHSGTEIVDLMCHATFTSRLLQPIRVPNYNLYIMDEAHFTDPSSIAARGYISTRVEMGEAAAIFMTATPPGTRDAFPDSNSPIMDTEVEVPERAWSSGFDWVTDHSGKTVWFVPSVRNGNEIAACLTKAGKRVIQLSRKTFETEFQKTKHQEWDFVVTTDISEMGANFKADRVIDSRRCLKPVILDGERVILAGPMPVTHASAAQRRGRIGRNPNKPGDEYLYGGGCAETDEDHAHWLEARMLLDNIYLQDGLIASLYRPEADKVAAIEGEFKLRTEQRKTFVELMKRGDLPVWLAYQVASAGITYTDRRWCFDGTTNNTIMEDSVPAEVWTRHGEKRVLKPRWMDARVCSDHAALKSFKEFAAGKRGAAFGVMEALGTLPGHMTERFQEAIDNLAVLMRAETGSRPYKAAAAQLPETLETIMLLGLLGTVSLGIFFVLMRNKGIGKMGFGMVTLGASAWLMWLSEIEPARIACVLIVVFLLLVVLIPEPEKQRSPQDNQMAIIIMVAVGLLGLITANELGWLERTKSDLSHLMGRREEGATIGFSMDIDLRPASAWAIYAALTTFITPAVQHAVTTSYNNYSLMAMATQAGVLFGMGKGMPFYAWDFGVPLLMIGCYSQLTPLTLIVAIILLVAHYMYLIPGLQAAAARAAQKRTAAGIMKNPVVDGIVVTDIDTMTIDPQVEKKMGQVLLIAVAVSSAILSRTAWGWGEAGALITAATSTLWEGSPNKYWNSSTATSLCNIFRGSYLAGASLIYTVTRNAGLVKRRGGGTGETLGEKWKARLNQMSALEFYSYKKSGITEVCREEARRALKDGVATGGHAVSRGSAKLRWLVERGYLQPYGKVIDLGCGRGGWSYYAATIRKVQEVKGYTKGGPGHEEPVLVQSYGWNIVRLKSGVDVFHMAAEPCDTLLCDIGESSSSPEVEEARTLRVLSMVGDWLEKRPGAFCIKVLCPYTSTMMETLERLQRRYGGGLVRVPLSRNSTHEMYWVSGAKSNTIKSVSTTSQLLLGRMDGPRRPVKYEEDVNLGSGTRAVVSCAEAPNMKIIGNRIERIRSEHAETWFFDENHPYRTWAYHGSYEAPTQGSASSLINGVVRLLSKPWDVVTGVTGIAMTDTTPYGQQRVFKEKVDTRVPDPQEGTRQVMSMVSSWLWKELGKHKRPRVCTKEEFINKVRSNAALGAIFEEEKEWKTAVEAVNDPRFWALVDKEREHHLRGECQSCVYNMMGKREKKQGEFGKAKGSRAIWYMWLGARFLEFEALGFLNEDHWMGRENSGGGVEGLGLQRLGYVLEEMSRIPGGRMYADDTAGWDTRISRFDLENEALITNQMEKGHRALALAIIKYTYQNKVVKVLRPAEKGKTVMDIISRQDQRGSGQVVTYALNTFTNLVVQLIRNMEAEEVLEMQDLWLLRRSEKVTNWLQSNGWDRLKRMAVSGDDCVVKPIDDRFAHALRFLNDMGKVRKDTQEWKPSTGWDNWEEVPFCSHHFNKLHLKDGRSIVVPCRHQDELIGRARVSPGAGWSIRETACLAKSYAQMWQLLYFHRRDLRLMANAICSSVPVDWVPTGRTTWSIHGKGEWMTTEDMLVVWNRVWIEENDHMEDKTPVTKWTDIPYLGKREDLWCGSLIGHRPRTTWAENIKNTVNMVRRIIGDEEKYMDYLSTQVRYLGEEGSTPGVL

SEQ ID NO: 10 - DNA, 10374 bp, Zika virus
acaggttttattttggatttggaaacgagagtttctggtcatgaaaaacccaaaaaagaaatccggaggattccggattgtcaatatgctaaaacgcggagtagcccgtgtgagcccctttgggggcttgaagaggctgccagccggacttctgctgggtcatgggcccatcaggatggtcttggcgattctagcctttttgagattcacggcaatcaagccatcactgggtctcatcaatagatggggttcagtggggaaaaaagaggctatggaaataataaagaagttcaagaaagatctggctgccatgctgagaataatcaatgctaggaaggagaagaagagacgaggcgcagatactagtgtcggaattgttggcctcctgctgaccacagctatggcagcggaggtcactagacgtgggagtgcatactatatgtacttggacagaaacgatgctggggaggccatatcttttccaaccacattggggatgaataagtgttatatacagatcatggatcttggacacacgtgtgatgccaccatgagctatgaatgccctatgctggatgagggggtggaaccagatgacgtcgattgttggtgcaacacgacgtcaacttgggttgtgtacggaacctgccatcacaaaaaaggtgaagcacggagatctagaagagctgtgacgctcccctcccattccactaggaagctgcaaacgcggtcgcaaacctggttggaatcaagagaatacacaaagcacttgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatgttgtcttggaacatggaggttgtgtcactgtaatggcacaggacaaaccgactgtcgacatagagctggttacaacaacagtcagcaacatggcggaggtaagatcctactgctatgaggcatcaatatcagacatggcttcggacagccgctgcccaacacaaggtgaagcctaccttgacaagcaatcagacactcaatatgtctgcaaaagaacgttagtggacagaggctggggaaatggatgtggactttttggcaaagggagcctggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaagttgagataacgcccaattcaccaagagccgaagccaccctgggggggtttggaagcctaggacttgattgtgaaccgaggacaggccttgacttttcagatttgtattacttgactatgaataacaagcactggctggttcacaaggagtggttccacgacattccattaccttggcacgctggggcagacaccggaactccacactggaacaacaaagaagcactggtagagttcaaggacgcacatgccaaaaggcaaactgtcgtggttctagggagtcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtactgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggcagcaccattggaaaagcatttgaagccactgtgagaggtgccaagagaatggcagtcttgggagacacagcctgggactttggatcagttggaggcgctctcaactcattgggcaagggcatccatcaaatctttggagcagctttcaaatcattgtttggaggaatgtcctggttctcacaaattctcattggaacgttgctgatgtggttgggtctgaacgcaaagaatggatctatttcccttatgtgcttggccttagggggagtgttgatcttcttatccacagccgtctctgctgatgtggggtgctcggtggacttctcaaagaaggagacgagatgcggtacaggggtgttcgtctataacgacgttgaagcctggagggacaggtacaagtaccatcctgactccccccgtagattggcagcagcagtcaagcaagcctgggaagatggtatctgcgggatctcctctgtttcaagaatggaaaacatcatgtggagatcagtagaaggggagctcaacgcaatcctggaagagaatggagttcaactgacggtcgttgtgggatctgtaaaaaaccccatgtggagaggtccacagagattgcccgtgcctgtgaacgagctgccccacggctggaaggcttgggggaaatcgtacttcgtcagagcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcattagagtgtgatccagccgttattggaacagctgttaagggaaaggaggctgtacacagtgatctaggctactggattgagagtgagaagaatgacacatggaggctgaagagggcccatctgatcgagatgaaaacatgtgaatggccaaagtcccacacattgtggacagatggaatagaagagagtgatctgatcatacccaagtctttagctgggccactcagccatcacaataccagagagggctacaggacccaaatgaaagggccatggcacagtgaagagcttgaaattcggtttgaggaatgcccaggcactaaggtccacgtggaggaaacatgtggaacgagaggaccatctctgagatcaaccactgcaagcggaagggtgatcgaggaatggtgctgcagggagtgcacaatgcccccactgtcgttccgggctaaagatggctgttggtatggaatggagataaggcccaggaaagaaccagaaagcaacttagtaaggtcaatggtgactgcaggatcaactgatcacatggaccacttctcccttggagtgcttgtgattctgctcatggtgcaggaagggttgaagaagagaatgaccacaaagatcatcataagcacatcaatggcagtgctggtagctatgatcctgggaggattttcaatgagtgacctggctaagcttgcaattttgatgggtgccaccttcgcggaaatgaacactggaggagatgtagctcatctggcgctgatagcggcattcaaagtcagaccagcgttgctggtatctttcatcttcagagctaattggacaccccgtgaaagcatgctgctggccttggcctcgtgtcttttgcaaactgcgatctccgccttggaaggcgacctgatggttctcatcaatggttttgctttggcctggttggcaatacgagcgatggttgttccacgcactgataacatcaccttggcaatcctggctgctctgacaccactggcccggggcacactgcttgtggcgtggagagcaggccttgctacttgcggggggtttatgctcctctctctgaagggaaaaggcagtgtgaagaagaacttaccatttgtcatggccctgggactaaccgctgtgaggctggtcgaccccatcaacgtggtgggactgctgttgctcacaaggagtgggaagcggagctggccccctagcgaagtactcacagctgttggcctgatatgcgcattggctggagggttcgccaaggcagatatagagatggctgggcccatggccgcggtcggtctgctaattgtcagttacgtggtctcaggaaagagtgtggacatgtacattgaaagagcaggtgacatcacatgggaaaaagatgcggaagtcactggaaacagtccccggctcgatgtggcgctagatgagagtggtgatttctccctggtggaggatgacggtccccccatgagagagatcatactcaaggtggtcctgatgaccatctgtggcatgaacccaatagccataccctttgcagctggagcgtggtacgtatacgtgaagactggaaaaaggagtggtgctctatgggatgtgcctgctcccaaggaagtaaaaaagggggagaccacagatggagtgtacagagtaatgactcgtagactgctaggttcaacacaagttggagtgggagttatgcaagagggggtctttcacactatgtggcacgtcacaaaaggatccgcgctgagaagcggtgaagggagacttgatccatactggggagatgtcaagcaggatctggtgtcatactgtggtccatggaagctagatgccgcctgggacgggcacagcgaggtgcagctcttggccgtgccccccggagagagagcgaggaacatccagactctgcccggaatatttaagacaaaggatggggacattggagcggttgcgctggattacccagcaggaacttcaggatctcctatcctagacaagtgtgggagagtgataggactttatggcaatggggtcgtgatcaaaaatgggagttatgttagtgccatcacccaagggaggagggaggaagagactcctgttgagtgcttcgagccttcgatgctgaagaagaagcagctaactgtcttagacttgcatcctggagctgggaaaaccaggagagttcttcctgaaatagtccgtgaagccataaaaacaagactccgtactgtgatcttagctccaaccagggttgtcgctgctgaaatggaggaggcccttagagggcttccagtgcgttatatgacaacagcagtcaatgtcacccactctggaacagaaatcgtcgacttaatgtgccatgccaccttcacttcgcgtctactacagccaatcagagtccccaactataatctgtatattatggatgaggcccacttcacagatccctcaagtatagcagcaagaggatacatttcaacaagggttgagatgggcgaggcggccgccatcttcatgaccgccacgccaccaggaacccgtgacgcatttccggactccaactcaccaattatggacaccgaagtggaagtcccagagagagcctggagctcaggctttgattgggtgacggatcattctggaaaaacagtttggtttgttccaagcgtgaggaacggcaatgagatcgcagcttgtctgacaaaggctggaaaacgggtcatacagctcagcagaaagacttttgagacagagttccagaaaacaaaacatcaagagtgggactttgtcgtgacaactgacatttcagagatgggcgccaactttaaagctgaccgtgtcatagattccaggagatgcctaaagccggtcatacttgatggcgagagagtcattctggctggacccatgcctgtcacacatgccagcgctgcccagaggagggggcgcataggcaggaatcccaacaaacctggagatgagtatctgtatggaggtgggtgcgcagagactgacgaagaccatgcacactggcttgaagcaagaatgctccttgacaatatttacctccaagatggcctcatagcctcgctctatcgacctgaggccgacaaagtagcagccattgagggagagttcaagcttaggacggagcaaaggaagacctttgtggaactcatgaaaagaggagatcttcctgtttggctggcctatcaggttgcatctgccggaataacctacacagatagaagatggtgctttgatggcacgaccaacaacaccataatggaagacagtgtgccggcagaagtgtggaccagacacggagagaaaagagtgctcaaaccgaggtggatggacgccagagtttgttcagatcatgcggccctgaagtcattcaaggagtttgccgctgggaaaagaggagcggcttttggagtgatggaagccctgggaacactgccaggacacatgacagagagattccaggaagccattgacaacctcgctgtgctcatgcgggcagagactggaagcaggccttacaaagccgcggcggcccaattgccggagaccctagagaccattatgcttttggggttgctgggaacagtctcgctgggaatcttcttcgtcttgatgaggaacaagggcatagggaagatgggctttggaatggtgactcttggggccagcgcatggctcatgtggctctcggaaattgagccagccagaattgcatgtgtcctcattgttgtgttcctattgctggtggtgctcatacctgagccagaaaagcaaagatctccccaggacaaccaaatggcaatcatcatcatggtagcagtaggtcttctgggcttgattaccgccaatgaactcggatggttggagagaacaaagagtgacctaagccatctaatgggaaggagagaggagggggcaaccataggattctcaatggacattgacctgcggccagcctcagcttgggccatctatgctgccttgacaactttcattaccccagccgtccaacatgcagtgaccacctcatacaacaactactccttaatggcgatggccacgcaagctggagtgttgtttggtatgggcaaagggatgccattctacgcatgggactttggagtcccgctgctaatgataggttgctactcacaattaacacccctgaccctaatagtggccatcattttgctcgtggcgcactacatgtacttgatcccagggctgcaggcagcagctgcgcgtgctgcccagaagagaacggcagctggcatcatgaagaaccctgttgtggatggaatagtggtgactgacattgacacaatgacaattgacccccaagtggagaaaaagatgggacaggtgctactcatagcagtagccgtctccagcgccatactgtcgcggaccgcctgggggtggggggaggctggggccctgatcacagccgcaacttccactttgtgggaaggctctccgaacaagtactggaactcctctacagccacttcactgtgtaacatttttaggggaagttacttggctggagcttctctaatctacacagtaacaagaaacgctggcttggtcaagagacgtgggggtggaacaggagagaccctgggagagaaatggaaggcccgcttgaaccagatgtcggccctggagttctactcctacaaaaagtcaggcatcaccgaggtgtgcagagaagaggcccgccgcgccctcaaggacggtgtggcaacgggaggccatgctgtgtcccgaggaagtgcaaagctgagatggttggtggagcggggatacctgcagccctatggaaaggtcattgatcttggatgtggcagagggggctggagttactacgccgccaccatccgcaaagttcaagaagtgaaaggatacacaaaaggaggccctggtcatgaagaacccgtgttggtgcaaagctatgggtggaacatagtccgtcttaagagtggggtggacgtctttcatatggcggctgagccgtgtgacacgttgctgtgtgacataggtgagtcatcatctagtcctgaagtggaagaagcacggacgctcagagtcctctccatggtgggggattggcttgaaaaaagaccaggagccttttgtataaaagtgttgtgcccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctcctcttggggcgcatggacgggcctaggaggccagtgaaatatgaggaggatgtgaatctcggctctggcacgcgggctgtggtaagctgcgctgaagctcccaacatgaagatcattggtaaccgcattgaaaggatccgcagtgagcacgcggaaacgtggttctttgacgagaaccacccatataggacatgggcttaccatggaagctatgaggcccccacacaagggtcagcgtcctctctaataaacggggttgtcaggctcctgtcaaaaccctgggatgtggtgactggagtcacaggaatagccatgaccgacaccacaccgtatggtcagcaaagagttttcaaggaaaaagtggacactagggtgccagacccccaagaaggcactcgtcaggttatgagcatggtctcttcctggttgtggaaagagctaggcaaacacaaacggccacgagtctgtaccaaagaagagttcatcaacaaggttcgtagcaatgcagcattaggggcaatatttgaagaggaaaaagagtggaagactgcagtggaagctgtgaacgatccaaggttctgggctctagtggacaaggaaagagagcaccacctgagaggagagtgccagagttgtgtgtacaacatgatgggaaaaagagaaaagaaacaaggggaatttggaaaggccaagggcagccgcgccatctggtatatgtggctaggggctagatttctagagttcgaagcccttggattcttgaacgaggatcactggatggggagagagaactcaggaggtggtgttgaagggctgggattacaaagactcggatatgtcctagaagagatgagtcgtataccaggaggaaggatgtatgcagatgacactgctggctgggacacccgcattagcaggtttgatctggagaatgaagctctaatcaccaaccaaatggagaaagggcacagggccttggcattggccataatcaagtacacataccaaaacaaagtggtaaaggtccttagaccagctgaaaaagggaaaacagttatggacattatttcgagacaagaccaaagggggagcggacaagttgtcacttacgctcttaacacatttaccaacctagtggtgcaactcattcggaatatggaggctgaggaagttctagagatgcaagacttgtggctgctgcggaggtcagagaaagtgactaactggttgcagagcaacggatgggataggctcaaacgaatggcagtcagtggagatgattgcgttgtgaagccaattgatgataggtttgcacatgccctcaggttcttgaatgatatgggaaaagttaggaaggacacacaagagtggaaaccctcaactggatgggacaactgggaagaagttccgttttgctcccaccacttcaacaagctccatctcaaggacgggaggtccattgtggttccctgccgccaccaagatgaactgattggccgggcccgcgtctctccaggggcgggatggagcatccgggagactgcttgcctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtggatctctcatagggcacagaccgcgcaccacctgggctgagaacattaaaaacacagtcaacatggtgcgcaggatcataggtgatgaagaaaagtacatggactacctatccacccaagttcgctacttgggtgaagaagggtctacacctggagtgctgtaagcaccaatcttaatgttgtcaggcctgctagtcagccacagcttggggaaagctgtgcagcc

SEQ ID NO: 11 - protein, 3392 aa, Dengue virus serotype 1
MNNQRKKTGRPSFNMLKRARNRVSTVSQLAKRFSKGLLSGQGPMKLVMAFIAFLRFLAIPPTAGILARWGSFKKNGAIKVLRGFKKEISNMLNIMNRRKRSVTMLLMLLPTALAFHLTTRGGEPHMIVSKQERGKSLLFKTSAGVNMCTLIAMDLGELCEDTMTYKCPRITETEPDDVDCWCNATETWVTYGTCSQTGEHRRDKRSVALAPHVGLGLETRTETWMSSEGAWKQIQKVETWVLRHPGFTVIALFLAHAIGTSITQKGIIFILLMLVTPSMAMRCVGIGNRDFVEGLSGATWVDVVLEHGSCVTTMAKDKPTLDIELLKTEVTNPAVLRKLCIEAKISNTTTDSRCPTQGEATLVEEQDTNFVCRRTFVDRGWGNGCGLFGKGSLITCAKFKCVTKLEGKIVQYENLKYSVIVTVHTGDQHQVGNETTEHGTTATITPQAPTSEIQLTDYGALTLDCSPRTGLDFNEMVLLTMEKKSWLVHKQWFLDLPLPWTSGASTSQETWNRQDLLVTFKTAHAKKQEVVVLGSQEGAMHTALTGATEIQTSGTTTIFAGHLKCRLKMDKLTLKGMSYVMCTGSFKLEKEVAETQHGTVLVQVKYEGTDAPCKIPFSSQDEKGVTQNGRLITANPIVTDKEKPVNIEAEPPFGESYIVVGAGEKALKLSWFKKGSSIGKMFEATARGARRMAILGDTAWDFGSIGGVFTSVGKLIHQIFGTAYGVLFSGVSWTMKIGIGILLTWLGLNSRSTSLSMTCIAVGMVTLYLGVMVQADSGCVINWKGRELKCGSGIFVTNEVHTWTEQYKFQADSPKRLSAAIGKAWEEGVCGIRSATRLENIMWKQISNELNHILLENDMKFTVVVGDVSGILAQGKKMIRPQPMEHKYSWKSWGKAKIIGADVQNTTFIIDGPNTPECPDNQRAWNIWEVEDYGFGIFTTNIWLKLRDSYTQVCDHRLMSAAIKDSKAVHADMGYWIESEKNETWKLARASFIEVKTCIWPKSHTLWSNGVLESEMIIPKIYGGPISQHNYRPGYFTQTAGPWHLGKLELDFDLCEGTTVVVDEHCGNRGPSLRTTTVTGKTIHEWCCRSCTLPPLRFKGEDGCWYGMEIRPVKEKEENLVKSMVSAGSGEVDSFSLGLLCISIMIEEVMRSRWSRKMLMTGTLAVFLLLTMGQLTWNDLIRLCIMVGANASDKMGMGTTYLALMATFRMRPMFAVGLLFRRLTSREVLLLTVGLSLVASVELPNSLEELGDGLAMGIMMLKLLTDFQSHQLWATLLSLTFVKTTFSLHYAWKTMAMILSIVSLFPLCLSTTSQKTTWLPVLLGSLGCKPLTMFLITENKIWGRKSWPLNEGIMAVGIVSILLSSLLKNDVPLAGPLIAGGMLIACYVISGSSADLSLEKAAEVSWEEEAEHSGASHNILVEVQDDGTMKIKDEERDDTLTILLKATLLAISGVYPMSIPATLFVWYFWQKKKQRSGVLWDTPSPPEVERAVLDDGIYRILQRGLLGRSQVGVGVFQEGVFHTMWHVTRGAVLMYQGKRLEPSWASVKKDLISYGGGWRFQGSWNAGEEVQVIAVEPGKNPKNVQTAPGTFKTPEGEVGAIALDFKPGTSGSPIVNREGKIVGLYGNGVVTTSGTYVSAIAQAKASQEGPLPEIEDEVFRKRNLTIMDLHPGSGKTRRYLPAIVREAIKRKLRTLVLAPTRVVASEMAEALKGMPIRYQTTAVKSEHTGKEIVDLMCHATFTMRLLSPVRVPNYNMIIMDEAHFTDPASIAARGYISTRVGMGEAAAIFMTATPPGSVEAFPQSNAVIQDEERDIPERSWNSGYDWITDFPGKTVWFVPSIKSGNDIANCLRKNGKRVVQLSRKTFDTEYQKTKNNDWDYVVTTDISEMGANFRADRVIDPRRCLKPVILKDGPERVILAGPMPVTVASAAQRRGRIGRNQNKEGDQYIYMGQPLKNDEDHAHWTEAKMLLDNINTPEGIIPALFEPEREKSAAIDGEYRLRGEARKTFVELMRRGDLPVWLSYKVASEGFQYSDRRWCFDGERNNQVLEENMDVEIWTKEGERKKLRPRWLDARTYSDPLALREFKEFAAGRRSVSGDLILEIGKLPQHLTQRAQNALDNLVMLHNSEQGGKAYRHAMEELPDTIETLMLLALIAVLTGGVTLFFLSGRGLGKTSIGLLCVIASSALLWMASVEPHWIAASIILEFFLMVLLIPEPDRQRTPQDNQLAYVVIGLLFMILTVAANEMGLLETTKKDLGIGHAAAENHHHAAMLDVDLHPASAWTLYAVATTIITPMMRHTIENTTANISLTAIANQAAILMGLDKGWPISKMDIGVPLLALGCYSQVNPLTLTAAVLMLAAHYAIIGPGLQAKATREAQKRTAAGIMKNPTVDGIVAIDLDPVVYDAKFEKQLGQIMLLILCTSQILLMRTTWALCESITLATGPLTTLWEGSPGKFWNTTIAVSMANIFRGSYLAGAGLAFSLMKSLGGGRRGTGAQGETLGEKWKRQLNQLSKSEFNTYKRSGIIEVDRSEAKEGLKRGETTKHAVSRGTAKLRWFVERNLVKPEGKVIDLGCGRGGWSYYCAGLKKVTEVKGYTKGGPGHEEPIPMATYGWNLVKLYSGKDVFFTPPEKCDTLLCDIGESSPNPTIEEGRTLRVLKMVEPWLRGNQFCIKILNPYMPSVVETLEQMQRKHGGMLVRNPLSRNSTHEMYWVSCGTGNIVSAVNMTSRMLLNRFTMAHRKPTYERDVDLGAGTRHVAVEPEVANLDIIGQRIENIKNEHKSTWHYDEDNPYKTWAYHGSYEVKPSGSASSMVNGVVRLLTKPWDVIPMVTQIAMTDTTPFGQQRVFKEKVDTRTPKAKRGTAQIMEVTARWLWGFLSRNKKPRICTREEFTRKVRSNAAIGAVFVDENQWNSAKEAVEDERFWDLVHRERELHKQGKCATCVYNMMGKREKKLGEFGKAKGSRAIWYMWLGARFLEFEALGFMNEDHWFSRENSLSGVEGEGLHKLGYILRDISKIPGGNMYADDTAGWDTRITEDDLQNEAKITDIMEPEHALLATSIFKLTYQNKVVRVQRPAKNGTVMDVISRRDQRGSGQVGTYGLNTFTNMEAQLIRQMESEGIFSPSELETPNLAERVLDWLKKHGTERLKRMAISGDDCVVKPIDDRFATALTALNDMGKVRKDIPQWEPSKGWNDWQQVPFCSHHFHQLIMKDGREIVVPCRNQDELVGRARVSQGAGWSLRETACLGKSYAQMWQLMYFHRRDLRLAANAICSAVPVDWVPTSRTTWSIHAHHQWMTTEDMLSVWNRVWIEENPWMEDKTHVSSWEDVPYLGKREDQWCGSLIGLTARATWATNIQVAINQVRRLIGNENYLDFMTSMKRFKNESDPEGALW

SEQ ID NO: 12 - DNA, 10735 bp, Dengue virus serotype 1
agttgttagtctacgtggaccgacaagaacagtttcgaatcggaagcttgcttaacgtagttctaacagttttttattagagagcagatctctgatgaacaaccaacggaaaaagacgggtcgaccgtctttcaatatgctgaaacgcgcgagaaaccgcgtgtcaactgtttcacagttggcgaagagattctcaaaaggattgctttcaggccaaggacccatgaaattggtgatggcttttatagcattcctaagatttctagccatacctccaacagcaggaattttggctagatggggctcattcaagaagaatggagcgatcaaagtgttacggggtttcaagaaagaaatctcaaacatgttgaacataatgaacaggaggaaaagatctgtgaccatgctcctcatgctgctgcccacagccctggcgttccatctgaccacccgagggggagagccgcacatgatagttagcaagcaggaaagaggaaaatcacttttgtttaagacctctgcaggtgtcaacatgtgcacccttattgcaatggatttgggagagttatgtgaggacacaatgacctacaaatgcccccggatcactgagacggaaccagatgacgttgactgttggtgcaatgccacggagacatgggtgacctatggaacatgttctcaaactggtgaacaccgacgagacaaacgttccgtcgcactggcaccacacgtagggcttggtctagaaacaagaaccgaaacgtggatgtcctctgaaggcgcttggaaacaaatacaaaaagtggagacctgggttctgagacacccaggattcacggtgatagccctttttctagcacatgccataggaacatccatcacccagaaagggatcatttttattttgctgatgctggtaactccatccatggccatgcggtgcgtgggaataggcaacagagacttcgtggaaggactgtcaggagctacgtgggtggatgtggtactggagcatggaagttgcgtcactaccatggcaaaagacaaaccaacactggacattgaactcttgaagacggaggtcacaaaccctgccgtcctgcgcaaactgtgcattgaagctaaaatatcaaacaccaccaccgattcgagatgtccaacacaaggagaagccacgctggtggaagaacaggacacgaactttgtgtgtcgacgaacgttcgtggacagaggctggggcaatggttgtgggctattcggaaaaggtagcttaataacgtgtgctaagtttaagtgtgtgacaaaactggaaggaaagatagtccaatatgaaaacttaaaatattcagtgatagtcaccgtacacactggagaccagcaccaagttggaaatgagaccacagaacatggaacaactgcaaccataacacctcaagctcccacgtcggaaatacagctgacagactacggagctctaacattggattgttcacctagaacagggctagactttaatgagatggtgttgttgacaatggaaaaaaaatcatggctcgtccacaaacaatggtttctagacttaccactgccttggacctcgggggcttcaacatcccaagagacttggaatagacaagacttgctggtcacatttaagacagctcatgcaaaaaagcaggaagtagtcgtactaggatcacaagaaggagcaatgcacactgcgttgactggagcgacagaaatccaaacgtctggaacgacaacaatttttgcaggacacctgaaatgcagactaaaaatggataaactgactttaaaagggatgtcatatgtaatgtgcacagggtcattcaagttagagaaggaagtggctgagacccagcatggaactgttctagtgcaggttaaatacgaaggaacagatgcaccatgcaagatccccttctcgtcccaagatgagaagggagtaacccagaatgggagattgataacagccaaccccatagtcactgacaaagaaaaaccagtcaacattgaagcggagccaccttttggtgagagctacattgtggtaggagcaggtgaaaaagctttgaaactaagctggttcaagaagggaagcagtatagggaaaatgtttgaagcaactgcccgtggagcacgaaggatggccatcctgggagatactgcatgggacttcggttctataggaggggtgttcacgtctgtgggaaaactgatacaccagatttttgggactgcgtatggagttttgttcagcggtgtttcttggaccatgaagataggaatagggattctgctgacatggctaggattaaactcaaggagcacgtccctttcaatgacgtgtatcgcagttggcatggtcacactgtacctaggagtcatggttcaggcggactcgggatgtgtaatcaactggaaaggcagagaactcaaatgtggaagcggcatttttgtcaccaatgaagtccacacctggacagagcaatataaattccaggccgactcccctaagagactatcagcggccattgggaaggcatgggaggagggtgtgtgtggaattcgatcagccactcgtctcgagaacatcatgtggaagcaaatatcaaatgaattaaaccacatcttacttgaaaatgacatgaaatttacagtggtcgtaggagacgttagtggaatcttggcccaaggaaagaaaatgattaggccacaacccatggaacacaaatactcgtggaaaagctggggaaaagccaaaatcataggagcagatgtacagaataccaccttcatcatcgacggcccaaacaccccagaatgccctgataaccaaagagcatggaacatttgggaagttgaagactatggatttggaattttcacgacaaacatatggttgaaattgcgtgactcctacactcaagtgtgtgaccaccggctaatgtcagctgccatcaaggatagcaaagcagtccatgctgacatggggtactggatagaaagtgaaaagaacgagacttggaagttggcaagagcctccttcatagaagttaagacatgcatctggccaaaatcccacactctatggagcaatggagtcctggaaagtgagatgataatcccaaagatatatggaggaccaatatctcagcacaactacagaccaggatatttcacacaaacagcagggccgtggcacttgggcaagttagaactagattttgatttatgtgaaggtaccactgttgttgtggatgaacattgtggaaatcgaggaccatctcttagaaccacaacagtcacaggaaagacaatccatgaatggtgctgtagatcttgcacgttaccccccctacgtttcaaaggagaagacgggtgctggtacggcatggaaatcagaccagtcaaggagaaggaagagaacctagttaagtcaatggtctctgcagggtcaggagaagtggacagtttttcactaggactgctatgcatatcaataatgatcgaagaggtaatgagatccagatggagcagaaaaatgctgatgactggaacattggctgtgttcctccttctcacaatgggacaattgacatggaatgatctgatcaggctatgtatcatggttggagccaacgcttcagacaagatggggatgggaacaacgtacctagctttgatggccactttcagaatgagaccaatgttcgcagtcgggctactgtttcgcagattaacatctagagaagttcttcttcttacagttggattgagtctggtggcatctgtagaactaccaaattccttagaggagctaggggatggacttgcaatgggcatcatgatgttgaaattactgactgattttcagtcacatcagctatgggctaccttgctgtctttaacatttgtcaaaacaactttttcattgcactatgcatggaagacaatggctatgatactgtcaattgtatctctcttccctttatgcctgtccacgacttctcaaaaaacaacatggcttccggtgttgctgggatctcttggatgcaaaccactaaccatgtttcttataacagaaaacaaaatctggggaaggaaaagctggcctctcaatgaaggaattatggctgttggaatagttagcattcttctaagttcacttctcaagaatgatgtgccactagctggcccactaatagctggaggcatgctaatagcatgttatgtcatatctggaagctcggccgatttatcactggagaaagcggctgaggtctcctgggaagaagaagcagaacactctggtgcctcacacaacatactagtggaggtccaagatgatggaaccatgaagataaaggatgaagagagagatgacacactcaccattctcctcaaagcaactctgctagcaatctcaggggtatacccaatgtcaataccggcgaccctctttgtgtggtatttttggcagaaaaagaaacagagatcaggagtgctatgggacacacccagccctccagaagtggaaagagcagtccttgatgatggcatttatagaattctccaaagaggattgttgggcaggtctcaagtaggagtaggagtttttcaagaaggcgtgttccacacaatgtggcacgtcaccaggggagctgtcctcatgtaccaagggaagagactggaaccaagttgggccagtgtcaaaaaagacttgatctcatatggaggaggttggaggtttcaaggatcctggaacgcgggagaagaagtgcaggtgattgctgttgaaccggggaagaaccccaaaaatgtacagacagcgccgggtaccttcaagacccctgaaggcgaagttggagccatagctctagactttaaacccggcacatctggatctcctatcgtgaacagagagggaaaaatagtaggtctttatggaaatggagtggtgacaacaagtggtacctacgtcagtgccatagctcaagctaaagcatcacaagaagggcctctaccagagattgaggacgaggtgtttaggaaaagaaacttaacaataatggacctacatccaggatcgggaaaaacaagaagataccttccagccatagtccgtgaggccataaaaagaaagctgcgcacgctagtcttagctcccacaagagttgtcgcttctgaaatggcagaggcgctcaagggaatgccaataaggtatcagacaacagcagtgaagagtgaacacacgggaaaggagatagttgaccttatgtgtcacgccactttcactatgcgtctcctgtctcctgtgagagttcccaattataatatgattatcatggatgaagcacatttcaccgatccagccagcatagcagccagagggtatatctcaacccgagtgggtatgggtgaagcagctgcgattttcatgacagccactccccccggatcggtggaggcctttccacagagcaatgcagttatccaagatgaggaaagagacattcctgaaagatcatggaactcaggctatgactggatcactgatttcccaggtaaaacagtctggtttgttccaagcatcaaatcaggaaatgacattgccaactgtttaagaaagaatgggaaacgggtggtccaattgagcagaaaaacttttgacactgagtaccagaaaacaaaaaataacgactgggactatgttgtcacaacagacatatccgaaatgggagcaaacttccgagccgacagggtaatagacccgaggcggtgcctgaaaccggtaatactaaaagatggcccagagcgtgtcattctagccggaccgatgccagtgactgtggctagcgccgcccagaggagaggaagaattggaaggaaccaaaataaggaaggcgatcagtatatttacatgggacagcctctaaaaaatgatgaggaccacgcccattggacagaagcaaaaatgctccttgacaacataaacacaccagaagggattatcccagccctctttgagccggagagagaaaagagtgcagcaatagacggggaatacagactacggggtgaagcgaggaaaacgttcgtggagctcatgagaagaggagatctacctgtctggctatcctacaaagttgcctcagaaggcttccagtactccgacagaaggtggtgctttgatggggaaaggaacaaccaggtgttggaggagaacatggacgtggagatctggacaaaagaaggagaaagaaagaaactacgaccccgctggctggatgccagaacatactctgacccactggctctgcgcgaattcaaagagttcgcagcaggaagaagaagcgtctcaggtgacctaatattagaaatagggaaacttccacaacatttaacgcaaagggcccagaacgccttggacaatctggttatgttgcacaactctgaacaaggaggaaaagcctatagacacgccatggaagaactaccagacaccatagaaacgttaatgctcctagctttgatagctgtgctgactggtggagtgacgttgttcttcctatcaggaaggggtctaggaaaaacatccattggcctactctgcgtgattgcctcaagtgcactgttatggatggccagtgtggaaccccattggatagcggcctctatcatactggagttctttctgatggtgttgcttattccagagccggacagacagcgcactccacaagacaaccagctagcatacgtggtgataggtctgttattcatgatattgacagtggcagccaatgagatgggattactggaaaccacaaagaaggacctggggattggtcatgcagctgctgaaaaccaccatcatgctgcaatgctggacgtagacctacatccagcttcagcctggactctctatgcagtggccacaacaattatcactcccatgatgagacacacaattgaaaacacaacggcaaatatttccctgacagctattgcaaaccaggcagctatattgatgggacttgacaagggatggccaatatcaaagatggacataggagttccacttctcgccttggggtgctattctcaggtgaacccgctgacgctgacagcggcggtattgatgctagcggctcattatgccataattggacccggactgcaagcaaaagctactagagaagctcaaaaaaggacagcagccggaataatgaaaaacccaactgtcgacgggatcgttgcaatagatttggaccctgtggtttacgatgcaaaatttgaaaaacagctaggccaaataatgttgttgatactttgcacatcacagatcctcctgatgcggaccacatgggccttgtgtgaatccatcacactagccactggacctctgactacgctttgggagggatctccaggaaaattctggaacaccacgatagcggtgtccatggcaaacatttttaggggaagttatctagcaggagcaggtctggccttttcattaatgaaatctctaggaggaggtaggagaggcacgggagcccaaggggaaacactgggagaaaaatggaaaagacagctaaaccaattgagcaagtcagaattcaacacttacaaaaggagtgggattatagaggtggatagatctgaagccaaagaggggttaaaaagaggagaaacgactaaacacgcagtgtcgagaggaacggccaaactgaggtggtttgtggagaggaaccttgtgaaaccagaagggaaagtcatagacctcggttgtggaagaggtggctggtcatattattgcgctgggctgaagaaagtcacagaagtgaaaggatacacgaaaggaggacctggacatgaggaaccaatcccaatggcaacctatggatggaacctagtaaagctatactccgggaaagatgtattctttacaccacctgagaaatgtgacaccctcttgtgtgatattggtgagtcctctccgaacccaactatagaagaaggaagaacgttacgtgttctaaagatggtggaaccatggctcagaggaaaccaattttgcataaaaattctaaatccctatatgccgagtgtggtagaaactttggagcaaatgcaaagaaaacatggaggaatgctagtgcgaaatccactctcaagaaactccactcatgaaatgtactgggtttcatgtggaacaggaaacattgtgtcagcagtaaacatgacatctagaatgctgctaaatcgattcacaatggctcacaggaagccaacatatgaaagagacgtggacttaggcgctggaacaagacatgtggcagtagaaccagaggtggccaacctagatatcattggccagaggatagagaatataaaaaatgaacacaaatcaacatggcattatgatgaggacaatccatacaaaacatgggcctatcatggatcatatgaggtcaagccatcaggatcagcctcatccatggtcaatggtgtggtgagactgctaaccaaaccatgggatgtcattcccatggtcacacaaatagccatgactgacaccacaccctttggacaacagagggtgtttaaagagaaagttgacacgcgtacaccaaaagcgaaacgaggcacagcacaaattatggaggtgacagccaggtggttatggggttttctctctagaaacaaaaaacccagaatctgcacaagagaggagttcacaagaaaagtcaggtcaaacgcagctattggagcagtgttcgttgatgaaaatcaatggaactcagcaaaagaggcagtggaagatgaacggttctgggaccttgtgcacagagagagggagcttcataaacaaggaaaatgtgccacgtgtgtctacaacatgatgggaaagagagagaaaaaattaggagagttcggaaaggcaaaaggaagtcgcgcaatatggtacatgtggttgggagcgcgctttttagagtttgaagcccttggtttcatgaatgaagatcactggttcagcagagagaattcactcagtggagtggaaggagaaggactccacaaacttggatacatactcagagacatatcaaagattccagggggaaatatgtatgcagatgacacagccggatgggacacaagaataacagaggatgatcttcagaatgaggccaaaatcactgacatcatggaacctgaacatgccctattggccacgtcaatctttaagctaacctaccaaaacaaggtagtaagggtgcagagaccagcgaaaaatggaaccgtgatggatgtcatatccagacgtgaccagagaggaagtggacaggttggaacctatggcttaaacaccttcaccaacatggaggcccaactaataagacaaatggagtctgagggaatcttttcacccagcgaattggaaaccccaaatctagccgaaagagtcctcgactggttgaaaaaacatggcaccgagaggctgaaaagaatggcaatcagtggagatgactgtgtggtgaaaccaattgatgacagatttgcaacagccttaacagctttgaatgacatgggaaaggtaagaaaagacataccgcaatgggaaccttcaaaaggatggaatgattggcaacaagtgcctttctgttcacaccatttccaccagctgattatgaaggatgggagggagatagtggtgccatgccgcaaccaagatgaacttgtaggtagggccagagtatcacaaggcgccggatggagcttgagagaaactgcatgcctaggcaagtcatatgcacaaatgtggcagctgatgtacttccacaggagagacttgagattagcggctaatgctatctgttcagccgttccagttgattgggtcccaaccagccgtaccacctggtcgatccatgcccaccatcaatggatgacaacagaagacatgttgtcagtgtggaatagggtttggatagaggaaaacccatggatggaggacaagactcatgtgtccagttgggaagacgttccatacctaggaaaaagggaagatcaatggtgtggatccctaataggcttaacagcacgagccacctgggccaccaacatacaagtggccataaaccaagtgagaaggctcattgggaatgagaattatctagacttcatgacatcaatgaagagattcaaaaacgagagtgatcccgaaggggcactctggtaagccaactcattcacaaaataaaggaaaataaaaaatcaaacaaggcaagaagtcaggccggattaagccatagcacggtaagagctatgctgcctgtgagccccgtccaaggacgtaaaatgaagtcaggccgaaagccacggttcgagcaagccgtgctgcctgtagctccatcgtggggatgtaaaaacccgggaggctgcaaaccatggaagctgtacgcatggggtagcagactagtggttagaggagacccctcccaagacacaacgcagcagcggggcccaagccaggaggaagctgtactcctggtggaaggactagaggttagaggagaccccccgcacaacaacaaacagcatattgacgctgggagagaccagagatcctgctgtctctacagcatcattccaggcacagaacgccagaaaatggaatggtgctgttgaatcaacaggttct

SEQ ID NO: 13 - protein, 358 aa, Artificial Sequence
  DENV1 NS1
DSGCVINWKGRELKCGSGIFVTNEVHTWTEQYKFQADSPKRLSAAIGKAWEEGVCGIRSATRLENIMWKQISNELNHILLENDMKFTVVVGDVSGILAQGKKMIRPQPMEHKYSWKSWGKAKIIGADVQNTTFIIDGPNTPECPDNQRAWNIWEVEDYGFGIFTTNIWLKLRDSYTQVCDHRLMSAAIKDSKAVHADMGYWIESEKNETWKLARASFIEVKTCIWPKSHTLWSNGVLESEMIIPKIYGGPISQHNYRPGYFTQTAGPWHLGKLELDFDLCEGTTVVVDEHCGNRGPSLRTTTVTGKTIHEWCCRSCTLPPLRFKGEDGCWYGMEIRPVKEKEENLVKSMVSAHHHHHH

SEQ ID NO: 14 - protein, 358 aa, Artificial Sequence
  DENV2 NS1
DSGCVVSWKNKELKCGSGIFITDNVHTWTEQYKFQPESPSKLASAIQKAHEEGICGIRSVTRLENLMWKQITPELNHILSENEVKLTIMTGDIKGIMQAGKRSLRPQPTELKYSWKTWGKAKMLSTESHNQTFLIDGPETAECPNTNRAWNSLEVEDYGFGVFTTNIWLKLKEKQDVFCDSKLMSAAIKDNRAVHADMGYWIESALNDTWKIEKASFIEVKNCHWPKSHTLWSNGVLESEMIIPKNLAGPVSQHNYRPGYHTQITGPWHLGKLEMDFDFCDGTTVVVTEDCGNRGPSLRTTTASGKLITEWCCRSCTLPPLRYRGEDGCWYGMEIRPLKEKEENLVNSLVTAHHHHHH

SEQ ID NO: 15 - protein, 358 aa, Artificial Sequence
  DENV3 NS1
DMGCVINWKGKELKCGSGIFVTNEVHTWTEQYKFQADSPKRLATAIAGAWENGVCGIRSTTRMENLLWKQIANELNYILWENNIKLTVVVGDTLGVLEQGKRTLTPQPMELKYSWKTWGKAKIVTAETQNSSFIIDGPNTPECPSASRAWNVWEVEDYGFGVFTTNIWLKLREVYTQLCDHRLMSAAVKDERAVHADMGYWIESQKNGSWKLEKASLIEVKTCTWPKSHTLWTNGVLESDMIIPKSLAGPISQHNYRPGYHTQTAGPWHLGKLELDFNYCEGTTVVITESCGTRGPSLRTTTVSGKLIHEWCCRSCTLPPLRYMGEDGCWYGMEIRPISEKEENMVKSLVSAHHHHHH

SEQ ID NO: 16 - protein, 362 aa, Artificial Sequence
  DENV4 NS1
DMGCVASWSGKELKCGSGIFVVDNVHTWTEQYKFQPESPARLASAILNAHKDGVCGIRSTTRLENVMWKQITNELNYVLWEGGHDLTVVAGDVKGVLTKGKRALTPPVSDLKYSWKTWGKAKIFTPEARNSTFLIDGPDTSECPNERRAWNSLEVEDYGFGMFTTNIWMKFREGSSEVCDHRLMSAAIKDQKAVHADMGYWIESSKNQTWQIEKASLIEVKTCLWPKTHTLWSNGVLESQMLIPKSYAGPFSQHNYRQGYATQTVGPWHLGKLEIDFGECPGTTVTIQEDCDHRGPSLRTTTASGKLVTQWCCRSCTMPPLRFLGEDGCWYGMEIRPLSEKEENMVKSQVTAHHHHHHHHHH

(19) United States
(12) Patent Application Publication
(10) Pub. No.: US 2024/0133887 A1
(43) Pub. Date: Apr. 25, 2024

(54) METHODS FOR DETERMINING COMPLEMENT-FIXING ANTIBODIES

(71) Applicant: TAKEDA VACCINES, INC., Cambridge, MA (US)

(72) Inventors: Hansi DEAN, Cambridge, MA (US); Eduardo NASCIMENTO, Cambridge, MA (US)

(21) Appl. No.: 18/030,647

(22) PCT Filed: Oct. 7, 2021
(86) PCT No.: PCT/US2021/054242

§ 371 Date: Apr. 5, 2023

Related U.S. Application Data
(60) Provisional application No. 63/212,194, filed on Jun. 18, 2021.

(30) Foreign Application Priority Data
Oct. 8, 2020 (EP) 20201091.4

(51) Int. Cl. G01N 33/569; G01N 33/58

(52) U.S. Cl. CPC G01N 33/56983; G01N 33/582; G01N 2333/185; G01N 2469/20

(57) ABSTRACT

The invention relates to methods for determining the presence and/or amount of flavivirus-reactive complement-fixing antibodies in a sample from a subject. Further, the invention is related to methods for the concomitant determination of the presence and/or amount of complement-fixing antibodies reactive to different flaviviruses in a sample from a subject. Moreover, the invention is related to in vitro methods for diagnosing a flavivirus infection in a subject. In addition, the present invention also provides kits for carrying out the methods.

### CROSS REFERENCE TO RELATED APPLICATIONS

This application is a 35 U.S.C. § 371 National Stage filing of International Application No. PCT/US2021/054242, filed Oct. 8, 2021, which claims the benefit of, European Patent Application No. 20201091.4, filed Oct. 9, 2020, and priority to U.S. Provisional Application No. 63/212,194, filed on Jun. 18, 2021, the entire contents of the aforementioned applications are incorporated herein by reference in their entireties.

### SEQUENCE LISTING

This application contains a Sequence Listing which is submitted herewith in the electronically readable ASCII format via EFS-Web, and is hereby incorporated by reference in its entirety. The electronic Sequence Listing was created on Oct. 7, 2021, and was filed on Oct. 8, 2021, and is named Sequence-Listing_Final_as_filed and is 271 KB in size.

### FIELD OF THE INVENTION

The present invention is related to methods and kits for determining complement-fixing antibodies, in particular flavivirus-reactive complement-fixing antibodies in a sample.

### BACKGROUND OF THE INVENTION

The complement system (CS), also known as complement cascade, is a part of the innate immune system that enhances (“complements”) the ability of antibodies (Abs) and phagocytic cells to clear microbes and damaged cells from an organism, promote inflammation, and attack the pathogen's cell membrane. The CS is composed of more than 50 plasma proteins and receptors, several of them are found in the blood to normally circulate as inactivated precursors. There are three distinct pathways through which the CS can be activated on a pathogen surface, i.e. the classical pathway, the lectin pathway, and the alternative pathway. Although these pathways depend on different molecules for their initiation, they converge to generate the same set of effector molecules.

The classical pathway plays a role in both innate and adaptive immunity. The first component of this pathway, C1 complex, links the adaptive humoral immune response to the CS by binding to Abs complexed with antigens. The C1 complex is composed of the subcomponents C1q, C1r, and C1s. Antibody-binding is mediated by recognition of the Fc region of the corresponding antibody (Ab) by C-terminal globular head regions of C1q. C1q-antibody complex formation initiates a cascade including proteolytic activations mediating downstream effects. The protective role of the classical pathway of the CS comprises improvement of pathogen opsonization, antibody production by influencing the development of memory B cells as well as inhibiting antibody-dependent enhancement (ADE) of infection. Abs capable of activating the complement cascade when reacted with antigen are referred to as “complement-fixing antibodies”. Complement-fixing Abs mainly involve the IgM class (or isotype), as well as the IgG subclasses 1, 2, and 3 within the IgG class (Cooper, The classical complement pathway: activation and regulation of the first complement component; Adv Immunol 1985; 37:151-216). While IgG1 is the most abundant subclass in serum and hence the most efficient in complement-mediated lysis, IgG3 is known to have the highest affinity for complement possibly because of its long hinge region enabling to bind more C1q. Regardless of IgG2 being a poor activator of complement under several circumstances, it can efficiently bind C1q and activate complement in case of high antigen density. However, IgG4 was shown to have no complement-activating capacity.

All three CS pathways (classical, lectin, and alternative) lead to the formation of C3 convertases, which cleave C3. Cleavage of C3 is a central step that leads to effector branches of the CS, i.e. recruitment of inflammatory cells, lysis and death of microorganisms by formation of the membrane attack complex, and opsonization of pathogens. In the context of opsonization, activated C3b (a cleavage product of C3) can covalently attach to available protein amine groups or hydroxyl groups present on carbohydrate-containing glycoproteins of invading microorganisms. When C3b has attached to the surface of microorganisms, it interacts with complement receptor 1 (CR1) and is converted to iC3b by fluid phase factor I and H. This fragment is subsequently cleaved to C3d(g) by factor I, and CR1 as a cofactor, which results in C3d-tagged microorganisms that have the capacity to bind to complement receptor 2 (CR2) present on the surface of follicular dendritic cells, B cells, and some T cells. This interaction regulates B cell differentiation by lowering the threshold of activation, promoting proliferation, somatic hypermutation and class switching as well as helping to maintain effector and memory phenotypes. Thus, C3d deposition can influence antibody production associated with B cell immunity and displays an important step in the context of complement activation (see, for instance, Toapanta and Ross. Complement-mediated activation of the adaptive immune response: role of C3d in linking the innate and adaptive immunity. Immunol. Res. 2006; 36(1-3): 197-210).

The Flaviviridae family consists of many viral pathogens that cause severe disease and mortality in humans and animals. The family comprises four genera, i.e. Flavivirus, Pestivirus, Pegvirus, and Hepacivirus. The Flavivirus genus, the largest of the four genera, comprises more than 70 viruses, including dengue virus (DENV), West Nile virus (WNV), Zika virus (ZIKV), Japanese Encephalitis virus (JEV), Yellow Fever virus (YFV), St. Louis Encephalitis virus (SLEV), and Tick-Borne Encephalitis virus (TBEV).

Flaviviruses are enveloped, with icosahedral and spherical geometries. The diameter is around 50 nm. Genomes (10-11 kb bases) consist of linear positive-sense RNA and are non-segmented. The RNA is complexed with multiple copies of the capsid protein (C), surrounded by an icosahedral shell consisting of 180 copies each of the envelope glycoprotein (E protein; ˜500 amino acids), and the membrane protein (M protein; ˜75 amino acids) or precursor membrane protein (prM protein; ˜165 amino acids), all anchored in a lipid membrane. The genome also codes for seven non-structural proteins (NS1, NS2A, NS2B, NS3, NS4A, NS4B and NS5).

The flaviviruses are primarily transmitted by arthropods such as mosquitoes of the Aedes genus and ticks. The rate of flavivirus infections has increased dramatically over the past years affecting hundreds of millions of people each year. The large geographical expansion of flaviviruses has been related to vector adaption of mosquitoes to domesticated environments, climate and genetic changes, increased air travel, extensive urbanization, as well as the lack of effective mosquito control. Although most people infected with flaviviruses do solely develop an asymptomatic infection, the clinical spectrum is broad. After a primarily incubation time of up to 6 days, there may be an abrupt onset of diverse symptoms such as headache, dizziness, vomiting, nausea, and/or high fever. For instance, patients infected with one of the four DENV serotypes (DENV1-4) can develop severe manifestations as dengue hemorrhagic fever (DHF) and dengue shock syndrome (DSS). Severe forms of the infection are mainly associated with secondary infection with a different serotype and primary infections of infants born to DENV-immune mothers.

As complement fixation and activation by Abs is an important mechanism of humoral immune defense against pathogens, reliable and fast determination of complement-fixing Abs induced by natural infection and vaccination is of utmost importance. In addition, the level of complement-fixing antibodies generated by a vaccine, may be an indicator for the quality of the vaccine.

Although DENY-reactive complement-fixing Abs have been evaluated already a couple of decades ago, the methods used in the past and up to date are time-consuming and cumbersome requiring long incubation times (up to 20 hours), as well as the use of sheep red blood cells and guinea pig whole serum, reagents that are difficult to standardize (Sabin and Young, A complement fixation test for dengue, Experimental Biology and Medicine 1948, 69(3): 478-480; https://doi.org/10.3181/00379727-69-16761P; Hatgi and Sweet, Complement Fixation Kinetic Studies with Dengue Viruses, Archiv für die gesamte Virusforschung 1971, 35:323-338). The complement fixation test according to the original reported format used to be cited as a diagnosis tool for dengue infection by the World Health Organization (WHO).

In addition, other immune response parameters such as neutralizing or binding antibodies, measured by different methods in the art such as plaque assays, do not always correlate with protection against a flavivirus infection and/or disease. However, until today, no improved method for detecting DENV-reactive, and more generally flavivirus-reactive complement-fixing Abs has been developed, underlining the need for novel techniques in the field.

### OBJECTS AND SUMMARY

It is an object of the present invention to provide methods for determining flavivirus-reactive complement-fixing antibodies, the methods providing good specificity.

It is a further object of the present invention to provide methods for determining flavivirus-reactive complement-fixing antibodies, the methods not showing cross-reactivity to antibodies, in particular complement-fixing antibodies, reactive to other flaviviruses.

It is a further object of the present invention to provide methods for determining flavivirus-reactive antibodies, the methods being specific for antibodies, in particular complement-fixing antibodies, reactive to a specific flavivirus.

It is a further object of the present invention to provide methods for determining flavivirus-reactive complement-fixing antibodies, the methods providing flexibility to measure complement-fixing antibodies directed to multiple antigens from different flaviviruses in one single experiment.

It is a further object of the present invention to provide methods for determining flavivirus-reactive complement-fixing antibodies directed to dengue 1 virus (DENV1), dengue 2 virus (DENV2), dengue 3 virus (DENV3), and dengue 4 virus (DENV4) in one single experiment.

It is a further object of the present invention to provide methods for determining flavivirus-reactive complement-fixing antibodies directed to dengue 1 virus (DENV1), dengue 2 virus (DENV2), dengue 3 virus (DENV3), dengue 4 virus (DENV4), and ZIKV in one single experiment.

It is a further object of the present invention to provide methods for determining flavivirus-reactive complement-fixing antibodies, the methods providing good sensitivity.

It is a further object of the present invention to provide methods for determining flavivirus-reactive complement-fixing antibodies, the methods providing high accuracy and high precision.

It is a further object of the present invention to provide a method for determining flavivirus-reactive complement-fixing antibodies, the methods providing the possibility for high-throughput application, due to cost-effectiveness (e.g. due to reduced sample consumption) and short turnaround times.

It is a further object of the present invention to provide a method for determining flavivirus-reactive complement fixing antibodies, the methods providing the possibility to monitor the quality of an immune response after vaccination against the flavivirus.

It is a further object of the present invention to provide a method for determining flavivirus-reactive complement fixing antibodies, the methods providing the possibility to distinguish between subjects that are seropositive or seronegative for the flavivirus.

It is a further object of the present invention to provide methods for determining flavivirus-reactive complement fixing antibodies, the method providing the possibility to determine cross-reactive complement-fixing Abs in a sample, wherein the cross-reactive complement-fixing Abs are produced upon infection with one flavivirus or upon vaccination with one flavivirus vaccine and are also reactive to another flavivirus.

It is a further object of the present invention to provide in vitro methods for diagnosing a flavivirus infection.

It is a further object of the present invention to provide in vitro methods for diagnosing a flavivirus infection, the methods providing good specificity.

It is a further object of the present invention to provide in vitro methods for diagnosing a flavivirus infection, the methods providing the possibility to distinguish between different flavivirus infections, independent of the flavivirus infection being acute or convalescent.

It is a further object of the present invention to provide in vitro methods for diagnosing a flavivirus infection, the methods providing the possibility to distinguish between dengue virus (DENV) infection and zika virus (ZIKV) infection.

It is a further object of the present invention to provide in vitro methods for diagnosing a flavivirus infection, the methods providing the possibility to distinguish between infections by different DENV serotypes.

A further object of the present invention is to provide kits for carrying out said methods.

According to a first aspect, the present invention is directed to a method for determining the presence and/or amount of flavivirus-reactive complement-fixing antibodies in a sample from a subject comprising the steps of:
Step 1: contacting an amount of a microsphere complex comprising a microsphere coupled to a flavivirus antigen with the sample to allow binding of the flavivirus-reactive complement-fixing antibodies in the sample to the flavivirus antigen;
Step 2: contacting an amount of complement component 1q (C1q) with the complement-fixing antibodies bound to the flavivirus antigen in step 1 to allow binding of the C1q to the heavy chain constant region of the complement-fixing antibodies;
Step 3: contacting an amount of a reporter antibody with the C1q bound to the complement-fixing antibodies in step 2 to allow binding of the reporter antibody to the C1q, wherein the reporter antibody binds to the C1q with the variable region of the reporter antibody and wherein the reporter antibody is attached to a detectable label; and
Step 4: detecting a signal from the reporter antibody bound to the C1q in step 3, wherein the signal is indicative for the presence and/or amount of the reporter antibody and wherein the presence and/or amount of the reporter antibody is indicative for the presence and/or amount of flavivirus-reactive complement-fixing antibodies in the sample.

The present invention is further directed to a method for determining the presence and/or amount of flavivirus-reactive complement-fixing antibodies as described above, the method further comprising the steps of:
Step 5: determining the presence and/or amount of the reporter antibody from the signal of step 4; and
Step 6: determining the presence and/or amount of flavivirus-reactive complement-fixing antibodies in the sample from the presence and/or amount of the reporter antibody determined in step 5.

The present invention is further directed to a method for the concomitant determination of the presence and/or amount of complement-fixing antibodies reactive to different flaviviruses in a sample from a subject comprising the steps of:
Step 1: contacting an amount of at least two microsphere complexes with the sample,
   wherein the first microsphere complex comprises a first microsphere coupled to a first flavivirus antigen and the second microsphere complex comprises a second microsphere coupled to a second flavivirus antigen
to allow binding of the complement-fixing antibodies reactive to the first and/or the second flavivirus to the first flavivirus antigen and/or the second flavivirus antigen,
   wherein the first microsphere comprises a first detectable label and the second microsphere comprises a second detectable label, and wherein the emission signal of the first detectable label differs from the emission signal of the second detectable label,
Step 2: contacting an amount of C1q with the complement-fixing antibodies bound to the first and/or the second flavivirus antigen in step 1 to allow binding of the C1q to the heavy chain constant region of the complement-fixing antibodies,
Step 3: contacting an amount of a reporter antibody with the C1q bound to the complement-fixing antibodies in step 2 to allow binding of the reporter antibody to the C1q, wherein the reporter antibody binds to the C1q with the variable region of the reporter antibody and wherein the reporter antibody is attached to a third detectable label,
Step 4: detecting the emission signal of the detectable label of at least one microsphere upon irradiation with a first light source and comparing the emission signal with the emission signal of the first detectable label and with the emission signal of the second detectable label thereby identifying the at least one microsphere and the flavivirus antigen the at least one microsphere is coupled to, and
simultaneously detecting a signal from the reporter antibody bound to the C1q in step 3 of the at least one microsphere upon irradiation with a second light source,
Step 5: repeating step 4 until at least 30 microspheres coupled to the same flavivirus antigen are identified, and
Step 6: summarizing the detected signal from the reporter antibody in step 4 for all identified microspheres coupled to the same flavivirus antigen, wherein the summarized signal is indicative for the presence and/or amount of the reporter antibody and wherein the presence and/or amount of the reporter antibody is indicative for the presence and/or amount of flavivirus-reactive complement-fixing antibodies in the sample.

The present invention is further directed to a method for the concomitant determination of the presence and/or amount of complement-fixing antibodies reactive to different flaviviruses in a sample from a subject as described above, comprising the further steps of:
Step 7: determining the presence and/or amount of the reporter antibody from the summarized signal in step 6, and
Step 8: determining the presence and/or amount of flavivirus-reactive complement-fixing antibodies in the sample from the presence and/or amount of the reporter antibody determined in step 7.

The present invention is further directed to an in vitro method for diagnosing a flavivirus infection in a subject comprising determining the amount of flavivirus-reactive complement-fixing antibodies in the sample according to the methods as described above, wherein the presence of flavivirus-reactive complement-fixing antibodies in the sample is indicative for a flavivirus infection.

The present invention is further directed to a kit for detecting flavivirus-reactive complement-fixing antibodies in a sample comprising:
an amount of at least one microsphere complex comprising a microsphere coupled to a flavivirus antigen,
an amount of C1q, and
an amount of a reporter antibody, wherein the reporter antibody binds to the C1q with the variable region of the reporter antibody, and wherein the reporter antibody is attached to a detectable label.

The present invention is further directed to a kit for detecting flavivirus-reactive complement-fixing antibodies in a sample comprising:
an amount of at least one microsphere complex comprising a microsphere coupled to a flavivirus antigen,
an amount of C1q, and
an amount of a reporter antibody, wherein the kit further comprises an amount of a pre-reporter antibody, and wherein the pre-reporter antibody binds to the C1q with the variable region of the pre-reporter antibody and the reporter antibody binds to the heavy chain constant region of the pre-reporter antibody with the variable region of the reporter antibody.

In a further aspect the present invention is further directed to a method for determining the presence and/or amount of flavivirus-reactive complement-fixing antibodies in a sample from a subject comprising the steps of:
Step 1: contacting an amount of a microsphere complex comprising a microsphere coupled to a flavivirus antigen with the sample to allow binding of the flavivirus-reactive complement-fixing antibodies in the sample to the flavivirus antigen;
Step 2: contacting an amount of a complement competent serum with the complement-fixing antibodies bound to the flavivirus antigen in step 1 to allow the formation of complement component fragment C3d (C3d);
Step 3: contacting an amount of a reporter antibody with the C3d formed in step 2 to allow binding of the reporter antibody to the C3d, wherein the reporter antibody binds to the C3d with the variable region of the reporter antibody and wherein the reporter antibody is attached to a detectable label; and
Step 4: detecting a signal from the reporter antibody bound to the C3d in step 3, wherein the signal is indicative for the presence and/or amount of the reporter antibody and wherein the presence and/or amount of the reporter antibody is indicative for the presence and/or amount of flavivirus-reactive complement-fixing antibodies in the sample.

In one embodiment the present invention provides a method for the concomitant determination of the presence and/or amount of complement-fixing antibodies reactive to different flaviviruses in a sample from a subject comprising the steps of:
Step 1: contacting an amount of at least two microsphere complexes with the sample,
   wherein the first microsphere complex comprises a first microsphere coupled to a first flavivirus antigen and the second microsphere complex comprises a second microsphere coupled to a second flavivirus antigen
to allow binding of the complement-fixing antibodies reactive to the first and/or the second flavivirus to the first flavivirus antigen and/or the second flavivirus antigen;
   wherein the first microsphere comprises a first detectable label and the second microsphere comprises a second detectable label, and wherein the emission signal of the first detectable label differs from the emission signal of the second detectable label,
Step 2: contacting an amount of a complement competent serum with the complement-fixing antibodies bound to the flavivirus antigen in step 1 to allow the formation of C3d,
Step 3: contacting an amount of a reporter antibody with the C3d formed in step 2 to allow binding of the reporter antibody to the C3d, wherein the reporter antibody binds to the C3d with the variable region of the reporter antibody and wherein the reporter antibody is attached to a third detectable label,
Step 4: detecting the emission signal of the detectable label of at least one microsphere upon irradiation with a first light source and comparing the emission signal with the emission signal of the first detectable label and with the emission signal of the second detectable label thereby identifying the at least one microsphere and the flavivirus antigen the at least one microsphere is coupled to, and
simultaneously detecting a signal from the reporter antibody bound to the C3d in step 3 of the at least one microsphere upon irradiation with a second light source,
Step 5: repeating step 4 until at least 30 microspheres coupled to the same flavivirus antigen are identified, and
Step 6: summarizing the detected signal from the reporter antibody in step 4 for all identified microspheres coupled to the same flavivirus antigen, wherein the summarized signal is indicative for the presence and/or amount of the reporter antibody and wherein the presence and/or amount of the reporter antibody is indicative for the presence and/or amount of flavivirus-reactive complement-fixing antibodies in the sample.

In a further embodiment the present invention is directed to a kit for detecting flavivirus-reactive complement-fixing antibodies in a sample comprising:
an amount of at least one microsphere complex comprising a microsphere coupled to a flavivirus antigen,
an amount of a complement competent serum, and
an amount of a reporter antibody, wherein the reporter antibody binds to the C3d with the variable region of the reporter antibody, and wherein the reporter antibody is attached to a detectable label.

In a further embodiment the present invention is directed to a kit for detecting flavivirus-reactive complement-fixing antibodies in a sample comprising:
an amount of at least one microsphere complex comprising a microsphere coupled to a flavivirus antigen,
an amount of a complement competent serum, and
an amount of a reporter antibody, wherein the reporter antibody is attached to a detectable label and
   wherein wherein the kit further comprises an amount of a pre-reporter antibody, and wherein the pre-reporter antibody binds to the C3d with the variable region of the pre-reporter antibody and the reporter antibody binds to the heavy chain constant region of the pre-reporter antibody with the variable region of the reporter antibody.

As outlined in detail in the examples described below, the inventors have developed and validated singleplex (using one antigen) and multiplex (using two or more antigens) assays for complement-fixing antibodies against different antigens from flaviviruses such as DENV. In some of the examples, a Dengue VLP was used as antigen. In comparison to the prior art complement-fixation assay using sheep red blood cells the new assays have a much higher sensitivity and do not require highly trained personnel. The assays may be used in the development and quality control of flavivirus vaccines such as DENV vaccines. In addition, the assays are useful in the diagnosis of flavivirus infection such as DENV infection. The multiplex assays provide for the simultaneous screen for antigens from different flaviviruses or subtypes thereof. Both assay formats, singleplex and multiplex, are characterized by high sensitivity and specificity. Further, both assay formats exhibit high repeatability even when carried out by different operators.

In addition, the DENV-quadruplex complement-fixing antibody assay was compared to the microneutralization test (MNT), which is a gold standard technique. Although the MNT is widely applied, the assay is time-consuming as for instance one step requires infection of and incubation with cells in culture. In general, a good concordance in serostatus classification between the results from the MNT and the complement-fixing assay was observed (Table 8). When compared to the MNT, specificity and sensitivity of the complement-fixing assay was above 80% for each serotype (Table 9), indicating that the performance of the complement-fixing assay is similar to the gold standard dengue MNT.

Compared to traditional assays such as ELISA set-ups, the complement-fixing assays of the present invention provide several advantages such as an increased sensitivity and specificity, the flexibility to single- or multi-plex antigens from different viruses and/or virus serotypes in one single experiment, the possibility for high-throughput application (e.g. simplified washing procedures due to magnetic microspheres), cost-effectiveness (e.g. due to reduced sample volume, consumables, and labor), and short turnaround times. A particular advantage over traditional assays such as ELISA set-ups is further that the complement-fixing assays of the present invention measure an antibody response with functional antiviral activity, whereas, for instance, the ELISA set-up only measures antibodies that bind to the virus, but may, however, be not functional.

In summary, robust and reliable methods for the simultaneous detection and quantification of flavivirus-reactive complement fixing Abs binding to different virus types in one sample have been developed. This was exemplarily and successfully demonstrated for a quadruplex complement-fixing assay set-up including DENV-1-4 VLPs, as well as a 10-plex complement-fixing assay set-up including DENV1-4 as well as ZIKV VLPs and NS1 proteins.

The assays of the present invention provide low detection and quantification limits, as well as high inter- and intra-assay precision.

The complement-fixing assays of the present application enable reliable and fast determination of complement-fixing antibodies directed to various viruses developed after infection or vaccination. Especially in the context of flaviviruses, which are often co-circulating, a method enabling the simultaneous detection of complement-fixing Abs against several different species in one sample is of high benefit as people living in flavivirus endemic regions often experience multiple flavivirus infection in their life time. Particularly in the context of dengue, secondary infection with another dengue serotype may lead to severe forms of dengue disease such as DSS or DHF, underlining the need for fast, reliable, and cost-effective Ab analysis.

Also in the context of vaccination, determination of complement-fixing Abs is an important aspect in order to characterize the immune response in a subject upon administering of the vaccine. As common complement-fixing assay set-ups (especially for flaviviruses) have been cumbersome, insensitive, and time-consuming, the assays developed in the present application satisfy the former urgent need for a simplified and cost-effective method.

Moreover, as the data received for one antigen are not affected by an increasing amount of different antigens in one single experiment, the number of antigens applied in the complement-fixing assays of the present invention can be increased to any desirable number.

The complement-fixing assays of the present invention can be used as a diagnostic tool. As shown by the data, the DENV quadruplex assay is able to reliably distinguish between samples from seropositive and seronegative subjects, as well as between infections by different flaviviruses and DENV serotypes, independent of the infection being acute or convalescent. Moreover, taking into consideration the sensitivity and specificity analysis relative to DENV MNT, the complement-fixing assay may even be used as a surrogate marker of virus neutralization. The advantage of the complement-fixing assays in this context is inter alia that the results would be ready in hours (not days as required for the MNT), that the assay does not rely on virus infectivity as the MNT, and that—compared to MNT—the complement-fixing assay of the present invention enables high-throughput sample screening. Further, the MNT requires an infrastructure that may not be widely available in most laboratories, let alone hospitals, as the test uses live virus and living cells in culture. Over and above, application of the ZIKV NS1 protein in the complement-fixing assay enables the reliable differentiation between ZIKV, WNV, and DENV infection.

Moreover, it was demonstrated that the complement-fixing antibodies, detected by interaction between C1q and DENV VLP-antibody immunocomplexes (i.e. by application of the C1q-based complement-fixing assay set-up as described below), showed high correlation with C3d deposition (cf. Example 12), indicating that the C1q-based assay read-out not only measures the ability of antibodies to fix complement, but also the associated downstream functional step of C3 deposition that can mediate B cell activation.

### Abbreviations and Definitions

### Abbreviations

“PE” stands for phycoerythrin. “DENY” refers to dengue virus. “DENV1” refers to dengue virus serotype 1 (dengue 1 virus). “DENV2” refers to dengue virus serotype 2 (dengue 2 virus). “DENV3” refers to dengue virus serotype 3 (dengue 4 virus). “DENV4” refers to dengue virus serotype 4 (dengue 4 virus). “ZIKV” refers to zika virus. “WNV” refers to West Nile virus. “JEV” refers to Japanese encephalitis virus. “YFV” refers to Yellow Fever virus. “SLEV” refers to St. Louis encephalitis virus. “TBEV” refers to Tick-Borne encephalitis virus. “MVEV” refers to Murray Valley encephalitis virus. “VLP” and “VLPs” refers to virus like particle and virus like particles. “E protein” refers to envelope glycoprotein. “M protein” refers to membrane protein. “prM” refers to precursor membrane protein. “NS1” refers to non-structural protein 1. “NS2A” refers to non-structural protein 2A. “NS2B” refers to non-structural protein 2B. “NS3” refers to non-structural protein 3. “NS4A” refers to non-structural protein 4A. “NS4B” refers to non-structural protein 4B. “NS5” refers to non-structural protein 5. “MFI” refers to mean fluorescent intensity. “Ab” and “Abs” stand for antibody and antibodies. “Ig” stands for immunoglobulin. “CDR” stands for complementary determining region. “ELISA” refers to enzyme linked immunosorbent assay. “C1q” refers to complement component 1q.

### Definitions

As used herein, the singular forms “a,” “an,” and “the” include plural references unless the context clearly indicates otherwise.

The term “and/or” as used in a phrase such as “A and/or B” herein is intended to include both “A and B” and “A or B”.

As used herein, the term “about”, when used to modify a numeric value or numeric range, indicate that deviations of up to 10% above and up to 10% below the value or range remain within the intended meaning of the recited value or range.

Open terms such as “include,” “including,” “contain,” “containing” and the like mean “comprising.” These open-ended transitional phrases are used to introduce an open ended list of elements, method steps, or the like that does not exclude additional, unrecited elements or method steps.

### Antibody, Complement, and Related Terms

As used herein, the terms “antibody (Ab)” or “antibodies (Abs)” refer to an immunoglobulin (Ig) molecule, generally comprised of four polypeptide chains, two heavy (H) chains and two light (L) chains interconnected by disulfide bonds (full length Ab) and includes any naturally occurring, enzymatically obtainable, synthetic, or genetically engineered polypeptide or glycoprotein that specifically binds an antigen to form a complex. Abs can be obtained using standard recombinant DNA techniques. In a full length Ab, each heavy chain is comprised of a heavy chain variable region (VH) and a heavy chain constant region (CH). The heavy chain constant region is comprised of three domains, CH1, CH2 and CH3. Each light chain is comprised of a light chain variable region (VL) and a light chain constant region (CL). The light chain constant region is comprised of one domain. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. In certain embodiments of the present invention, the FRs of the Ab may be identical to the human germline sequences, or may be naturally or artificially modified. The terms Ab or Abs may also refer to any functional fragment, mutant, variant, or derivative thereof. Such functional fragment, mutant, variant, or derivative antibody formats are known in the art. Ab fragments such as Fab or F(ab′)2 fragments, can be prepared from full length Abs using conventional techniques such as papain or pepsin digestion, respectively, of full length Abs. Functional fragments are in particular (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546, Winter et al., PCT publication WO 90/05144 A1), which comprises a single variable domain; and (vi) an isolated CDR. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). In certain embodiments, scFv molecules may be incorporated into a fusion protein. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Such functional fragments are known in the art (Kontermann and Dubel eds., Antibody Engineering (2001) Springer-Verlag. New York. 790 pp. (ISBN 3-540-41354-5)). The Ab may be described by the term “anti-antigen Ab” to express to which antigen the Ab is able to bind. For instance, an “anti-DENV Ab” refers to an Ab that binds to a DENV antigen. Ab or Abs may be mono-specific, bi-specific, or multi-specific. Multi-specific Abs may specifically bind different epitopes of one antigen or may specifically bind two or more unrelated antigens. See, e.g., Tutt et al., 1991, J. Immunol. 147:60-69; Kufer et al., 2004, Trends Biotechnol. 22:238-244. Abs including any of the multi-specific antigen-binding molecules of the present invention, or variants thereof, may be constructed using standard molecular biological techniques (e.g., recombinant DNA and protein expression technology), as will be known to a person of ordinary skill in the art, for instance intracellular expression systems. Abs may be multivalent Abs comprising two or more antigen binding sites. Substitution of one or more CDR residues or omission of one or more CDRs is also possible. Abs have been described in the scientific literature where one or two CDRs can be dispensed with barely an effect for binding. Analysis of the contact regions between Abs and their antigens, based on published crystal structures, revealed that only about one fifth to one third of CDR residues actually contact the antigen. Moreover, many Abs have one or two CDRs were no amino acids are in contact with an antigen (Padlan et al. FASEB J. 1995, 9: 133-139, Vajdos et al., J Mol Biol 2002, 320:415-428). CDR residues not contacting antigen can be identified based on previous studies (for example residues H60-H65 in CDR2 of the heavy chain are often not required), from regions of Kabat CDRs lying outside Chothia CDRs, by molecular modeling and/or empirically. If a CDR or residue(s) thereof is omitted, it is usually substituted with an amino acid occupying the corresponding position in another human Ab sequence or a consensus of such sequences. Positions for substitution within CDRs and amino acids to substitute can also be selected empirically. Empirical substitutions can be conservative or non-conservative substitutions. The terms Ab or Abs may refer to Ab or Abs that originate from certain origin species that for example include rabbit, mouse, human, monkey, or rat (rabbit Ab, mouse Ab, human Ab, monkey Ab, or rat Ab). For instance, rabbit origin may be intended to include Abs having variable and constant regions derived from rabbit germline immunoglobulin sequences. Abs may comprise one or more amino acid substitution, insertion, and/or deletion as compared to corresponding germline sequences. The Abs may also include amino acid residues not encoded by the origin species germline immunoglobulin sequences (e.g. mutations introduced by random or site-specific mutagenesis in vitro or in vivo), for example in the CDRs. As used herein, an Ab or Abs originating from a certain origin species (e.g. rabbit) may also refer to an Ab or Abs in which CDR or other sequences derived from the germline of another mammalian species (e.g. mouse) have been grafted onto the origin species (e.g. rabbit) framework region (FR) sequences. Abs may be chimeric Abs. Chimeric Abs may encompass sequences derived from the germline of different species and may also include further amino acid substitutions or insertions. Abs may be humanized Abs that are human immunoglobulins that contain minimal non-human (e.g., murine) sequences. Typically, in humanized antibodies residues from the human CDR are replaced by residues from the CDR of a non-human species (e.g., mouse, rat, rabbit, and hamster, etc.; Jones et al., Nature 1986; 321:522-525; Riechmann et al., Nature 1988, 332:323-327; Verhoeyen et al., Science 1988, 239:1534-153). Non-limiting examples of methods used to generate humanized antibodies are described in U.S. Pat. No. 5,225,539; Roguska et al., Proc. Natl. Acad. Sci. 1994, USA 91:969-973; and Roguska et al., Protein Eng. 1996; 9:895-904. Abs can be of any class (isotype) (e.g., IgG, IgE, IgM, IgD, IgA and IgY) and subclass (e.g., IgG1, IgG2, IgG3, IgG4, IgA1 and IgA2). In some embodiments, the immunoglobulin is an IgG1 subclass. In some embodiments, the immunoglobulin is an IgG2 subclass. The different classes of immunoglobulins have different and well-known subunit structures and three-dimensional configurations. Abs may comprise sequences from more than one class or subclass. Abs may be free of other Abs having different antigenic specificities (e.g. an Ab that binds DENV is substantially free of Abs that bind antigens other than DENV). The Ab may be free of other cellular material and/or chemicals. The terms Ab or Abs may refer to a monoclonal Ab. The term Ab or Abs may refer to a reporter Ab. The term Ab or Abs may refer to a pre-reporter Ab. The term Ab or Abs may refer to a complement-fixing Ab.

As used herein, the term “complementary determining region (CDR)” refers to the CDR within the Ab variable sequences. There are three CDRs in each of the variable regions of the heavy chain (VH) and the light chain (VL), which are designated CDR1, CDR2 and CDR3 (or specifically VH-CDR1, VH-CDR2, VH-CDR3, VL-CDR1, VL-CDR2, and VL-CDR3), for each of the variable regions. The term CDR may refer to a group of three CDRs that occur in a single variable region capable of binding the antigen. The exact boundaries of these CDRs can be defined differently according to different systems. The system described by Kabat (Kabat et al., Sequences of Proteins of Immunological Interest (National Institutes of Health, Bethesda, Md. (1987) and (1991)) refers to an unambiguous residue system applicable to any variable region of an antibody, but also provides precise residue boundaries defining the three CDRs. These CDRs may be referred to as Kabat CDRs. For the VH region, the hypervariable region ranges from amino acid positions 31 to 35 for VH-CDR1, amino acid positions 50 to 65 for VH-CDR2, and amino acid positions 95 to 102 for VH-CDR3. For the VL region, the hypervariable region ranges from amino acid positions 24 to 34 for VL-CDR1, amino acid positions 50 to 56 for VL-CDR2, and amino acid positions 89 to 97 for VL-CDR3. Chothia and coworkers (Chothia &Lesk, J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (3 Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

As used herein, the term “framework”, “framework region (FR)” or “framework sequence” refers to the remaining sequences of a variable region minus the CDRs. Because the exact definition of a CDR sequence can be determined by different systems, the meaning of a framework sequence is subject to correspondingly different interpretations. The six CDRs (VL-CDR1, VL-CDR2, and VL-CDR3 and VH-CDR1, VH-CDR2, and VH-CDR3) also divide the framework regions on the light chain (L) and the heavy chain (H) into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3, or FR4, a framework region, as referred by others, represents the combined FR's within the variable region of a single, naturally occurring immunoglobulin chain. As used herein, a FR represents one of the four sub-regions, and FRs represents two or more of the four sub-regions constituting a framework region.

As used herein, the term “constant region” of an Ab refers to the heavy chain constant region (CH) and/or the light chain constant region (CL).

As used herein, the term “variable region” of an Ab refers to the heavy chain variable region (VH) and/or the light chain variable region (VL).

As used herein, the term “C1 complex” refers to the first component of the classical complement pathway and is composed of the subcomponents C1q, C1r, and C1s. C1q is responsible for binding to complement-fixing Abs. Upon Ab binding, C1r is autoactivated. Activated C1r cleaves C1s, which is thereby activated and able to cleave its substrates i.e. further components of the complement cascade including C4.

As used herein, the term “complement component 1q (C1q)” refers to the pattern recognition molecule C1q which is a subcomponent of the C1 complex that initiates the classical pathway of complement activation. C1q consists of six heterotrimers each containing the C1qA, C1qB, and C1qC chains. Each chain has a central region forming a collagen stem together with the equivalent regions of the other two chains. The C-terminal parts of the three chains together form a globular head region responsible for Ab binding. C1q can be obtained by purification from plasma. Alternatively C1q can be applied as a recombinant protein. In preferred embodiments, C1q is obtained by purification from plasma.

As used herein, the term “complement competent serum” refers to serum, which comprises complement activity to go through at least a fully functional classical complement pathway when contacted with suitable antigen-antibody complexes (immunocomplexes). The complement competent serum may also comprise such a complement activity to go through fully functional lectin and alternative complement pathways. Consequently, a complement competent serum comprises all complement components (such a C1, C2, C4, and C3) necessary for at least a fully functional classical complement pathway. In certain embodiments, complement component serum is a source of all complement proteins from all activation pathways (lectin, alternative, and classical complement pathways), i.e. comprises all components necessary to go through fully functional lectin, alternative, and classical complement pathways. Complement competent serum is commercially available. Alternatively, serum which is not further purified may be used as complement competent serum. In certain embodiments, complement component serum is normal human serum that was collected and frozen to maintain all the complement proteins intact and functional.

As used herein, “complement component fragment C3d” or “C3d” is a cleavage product of C3b, which itself is a cleavage product of complement component C3 (see, for instance, also FIG. 30A).

As used herein, the term “complement-fixing Ab” refers to an Ab that is capable of binding to complement component 1q (C1q) and thereby activating the classical complement pathway. Complement-fixing Abs are for instance produced upon infection with a pathogen or upon vaccination with a vaccine against a pathogen.

As used herein, the term “binds to”, “is binding to”, or “capable of binding to” refers within the context of an Ab that binds to or is binding to or is capable of binding to, to an Ab that is able to bind a certain antigen. An antigen within that context may be another Ab. Ability of binding to a certain antigen can be investigated by methods well known in the art including ELISA, or bio-layer interferometry (BLI). Thereby, the Ab provides a signal above the background or noise of the method when tested for binding to the antigen. In preferred embodiments, the Ab provides a signal when tested for binding to the antigen, which is at least 10%, at least 25%, at least 35%, at least 50%, at least 60%, at least 75%, at least 85%, at least 90%, at least 95%, or at least 100% higher than the signal the Ab provides when tested for binding to other antigens. For instance, an antibody that binds to complement component 1q (C1q) provides a signal when tested for binding to C1q, which is at least 10%, at least 25%, at least 35%, at least 50%, at least 60%, at least 75%, at least 85%, at least 90%, at least 95%, or at least 100% higher than the signal the Ab provides when tested for binding to complement component 3. In a preferred embodiment, the Ab is able to bind to the antigen with the Ab variable region.

As used herein, the term “allow binding” refers within the context of an Ab to a situation, wherein an Ab is incubated with a certain molecule e.g. an antigen like a DENV VLP coupled to a microsphere for a certain time to enable the Ab to bind to the molecule. If an Ab does not bind to a certain molecule, no binding will occur. The term “allow binding” refers within the context of complement component 1q (C1q) to a situation, wherein C1q is incubated with a certain molecule e.g. an antibody for a certain time to enable the C1q to bind to the molecule. If C1q does not bind to a certain molecule, no binding will occur.

As used herein, the term “bound to” refers within the context of an Ab that is bound to, to an Ab that is bound to a molecule e.g. an antigen. The Ab can be bound to said molecule with the antibody constant or variable region. In the case that the molecule is an antigen the Ab is bound to the antigen with the antibody variable region. The antigen within that context may be another antibody. For instance, a reporter Ab is bound to pre-reporter Ab with the reporter Ab variable region. The term “bound to” refers within the context of complement component 1q (C1q) that is bound to, to C1q that is bound to a molecule e.g. an antibody. In the case that the molecule is an Ab the C1q is bound to the Ab with the C-terminal globular head region of C1q.

A “recombinant Ab”, as used herein, refers to an Ab which is created, expressed, isolated or obtained by technologies or methods known in the art as recombinant DNA technology which include, e.g. DNA splicing and transgenic expression. The term may refer to Abs expressed in a non-human mammal (including transgenic non-human mammals e.g. transgenic mice), or a cell (e.g. CHO cells) expression system or isolated from a recombinant combinatorial human antibody library.

As used herein, the term “monoclonal Ab” (“mAb”) refers to an Ab obtained from a population of substantially homogenous Abs that bind to the same antigenic determinants (epitopes). “Substantially homogeneous” means that the individual Abs are identical except for possibly naturally-occurring mutations that may be present in minor amounts. This is in contrast to polyclonal antibodies that typically include different antibodies directed against various, different antigenic determinants (epitopes). A monoclonal Ab may be generated by hybridoma technology according to methods known in the art (Kohler and Milstein, Nature 1975, 256:495-497), phage selection, recombinant expression, and transgenic animals.

As used herein, the term “polyclonal Ab” refers to an Ab obtained from an immunized animal (e.g. mouse or rabbit serum). A characteristic of a mixture of polyclonal Abs is that the Abs do not all bind to the same epitope.

As used herein, the term “reporter Ab” refers to an Ab that is applied in the methods of the present application, as well as to an Ab that is part of the kits of the present invention. In a first aspect, when the assay is based on the detection of binding of C1q to flavivirus antigen/complement-fixing antibody complexes, the reporter Ab is capable of binding to the C1q with the variable region of the reporter antibody. Preferably, the reporter Ab is specific for C1q, more preferably for human C1q. Antibodies specific for C1q are commercially available. Alternatively, antibodies specific for C1q can be produced by methods known in the art such as immunization of laboratory animals and selection of C1q-specific hybridoma.

In a second aspect, when the assay is based on the detection of the formation of C3d due to the formation of flavivirus antigen/complement-fixing antibody complexes in the presence of complement competent serum, the reporter Ab is capable of binding to the formed C3d. Preferably, the reporter Ab is specific for C3d, more preferably for human C3d. Antibodies specific for C3d are commercially available. Alternatively, antibodies specific for C3d can be produced by methods known in the art such as immunization of laboratory animals and selection of C3d-specific hybridoma.

In a third aspect, when applied together with a pre-reporter Ab, the reporter Ab is capable of binding to the heavy chain constant region of the pre-reporter antibody with the variable region of the reporter antibody. The reporter Ab is attached to a detectable label, preferably with the heavy chain constant region of the reporter antibody.

As used herein, the term “pre-reporter Ab” refers to an Ab that is applied in the methods of the present application, as well as to an Ab that is part of the kits of the present invention. The pre-reporter Ab is applied together with a reporter Ab. In a first aspect, when the assay is based on the detection of binding of C1q to flavivirus antigen/complement-fixing antibody complexes, the pre-reporter Ab is capable of binding to the C1q with the variable region of the pre-reporter antibody, more preferably specific for C1q, even more preferred human C1q. In a second aspect, when the assay is based on the detection of the formation of C3d due to the formation of flavivirus antigen/complement-fixing antibody complexes in the presence of complement competent serum, the pre-reporter Ab is capable of binding to the formed C3d. Preferably, the pre-reporter Ab is specific for C3d, more preferably for human C3d.

As used herein, the terms “flavivirus-reactive Ab” or “Ab reactive to a flavivirus” refers to an Ab that is capable of binding to a flavivirus.

### Detection System and Label

The term “detectable label”, as used herein, refers to any compound or moiety that comprises one or more appropriate chemical substances or enzymes, which directly or indirectly generate a detectable compound or signal in a chemical, physical or enzymatic reaction. Labeling can be achieved by methods well known in the art (see, for example, Lottspeich, F., and Zorbas H., Springer Spektrum 2012, Bioanalytik).

As used herein, the term “antibody is attached to a detectable label”, refers to an Ab that is connected to a detectable label. The connection can be a covalent connection, which occurs for instance upon formation of an amide bound between the antibody and the detectable label. The type of connection is dependent on the functional groups available on the Ab and the detectable label. In preferred embodiments, the antibody is attached to the detectable label with the heavy chain constant region of the antibody.

As used herein, the term “detection system” refers to any system which is suitable for determining values indicative for the presence and/or amount of reporter antibody bound to C1q or a pre-reporter Ab. The detection system may also be able to determine values indicative for the presence and/or amount of a microsphere. The microsphere may be by individually identified by a detectable label. The detection system comprises one or more light sources.

As used herein, the term “light source” refers to any light source that is suitable to excite a detectable label as for instance a fluorescence dye. In preferred embodiments the light source may be a laser. In other preferred embodiments the light source may be a light emitting diode (LED).

### Microsphere Complex

As used herein, the terms “microsphere” or “microspheres” refer to a small particles to which molecules like antigens (i.e. VLPs) can be attached to for use in the methods of the present invention. The terms microsphere, microparticle, bead, or microbead can be used interchangeably and bear equivalent meanings. A microsphere may be identified by a detectable label.

As used herein, the term “microsphere complex” refers to a complex of microsphere and antigen. The antigen may be covalently attached to the microsphere. The antigen may be a VLP e.g. a DENV1-4 VLP. The antigen may be NS1. The antigen may be attached to the microsphere by carbodiimide coupling using 1-Ethyl-3-(3-dimethylaminopropyl)-carbodiimide (EDC) and N-Hydroxysulfosuccinimide (Sulfo-NHS).

### Antigen and Flavivirus

As used herein, the term “antigen” refers to any substance which can be bound by an Ab. Antigens may induce an immune response within a subject. An antigen may have one or more epitopes. An antigen may be a protein, polypeptide, carbohydrate, polynucleotide, lipid, or combinations thereof. An antigen may be a truncated version of a protein, a protein tagged with an affinity tag such as a His- or a STREP-tag, or a single domain of a protein. As used herein, antigen may e.g. refer to a DENV1 VLP, DENV2 VLP, DENV3 VLP, DENV4 VLP, ZIKV VLP; ZIKV NS1, DENV1 NS1, DENV2 NS1, DENV3 NS1, and/or DENV4 NS1. Consequently, the term flavivirus antigen refers to an antigen from a flavivirus.

As used herein, the terms “virus like particle (VLP)” or “virus like particles (VLPs)” refer to molecules that closely resemble viruses, but are non-infectious because they do not contain viral genetic material. VLPs can be prepared recombinant through the expression of viral structural proteins, which can then self-assemble into the VLPs. Examples of VLPs are ZIKV VLPs and DENV VLPs.

As used herein, the term “DENV VLP” refers to a VLP comprising at least one of the structural proteins (prM, M, and E protein) of at least one DENV strain. The term DENV VLP may refer to a VLP comprising at least one of the structural proteins of a DENV1, DENV2, DENV3, or DENV4 strain, consequently being described by the term DENV1 VLP, DENV2 VLP, DENV3 VLP, or DENV4 VLP. The structural proteins may be at least 80%, or at least 85%, or at least 90%, or at least 95% or at least 100% identical to sequences of naturally occurring DENV strains. In preferred embodiments DENV structural proteins are at least 80%, or at least 85%, or at least 90%, or at least 95% or at least 100% identical to sequences of DENV1 strain Puerto Rico/US/BID-V853/1998 (GenBank accession No. EU482592.1; SEQ ID NO: 1 and 5), DENV2 strain Thailand/16681/84 (EMBL-EBI accession No: U87411.1; SEQ ID NO: 2 and 6), DENV3 strain Sri Lanka D3/H/IMTSSA-SRI/2000/1266 (GenBank accession No. AY099336.1; SEQ ID NO: 3 and 7), and DENV4 strain Dominica/814669/1981 (EMBL-EBI accession No: AF326825.1; SEQ ID NO: 4 and 8) and DENV VLPs are produced in HEK293 cells. For production of this preferred DENV1-4 VLPs, the C-terminal 20% of DENV E protein were replaced by the corresponding Japanese encephalitis virus (JEV) SA-14 sequence (EMBL-EBI accession No: M55506.1, E protein amino acids 399-497 (DENV1 VLP), 397-495 (DENV2 VLP), 399-492 (DENV3 VLP), 400-495 (DENV4 VLP)). The replaced sequence corresponds to the transmembrane and intraparticle portion of the protein.

As used herein, the term “epitope” or “antigenic determinant” refers to the part of an antigen that interacts with a specific antigen-binding site in the variable region of an Ab molecule known as a paratope. Conversely, the “epitope” can also interact with a specific cellular receptor or binding site on a host. A single antigen may have more than one epitope. Thus, different Abs may bind to different areas on an antigen and may have different biological effects. For example, the term “epitope” also refers to a site on an antigen to which B and/or T cells respond. Epitopes may be defined as structural or functional. Functional epitopes are generally a subset of the structural epitopes and have those residues that directly contribute to the affinity of the interaction. The epitope to which the antibodies bind may consist of a single contiguous sequence of 2 or more (e.g., 2, 3, 4, 5, 6, 7, 8, 9, 10, 11, 12, 13, 14, 15, 16, 17, 18, 19, 20 or more) amino acids located within an antigen i.e. a linear epitope for instance in a domain of a ZIKV E protein. Epitopes may also be conformational, that is, composed of a plurality of non-contiguous amino acids, i.e., non-linear amino acid sequence. A conformational epitope typically includes at least 3 amino acids, and more commonly, at least 5 amino acids, e.g., 7-10 amino acids in a unique spatial conformation. In certain embodiments, epitopes may include determinants that are chemically active surface groupings of molecules such as amino acids, sugar side chains, phosphoryl groups, or sulfonyl groups, and, in certain embodiments, may have specific charge characteristics. Epitopes formed from contiguous amino acids are typically retained on exposure to denaturing solvents, whereas epitopes formed by tertiary folding are typically lost on treatment with denaturing solvents. Various techniques known to persons of ordinary skill in the art can be used to determine whether an antibody interacts with one or more amino acids within a polypeptide or protein. Exemplary techniques include, for example, site-directed mutagenesis (e.g., alanine scanning mutational analysis). Other methods include routine cross-blocking assays (such as that described in Antibodies, Harlow and Lane, Cold Spring Harbor Press, Cold Spring Harbor, NY), peptide blot analysis (Reineke (2004) Methods Mol. Biol. 248: 443-63), peptide cleavage analysis crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues that correspond to the specific amino acids with which the antibody interacts. See, e.g., Ehring (1999) Analytical Biochemistry 267: 252-259; Engen and Smith (2001) Anal. Chem. 73: 256A-265A. Modification-Assisted Profiling (MAP), also known as Antigen Structure-based Antibody Profiling (ASAP) may be used to sort Abs binding the same antigen into groups of Abs binding different epitopes. MAP is a method that categorizes large numbers of Abs directed against the same antigen according to the similarities of the binding profile of each antibody to chemically or enzymatically modified antigen surfaces (see US 2004/0101920). Each category may reflect a unique epitope either distinctly different from or partially overlapping with epitope represented by another category. This technology allows rapid filtering of genetically identical antibodies, such that characterization can be focused on genetically distinct antibodies. When applied to hybridoma screening, MAP may facilitate identification of rare hybridoma clones that produce mAbs having the desired characteristics.

As used herein, the term “flavivirus” refers to viruses belonging to the genus Flavivirus of the family Flaviviridae. According to virus taxonomy, about 50 viruses including ZIKV, DENV, YFV, JEV, WNV, SLEV, TBEV, MVEV, and related flaviviruses are members of this genus. The viruses belonging to the genus Flavivirus are referred to herein as flaviviruses. Currently, these viruses are predominantly in East, Southeast and South Asia and Africa, although they may be found in other parts of the world. Flaviviruses are spread by mosquitoes. Flaviviruses possess a positive sense, single-stranded RNA genome encoding both structural and nonstructural polypeptides. The genome also contains non-coding sequences at both the 5′- and 3′-terminal regions that play a role in virus replication. Structural polypeptides encoded by these viruses include, without limitation, capsid (C), precursor membrane (prM), membrane (M), and envelope (E) protein. Non-structural (NS) polypeptides encoded by these viruses include NS1, NS2A, NS2B, NS3, NS4A, NS4B, and NS5.

A “recombinant protein”, as used herein, refers to a protein which is created, expressed, isolated or obtained by technologies or methods known in the art such as recombinant DNA technology which include, e.g. polymerase chain reaction (PCR), DNA splicing and transgenic expression. The term may refer to proteins expressed in a non-human mammal (including transgenic non-human mammals e.g. transgenic mice), or a cell (e.g. human embryonic kidney cells (HEK293), Chinese hamster ovary (CHO) cells, or bacterial cells like Escherichia co/i) expression system. The recombinant protein may be purified by protein purification methods known in the art such as immobilized metal affinity chromatography (IMAC; e.g. His-purification) and size-exclusion chromatography. The protein may be characterized by methods known in the art such as e. g. Bradford or bicinchoninic acid (BCA) assays for determination of protein concentration, or biolayer interferometry (BLI) for determination of binding properties of the protein.

As used herein, the term “Zika virus (ZIKV)” refers to a flavivirus which has been linked to microcephaly and other developmental abnormalities in the fetuses of pregnant women exposed to the virus (Schuler-Faccini et al., MMWR Morb. Mortal. Wkly. Rep. 2016, 65:59-62) as well as Guillian-Barre syndrome in adults (Cao-Lormeau et al., Lancet 2016, 387(10027):1531-9). The ZIKV may be from African or Asian genotype.

As used herein, the term “Dengue virus (DENV)” refers to a flavivirus which can be divided in different dengue serotypes. The term “DENV” may refer to all dengue serotypes. In humans, DENV can cause severe disease forms such as dengue hemorrhagic fever (DHF) or dengue shock syndrome (DSS).

The term “dengue serotype” as used herein, refers to a species of dengue virus which is defined by its cell surface antigens and therefore can be distinguished by serological methods known in the art. Four serotypes of dengue virus are known, i.e. dengue serotype 1 (DENV1, dengue 1 virus), dengue serotype 2 (DENV2, dengue 2 virus), dengue serotype 3 (DENV3, dengue 3 virus), dengue serotype 4 (DENV4, dengue 4 virus). The term “dengue serotype” includes strains of DENV isolated from different DENV isolates, for instance DENV1 strain Puerto Rico/US/BID-V853/1998 (GenBank accession No. EU482592.1; SEQ ID NO: 1 and 5), DENV2 strain Thailand/16681/84 (EMBL-EBI accession No: U87411.1; SEQ ID NO: 2 and 6), DENV3 strain Sri Lanka D3/H/IMTSSA-SRI/2000/1266 (GenBank accession No. AY099336.1; SEQ ID NO: 3 and 7), and DENV4 strain Dominica/814669/1981 (EMBL-EBI accession No: AF326825.1; SEQ ID NO: 4 and 8).

As used herein, the term “West Nile virus (WNV)” refers to a flavivirus which can cause a disease known as West Nile fever in humans.

As used herein, the term “Japanese encephalitis virus (JEV)” refers to a flavivirus which can cause Japanese encephalitis in humans, an infection of the brain.

As used herein, the term “Yellow Fever virus (YFV)” refers to flavivirus which can cause Yellow Fever in humans.

As used herein, the term “St. Louis encephalitis virus (SLEV)” refers to a flavivirus which can cause St. Louis encephalitis in humans.

As used herein, the term “Tick-borne encephalitis virus (TBEV)” refers to a flavivirus which can cause Tick-borne encephalitis in humans.

As used herein, the term “Murray Valley encephalitis virus (MVEV)” refers to a flavivirus which can cause Murray Valley encephalitis (previously known as Australian encephalitis or Australian X disease).

As used herein, the term “structural protein” refers to one or more of C, E, prM, and M.

As used herein, the term “non-structural protein” refers to one or more of NS1, NS2A, NS2B, NS3, NS4A, NS4B, and NS5.

### Flavivirus Infection and Subject

As used herein, the terms “subject” or “subjects” can include any individual. A subject may be, but is not limited to, a mouse, a primate, a non-human primate (NHP), a human, a rabbit, a cat, a rat, a horse, a sheep. In certain embodiment the subject can be a pregnant mammal, and in particular embodiments a pregnant human female. In some embodiments the subject is a patient, for whom prophylaxis or therapy is desired. The subject may be seronegative or seropositive for a certain flavivirus. The subject may be from a flavivirus endemic region. The subject may be vaccinated with a flavivirus vaccine.

As used herein, “endemic region” refers to a region where a disease or infectious agent is constantly present and/or usually prevalent in a population within this region. As used herein, “non-endemic region” refers to a region from which the disease is absent or in which it is usually not prevalent. Accordingly, a “flavivirus endemic region” refers to geographic areas in which an infection with flavivirus is constantly maintained at a baseline level. A “flavivirus non-endemic region” is a geographic area in which an infection with flavivirus is not constantly maintained at a baseline level. Accordingly, subject populations or subjects “from a flavivirus endemic region” or “from a flavivirus non-endemic region” refer to subject populations or subjects living in geographic areas as defined above.

As used herein, the term “seropositive subject” or “subject which is seropositive for a flavivirus” refers to a subject which has Abs against a certain flavivirus. Abs against the flavivirus may have been raised upon infection with the flavivirus or vaccination with a flavivirus-directed vaccine.

As used herein, the term “seronegative subject” or “subject which is seronegative for a flavivirus” refers to a subject which has no Abs against a certain flavivirus. Consequently, the subject has not been infected with the flavivirus or vaccinated with a flavivirus-directed vaccine.

The term “flavivirus infection” as used herein, refers to the disease or condition which results from contact to a flavivirus (e.g. after being bitten by a mosquito harboring the virus), or to an infected animal, or to an infected human patient, or contact with the body fluids or tissues from an animal or human patient having a flavivirus infection. The flavivirus may be a DENV or a ZIKV. A flavivirus infection may also not be accompanied by flavivirus specific symptoms, in such a case the infection may be asymptomatic or inapparent. A flavivirus infection may be acute or convalescent.

As used herein, the term “acute flavivirus infection” refers to a flavivirus infection that is characterized by rapid onset of disease, a relatively brief period of symptoms, and resolution within days. An acute flavivirus infection is usually accompanied by early production of infectious virions and elimination of infection by the host immune system. Within an acute flavivirus infection Ab titers in body fluids are high compared to a convalescent flavivirus infection. The flavivirus within that context may be a DENV (“acute DENV infection”). An “acute flavivirus infection” may refer to the period of viremia.

As used herein, the term “viremia” or “presence of viremia” refers to a medical condition were viruses have entered the bloodstream thereby spreading throughout the body. A viremia can be primary or secondary. A primary viremia refers to a virus entering the bloodstream from the first site of infection. A secondary viremia occurs when primary viremia has resulted in infection of additional tissues in which the virus has replicated and once more entered the bloodstream circulation. Viremia may induce typical symptoms caused by an infection with the virus such as fever or headache. Viremia can be determined by monitoring the typical symptoms caused by an infection and/or determining the amount of virus in samples as blood, blood plasma, or urine e.g. by diagnostic methods like RT-PCRs, PRNT, FFA, MNT, or RVP assay.

As used herein, the term “convalescent flavivirus infection” refers to a flavivirus infection that has been eliminated by the host immune system. A characteristic of a convalescent flavivirus infection is the existence of memory B-cells encoding for Abs against the flavivirus that has caused the infection. Within a convalescent flavivirus infection Ab titers in body fluids are low compared to an acute flavivirus infection. The flavivirus within that context may be a DENV (“convalescent DENV infection”). A “convalescent flavivirus infection” may refer to the period after viremia.

As used herein, an “immune response” refers to a subject's immune response to flavivirus exposure. In particular, the immune response includes the formation of Abs to the flavivirus. The term immune response may also include formation of complement-fixing Abs to the flavivirus. It may also include the stimulation of a cell-mediated response or the formation of Abs to structural proteins such as E protein. It may also include the stimulation of a cell-mediated response.

As used herein, the term “sample” refers to any sample. The sample may be derived from a subject. Within the meaning of this invention the sample is present outside the human or animal body. Preferably, the sample may be serum, blood plasma, blood, urine, cerebrospinal fluid, lymph fluid. In some embodiments, the sample contains flavivirus-reactive complement-fixing Abs. The sample can be pre-treated prior to use, such as preparing serum or plasma from blood, diluting fluids, or the like. Methods for pre-treating can involve purification, filtration, distillation, concentration, inactivation of interfering compounds, and the addition of reagents. In some embodiment the sample is heat-inactivated. Heat-inactivation may be carried out for about 30 minutes at about 56° C. Incubation time and temperature may be varied according to the individual sample type. Within this invention, the term “serum” refers to blood serum. Within this invention, the term “plasma” refers to blood plasma.

As used herein, the term “diagnosis” or “diagnosing” refers to methods that can be used to confirm or determine the likelihood of whether a patient is suffering from or had previously suffered from a given disease or condition i.e. a flavivirus infection. The term “diagnosis” or “diagnosing” may also refer to a concomitant diagnosis of two different flavivirus infections, e.g. a DENV and ZIKV infection or infections by two different serotypes of the same flavivirus e.g. an infection by DENV serotype 1 and DENV serotype 2. The flavivirus infection may be concomitant or convalescent.

As used herein, the term “flavivirus vaccine” refers to a vaccine comprising one or more antigens of a flavivirus. Upon administration to a subject, the flavivirus vaccine induces flavivirus-reactive Abs as for instance flavivirus-reactive complement-fixing Abs. Examples of flavivirus vaccines are purified inactivated vaccines or live attenuated vaccines.

### BRIEF DESCRIPTION OF THE DRAWINGS

FIG. 1 Optimization of coupling pH exemplarily shown for DENV2 VLP (A) and DENV2 NS1 (B). Mean fluorescence intensity (MFI) is shown for the negative control sample that did not contain DENV-reactive complement-fixing Abs, as well as the high control sample, comprising a high amount of DENV-reactive complement-fixing Abs in dependence of the coupling pH and sample dilution. For instance, a 10-fold sample dilution refers to a logo (sample dilution) of 1.

FIG. 2 Schematic overview of the DENV-quadruplex complement-fixing assay. The DENV-quadruplex complement-fixing assay according to the present invention comprises application of microspheres coupled to dengue 1 virus (DV1), dengue 2 virus (DV2), dengue 3 virus (DV3), and dengue 4 virus (DV4) virus like particles (VLPs). The DV1, DV2, DV3, and DV4 VLP-coupled microspheres are applied as a mixture allowing detection of complement-fixing Abs reactive to the different DENV serotypes in one single experiment. The VLP-coupled microspheres are incubated with a sample comprising complement-fixing Abs to allow binding of these Abs to the VLPs. Afterwards, purified human C1q is added to the mixture to bind complement-fixing Abs from the sample bound to the VLPs. Bound human C1q is then further detected using an anti-human C1q pre-reporter antibody and an anti-sheep IgG phycoerythrin-coupled reporter antibody generating a fluorescence signal upon irradiation with a light source.

FIGS. 3 and 4 Validation of the DENV-quadruplex complement-fixing assay set-up. In order to validate the DENV-quadruplex complement-fixing assay set-up, a reference sample, a negative control sample, as well as control samples comprising a high, medium, and low concentration of DENV-reactive complement-fixing Abs were analyzed. Mean fluorescence intensity (MFI) depending on the sample dilution (presented as log₁₀ sample dilution) and on the DENV serotype i.e. DENV1 (FIG. 3A), DENV2 (FIG. 3B), DENV3 (FIG. 4A) and DENV4 (FIG. 4B) was fitted according to a sigmoidal curve. For instance, a 10-fold sample dilution refers to a logo (sample dilution) of 1.

FIGS. 5 and 6 Validation of the DENV-quadruplex complement-fixing assay set-up (linearity). The obtained complement-fixing antibody concentration was plotted against the expected concentration for each DENV VLP, i.e. DENV1 VLP (FIG. 5A), DENV2 VLP (FIG. 5B), DENV3 VLP (FIG. 6A) and DENV4 VLP (FIG. 6B) and was fitted by a linear regression.

FIGS. 7 and 8 Validation of the DENV-quadruplex complement-fixing assay set-up. Mean fluorescence signal (MFI) is exemplarily presented for DENV2 VLP in dependence of the sample dilution, as well as sample type. High control sample (FIG. 7A), medium control sample (FIG. 7B), as well as low control sample (FIG. 8), comprising a high, medium, and low concentration of DENV-reactive complement-fixing Abs were examined in five replicates together with the reference sample. For instance, a 10-fold sample dilution refers to a logo (sample dilution) of 1.

FIGS. 9 and 10 Validation of the DENV-quadruplex complement-fixing assay set-up. Complement-fixing antibody titers in EU/mL (arbitrary unit) are presented dependent on the operator, sample type (high, medium, and low control sample, comprising high, medium, and low amounts of DENV-reactive complement-fixing Abs), as well as DENV VLP. Results for DENV1 VLP are shown in FIG. 9A, for DENV2 VLP in FIG. 9B, for DENV3 VLP in FIG. 10A, and for DENV4 VLP in FIG. 10B.

FIGS. 11 and 12 Validation of the DENV-quadruplex complement-fixing assay set-up (intra-assay precision). The percent coefficient of variation (% CV) was calculated for the replicates dependent on the operator, sample type (high, medium, and low control sample, comprising high, medium, and low amounts of DENV-reactive complement-fixing Abs), as well as DENV VLP. Results for DENV1 VLP are shown in FIG. 11A, for DENV2 VLP in FIG. 11B, for DENV3 VLP in FIG. 12A, and for DENV4 VLP in FIG. 12B.

FIG. 13 Validation of the DENV-quadruplex complement-fixing assay set-up (inter-assay precision). The percent coefficient of variation (% CV) was calculated for the replicates carried out by the two operators dependent on the sample type (high, medium, and low control sample, comprising high, medium, and low amounts of DENV-reactive complement-fixing Abs), as well as DENV VLP.

FIG. 14 Analysis of human serum samples with the DENV-quadruplex complement-fixing assay set-up. Mean fluorescence intensity (MFI) resulting from analysis of DENV-1 VLP-coupled microspheres in the quadruplex assay set-up depending on the sample dilution (presented as logo sample dilution) was fitted according to a sigmoidal curve. Besides the data concerning human serum samples 1-5, the results from analysis of a reference sample are further included. For instance, a 10-fold sample dilution refers to a logo (sample dilution) of 1.

FIGS. 15 to 18 Analysis of samples from human subjects seropositive and seronegative for DENV. The DENV-quadruplex complement fixing-assay was applied to evaluate complement-fixing Abs in seronegative (FIGS. 15 and 17) and seropositive (FIGS. 16 and 18) subjects from two clinical studies (#1 and #2) prior (day 0 in FIGS. 15 and 16, or day 1 in FIGS. 17 and 18, respectively) and post vaccination with a tetravalent DENV vaccine, comprising live attenuated viruses of DENV1, DENV2, DENV3, and DENV4. Data are presented as median values with error bars (upper panels) and box plots (lower panels). As “day 0” in FIGS. 15 and 16 refers to samples taken prior to vaccination, day 0 can be referred to as the starting day of the study. As “day 1” in FIGS. 17 and 18 refers to samples taken prior to vaccination, day 1 can be referred to as the starting day of the study.

FIG. 19 Receiver Operator Curve (ROC) to evaluate accuracy of the DENV-quadruplex complement fixing-assay for diagnosis. The sensitivity, meaning the percentage of true positive samples i.e. samples from seropositive subjects with an MNT50 titer of 10 being also classified as positive in the complement assay is plotted on the y-axis and 1-specificity, meaning the percentage of true negative samples i.e. samples from seronegative subjects with an MNT50 titer of <10 being also classified as negative in the complement assay is plotted on the x-axis for several thresholds (or complement-fixing antibody titers).

FIGS. 20 and 21 Analysis of human samples by a 10-plex complement-fixing assay set-up using ZIKV VLP, DENV1-4 VLPs, ZIKV NS1, and DENV1-4 NS1 as antigens coupled to the microspheres. Mean fluorescence intensity (MFI) is presented for all VLPs (FIG. 20) and NS1 (FIG. 21). Complement-fixing Abs were determined in samples collected in the Dominican Republic in 2016 to 2017 from subjects suffering from febrile illness consistent with DENV and ZIKV infections (panel #1), as well as in samples collected in Colombia either before or after 2016 from subjects 8-40 days post febrile illness consistent with DENV and ZIKV infection (panels #2 and #3). Of note, zika seroprevalence in Colombia reached around 50% after 2016. In addition, a negative control lacking flavivirus-reactive Abs and a blank was included.

FIG. 22 Evaluation of non-human primate samples by the DENV-quadruplex complement-fixing assay set-up. (A) A phylogenetic tree was prepared based on the envelope amino acid sequence of the DENV serotype strains used to infect non-human primates. (B) A total of 16 animals were infected with each one DENV serotype (indicated above the graphics) and complement-fixing Ab titers were determined against each serotype for 1 year after infection (at days 0, 69, and 341) and presented for each animal. The samples on day 0 are taken prior to vaccination.

FIG. 23 Evaluation of non-human primate samples by the DENV-quadruplex complement-fixing assay set-up. A total of 16 animals were infected with each one DENV serotype (indicated above the graphics) and complement-fixing Ab titers were determined against each serotype for 1 year after infection (at days 0, 69, and 341). Complement-fixing Ab titers are averaged for all animals infected with the same serotype. The samples on day 0 are taken prior to vaccination.

FIG. 24 ZIKV NS1 complement-fixing Abs evaluated by the 10-plex assay set-up. (A) A total of 15 non-human primates were infected with each one DENV serotype (indicated next to the animal numbers), followed by ZIKV infection 1 year later. ZIKV NS1-reactive complement-fixing Ab titers in mean fluorescence intensity (MFI) were determined with the 10-plex complement-fixing assay. (B) ZIKV NS1-reactive complement-fixing Ab titers in mean fluorescence intensity (MFI) were determined with the 10-plex complement-fixing assay in human samples. Human samples analyzed were negative controls lacking ZIKV-reactive complement-fixing Abs, WNV positive samples lacking ZIKV-reactive complement-fixing Abs but containing WNV-reactive complement-fixing Abs, as well as ZIKV positive samples containing ZIKV-reactive complement-fixing Abs.

FIG. 25 Complement fixing antibody titers measured by the DENV-quadruplex complement-fixing assay in non-human primates that were vaccinated with either YFV, ZIKV, WNV, TBEV, or JEV vaccine (indicated above the graphics). The titers are shown for all four DENV VLPs (DENV1-4) for each animal. Titers were determined at days 1, 57, and 169. PIZV=Purified inactivated Zika vaccine.

FIG. 26 Neutralizing and complement-fixing antibody levels against all four dengue virus serotypes in subjects living in dengue endemic areas. Microneutralization (MNT50) antibody (A) and complement-fixing antibody (B) levels are shown per analyzed sample for each dengue serotype. In total, 53 samples were analyzed. Horizontal lines represent the threshold for considering a subject as seropositive (≥10 for MNT50 and ≥3 EU/mL for complement-fixing antibodies).

FIG. 27 Correlation analysis between neutralizing (MNT50) and complement-fixing antibodies against all four dengue virus (DENV) serotypes in subjects living in dengue endemic areas. Correlation analysis was performed using Log₁₀-transformed MNT50 and complement-fixing antibody titers and the correlation coefficient (R²) was calculated for each DENV serotype. Vertical and horizontal lines represent LLOQ of the MNT50 (10) and complement-fixing antibody assays (3 EU/mL), respectively.

FIG. 28 Total IgG titers against all four dengue virus serotypes in subjects living in dengue endemic areas. The horizontal dotted line represents the assay LLOQ (200 RU/mL; RU=relative units).

FIG. 29 Correlation analysis between total IgG titers and complement-fixing antibody titers against all four dengue virus serotypes in subjects living in endemic areas. Correlation analysis was performed using Log₁₀-transformed total IgG titers and complement-fixing antibody titers and the correlation coefficient (R²) was calculated for each DENV serotype. Vertical and horizontal lines represent LLOQ of the total IgG binding (200 RU/mL) and complement-fixing antibody assays (3 EU/mL), respectively.

FIG. 30 Schematic overview of C3d production by the classical complement activation pathway (A) and C3d deposition assay set-up (B). For C3d production, C1q binds to the immunocomplex and changes its conformation, which in turn activates the enzymes C1r and C1s. These enzymes cleave C4 and the larger fraction (C4b) remains covalently bound to C1q(rs)2. Then, C2 is cleaved and the larger fraction (C2a) remains bound to the C4b forming an enzyme C3 convertase (C4b2a). The convertase cleaves C3 and the larger fraction (C3b) binds to C4b2a, as well as the surroundings. C3b is then finally further cleaved into C3d, which is detected in the C3d deposition assay set-up.

FIG. 31-34 Complement fixing antibody titers measured by the C1q-based DENV-quadruplex complement-fixing assay set-up (FIGS. 31 and 32) and the C3d deposition DENV-quadruplex complement-fixing assay set-up (C3d—fixing antibody titers; FIGS. 33 and 34) in a panel of 9 serum samples collected from DENV seropositive healthy adults living in dengue-endemic areas in Colombia from 2015-2016. In addition, high, medium, and low control samples comprising high, medium, and low number of DENV-reactive complement-fixing Abs were included.

FIG. 35 Correlation between C1q-based complement-fixing antibody titers and C3d-based complement-fixing antibody titers as determined in FIG. 31-34 (R²=correlation coefficient; CI=confidence interval).

### DETAILED DESCRIPTION

In the following sections, various exemplary compositions and methods are described in order to detail various embodiments. It will be obvious to one skilled in the art that practicing the various embodiments does not require the employment of all or even some of the specific details outlined herein, but rather that concentrations, times and other specific details may be modified through routine experimentation.

### Microsphere Complex

The microsphere complex for use in the methods and kits of the present application comprises a microsphere coupled to a flavivirus antigen.

### Microsphere

The microsphere useful for the invention ranges in the size from about 0.01 to about 100 μm in diameter, more preferably from about 1 to about 20 μm, and most preferably a microsphere has a diameter from about 5 to about 7 μm. In a preferred embodiment the microsphere has a diameter of about 6.5 μm. The size of a microsphere can be determined in practically any flow cytometry apparatus by so-called forward or small-angle scatter light.

The microsphere may be constructed of any material to which molecules like VLPs or NS1 may be attached to. For example, acceptable materials for the construction of microspheres include but are not limited to: polystyrene, polyacrylic acid, polyacrylonitrile, polyacrylamide, polyacrolein, polybutadiene, polydimethylsiloxane, polyisoprene, polyurethane, polyvinylacetate, polyvinylchloride, polyvinylpyridine, polyvinylbenzylchloride, polyvinyltoluene, polyvinylidene chloride, polydivinylbenzene, polymethylmethacrylate, or combinations thereof. In a preferred embodiment of the present invention, microspheres are constructed of polystyrene.

The microsphere may comprise surface affinity groups for attachment of molecules. Said affinity groups may be, but are not limited to, Ni 2+(for immobilization of His-tagged molecules), Protein A, Protein G, Protein L, anti-human IgG Ab, anti-rabbit IgG Ab, anti-mouse IgG Ab, anti-goat IgG Ab, anti-FLAG Ab, streptavidin, avidin, and glutathione.

The microsphere may comprise functional groups on the surface useful for attachment of molecules, such as the antigens of the present invention. Said functional groups may be, but are not limited to, carboxylates, esters, alcohols, carbamides, aldehydes, amines, sulfur oxides, nitrogen oxides, maleimides, or halides. In a preferred embodiment the microsphere comprises carboxylates on the surface. Molecules like antigens can be covalently coupled to the microspheres using chemical techniques described herein. In a preferred embodiment molecules like antigens (i.e. VLPs or NS1) can be coupled to the microsphere by carbodiimide coupling using 1-Ethyl-3-(3-dimethylaminopropyl)carbodiimide (EDC) and N-hydroxysulfosuccinimide (Sulfo-NHS). Thereby, the EDC is reacting to an unstable o-acylisourea ester with a carboxylate on the surface of the microspheres. The unstable o-acylisourea ester readily reacts with Sulfo-NHS to form a semi-stable amine reactive NHS-ester. The NHS-ester finally reacts with an amine group provided by an antigen, thereby forming a stable amide bond.

As amine-containing compounds other than those provided by the antigen, glycerol, urea, imidazole, azide, and some detergents may interfere with the carbodiimide coupling, they should be removed from the antigen preparation with a suitable buffer exchange method. For instance, a suitable buffer for carbodiimide coupling is 50 mM 2-(N-morpholino)ethanesulfonic acid (MES) buffer. The pH value for coupling may be between about 5 and about 9. Coupling of the antigen to the microsphere may be carried out by incubation for about 2 hours.

The microsphere may be magnetic. In a preferred embodiment the microsphere may be superparamagnetic. Magnetic microspheres can be easily captured by a magnetic plate separator for instance to wash the microspheres.

A magnetic plate separator can be used for separating the microspheres within the 96-well plate from the solution within the wells of the 96-well plate by magnetic capture and refers to a construction for holding a 96-well plate. A magnetic plate separator enables the user to quickly decant the supernatant within the wells and washing of the wells, while fixing the microspheres at the bottom of the 96-well plate by magnetic capture. Application of a magnetic plate separator reduces the risk that microspheres are getting lost during washing procedures.

The microsphere may comprise a detectable label by which the microsphere can be identified with the help of a detection system. Identification of a microsphere likewise allows identification of the antigen which is coupled to the microsphere.

Concerning the detection of such labels with suitable detection systems, reference is also made to the section “Detection system”.

In preferred embodiments the detectable label is at least one fluorescent dye. The fluorescent dye may be for instance selected from the group consisting of squaraine, phthalocyanine, naphthalocyanine, and any derivative thereof. For instance, a derivative of a fluorescent dye may be the dye further comprising a methyl group at any position.

In some embodiments the microsphere comprises one fluorescent dye. The microsphere can be identified by the emission signal of the one fluorescent dye upon irradiation with a suitable light source.

In other embodiments different microspheres comprise different concentrations of the same fluorescent dye. In such embodiments, the microsphere can be identified by the intensity of the emission signal of the one fluorescent dye upon irradiation with a suitable light source.

In some embodiments the microsphere comprises two or more fluorescent dyes. The microsphere can be identified by the emission signal of the two or more fluorescent dyes upon irradiation with a suitable light source.

In some embodiments different microspheres comprise different concentrations of the two or more fluorescent dyes. In such embodiments, the microsphere can be identified by the intensity of the emission signal of the two or more fluorescent dyes (indicative for the ratio of the two or more fluorescent dyes) upon irradiation with a suitable light source.

In certain embodiments where the microsphere comprises two or more fluorescent dyes, the emission signal of the two or more fluorescent dyes is resulting from an overlay of the emission signal of the single fluorescent dyes. The intensity of the emission signal is therefore indicative for the ratio of the two or more fluorescent dyes.

The at least one fluorescent dye can be excited with any suitable light source as for instance a laser or a light emitting diode (LED) using a suitable detection system.

The at least one fluorescent dye can be covalently attached onto the surface of the microsphere, or can be internally incorporated during microsphere production (i.e. polystyrene polymerization), or the microsphere can be dyed after production by placing the microsphere in a suitable solution comprising the at least one fluorescent dye. A suitable solution comprising the at least one fluorescent dye is for instance an organic solution.

In one embodiment, one microsphere may comprise two fluorescent dyes having an emission signal maximum at 675 nm, another microsphere may comprise two different fluorescent dyes having an emission signal maximum at 700 nm.

In another embodiment, one microsphere may comprise a fluorescent dye at one specific concentration, another microsphere may comprise the same fluorescent dye at another specific concentration, wherein the emission signal maxima of the two microspheres occur at the same wavelength but with different emission signal intensities.

In certain embodiments different microspheres comprising different concentrations of fluorescent dyes can be excited by the same light source (e.g. the one or more fluorescent dyes at specific concentrations in the different microspheres are excitable by the same wavelength). In specific embodiments the different microspheres are excitable with a wavelength within the range from about 600 to about 650 nm, more preferably with a wavelength of about 615 nm to about 640 nm, and even more preferably with a wavelength of about 620 to about 635 nm. In one embodiment the different microspheres are excitable with a wavelength of about 635 nm. An advantage of such a set-up is, that only one light source is needed for distinguishing all microspheres present within a microsphere mixture and thereby further simplifying a set-up in which multiple antigens can be analyzed in one single experiment.

The microspheres may also be identified by their size, if different microspheres are of a different size using a suitable detection system. The size of the microspheres ranges from 0.01 to 100 μm in diameter, more preferably from about 1 to about 10 μm in diameter. For instance, one microsphere may be about 6 μm in diameter, another microsphere may be about 6.5 μm in diameter.

The microsphere may also be identified by a specific shape of the microsphere, if different microspheres are of a different shape using a suitable detection system.

To allow the simultaneous detection of complement-fixing antibodies reactive to different antigens in one single experiment, microspheres with a different size or a different detectable label or a different shape are coupled to different antigens and mixed. Microspheres coupled to the same antigen have the same size or the same detectable label or the same shape. Although the microspheres are mixed each microsphere can be identified by the specific size or detectable label or shape of the microsphere. Thereby, the antigen coupled to the microsphere can be simultaneously identified.

Microspheres may be one out of the list consisting of MagPlex® microspheres, MicroPlex® microspheres, LumAvidin® microspheres, MagPlex®-Avidin microspheres, and SeroMAP® microspheres produced by the Luminex Corporation (Austin, Texas). The type of microsphere which can be used depends on the detection system applied (reference is also made to the section “Detection system”).

In a preferred embodiment the microspheres are the MagPlex® microspheres, which are superparamagnetic polystyrene microspheres with surface carboxyl groups and a diameter of about 6.5 μm produced by Luminex Corporation (Austin, Texas). MagPlex® microspheres comprise two or more fluorescent dyes at a specific concentration allowing each microsphere to be identified by a detection system as for instance a MAGPIX® instrument as produced by the Luminex Corporation (Austin, Texas). Microspheres of different MagPlex® microsphere catalog numbers (Luminex Corporation, Austin, Texas) comprise the two or more fluorescent dyes at different concentrations. The MagPlex® microspheres can be excited by the same excitation wavelength and therefore only one light source is required for microsphere identification.

In specific embodiments the excitation wavelength is from about 600 to about 650 nm, preferably from about 615 to about 640 nm, more preferably from about 620 to about 635 nm. For instance, the excitation wavelength is about 635 nm.

### Flavivirus Antigen

The flavivirus antigen to which the microsphere is coupled to can be a whole virion surrogate, such as virus like particle (VLP). Virus like particles comprise one or more of the structural proteins of the flavivirus from which they are derived i.e. prM, M, and E protein. The flavivirus VLP may be for instance a ZIKV VLP or a DENV VLP. The DENV VLP may be of any serotype (DENV1-4).

Flavivirus VLPs of the present invention may be derived from any flavivirus strain, including any DENV or ZIKV strain. Flavivirus VLPs may be produced in any suitable cell such as a mammalian cell or an insect cell (such as cells from Aedes albopictus (C6/36) or Spodoptera frugiperda (Sf9). In some embodiments, the cells used for VLP production are human cells. In specific embodiments, the cells used for VLP production are HEK293 cells.

In one embodiment, the flavivirus antigen is a DENV1 VLP derived from DENV1 strain Puerto Rico/US/BID-V853/1998 (GenBank accession No. EU482592.1; SEQ ID NO: 1 and 5). DENV1 VLP may be produced in HEK293 cells. In more specific embodiments the DENV1 VLP comprises structural proteins from DENV1 strain Puerto Rico/US/BID-V853/1998 (GenBank accession No. EU482592.1; SEQ ID NO: 1 and 5). In even more specific embodiments the DENV1 VLP comprises the E protein, M protein, and prM protein which are at least 80%, or at least 85%, or at least 90%, or at least 95% or at least 100% identical to those encoded by DENV1 strain Puerto Rico/US/BID-V853/1998 (GenBank accession No. EU482592.1; SEQ ID NO: 1 and 5).

In one embodiment, the flavivirus antigen is a DENV2 VLP derived from DENV2 strain Thailand/16681/84 (EMBL-EBI accession No: U87411.1; SEQ ID NO: 2 and 6). DENV2 VLP may be produced in HEK293 cells. In more specific embodiments the DENV2 VLP comprises structural proteins from DENV2 strain Thailand/16681/84 (EMBL-EBI accession No: U87411.1; SEQ ID NO: 2 and 6). In even more specific embodiments the DENV2 VLP comprises the E protein, M protein, and prM protein which are at least 80%, or at least 85%, or at least 90%, or at least 95% or at least 100% identical to those encoded by DENV2 strain Thailand/16681/84 (EMBL-EBI accession No: U87411.1; SEQ ID NO: 2 and 6).

In one embodiment, the flavivirus antigen is a DENV3 VLP derived from DENV3 strain Sri Lanka D3/H/IMTSSA-SRI/2000/1266 (GenBank accession No. AY099336.1; SEQ ID NO: 3 and 7). DENV3 VLP may be produced in HEK293 cells. In more specific embodiments the DENV3 VLP comprises structural proteins from DENV3 strain Sri Lanka D3/H/IMTSSA-SRI/2000/1266 (Gen Bank accession No. AY099336.1; SEQ ID NO: 3 and 7). In even more specific embodiments the DENV3 VLP comprises the E protein, M protein, and prM protein which are at least 80%, or at least 85%, or at least 90%, or at least 95% or at least 100% identical to those encoded by DENV3 strain Sri Lanka D3/H/IMTSSA-SRI/2000/1266 (GenBank accession No. AY099336.1; SEQ ID NO: 3 and 7).

In one embodiment, the flavivirus antigen is a DENV4 VLP derived from DENV4 strain Dominica/814669/1981 (EMBL-EBI accession No: AF326825.1; SEQ ID NO: 4 and 8). DENV4 VLP may be produced in HEK293 cells. In more specific embodiments the DENV4 VLP comprises structural proteins from DENV4 strain Dominica/814669/1981 (EMBL-EBI accession No: AF326825.1; SEQ ID NO: 4 and 8). In even more specific embodiments the DENV4 VLP comprises the E protein, M protein, and prM protein which are at least 80%, or at least 85%, or at least 90%, or at least 95% or at least 100% identical to those encoded by DENV4 strain Dominica/814669/1981 (EMBL-EBI accession No: AF326825.1; SEQ ID NO: 4 and 8).

In specific embodiments, for production of the DENV1-4 VLPs, the C-terminal 20% of DENV E protein were replaced by the corresponding Japanese encephalitis virus (JEV) SA-14 sequence (EMBL-EBI accession No: M55506.1, E protein amino acids 399-497 (DENV1 VLP), 397-495 (DENV2 VLP), 399-492 (DENV3 VLP), 400-495 (DENV4 VLP). The replaced sequence corresponds to the transmembrane and intraparticle portion of the protein.

In one embodiment, the flavivirus antigen is a ZIKV VLP derived from ZIKV strain Suriname/Z1106033 (GenBank Accession No. KU312312.1 and ALX35659.1; SEQ ID NO: 9 and 10). ZIKV VLP may be produced in HEK293 cells. In more specific embodiments the ZIKV VLP comprises structural proteins from ZIKV strain Suriname/Z1106033 (GenBank Accession No. KU312312.1 and ALX35659.1; SEQ ID NO: 9 and 10). In even more specific embodiments the ZIKV VLP comprises the E protein, M protein, and prM protein which are at least 80%, or at least 85%, or at least 90%, or at least 95% or at least 100% identical to those encoded by ZIKV strain Suriname/Z1106033 (GenBank Accession No. KU312312.1 and ALX35659.1; SEQ ID NO: 9 and 10).

The flavivirus antigen to which the microsphere is coupled to can be a single protein, such as a structural or non-structural protein as for instance non-structural protein 1 (NS1). Further proteins suitable for coupling to the microsphere are prM, M, E, C, NS1, NS2A, NS2B, NS3, NS4A, NS4B, and NS5, or fragments or variants thereof.

The flavivirus antigen to which the microsphere is coupled to can be any derivative of a structural and non-structural protein such as a protein domain or a part of a protein, such as the domain III of the envelope protein (E).

The protein or protein domain or part of a protein can be produced recombinantly (recombinant protein) in any suitable cells such as mammalian cells, insect cells, or bacterial cells.

In one embodiment, the flavivirus antigen is a DENV1 NS1 derived from DENV1 strain Nauru/Western Pacific/1974 (SEQ ID NO: 11 and 12; GenBank accession No. AY145121). DENV1 NS1 may be produced in HEK293 cells. In more specific embodiments the DENV1 NS1 is at least 80%, or at least 85%, or at least 90%, or at least 95% or at least 100% identical to the NS1 encoded by DENV1 strain Nauru/Western Pacific/1974 (SEQ ID NO: 11 and 12; GenBank accession No. AY145121). In even more specific embodiments the sequence of the DENV1 NS1 the microspheres are coupled to is shown in SEQ ID NO: 13.

In one embodiment, the flavivirus antigen is a DENV2 NS1 derived from DENV2 strain Thailand/16681/84 (EMBL-EBI accession No: U87411.1; SEQ ID NO: 2 and 6). DENV2 NS1 may be produced in HEK293 cells. In more specific embodiments the DENV2 NS1 is at least 80%, or at least 85%, or at least 90%, or at least 95% or at least 100% identical to the NS1 encoded by DENV2 strain Thailand/16681/84 (EMBL-EBI accession No: U87411.1; SEQ ID NO: 2 and 6). In even more specific embodiments the sequence of the DENV2 NS1 the microspheres are coupled to is shown in SEQ ID NO: 14.

In one embodiment, the flavivirus antigen is a DENV3 NS1 derived from DENV3 strain Sri Lanka D3/H/IMTSSA-SRI/2000/1266 (GenBank accession No. AY099336.1; SEQ ID NO: 3 and 7). DENV3 NS1 may be produced in HEK293 cells. In more specific embodiments the DENV3 NS1 is at least 80%, or at least 85%, or at least 90%, or at least 95% or at least 100% identical to the NS1 encoded by DENV3 strain Sri Lanka D3/H/IMTSSA-SRI/2000/1266 (GenBank accession No. AY099336.1; SEQ ID NO: 3 and 7). In even more specific embodiments the sequence of the DENV3 NS1 the microspheres are coupled to is shown in SEQ ID NO: 15.

In one embodiment, the flavivirus antigen is a DENV4 NS1 derived from DENV4 strain Dominica/814669/1981 (EMBL-EBI accession No: AF326825.1; SEQ ID NO: 4 and 8). DENV4 NS1 may be produced in HEK293 cells. In more specific embodiments the DENV4 NS1 is at least 80%, or at least 85%, or at least 90%, or at least 95% or at least 100% identical to those encoded by DENV4 strain Dominica/814669/1981 (EMBL-EBI accession No: AF326825.1; SEQ ID NO: 4 and 8). In even more specific embodiments the sequence of the DENV4 NS1 the microspheres are coupled to is shown in SEQ ID NO: 16.

In one embodiment, the flavivirus antigen is a ZIKV NS1 derived from ZIKV strain Suriname/Z1106033 (GenBank Accession No. KU312312.1 and ALX35659.1; SEQ ID NO: 9 and 10). ZIKV NS1 may be produced in HEK293 cells. In more specific embodiments the ZIKV NS1 is at least 80%, or at least 85%, or at least 90%, or at least 95% or at least 100% identical to those encoded by ZIKV strain Suriname/Z1106033 (GenBank Accession No. KU312312.1 and ALX35659.1; SEQ ID NO: 9 and 10).

### Reporter and Pre-Reporter Antibody

The reporter and pre-reporter Ab for use in the methods and kits of the present invention may be any Abs suitable. The reporter and pre-reporter Ab may be recombinant Abs, monoclonal Abs, or polyclonal Abs.

According to certain embodiments of the invention the reporter and the pre-reporter antibody are immunoglobulin (Ig) molecules, comprised of four polypeptide chains, two heavy (H) chains and two light (L) chains interconnected by disulfide bonds.

The reporter and pre-reporter Ab may be derived from any origin. According to certain embodiments of the invention the reporter and the pre-reporter Ab are derived from a non-human origin such as sheep, mouse, rabbit, goat, or donkey.

In a first aspect, when the assay is based on the detection of binding of C1q to flavivirus antigen/complement-fixing antibody complexes, the reporter Ab is capable of binding to the C1q with the variable region of the reporter antibody. Preferably, the reporter Ab is specific for C1q, more preferably for human C1q. Antibodies specific for C1q are commercially available. Alternatively, antibodies specific for C1q can be produced by methods known in the art such as immunization of laboratory animals and selection of C1q-specific hybridoma.

In a second aspect, when the assay is based on the detection of the formation of C3d due to the formation of flavivirus antigen/complement-fixing antibody complexes in the presence of complement competent serum, the reporter Ab is capable of binding to the formed C3d. Preferably, the reporter Ab is specific for C3d, more preferably for human C3d. Antibodies specific for C3d are commercially available. Alternatively, antibodies specific for C3d can be produced by methods known in the art such as immunization of laboratory animals and selection of C3d-specific hybridoma.

Within the embodiments of the present invention, the reporter Ab is attached to a detectable label. In preferred embodiments, the reporter Ab is attached to the detectable label by the heavy chain constant region of the reporter Ab.

In embodiments wherein the reporter Ab is used together with a pre-reporter Ab in the methods of the present invention, the reporter Ab is capable of binding to the heavy chain constant region of the pre-reporter Ab with the variable region of the reporter Ab. In a third aspect, when applied together with a pre-reporter Ab, the reporter Ab is capable of binding to the heavy chain constant region of the pre-reporter antibody with the variable region of the reporter antibody. The reporter Ab is attached to a detectable label, preferably with the heavy chain constant region of the reporter antibody.

According to one embodiment of the invention the detectable label to which the reporter antibody is attached to is a compound or moiety that comprises one or more appropriate chemical substances or enzymes, which directly or indirectly generate a detectable compound or signal in a chemical, physical or enzymatic reaction. Labeling can be achieved by methods well known in the art (see, for example, Lottspeich, F., and Zorbas H., Springer Spektrum 2012, Bioanalytik).

According to one embodiment of the invention the detectable label is selected from the group consisting of fluorescent labels, magnetic labels, enzyme labels, colored labels, chromogenic labels, luminescent labels, radioactive labels, haptens, biotin, metal complexes, metals, and colloidal gold. All these types of labels are well established in the art.

According to one embodiment of the invention the label is selected from such which provide the emission of fluorescence or phosphorescence upon irradiation or excitation or the emission of X-rays when using a radioactive label.

According to one embodiment of the invention the label is an enzyme label, which include but are not limited to alkaline phosphatase, horseradish peroxidase (HRP), β-galactosidase, and β-lactamase. Enzyme labels catalyze the formation of chromogenic reaction products.

In specific embodiments the detectable labels are fluorescent labels. Numerous fluorescent labels are well established in the art and commercially available from different suppliers (see, for example, The Handbook—A Guide to Fluorescent Probes and Labeling Technologies, 10th ed. (2006), Molecular Probes, Invitrogen Corporation, Carlsbad, CA, USA). Examples of fluorescent labels include but are not limited to xanthene, fluorescein isothiocyanate, rhodamine, phycoerythrin (PE), cyanine, coumarin, and any derivative thereof.

According to preferred embodiments of the invention, the fluorescent label is PE.

Concerning the detection of such labels with suitable detection systems, reference is also made to the section “Detection system”.

In the embodiments wherein the detectable label to which the reporter Ab is attached to is a fluorescent label the fluorescent label can be irradiated/excited with any suitable light source present within a detection system. The light source may be a laser or a LED. In the case the fluorescent label is PE, the excitation wavelength of the light source is in the range of about 505 to about 535 nm, for instance about 511 nm.

### Detection System

According to the invention the detection system refers to any system which is suitable for determining values indicative for the presence and/or amount of reporter antibody.

According to the invention the detection system may also be able to determine values indicative for the presence and/or amount of a specific microsphere.

The selection of a suitable detection system depends on several parameters such as the type of detectable labels used for detection or the kind of analysis performed. Various optical and non-optical detection systems are well established in the art. A general description of detection systems that can be used with the method can be found, e.g., in Lottspeich, F., and Zorbas H., Springer Spektrum 2012, Bioanalytik.

According to one embodiment of the invention, the detection system is an optical detection system. In some embodiments, performing the method involves simple detection systems, which may be based on the measurement of parameters such as fluorescence, optical absorption, resonance transfer, and the like.

According to one embodiment of the invention the detection system measures fluorescence. Such systems measure the capacity of particular molecules to emit their own light when excited by light of a particular wavelength resulting in a characteristic absorption and emission behavior. In particular, quantitative detection of fluorescence signals is performed by means of modified methods of fluorescence microscopy (for review see, e.g., Lichtman, J. W., and Conchello, J. A. (2005) Nature Methods 2, 910-919; Zimmermann, T. (2005) Adv. Biochem. Eng. Biotechnol. 95, 245-265). Thereby, the signals resulting from light absorption and light emission, respectively, are separated by one or more filters and/or dichroites and imaged on suitable detectors. Data analysis is performed by means of digital image processing. Image processing may be achieved with several software packages well known in the art (such as Mathematica Digital Image Processing, EIKONA, or Image-PRO). Another suitable software for such purposes is the Iconoclust software (Clondiag Chip Technologies GmbH, Jena, Germany). Suitable detection systems may be based on “classical” methods for measuring a fluorescent signal such as epifluorescence or darkfield fluorescence microscopy (reviewed, e.g., in: Lakowicz, J. R. (1999) Principles of Fluorescence Spectroscopy, 2nd ed., Plenum Publishing Corp., NY). Another optical detection system that may be used is confocal fluorescence microscopy, wherein the object is illuminated in the focal plane of the lens by a point light source. Importantly, the point light source, object and point light detector are located on optically conjugated planes. Examples of such confocal systems are described in detail, for example, in Diaspro, A. (2002) Confocal and 2-photon-microscopy: Foundations, Applications and Advances, Wiley-Liss, Hobroken, NJ. The fluorescence-optical system is usually a fluorescence microscope without an autofocus, for example a fluorescence microscope having a fixed focus. Further fluorescence detection methods that may also be used include inter alia total internal fluorescence microscopy (see, e.g., Axelrod, D. (1999) Surface fluorescence microscopy with evanescent illumination, in: Lacey, A. (ed.) Light Microscopy in Biology, Oxford University Press, New York, 399-423), fluorescence lifetime imaging microscopy (see, for example, Dowling, K. et al. (1999) J. Mod. Optics 46, 199-209), fluorescence resonance energy transfer (FRET; see, for example, Periasamy, A. (2001) J. Biomed. Optics 6, 287-291), bioluminescence resonance energy transfer (BRET; see, e.g., Wilson, T., and Hastings, J. W. (1998) Annu. Rev. Cell Dev. Biol. 14, 197-230), and fluorescence correlation spectroscopy (see, e.g., Hess, S. T. et al. (2002) Biochemistry 41, 697-705). In specific embodiments, detection is performed using FRET or BRET, which are based on the respective formation of fluorescence or bioluminescence quencher pairs. The use of FRET is also described, e.g., in Liu, B. et al. (2005) Proc. Natl. Acad. Sci. USA 102, 589-593; and Szollosi, J. et al. (2002) J. Biotechnol. 82, 251-266. The use of BRET is detailed, for example, in Prinz, A. et al. (2006) Chembiochem. 7, 1007-1012; and Xu, Y. et al. (1999) Proc. Natl. Acad. Sci. USA 96, 151-156.

In one embodiment the detection system comprises a first light source, e.g. an argon laser or a light emitting diode (LED), which has an excitation wavelength in the range of about 400 to about 700 nm and a second light source, e.g. an argon laser or a LED, which has an excitation wavelength in the range of about 300 to about 700 nm and a suitable detection component as for instance a photodiode such as an avalanche photodiode (APD) in combination with a photomultiplier or a charge-coupled device (CCD) sensor. The first light source may be used for the identification of the detectable label of a microsphere, wherein the detectable label may be one or more fluorescent dyes at a specific concentration. The second light source may be used for excitation of the detectable label of a reporter antibody.

In a preferred embodiment the first light source, e.g. the argon laser or LED has an excitation wavelength in the range of about 600 to about 650 nm and the second light source, e.g. the argon laser or LED has an excitation wavelength in the range of about 500 to about 600 nm. In a more preferred embodiment the first light source, e.g. the argon laser or LED has an excitation wavelength in the range of about 615 to about 640 nm and the second light source, e.g. the argon laser or LED has an excitation wavelength in the range of about 505 to about 540 nm. In an even more preferred embodiment the first light source, e.g. the argon laser or LED has an excitation wavelength in the range of about 620 to about 635 nm and the second light source, e.g. the argon laser or LED has an excitation wavelength in the range of about 510 to about 535 nm. For instance, the detection system comprises a first light source, e.g. an argon laser or a LED, which has an excitation wavelength of about 635 nm and a second light source, e.g. an argon laser or a LED, which has an excitation wavelength of about 525 nm.

The detection system may be also capable of distinguishing the individual size or shape of a microsphere from the individual size or shape of another microsphere, thereby allowing individual identification of the microsphere.

The detection system may be one of the group consisting of MAGPIX®, Luminex 200®, and FLEXMAP 3D® (Luminex Corp. Austin, Tex.). In a preferred embodiment the detection system is the MAGPIX® (Luminex Corp. Austin, Tex.).

The detection system may be operated by a specific software, including the xPONENT® software (Luminex Corp. Austin, Tex.).

The detection system may be capable of detecting both, the signal from the detectable label of the reporter Ab, as well as the signal from the detectable label of the microsphere.

The detection system may be capable of analyzing one microsphere after the other thereby identifying the microsphere by detecting the signal from the detectable label of the microsphere and detecting the signal from the detectable label of the reporter antibody such as flow cytometry based detection systems (e.g. Luminex 200° and FLEXMAP 3D®). The flow cytometry based detection systems Luminex 200° and FLEXMAP 3D® include two lasers each one for irradiation of the detectable label of the microsphere and the detectable label of the reporter Ab. As flow cytometry based detection systems are not capturing the microspheres with a magnet, the Luminex 200° and FLEXMAP 3D® systems are compatible with both, magnetic microspheres such as the MagPlex® microspheres and non-magnetic microspheres such as the Microplex® microspheres. The Luminex 200° and FLEXMAP 3D® systems detect signals from the microspheres and reporter Abs by avalanche photodiodes (APD) in combination with photomultipliers (PMT).

Alternatively, the detection system may be capable of analyzing multiple microspheres at once. Therefore, a monolayer of magnetic microspheres is captured by a magnet and the microspheres are excited with two LEDs, one LED for excitement of the detectable label of the microsphere and the other LED for excitement of the detectable label of the reporter Ab. The signals from the microspheres and reporter Abs are recorded by a CCD imager, which allows identification of each microsphere and the corresponding antigen to which the microsphere is coupled to. An example for a LED-based detection system is the MAGPIX® instrument. As analyses with the MAGPIX® instrument involves capture of the microspheres with a magnet, the MAGPIX® instrument is solely compatible with magnetic microspheres such as MagPlex® microspheres.

### Methods for Determining Flavivirus-Reactive Complement-Fixing Antibodies

The present invention is directed to methods for determining flavivirus-reactive complement-fixing Abs. Concerning the microsphere complex, the reporter and pre-reporter Ab, as well as the detection system reference is made to the sections above termed “Microsphere complex”, “Reporter and Pre-reporter Ab”, and “Detection system”. In addition, certain specific embodiments are also outlined in this section and shall, however, not be taken as limiting.

In one aspect the method for determining the presence and/or amount of flavivirus-reactive complement-fixing antibodies in a sample from a subject according to the present invention comprises the steps of:
Step 1: contacting an amount of a microsphere complex comprising a microsphere coupled to a flavivirus antigen with the sample to allow binding of the flavivirus-reactive complement-fixing antibodies in the sample to the flavivirus antigen;
Step 2: contacting an amount of complement component 1q (C1q) with the complement-fixing antibodies bound to the flavivirus antigen in step 1 to allow binding of the C1q to the heavy chain constant region of the complement-fixing antibodies;
Step 3: contacting an amount of a reporter antibody with the C1q bound to the complement-fixing antibodies in step 2 to allow binding of the reporter antibody to the C1q, wherein the reporter antibody binds to the C1q with the variable region of the reporter antibody and wherein the reporter antibody is attached to a detectable label; and
Step 4: detecting a signal from the reporter antibody bound to the C1q in step 3, wherein the signal is indicative for the presence and/or amount of the reporter antibody and wherein the presence and/or amount of the reporter antibody is indicative for the presence and/or amount of flavivirus-reactive complement-fixing antibodies in the sample.

In one embodiment of the first aspect, the method for determining the presence and/or amount of flavivirus-reactive complement-fixing antibodies in a sample from a subject comprises the steps of:
Step 1: contacting an amount of a microsphere complex comprising a microsphere coupled to a flavivirus antigen with the sample to allow binding of the flavivirus-reactive complement-fixing antibodies in the sample to the flavivirus antigen;
Step 2: contacting an amount of C1q with the complement-fixing antibodies bound to the flavivirus antigen in step 1 to allow binding of the C1q to the heavy chain constant region of the complement-fixing antibodies;
Step 3.1: contacting an amount of a pre-reporter antibody with the C1q bound to the complement-fixing antibodies in step 2 to allow binding of the pre-reporter antibody to the C1q, wherein the pre-reporter antibody binds to the C1q with the variable region of the pre-reporter antibody;
Step 3.2: contacting an amount of a reporter antibody with the pre-reporter antibody bound to the C1q in step 3.1 to allow binding of the reporter antibody to the pre-reporter antibody, wherein the reporter antibody binds to the heavy chain constant region of the pre-reporter antibody with the variable region of the reporter antibody and wherein the reporter antibody is attached to a detectable label; and
Step 4: detecting a signal from the reporter antibody bound to the pre-reporter antibody in step 3.2, wherein the signal is indicative for the presence and/or amount of the reporter antibody and wherein the presence and/or amount of the reporter antibody is indicative for the presence and/or amount of flavivirus-reactive complement-fixing antibodies in the sample.

According to one embodiment of the present invention, the method for determining the presence and/or amount of flavivirus-reactive complement-fixing antibodies in a sample from a subject comprises the further steps of:
Step 5: determining the presence and/or amount of the reporter antibody from the signal of step 4; and
Step 6: determining the presence and/or amount of flavivirus-reactive complement-fixing antibodies in the sample from the presence and/or amount of the reporter antibody determined in step 5.

In a second aspect, the present invention provides a method for determining the presence and/or amount of flavivirus-reactive complement-fixing antibodies in a sample from a subject comprising the steps of:
Step 1: contacting an amount of a microsphere complex comprising a microsphere coupled to a flavivirus antigen with the sample to allow binding of the flavivirus-reactive complement-fixing antibodies in the sample to the flavivirus antigen;
Step 2: contacting an amount of a complement competent serum with the complement-fixing antibodies bound to the flavivirus antigen in step 1 to allow the formation of complement component fragment C3d (C3d);
Step 3: contacting an amount of a reporter antibody with the C3d formed in step 2 to allow binding of the reporter antibody to the C3d, wherein the reporter antibody binds to the C3d with the variable region of the reporter antibody and wherein the reporter antibody is attached to a detectable label; and
Step 4: detecting a signal from the reporter antibody bound to the C3d in step 3, wherein the signal is indicative for the presence and/or amount of the reporter antibody and wherein the presence and/or amount of the reporter antibody is indicative for the presence and/or amount of flavivirus-reactive complement-fixing antibodies in the sample.

In one embodiment of the second aspect the method comprises the steps of:
Step 1: contacting an amount of a microsphere complex comprising a microsphere coupled to a flavivirus antigen with the sample to allow binding of the flavivirus-reactive complement-fixing antibodies in the sample to the flavivirus antigen;
Step 2: contacting an amount of a complement competent serum with the complement-fixing antibodies bound to the flavivirus antigen in step 1 to allow the formation of C3d;
Step 3.1: contacting an amount of a pre-reporter antibody with the C3d formed in step 2 to allow binding of the pre-reporter antibody to the C3d, wherein the pre-reporter antibody binds to the C3d with the variable region of the pre-reporter antibody;
Step 3.2: contacting an amount of a reporter antibody with the pre-reporter antibody bound to the C3d in step 3.1 to allow binding of the reporter antibody to the pre-reporter antibody, wherein the reporter antibody binds to the heavy chain constant region of the pre-reporter antibody with the variable region of the reporter antibody and wherein the reporter antibody is attached to a detectable label; and
Step 4: detecting a signal from the reporter antibody bound to the pre-reporter antibody in step 3.2, wherein the signal is indicative for the presence and/or amount of the reporter antibody and wherein the presence and/or amount of the reporter antibody is indicative for the presence and/or amount of flavivirus-reactive complement-fixing antibodies in the sample.

In a more preferred embodiment the method comprises the further steps of:
Step 5: determining the presence and/or amount of the reporter antibody from the signal of step 4; and
Step 6: determining the presence and/or amount of flavivirus-reactive complement-fixing antibodies in the sample from the presence and/or amount of the reporter antibody determined in step 5.

According to one embodiment contacting in step 1 is carried out for about 30 to 90 minutes. In specific embodiments contacting in step 1 is carried out for about 60 minutes.

According to one embodiment contacting in step 2 is carried out for about 10 to 50 minutes. In specific embodiments contacting in step 2 is carried out for about 30 minutes.

According to one embodiment contacting in step 3.1 is carried out for about 10 to 50 minutes. In specific embodiments contacting in step 3.1 is carried out for about 30 minutes.

According to one embodiment contacting in step 3.2 is carried out for about 10 to 50 minutes. In specific embodiments contacting in step 3.2 is carried out for about 30 minutes.

In one specific embodiment contacting in step 1 is carried out for about 30 to 90 minutes, contacting in step 2 is carried out for about 10 to 50 minutes, contacting in step 3.1 is carried out for about 10 to 50 minutes, and contacting in step 3.2 is carried out for about 10 to 50 minutes.

In a more specific embodiment contacting in step 1 is carried out for about 60 minutes, contacting in step 2 is carried out for about 30 minutes, contacting in step 3.1 is carried out for about 30 minutes, and contacting in step 3.2 is carried out for about 30 minutes.

In one embodiment of the present invention, the detectable label to which the reporter antibody is attached to is a fluorescence label selected from the group consisting of xanthene, fluorescein isothiocyanate, rhodamine, phycoerythrin, cyanine, coumarin, and any derivative thereof. In a preferred embodiment the detectable label is phycoerythrin.

In one embodiment the flavivirus is selected from the group consisting of dengue virus, zika virus, West Nile virus, Japanese encephalitis virus, Tick-Borne encephalitis virus, Yellow Fever virus, Murray Valley encephalitis virus, and St. Louis encephalitis virus.

In other embodiments the antigen is selected from the group consisting of virus like particle (VLP), non-structural protein 1, envelope protein, pre-membrane protein, membrane protein, capsid protein, non-structural protein 2A, non-structural protein 2B, non-structural protein 3, non-structural protein 4A, non-structural protein 4B, and non-structural protein 5 and any derivative thereof.

In specific embodiments the flavivirus antigen is a DENV VLP. The DENV VLP can be of any serotype (DENV1-4). In other specific embodiments the flavivirus antigen is DENV NS1. The DENV NS1 can be of any serotype (DENV1-4).

In other specific embodiments the flavivirus antigen is a ZIKV VLP. In other specific embodiments the flavivirus antigen is ZIKV NS1.

In certain embodiments, the C1q is present within a complement-component serum, which may be human complement-component serum. In certain embodiments, the C1q is purified from plasma. Purification can be carried out by any protein purification method known in the art, such as filtration, centrifugation, chromatographic separation, or buffer exchange. In other embodiments, the C1q is of human origin. In specific embodiments the C1q is purified human C1q. In general, the origin of the C1q is dependent on the origin of complement-fixing Abs in the sample. For instance, for detection of human complement-fixing Abs, human C1q is suitable.

In certain embodiments, the complement competent serum is a commercially obtainable complement competent serum. Alternatively, a human serum may be obtained and used.

In certain embodiments, the sample is heat-inactivated. Heat-inactivation can be carried out at about 56° C. for about 30 minutes. In general, heat-inactivation can be varied according to the type of sample to be analyzed.

In other embodiments the sample is a blood sample, in particular a plasma or serum sample. In general, the method can be carried out with either matrices i.e. serum or plasma. Preferably, the sample was obtained from an individual immunized with a Dengue vaccine. Alternatively, the sample may have been obtained from a patient suffering from Dengue disease. The sample may have been obtained from the patient suffering from Dengue disease at any time after infection, such as for instance, 1 or 2 weeks after infection.

In other embodiments the sample is a urine sample.

In specific embodiments, the sample is a heat-inactivated serum or plasma sample.

In one embodiment the subject is selected from the group consisting of mouse, primate, non-human primate, human, rabbit, cat, rat, horse, and sheep. In preferred embodiments the subject is a non-human primate. In more preferred embodiments the subject is human.

In other embodiments the subject is seronegative for the flavivirus. In other embodiments the subject is seropositive for the flavivirus. In specific embodiments the subject is seronegative for DENY. In other specific embodiments the subject is seropositive for DENY. In other specific embodiments the subject is seronegative for ZIKV. In other specific embodiments the subject is seropositive for ZIKV.

In some embodiments of the present invention the subject is vaccinated with a flavivirus vaccine.

In one embodiment of the present invention, the signal from the reporter antibody detected in step 4 is resulting from the detectable label to which the reporter antibody is attached. In a specific embodiment, the signal in step 4 is a fluorescence signal. In even more specific embodiments, the signal in step 4 is a fluorescence signal resulting from phycoerythrin. The signal in step 4 can be detected upon irradiation with a light source as present in any suitable detection system.

Within the meaning of the invention, in embodiments of the first aspect of the present invention wherein no pre-reporter Ab is applied, the signal from the reporter antibody in step 4 is resulting from a reporter antibody which is bound to C1q, wherein C1q is bound to the complement-fixing Abs bound to the flavivirus antigen coupled to microspheres in a microsphere complex.

Within the meaning of the invention, in embodiments of the first aspect of the present invention wherein a pre-reporter Ab is applied, the signal from the reporter antibody in step 4 is resulting from a reporter antibody which is bound to the pre-reporter Ab, wherein the pre-reporter Ab is bound to C1q, wherein C1q is bound to the complement-fixing Abs bound to the flavivirus antigen coupled to microspheres in a microsphere complex.

Within the meaning of the invention, in embodiments of the second aspect of the present invention wherein no pre-reporter Ab is applied, the signal from the reporter antibody in step 4 is resulting from a reporter antibody which is bound to the formed C3d. Within the meaning of the invention, in embodiments of the first aspect of the present invention wherein a pre-reporter Ab is applied, the signal from the reporter antibody in step 4 is resulting from a reporter antibody which is bound to the pre-reporter Ab, wherein the pre-reporter Ab is bound to the formed C3d. In specific embodiments of the present invention the presence and/or amount of reporter Ab in step 5 is determined by comparing the signal of step 4 to a standard curve, wherein the standard curve comprises signals resulting from known amounts of reporter Ab.

In specific embodiments of the invention the presence and/or amount of flavivirus-reactive complement-fixing Abs in the sample in step 6 is determined based on the amount of the reporter Ab determined in step 5 as the amount of reporter Ab is in direct proportion to the complement-fixing Abs present within a sample.

### Methods for the Concomitant Determination of Complement-Fixing Antibodies Reactive to Different Flaviviruses

The present invention is further directed to methods for the concomitant determination complement-fixing Abs reactive to different flaviviruses. Concerning the microsphere complex, the reporter and pre-reporter Ab, as well as the detection system reference is made to the sections above termed “Microsphere complex”, “Reporter and Pre-reporter Ab”, and “Detection system”. In addition, certain specific embodiments are also outlined in this section and shall, however, not be taken as limiting.

In one aspect of the present invention the method for the concomitant determination of the presence and/or amount of complement-fixing antibodies reactive to different flaviviruses in a sample from a subject comprises the steps of:
Step 1: contacting an amount of at least two microsphere complexes with the sample,
   wherein the first microsphere complex comprises a first microsphere coupled to a first flavivirus antigen and the second microsphere complex comprises a second microsphere coupled to a second flavivirus antigen
to allow binding of the complement-fixing antibodies reactive to the first and/or the second flavivirus to the first flavivirus antigen and/or the second flavivirus antigen,
   wherein the first microsphere comprises a first detectable label and the second microsphere comprises a second detectable label, and wherein the emission signal of the first detectable label differs from the emission signal of the second detectable label,
Step 2: contacting an amount of C1q with the complement-fixing antibodies bound to the first and/or the second flavivirus antigen in step 1 to allow binding of the C1q to the heavy chain constant region of the complement-fixing antibodies,
Step 3: contacting an amount of a reporter antibody with the C1q bound to the complement-fixing antibodies in step 2 to allow binding of the reporter antibody to the C1q, wherein the reporter antibody binds to the C1q with the variable region of the reporter antibody and wherein the reporter antibody is attached to a third detectable label,
Step 4: detecting the emission signal of the detectable label of at least one microsphere upon irradiation with a first light source and comparing the emission signal with the emission signal of the first detectable label and with the emission signal of the second detectable label thereby identifying the at least one microsphere and the flavivirus antigen the at least one microsphere is coupled to, and
simultaneously detecting a signal from the reporter antibody bound to the C1q in step 3 of the at least one microsphere upon irradiation with a second light source,
Step 5: repeating step 4 until at least 30 microspheres coupled to the same flavivirus antigen are identified, and
Step 6: summarizing the detected signal from the reporter antibody in step 4 for all identified microspheres coupled to the same flavivirus antigen, wherein the summarized signal is indicative for the presence and/or amount of the reporter antibody and wherein the presence and/or amount of the reporter antibody is indicative for the presence and/or amount of flavivirus-reactive complement-fixing antibodies in the sample.

In certain embodiments the method for the concomitant determination of the presence and/or amount of complement-fixing antibodies reactive to different flaviviruses in a sample from a subject comprises the steps of:
Step 1: contacting an amount of at least two microsphere complexes with the sample,
   wherein the first microsphere complex comprises a first microsphere coupled to a first flavivirus antigen and the second microsphere complex comprises a second microsphere coupled to a second flavivirus antigen
to allow binding of the complement-fixing antibodies reactive to the first and/or the second flavivirus to the first flavivirus antigen and/or the second flavivirus antigen,
   wherein the first microsphere comprises a first detectable label and the second microsphere comprises a second detectable label, and wherein the emission signal of the first detectable label differs from the emission signal of the second detectable label,
Step 2: contacting an amount of C1q with the complement-fixing antibodies bound to the first and/or the second flavivirus antigen in step 1 to allow binding of the C1q to the heavy chain constant region of the complement-fixing antibodies,
Step 3.1: contacting an amount of a pre-reporter antibody with the C1q bound to the complement-fixing antibodies in step 2 to allow binding of the pre-reporter antibody to the C1q, wherein the pre-reporter antibody binds to the C1q with the variable region of the pre-reporter antibody,
Step 3.2: contacting an amount of a reporter antibody with the pre-reporter antibody bound to the C1q in step 3.1 to allow binding of the reporter antibody to the pre-reporter antibody, wherein the reporter antibody binds to the heavy chain constant region of the pre-reporter antibody with the variable region of the reporter antibody and wherein the reporter antibody is attached to a third detectable label,
Step 4: detecting the emission signal of the detectable label of at least one microsphere upon irradiation with a first light source and comparing the emission signal with the emission signal of the first detectable label and with the emission signal of the second detectable label thereby identifying the at least one microsphere and the flavivirus antigen the at least one microsphere is coupled to, and
simultaneously detecting a signal from the reporter antibody bound to the C1q in step 3 of the at least one microsphere upon irradiation with a second light source,
Step 5: repeating step 4 until at least 30 microspheres coupled to the same flavivirus antigen are identified, and
Step 6: summarizing the detected signal from the reporter antibody in step 4 for all identified microspheres coupled to the same flavivirus antigen, wherein the summarized signal is indicative for the presence and/or amount of the reporter antibody and wherein the presence and/or amount of the reporter antibody is indicative for the presence and/or amount of flavivirus-reactive complement-fixing antibodies in the sample.

In some embodiments the method for the concomitant determination of the presence and/or amount of complement-fixing antibodies reactive to different flaviviruses in a sample from a subject comprises the further steps of:
Step 7: determining the presence and/or amount of reporter antibody from the summarized signal in step 6, and
Step 8: determining the presence and/or amount of flavivirus-reactive complement-fixing antibodies in the sample from the presence and/or amount of reporter antibody determined in step 7.

In a second aspect of the present invention, the method for the concomitant determination of the presence and/or amount of complement-fixing antibodies reactive to different flaviviruses in a sample from a subject comprises the steps of:
Step 1: contacting an amount of at least two microsphere complexes with the sample,
   wherein the first microsphere complex comprises a first microsphere coupled to a first flavivirus antigen and the second microsphere complex comprises a second microsphere coupled to a second flavivirus antigen
to allow binding of the complement-fixing antibodies reactive to the first and/or the second flavivirus to the first flavivirus antigen and/or the second flavivirus antigen,
   wherein the first microsphere comprises a first detectable label and the second microsphere comprises a second detectable label, and wherein the emission signal of the first detectable label differs from the emission signal of the second detectable label,
Step 2: contacting an amount of a complement competent serum with the complement-fixing antibodies bound to the flavivirus antigen in step 1 to allow the formation of C3d,
Step 3: contacting an amount of a reporter antibody with the C3d formed in step 2 to allow binding of the reporter antibody to the C3d, wherein the reporter antibody binds to the C3d with the variable region of the reporter antibody and wherein the reporter antibody is attached to a third detectable label,
Step 4: detecting the emission signal of the detectable label of at least one microsphere upon irradiation with a first light source and comparing the emission signal with the emission signal of the first detectable label and with the emission signal of the second detectable label thereby identifying the at least one microsphere and the flavivirus antigen the at least one microsphere is coupled to, and
simultaneously detecting a signal from the reporter antibody bound to the C3d in step 3 of the at least one microsphere upon irradiation with a second light source,
Step 5: repeating step 4 until at least 30 microspheres coupled to the same flavivirus antigen are identified, and
Step 6: summarizing the detected signal from the reporter antibody in step 4 for all identified microspheres coupled to the same flavivirus antigen, wherein the summarized signal is indicative for the presence and/or amount of the reporter antibody and wherein the presence and/or amount of the reporter antibody is indicative for the presence and/or amount of flavivirus-reactive complement-fixing antibodies in the sample.

In one embodiment of this aspect the method comprises the steps of:
Step 1: contacting an amount of at least two microsphere complexes with the sample,
   wherein the first microsphere complex comprises a first microsphere coupled to a first flavivirus antigen and the second microsphere complex comprises a second microsphere coupled to a second flavivirus antigen
to allow binding of the complement-fixing antibodies reactive to the first and/or the second flavivirus to the first flavivirus antigen and/or the second flavivirus antigen,
   wherein the first microsphere comprises a first detectable label and the second microsphere comprises a second detectable label, and wherein the emission signal of the first detectable label differs from the emission signal of the second detectable label,
Step 2: contacting an amount of a complement competent serum with the complement-fixing antibodies bound to the first and/or the second flavivirus antigen in step 1 to allow the formation of C3d,
Step 3.1: contacting an amount of a pre-reporter antibody with the C3d formed in step 2 to allow the formation of C3d and to allow binding of the pre-reporter antibody to the C3d, wherein the pre-reporter antibody binds to the C3d with the variable region of the pre-reporter antibody,
Step 3.2: contacting an amount of a reporter antibody with the pre-reporter antibody bound to the C3d in step 3.1 to allow binding of the reporter antibody to the pre-reporter antibody, wherein the reporter antibody binds to the heavy chain constant region of the pre-reporter antibody with the variable region of the reporter antibody and wherein the reporter antibody is attached to a third detectable label,
Step 4: detecting the emission signal of the detectable label of at least one microsphere upon irradiation with a first light source and comparing the emission signal with the emission signal of the first detectable label and with the emission signal of the second detectable label thereby identifying the at least one microsphere and the flavivirus antigen the at least one microsphere is coupled to, and simultaneously detecting a signal from the reporter antibody bound to the C3d in step 3 of the at least one microsphere upon irradiation with a second light source,
Step 5: repeating step 4 until at least 30 microspheres coupled to the same flavivirus antigen are identified, and
Step 6: summarizing the detected signal from the reporter antibody in step 4 for all identified microspheres coupled to the same flavivirus antigen, wherein the summarized signal is indicative for the presence and/or amount of the reporter antibody and wherein the presence and/or amount of the reporter antibody is indicative for the presence and/or amount of flavivirus-reactive complement-fixing antibodies in the sample.

In one preferred embodiment of this aspect the method further comprises the steps of:
Step 7: determining the presence and/or amount of the reporter antibody from the summarized signal in step 6, and
Step 8: determining the presence and/or amount of flavivirus-reactive complement-fixing antibodies in the sample from the presence and/or amount of the reporter antibody determined in step 7.

In certain embodiments the first detectable label is at least one fluorescent dye. In other embodiments the second detectable label is at least one fluorescent dye. In other embodiments the first and the second detectable labels are each at least one fluorescent dye. Within the meaning of the invention the first and the second detectable label are different labels. In preferred embodiments the at least one fluorescent dye is selected from the group consisting of squaraine, phthalocyanine, naphthalocyanine, and any derivative thereof. For instance, the first microsphere comprises squarine, the second microsphere comprises phthalocyanine.

In certain embodiments the emission signal of the detectable label of the at least one microsphere detected under step 4 upon irradiation with a first light source corresponds to the emission signal of the first detectable label.

In certain embodiments the emission signal of the detectable label of the at least one microsphere detected under step 4 upon irradiation with a first light source corresponds to the emission signal of the second detectable label.

In specific embodiments the first light source is a laser or a LED. The wavelength of the first light source may be in the range of about 400 to about 700 nm. In a preferred embodiment the wavelength of the first light source may be in the range of about 600 to about 650 nm. In a more preferred embodiment the wavelength of the first light source may be in the range of about 615 to about 640 nm. In an even more preferred embodiment the wavelength of the first light source may be in the range of about 620 to about 635 nm. For instance, the wavelength of the first light source is about 635 nm.

In certain embodiments the third detectable label is a fluorescence label selected from the group consisting of xanthene, fluorescein isothiocyanate, rhodamine, phycoerythrin, cyanine, coumarin, and any derivative thereof. In preferred embodiments the fluorescence label is phycoerythrin.

In one embodiment of the present invention, the signal from the reporter antibody detected in step 4 is resulting from the third detectable label to which the reporter antibody is attached. In a specific embodiment, the signal in step 4 is a fluorescence signal. In even more specific embodiments, the signal in step 4 is a fluorescence signal resulting from phycoerythrin.

The signal from the reporter antibody in step 4 can be detected upon irradiation with a second light source. In specific embodiments the second light source is a laser or a LED. The wavelength of the second light source may be in the range of about 300 to about 700 nm. In a preferred embodiment the wavelength of the first light source may be in the range of about 500 to about 600 nm. In a more preferred embodiment the wavelength of the first light source may be in the range of about 505 to about 540 nm. In an even more preferred embodiment the wavelength of the first light source may be in the range of about 510 to about 535 nm. For instance, the wavelength of the first light source is about 525 nm.

Detection of the signal from the reporter antibody and recording of the emission signal of the detectable label of at least one microsphere is carried out simultaneously, i.e. in one analysis step. Therefore, the at least one microsphere is irradiated with the first light source followed by detecting the emission signal of the detectable label of the at least one microsphere in a first step, directly followed by irradiation of the at least one microsphere with the second light source followed by detecting the signal from the reporter antibody in a second step. It is also possible, that the at least one microsphere is irradiated with the second light source followed by detecting the signal from the reporter antibody in a first step, directly followed by irradiation of the at least one microsphere with the first light source followed by detecting the emission signal of the detectable label of the at least one microsphere in a second step.

Within the meaning of the invention, in embodiments of the first aspect of the present invention, wherein no pre-reporter Ab is applied, the signal from the reporter antibody in step 4 is resulting from a reporter antibody which is bound to C1q, wherein C1q is bound to the complement-fixing Abs bound to the flavivirus antigen coupled to microspheres in a microsphere complex.

Within the meaning of the invention, in embodiments of the first aspect of the present invention wherein a pre-reporter Ab is applied, the signal from the reporter antibody in step 4 is resulting from a reporter antibody which is bound to the pre-reporter Ab, wherein the pre-reporter Ab is bound to C1q, wherein C1q is bound to the complement-fixing Abs bound to the flavivirus antigen coupled to microspheres in a microsphere complex.

Within the meaning of the invention, in embodiments of the second aspect of the present invention wherein no pre-reporter Ab is applied, the signal from the reporter antibody in step 4 is resulting from a reporter antibody which is bound to the formed C3d. Within the meaning of the invention, in embodiments of the first aspect of the present invention wherein a pre-reporter Ab is applied, the signal from the reporter antibody in step 4 is resulting from a reporter antibody which is bound to the pre-reporter Ab, wherein the pre-reporter Ab is bound to the formed C3d.

In one embodiment step 4 is repeated until at least 30 microspheres coupled to the same flavivirus antigen are identified. In another embodiment step 4 is repeated until at least 40 microspheres coupled to the same flavivirus antigen are identified. In another embodiment step 4 is repeated until at least 50 microspheres coupled to the same flavivirus antigen are identified. In another embodiment step 4 is repeated until at least 60 microspheres coupled to the same flavivirus antigen are identified. In another embodiment step 4 is repeated until at least 80 microspheres coupled to the same flavivirus antigen are identified. In another embodiment step 4 is repeated until at least 100 microspheres coupled to the same flavivirus antigen are identified.

For instance, if one microsphere is coupled to a DENV1 VLP and another microsphere is coupled to a DENV2 VLP, step 4 is repeated until at least 30, or at least 40, or at least 50, or at least 60, or at least 80, or at least 100 microspheres that are coupled to DENV1 VLP and until at least 30, or at least 40, or at least 50, or at least 60, or at least 80, or at least 100 microspheres that are coupled to DENV2 VLP are identified.

Within the meaning of step 6, summarizing the detected signal from the reporter antibody in step 4 for all identified microspheres coupled to the same flavivirus antigen refers to the addition of the detected signals for all single microspheres coupled to the same flavivirus antigen.

The summarized signal from the reporter antibody of step 5 for the number of microspheres coupled to the same flavivirus antigen identified under step 4 is representative for all microspheres coupled to the same flavivirus antigen in the experiment.

In certain embodiments, the flaviviruses are selected from the group consisting of dengue virus, zika virus, West Nile virus, Japanese encephalitis virus, Tick-Borne encephalitis virus, Yellow Fever virus, Murray Valley encephalitis virus, and St. Louis encephalitis virus.

In certain embodiments, the antigens are selected from the group consisting of virus like particle (VLP), non-structural protein 1, envelope protein, pre-membrane protein, membrane protein, capsid protein, non-structural protein 2A, non-structural protein 2B, non-structural protein 3, non-structural protein 4A, non-structural protein 4B, and non-structural protein 5 and any derivative thereof.

Within the meaning of the invention the first flavivirus antigen and the second flavivirus antigen are different. For instance, the first flavivirus antigen is a DENV VLP and the second flavivirus antigen is a ZIKV VLP. Another example would be that the first flavivirus antigen is a DENV VLP and the second flavivirus antigen is a DENV NS1. Another example would be that the first flavivirus antigen is a DENV serotype 1 VLP and the second flavivirus antigen is a DENV serotype 2 VLP.

The method of the present invention provides the possibility to contact any number of microsphere complexes with the sample in step 1, wherein each microsphere complex comprises a microsphere coupled to a different flavivirus antigen and wherein each microsphere complex comprises a microsphere with a different detectable label resulting in a different emission signal e.g. a different emission intensity upon irradiation thereby providing the possibility to identify the different microspheres and thereby the different antigens.

In a specific embodiment, in step 1, an amount of a first microsphere complex comprising a first microsphere coupled to a dengue 1 virus VLP, an amount of a second microsphere complex comprising a second microsphere coupled to a dengue 2 virus VLP, an amount of a third microsphere complex comprising a third microsphere coupled to a dengue 3 virus VLP, and an amount of a fourth microsphere complex comprising a fourth microsphere coupled to a dengue 4 virus VLP is contacted with the sample.

In other specific embodiments, in step 1, an amount of a first microsphere complex comprising a first microsphere coupled to a dengue 1 virus VLP, an amount of a second microsphere complex comprising a second microsphere coupled to a dengue 2 virus VLP, an amount of a third microsphere complex comprising a third microsphere coupled to a dengue 3 virus VLP, an amount of a fourth microsphere complex comprising a fourth microsphere coupled to a dengue 4 virus VLP, an amount of a fifth microsphere complex comprising a fifth microsphere coupled to a zika virus VLP, an amount of a sixth microsphere complex comprising a sixth microsphere coupled to a dengue 1 virus non-structural protein 1, an amount of a seventh microsphere complex comprising a seventh microsphere coupled to a dengue 2 virus non-structural protein 1, an amount of an eighth microsphere complex comprising an eighth microsphere coupled to a dengue 3 virus non-structural protein 1, an amount of a ninth microsphere complex comprising a ninth microsphere coupled to a dengue 4 virus non-structural protein 1, and an amount of a tenth microsphere complex comprising a tenth microsphere coupled to a zika virus non-structural protein 1 is contacted with the sample.

According to one embodiment contacting in step 1 is carried out for about 30 to 90 minutes. In specific embodiments contacting in step 1 is carried out for about 60 minutes.

According to one embodiment contacting in step 2 is carried out for about 10 to 50 minutes. In specific embodiments contacting in step 2 is carried out for about 30 minutes.

According to one embodiment contacting in step 3.1 is carried out for about 10 to 50 minutes. In specific embodiments contacting in step 2 is carried out for about 30 minutes.

According to one embodiment contacting in step 3.2 is carried out for about 10 to 50 minutes. In specific embodiments contacting in step 2 is carried out for about 30 minutes.

In one specific embodiment contacting in step 1 is carried out for about 30 to 90 minutes, contacting in step 2 is carried out for about 10 to 50 minutes, contacting in step 3.1 is carried out for about 10 to 50 minutes, and contacting in step 3.2 is carried out for about 10 to 50 minutes.

In a more specific embodiment contacting in step 1 is carried out for about 60 minutes, contacting in step 2 is carried out for about 30 minutes, contacting in step 3.1 is carried out for about 30 minutes, and contacting in step 3.2 is carried out for about 30 minutes.

In certain embodiments, the C1q is present within a complement-component serum, which may be human complement-component serum. In certain embodiments, the C1q is purified from plasma. Purification can be carried out by any protein purification method known in the art, such as filtration, centrifugation, chromatographic separation, or buffer exchange. In other embodiments, the C1q is of human origin. In specific embodiments the C1q is purified human C1q. In general, the origin of the C1q is dependent on the origin of complement-fixing Abs in the sample. For instance, for detection of human complement-fixing Abs, human C1q is suitable.

In certain embodiments, the complement competent serum is a commercially obtainable complement competent serum. Alternatively, a human serum may be obtained and used.

In certain embodiments, the sample is heat-inactivated. Heat-inactivation can be carried out at about 56° C. for about 30 minutes. In general, heat-inactivation can be varied according to the type of sample to be analyzed.

In some embodiments the sample is a blood sample, in particular a plasma or serum sample. In general, the method can be carried out with either matrices i.e. serum or plasma.

In other embodiments the sample is a urine sample.

In specific embodiments, the sample is a heat-inactivated serum or plasma sample.

In one embodiment the subject is selected from the group consisting of mouse, primate, non-human primate, human, rabbit, cat, rat, horse, and sheep. In preferred embodiments the subject is a non-human primate. In more preferred embodiments the subject is human.

In other embodiments the subject is seronegative for the flavivirus. In other embodiments the subject is seropositive for the flavivirus. In specific embodiments the subject is seronegative for DENY. In other specific embodiments the subject is seropositive for DENY.

In some embodiments of the present invention the subject is vaccinated with a flavivirus vaccine.

In specific embodiments of the present invention the presence and/or amount of reporter Ab in step 7 is determined by comparing the summarized signal of step 6 to a standard curve, wherein the standard curve comprises signals resulting from known amounts of reporter Ab.

In specific embodiments of the invention the presence and/or amount of flavivirus-reactive complement-fixing Abs in the sample in step 8 is determined based on the amount of the reporter Ab determined in step 7 as the amount of reporter Ab is in direct proportion to the complement-fixing Abs present within a sample. Thereby, the amount of complement-fixing Abs reactive to the different flaviviruses can be determined.

### In Vitro Methods for Diagnosing a Flavivirus Infection

The present invention is further directed to in vitro methods for diagnosing a flavivirus infection. Concerning the determination of the amount of flavivirus-reactive complement-fixing Abs, reference is made to the sections above termed “Methods for determining flavivirus-reactive complement-fixing antibodies” and “Methods for the concomitant determination of complement-fixing antibodies reactive to different flaviviruses”. In addition, certain specific embodiments are also outlined in this section and shall, however, not be taken as limiting.

In one aspect of the present invention the in vitro method for diagnosing a flavivirus infection in a subject comprises determining the amount of flavivirus-reactive complement-fixing antibodies in the sample according to the methods of the first aspect of the present invention as described above, wherein the presence of flavivirus-reactive complement-fixing antibodies in the sample is indicative for a flavivirus infection.

In a second aspect of the present invention the in vitro method for diagnosing a flavivirus infection in a subject comprises determining the amount of flavivirus-reactive complement-fixing antibodies in the sample according to the methods of the second aspect of the present invention as described above, wherein the presence of flavivirus-reactive complement-fixing antibodies in the sample is indicative for a flavivirus infection.

In some embodiments, the flavivirus is selected from the group consisting of dengue virus, zika virus, West Nile virus, Japanese encephalitis virus, Tick-Borne encephalitis virus, Yellow Fever virus, Murray Valley encephalitis virus, and St. Louis encephalitis virus. In preferred embodiments the flavivirus is DENV or ZIKV.

In some embodiments, the subject is infected by a dengue virus serotype selected from the group of dengue virus serotype 1, dengue virus serotype 2, dengue virus serotype 3, and dengue virus serotype 4.

In some embodiments, the subject is infected by at least two different flaviviruses. For instance, the subject can be infected by DENV and ZIKV. The infection may be acute or convalescent. For instance, the subject has been infected by DENV first and was then infected by ZIKV several months later. The in vitro method for diagnosing of the present application is capable of diagnosing the at least two different flavivirus infections e.g. the DENV and the ZIKV infection. Consequently, the in vitro method for diagnosing of the present application is capable of determining whether a subject was infected with one or more flaviviruses and by which flaviviruses the subject was infected.

In some embodiments, the subject is infected by at least two different dengue virus serotypes selected from the group of dengue virus serotype 1, dengue virus serotype 2, dengue virus serotype 3, and dengue virus serotype 4. For instance, the subject can be infected by DENV serotype 1 and DENV serotype 2. The infection may be acute or convalescent. For instance, the subject has been infected by DENV serotype 1 first and was then infected by DENV serotype 2 several months later. The in vitro method for diagnosing of the present application is capable of diagnosing the at least two different dengue virus serotype infections e.g. the DENV serotype 1 and the DENV serotype 2 infection. Consequently, the in vitro method for diagnosing of the present application is capable of determining whether a subject was infected with one or more DENV serotypes and by which DENV serotypes the subject was infected.

In certain embodiments, the sample is heat-inactivated. Heat-inactivation can be carried out at about 56° C. for about 30 minutes. In general, heat-inactivation can be varied according to the type of sample to be analyzed.

In other embodiments the sample is a blood sample, in particular a plasma or serum sample. In general, the method can be carried out with either matrices i.e. serum or plasma.

In other embodiments the sample is a urine sample.

In specific embodiments, the sample is a heat-inactivated serum or plasma sample.

In one embodiment the subject is selected from the group consisting of mouse, primate, non-human primate, human, rabbit, cat, rat, horse, and sheep. In preferred embodiments the subject is a non-human primate. In more preferred embodiments the subject is human.

In other embodiments the subject is seronegative for the flavivirus. In other embodiments the subject is seropositive for the flavivirus. In specific embodiments the subject is seronegative for DENV. In other specific embodiments the subject is seropositive for DENV.

In some embodiments of the present invention the subject is vaccinated with a flavivirus vaccine.

In one embodiment the flavivirus infection is acute. In another embodiment the flavivirus infection is convalescent.

### Kits for Detecting Flavivirus-Reactive Complement-Fixing Antibodies

The present invention is further directed to kits for detecting flavivirus-reactive complement-fixing antibodies. Concerning the microsphere complex, as well as the reporter and pre-reporter Ab, reference is made to the sections above termed “microsphere complex” and “Reporter and Pre-reporter Ab”. In addition, certain specific embodiments are also outlined in this section and shall, however, not be taken as limiting.

In a first aspect of the invention, the kit for detecting flavivirus-reactive complement-fixing antibodies in a sample comprises:
an amount of at least one microsphere complex comprising a microsphere coupled to a flavivirus antigen as described above,
an amount of C1q, and
an amount of a reporter antibody, wherein the reporter antibody is attached to a detectable label.

In one embodiment, the kit for detecting flavivirus-reactive complement-fixing antibodies in a sample comprises:
an amount of at least two microsphere complexes,
   wherein the first microsphere complex comprises a first microsphere coupled to a first flavivirus antigen as described above and the second microsphere complex comprises a second microsphere coupled to a second flavivirus antigen as described above,
   wherein the first microsphere comprises a first detectable label and the second microsphere comprises a second detectable label, and
   wherein the emission signal of the first detectable label differs from the emission signal of the second detectable label
an amount of C1q, and
an amount of a reporter antibody, wherein the reporter antibody binds to the C1q with the variable region of the reporter antibody, and wherein the reporter antibody is attached to a detectable label.

In a second aspect the present invention provides a kit for detecting flavivirus-reactive complement-fixing antibodies in a sample comprising:
an amount of at least one microsphere complex comprising a microsphere coupled to a flavivirus antigen,
an amount of a complement competent serum, and
an amount of a reporter antibody, wherein the reporter antibody binds to the C3d with the variable region of the reporter antibody, and wherein the reporter antibody is attached to a detectable label.

In an embodiment of the second aspect the kit comprises an amount of at least two microspheres complexes,
wherein the first microsphere complex comprises a first microsphere coupled to a first flavivirus antigen and the second microsphere complex comprises a second microsphere coupled to a second flavivirus antigen,
wherein the first microsphere comprises a first detectable label and the second microsphere comprises a second detectable label, and
wherein the emission signal of the first detectable label differs from the emission signal of the second detectable label.

In some embodiments, the flavivirus is selected from the group consisting of dengue virus, Zika virus, West Nile virus, Japanese encephalitis virus, Tick-Borne encephalitis virus, Yellow Fever virus, Murray Valley encephalitis virus, and St. Louis encephalitis virus.

In some embodiments, the antigen is selected from the group consisting of virus like particle (VLP), non-structural protein 1, envelope protein, pre-membrane protein, membrane protein, capsid protein, non-structural protein 2A, non-structural protein 2B, non-structural protein 3, non-structural protein 4A, non-structural protein 4B, and non-structural protein 5 and any derivative thereof.

The kit of the present invention may comprise any number of microsphere complexes, wherein each microsphere complex comprises a microsphere coupled to a different flavivirus antigen and wherein each microsphere complex comprises a microsphere with a different detectable label resulting in a different emission signal e.g. a different emission intensity upon irradiation thereby providing the possibility to identify the different microspheres and thereby the different antigens.

In specific embodiments the kit comprises an amount of a first microsphere complex comprising a first microsphere coupled to a dengue-1 virus VLP, an amount of a second microsphere complex comprising a second microsphere coupled to a dengue-2 virus VLP, an amount of a third microsphere complex comprising a third microsphere coupled to a dengue-3 virus VLP, and an amount of a fourth microsphere complex comprising a fourth microsphere coupled to a dengue-4 virus VLP.

In other specific embodiments the kit comprises an amount of a first microsphere complex comprising a first microsphere coupled to a dengue-1 virus VLP, an amount of a second microsphere complex comprising a second microsphere coupled to a dengue-2 virus VLP, an amount of a third microsphere complex comprising a third microsphere coupled to a dengue-3 virus VLP, an amount of a fourth microsphere complex comprising a fourth microsphere coupled to a dengue-4 virus VLP, an amount of a fifth microsphere complex comprising a fifth microsphere coupled to a zika virus VLP, an amount of a sixth microsphere complex comprising a sixth microsphere coupled to a dengue-1 virus non-structural protein 1, an amount of a seventh microsphere complex comprising a seventh microsphere coupled to a dengue-2 virus non-structural protein 1, an amount of an eighth microsphere complex comprising an eighth microsphere coupled to a dengue-3 virus non-structural protein 1, an amount of a ninth microsphere complex comprising a ninth microsphere coupled to a dengue-4 virus non-structural protein 1, and an amount of a tenth microsphere complex comprising a tenth microsphere coupled to a zika virus non-structural protein 1.

In certain embodiments, the C1q is present within a complement-component serum, which may be human complement-component serum. In certain embodiments, the C1q is purified from plasma. Purification can be carried out by any protein purification method known in the art, such as filtration, centrifugation, chromatographic separation, or buffer exchange. In other embodiments, the C1q is of human origin. In specific embodiments the C1q is purified human C1q. In general, the origin of the C1q is dependent on the origin of complement-fixing Abs in the sample. For instance, for detection of human complement-fixing Abs, human C1q is suitable.

In certain embodiments, the complement competent serum is a commercially obtainable complement competent serum. Alternatively, a human serum may be obtained and used. In certain embodiments of the first aspect of the invention, the reporter antibody binds to the C1q with the variable region of the reporter antibody, when the kit does not further comprise a pre-reporter antibody.

In other embodiments the kit further comprises an amount of a pre-reporter antibody, wherein the pre-reporter antibody binds to the C1q with the variable region of the pre-reporter antibody and the reporter antibody binds to the heavy chain constant region of the pre-reporter antibody with the variable region of the reporter antibody.

In a second aspect, when the assay is based on the detection of the formation of C3d due to the formation of flavivirus antigen/complement-fixing antibody complexes in the presence of complement competent serum, the reporter Ab is capable of binding to the formed C3d. Preferably, the reporter Ab is specific for C3d, more preferably for human C3d.

Within the embodiments of the present invention, the reporter Ab is attached to a detectable label. In preferred embodiments, the reporter Ab is attached to the detectable label by the heavy chain constant region of the reporter Ab.

In a third aspect, when applied together with a pre-reporter Ab, the reporter Ab is capable of binding to the heavy chain constant region of the pre-reporter antibody with the variable region of the reporter antibody. The reporter Ab is attached to a detectable label, preferably with the heavy chain constant region of the reporter antibody.

In some embodiments the detectable label to which the reporter antibody is attached to is a fluorescence label selected from the group consisting of xanthene, fluorescein isothiocyanate, rhodamine, phycoerythrin, cyanine, coumarin, and any derivative thereof. In preferred embodiments the fluorescence label is phycoerythrin.

In certain embodiments the first detectable label is at least one fluorescent dye. In other embodiments the second detectable label is at least one fluorescent dye.

In other embodiments the first and the second detectable labels are each at least one fluorescent dye. Within the meaning of the invention the first and the second detectable label are different labels. In preferred embodiments the fluorescent dye is selected from the group consisting of squaraine, phthalocyanine, naphthalocyanine, and any derivative thereof. For instance, the first microsphere comprises squarine, the second microsphere comprises phthalocyanine.

The kits may further contain a suitable container for the mixture of the components of the kit.

### EXAMPLES

The following Examples are included to demonstrate certain aspects and embodiments of the invention as described in the claims. It should be appreciated by those of skill in the art, however, that the following description is illustrative only and should not be taken in any way as a restriction of the invention.

### Example 1: Coupling of Flavivirus Antigens to Microspheres

Microspheres used for coupling were MagPlex® microspheres (Luminex Corporation, Austin, Texas). MagPlex® microspheres are superparamagnetic polystyrene microspheres with surface carboxylic acid groups. The microspheres were delivered in a volume of 4 to 4.1 mL with an average concentration of 1.2 to 1.3×10⁷ microspheres per mL (microspheres/mL). MagPlex® microspheres are available in several unique regions, i.e. the microspheres comprise one or more fluorescent dyes having a defined emission signal (the detectable label) in order to distinguish the microspheres from microspheres of other unique regions. As the coupling mechanism involving the surface carboxyl groups is independent of the specific feature of the microspheres, MagPlex® microspheres of different unique regions may be exchanged according to variations in experimental set-ups.

DENV antigens for coupling to microspheres were DENV1 VLP (0.46 mg/mL liquid stock in 10 mM sodium phosphate, 20 mM sodium citrate, 154 mM sodium chloride pH 7.4; The Native Antigen Company, Product Code: DENV1-VLP-500, Batch No. 19040109), DENV2 VLP (0.52 mg/mL liquid stock in 10 mM sodium phosphate, 20 mM sodium citrate, 154 mM sodium chloride pH 7.4; The Native Antigen Company, Product Code: DENV2-VLP-500, Batch No. 19040816), DENV3 VLP (0.72 mg/mL liquid stock in 10 mM sodium phosphate, 20 mM sodium citrate, 154 mM sodium chloride pH 7.4; The Native Antigen Company, Product Code: DENV3-VLP-500, Batch No. 18111415), and DENV4 VLP (0.53 mg/mL liquid stock in Dulbecco's phosphate-buffered saline (DPBS) pH 7.4, 30% sucrose; The Native Antigen Company, Product Code: DENV4-VLP-500, Batch No. 19061911).

DENV1-4 VLPs are consisting of DENV prM, M, and E protein produced in human embryonic kidney (HEK 293) cells. For production of DENV1-4 VLPs, the C-terminal 20% of DENV E protein were replaced by the corresponding Japanese encephalitis virus (JEV) SA-14 sequence (EMBL-EBI accession No: M55506.1; E protein amino acids 399-497 (DENV1 VLP), 397-495 (DENV2 VLP), 399-492 (DENV3 VLP), 400-495 (DENV4 VLP)). The replaced sequence corresponds to the transmembrane and intraparticle portion of the protein. DENV1 VLP was produced using the sequence from strain Puerto Rico/US/BID-V853/1998 (SEQ ID NO: 1 and 5; GenBank accession No. EU482592.1, Uniprot No. B1PNU3). DENV2 VLP was produced using the sequence from strain Thailand/16681/84 (SEQ ID NO: 2 and 6; EMBL-EBI accession No: U87411.1, Uniprot No. P29990.1). DENV3 VLP was produced using the sequence from strain Sri Lanka D3/H/IMTSSA-SRI/2000/1266 (SEQ ID NO: 3 and 7; GenBank accession No. AY099336.1, Uniprot No. Q6YMS4.1). DENV4 VLP was produced using the sequence from strain Dominica/814669/1981 (SEQ ID NO: 4 and 8; EMBL-EBI accession No: AF326825.1, Uniprot No. P09866.2).

ZIKV VLPs (The Native Antigen Company, Product Code: ZIKV-VLP) comprise prM, M, and E protein of ZIKV strain Z1106033 isolated in Suriname (Asian genotype; Enfissi et al., Lancet 2016, 387(10015):227-228; GenBank Accession No. KU312312.1 and ALX35659.1, SEQ ID NO: 9 and 10), include amino acids 123-819 and are produced in HEK 293 cells.

DENV1-4 NS1 (The Native Antigen Company, Product Code: DENVX4-NS1) and ZIKV NS1 (The Native Antigen Company, Product Code: ZIKVSU-NS1) are produced in human embryonic kidney (HEK 293) cells. DENV1 NS1 was produced using the sequence from strain Nauru/Western Pacific/1974 (SEQ ID NO: 11 and 12; GenBank accession No. AY145121). The sequence of DENV1 NS1 as coupled to the microspheres is shown in SEQ ID NO: 13. DENV2 NS1 was produced using the sequence from strain Thailand/16681/84 (SEQ ID NO: 2 and 6; EMBL-EBI accession No: U87411.1, Uniprot No. P29990.1). The sequence of DENV2 NS1 as coupled to the microspheres is shown in SEQ ID NO: 14. DENV3 NS1 was produced using the sequence from strain Sri Lanka D3/H/IMTSSA-SRI/2000/1266 (SEQ ID NO: 3 and 7; GenBank accession No. AY099336.1, Uniprot No. Q6YMS4.1). The sequence of DENV3 NS1 as coupled to the microspheres is shown in SEQ ID NO: 15. DENV4 NS1 was produced using the sequence from strain Dominica/814669/1981 (SEQ ID NO: 4 and 8; EMBL-EBI accession No: AF326825.1, Uniprot No. P09866.2). The sequence of DENV4 NS1 as coupled to the microspheres is shown in SEQ ID NO: 16. ZIKV NS1 was produced using the sequence (amino acids 795-1146) from strain Suriname Z110603 (SEQ ID NO: 9 and 10; GenBank Accession No. KU312312.1 and ALX35659.1) and buffered in PBS, pH 7.4.

VLPs are a desirable reagent for coupling to the microspheres because of their ease of manufacture, antigenic fidelity, and lack of safety concerns. Further, for evaluation of complement fixing antibodies against all DENV serotypes produced upon vaccination with live attenuated viruses comprising E and prM structural proteins, VLPs are favorable as they are a good surrogate for the whole virion. Moreover, antibodies against structural antigens i.e. E and prM protein can be detected by the application of one single antigen (the VLP).

Different microspheres comprising one or more fluorescent dyes having a specific emission signal (different unique regions) were applied for coupling of the different antigens to provide the possibility to distinguish the microspheres according to their coupled antigens when analyzed within one sample (capability to multi-plex). For example, DENV1 VLP was coupled to a set of MagPlex® microspheres of region 76, DENV2 VLP was coupled to a set of MagPlex® microspheres of region 14, DENV3 VLP was coupled to a set of MagPlex® microspheres of region 25, and DENV4 VLP was coupled to a set of MagPlex® microspheres of region 33. DENV1 NS1 was coupled to a set of MagPlex® microspheres of region 45, DENV2 NS1 was coupled to a set of MagPlex® microspheres of region 65, DENV3 NS1 was coupled to a set of MagPlex® microspheres of region 66, and DENV4 NS1 was coupled to a set of MagPlex® microspheres of region 67. ZIKV VLP was coupled to a set of MagPlex® microspheres of region 47, ZIKV NS1 was coupled to a set of MagPlex® microspheres of region 36.

### Coupling of Flavivirus Antigens to Microspheres

The uncoupled stocks of MagPlex® microsphere suspensions (1.2 to 1.3×10⁷ microspheres/mL, Luminex Corporation, Austin, Texas) were resuspended by vortexing (30 sec) and 12.5×10⁶ microspheres of each stock were transferred to 5 mL microcentrifuge tubes and placed into a 5 mL tubes magnetic separator (Life Technologies). Separation of the microspheres from the suspension occurred for 30-60 sec. Supernatant was carefully removed without disrupting the microsphere pellet while the tubes were still positioned in the magnetic separator. Afterwards, the tubes were removed from the magnetic separator and the microspheres were resuspended in 500 μL distilled H₂O (dH₂O) by vortexing and sonication for approximately 20 sec. The tubes were again placed into the magnetic separator and separation of the microspheres from the suspension occurred for 30-60 sec. Supernatant was removed without disrupting the microsphere pellet while the tubes were still positioned in the magnetic separator. The microspheres were resuspended in 400 μl of activation buffer (0.1 M sodium phosphate (monobasic) pH 6.2) and mixed by vortexing and sonication for 20 sec. Then, 50 μL of 50 mg/mL N-hydroxysulfosuccinimide (Sulfo-NHS; 50 mg of Sulfo-NHS in 1000 μL of dH₂O; Thermo Fisher Scientific) were added to each microsphere tube and gentle mixing was carried out by vortexing (5 sec). Further, 50 μL of 50 mg/mL 1-Ethyl-3-(3-dimethylaminopropyl)carbodiimide (EDC; 10 mg EDC in 200 μL of dH₂O; Thermo Fisher Scientific) were added to each microsphere tube and gentle mixing was carried out by vortexing (5 sec). Samples were incubated for 20 min at room temperature (18-24° C.) with gentle mixing by vortexing after 10 min. The tubes were placed into the magnetic separator and separation of the microspheres from the suspension occurred for 30-60 sec. Supernatant was removed without disrupting the microsphere pellet while the tubes were still positioned in the magnetic separator. The tubes were removed from the magnetic separator and the microspheres were resuspended in 500 μL of 50 mM 2-(N-morpholino)ethanesulfonic acid (MES) buffer at pH 6.00 for DENV1-4 VLP, DENV1-4 NS1 proteins, and ZIKV NS1 protein (Boston Bioproducts, Cat. No. BBMS-60, Lot. No. F03K118) or at pH 7.00 for ZIKV VLP by vortexing and sonication for 20 sec. The tubes were placed into the magnetic separator and separation of the microspheres from the suspension occurred for 30-60 sec. Supernatant was removed without disrupting the microsphere pellet while the tubes were still positioned in the magnetic separator. The tubes were removed from the magnetic separator and the microspheres were resuspended in 500 μL of corresponding 50 mM MES buffer by vortexing and sonication for 20 sec. The tubes were placed into the magnetic separator and separation of the microspheres from the suspension occurred for 30-60 sec. Supernatant was removed without disrupting the microsphere pellet while the tubes were still positioned in the magnetic separator.

Afterwards, 1000 μL of a solution of each one of the DENV1-4 VLPs, DENV1-4 NS1 proteins, ZIKV VLP and ZIKV NS1 (diluted in 50 mM MES buffer at pH 6.0 for all NS1 proteins (DENV and ZIKV), as well as DENV1-4 VLPs and at pH 7.0 for ZIKV VLP) were transferred to a different 5 mL tube containing the activated microspheres to result in a ratio of 5 μg antigen per 10⁶ microspheres in a total volume of 1000 μL. The mixture was vortex for 20 sec. For coupling, samples were incubated for 2 hours under rotation at room temperature. The tubes were placed into the magnetic separator and separation of the microspheres from the suspension occurred for 30-60 sec. Supernatant was removed without disrupting the microsphere pellet while the tubes were still positioned in the magnetic separator. The tubes were removed from the magnetic separator and the microspheres were resuspended in 1 mL of 1% (v/v) bovine serum albumin (BSA) in 1-fold PBS pH 7.4 (prepared by diluting a 10% BSA stock) by vortexing for approximately sec. The tubes were placed into the magnetic separator and separation of the microspheres from the suspension occurred for 30-60 sec. Supernatant was removed without disrupting the microsphere pellet while the tubes were still positioned in the magnetic separator. The tubes were removed from the magnetic separator and the microspheres were resuspended in 1 mL of 1% BSA in 1-fold PBS pH 7.4 by vortexing for approximately 20 sec. The tubes were placed into the magnetic separator and separation of the microspheres from the suspension occurred for 30-60 sec. Supernatant was removed without disrupting the microsphere pellet while the tubes were still positioned in the magnetic separator. The tubes were removed from the magnetic separator and the microspheres were resuspended in 2000 μL of 1% BSA in 1-fold PBS pH 7.4 by vortexing for approximately 20 sec. The microspheres were kept in the 2.0 mL tubes. In order to count the microspheres recovered after the coupling reaction, the microsphere suspension was diluted 2-fold in 1% BSA in 1-fold PBS pH 7.4 (e.g. 15 μL microsphere suspension diluted with 15 μL of 1% BSA in 1-fold PBS pH 7.4). The number of microspheres recovered after the coupling reaction was determined using an automated cell counter (Countes II, Thermo Fisher Scientific, Cat. No. AMQAX1000) by correlating the determined “dead cells” concentration provided by the cell counter to the microspheres. The coupled microspheres were stored at 2-8° C. in the dark (blocking step) separately for each antigen. Previous to use, the coupled microspheres were allowed to pre-warm for at least 30 min at room temperature.

### Optimization of Coupling Conditions

As the coupling efficiency, as well as the integrity of the antigen after the coupling procedure is dependent on the buffer used, different buffer conditions were evaluated for coupling of the VLPs and NS1 proteins to the microspheres. Coupling carried out as described above, except that the buffer was varied. Optimization of the coupling procedure is important in order to ensure that the three-dimensional structure of the antigen is not disturbed. The buffer conditions may vary dependent on the type of antigen used.

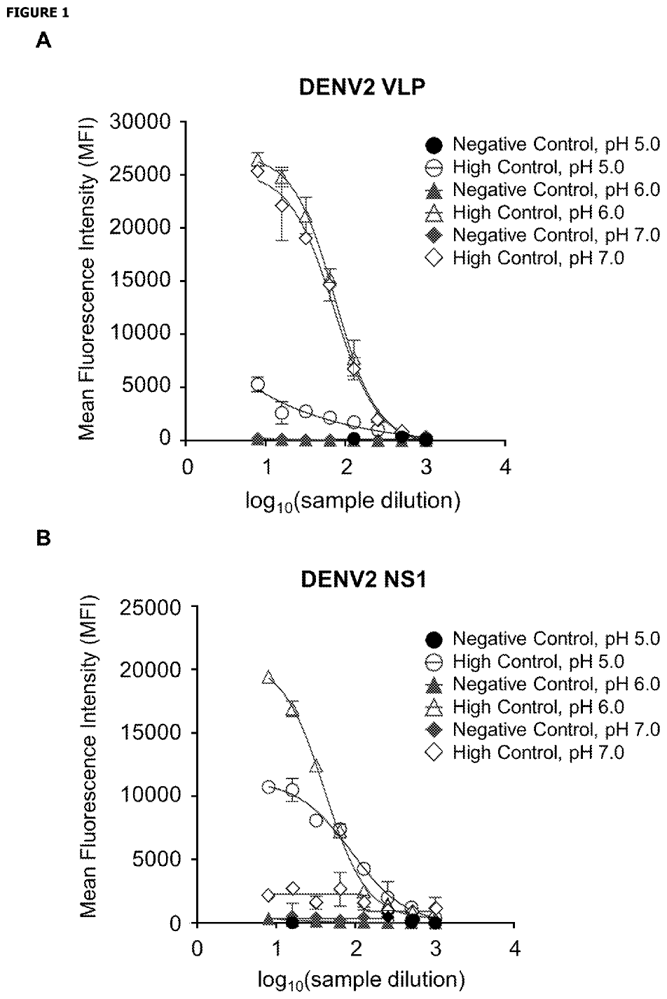

Different buffer conditions (50 mM MES at pH 5.00, 6.00, and 7.00) for coupling were examined and the signal to noise ratio was evaluated in a DENV-quadruplex complement-fixing assay set-up essentially as described under Example 2. Therefore, a negative control, i.e. a serum sample lacking DENV antibodies, and a control sample, i.e. a plasma sample derived from a human subject living in DENV endemic areas in Colombia with high amounts of anti-DENV complement fixing Abs were tested (FIG. 1A, exemplarily shown for DENV2 VLP). The assay shows, that a pH of 6.00 results in the highest fluorescence signal from the high control sample. In addition, also a pH of 7.00 shows a high signal to noise ratio. In contrast, a lower pH of 5.00 did not result in high signals indicating that the pH is not suitable for VLP coupling. For comparison, when coupling single proteins such as NS1, the optimum pH values seem to be different (FIG. 1B, exemplarily shown for DENV2 NS1). While for the VLP a coupling pH of 7.00 results in high fluorescence signals, for the NS1 protein the same pH does solely result in low fluorescence signals, indicating significant differences in coupling efficacies depending on the size, as well as three-dimensional structure of the antigen. Satisfyingly, no background signal was observed from the negative control sample neither for the VLP, nor for the NS1 protein independent of the buffer pH.

As described above, for routine coupling of DENV1-4 VLPs and DENV1-4 NS1 50 mM MES at pH 6.00 was applied. Of note, the optimum coupling pH for ZIKV VLP was 50 mM MES at pH 7.00 indicating that also virus-specific effects have an impact on the coupling efficiency.

### Example 2: Evaluation of a DENV-Quadruplex Complement-Fixing Assay Set-Up

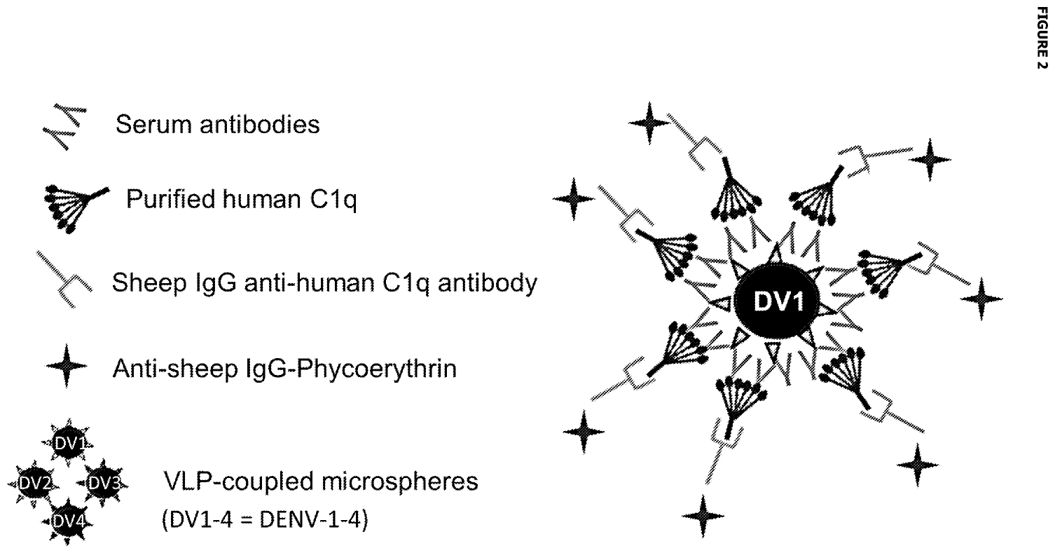

The DENV1-4 VLP-coupled microspheres of Example 1 were applied to develop a DENV-quadruplex complement-fixing assay set-up (FIG. 2) as described in the following.

For evaluation of the complement-fixing assay, a reference sample, as well as control samples were analyzed. The reference sample consists of pooled plasma samples from human subjects living in DENV endemic areas in Colombia with high levels of anti-DENV complement-fixing Abs (ABO Pharmaceuticals, Lot. VBU-01140-148). Control samples comprised plasma derived from human subjects living in DENV endemic areas in Colombia, including high, medium, and low amounts of anti-DENV complement fixing Abs (ABO Pharmaceuticals, Lot. No. PARS_82 (high control), Lot. No. PARS_96 (medium control), Lot. No. VBU-01140-189 (low control)). In addition, a negative control sample was included consisting of serum lacking any anti-DENV Abs (Bioreclamation, Lot.-No. BRH1140253). In general, the assay can be performed with either matrices i.e. serum or plasma samples. All samples were stored at −80° C. prior to use. The samples were thawed and heat inactivated in a 56±1° C. water bath (Thermo Fisher, Isotemp 210, Cat.-No. 15-462-10Q) for 30±5 minutes prior to testing. Heat-inactivation is important to denature the temperature sensitive complement proteins within the sample and thereby to avoid assay interference.

In a next step, the DENV1-4 VLP-coupled microspheres of Example 1 were vortexed gently to break up clumping of the microspheres and ensure a uniform suspension. The microspheres were combined by dilution in assay buffer to result in a final concentration of 25 microspheres/μL for each DENV serotype and vortexed gently. Assay buffer consisted of phosphate buffered saline (PBS) with 1% (v/v) bovine serum albumin (BSA), diluted from a 10% stock (Fisher Scientific, Cat-No. 37525) and was stored at 2-8° C. for up to one month. The assay buffer was allowed to pre-warm for at least 30 minutes at room temperature prior to dilution of the microspheres. 50 μL of the microsphere suspension containing all four DENV serotype antigens were pipetted per well into a 96-well polystyrene microplate (solid black flat bottom plate, in the following referred to as “assay plate”; Corning, Cat.-No. 3915) resulting in 1250 microspheres per DENV-serotype per well. The plate was sealed with a foil plate seal (ThermoFisher, Cat.-No. AB0558) and stored at room temperature until the samples were diluted.

Heat-inactivated samples were serially diluted (8 dilutions final) using assay buffer pre-warmed to room temperature at least 30 minutes before testing. 50 μL per sample dilution were transferred into the assay plate per well to the microspheres in duplicates. Sample and microsphere suspension were mixed thoroughly by pipetting up and down 3-5 times. Next, the plate was sealed with a foil plate seal and incubated for 60±5 min at room temperature on a plate shaker (Heidolph, Titramax 1000, Cat.-No. 544-12200-00) at 600 rpm.

Afterwards, the plate was washed with wash buffer (PBS with 0.05% (v/v) Tween-20) using the Luminex Flat 96 Mag setting on a plate washer (BioTek ELx405, Cat.-No. 7100745S). After decanting of residual wash buffer, 50 μL/well of purified human C1q at a concentration of 4.0 μg/mL in assay buffer were added. Human C1q (Quidel, Cat.-No. A400, Lot.-No. 142550) is purified (>95%) from plasma, reconstituted with 40% (v/v) glycerol in 4-(2-hydroxyethyl)-1-piperazineethanesulfonic acid (HEPES) buffer at 1 mg/mL and stored at −80° C. prior to use. After C1q addition, the plate was sealed with a foil plate seal and incubated for 30±5 min at room temperature on a plate shaker at 600 rpm. By using purified C1q the exact amount of C1q applied in the assay can be controlled. In contrast, by using complement-component human serum the C1q concentration in the serum could vary between different lots of the serum. In addition, also the binding of C1q could be interfered by other serum components. Thus, the use of purified C1q improves reproducibility of the assay.

After incubation with C1q, the plate was washed with wash buffer as described above. After decanting of residual wash buffer, a pre-reporter Ab, i.e. sheep IgG anti-human C1q was added 50 μL/well at a 6.4 μg/mL dilution in assay buffer. Polyclonal purified sheep IgG anti-human C1q Ab (Bio-Rad, Cat.-No. AHP033, Lot.-No. 148553) recognizes human C1q. The Ab is solubilized in glycine buffer saline from manufacturer to 5.1 mg/mL and stored at 4° C. prior to use. After Ab addition, the plate was sealed with a foil plate seal and incubated for 30±5 min at room temperature on a plate shaker at 600 rpm.

After incubation with anti-C1q Ab, the plate was washed with wash buffer as described above. After decanting of residual wash buffer, a reporter Ab, i.e. PE-conjugated donkey IgG anti-sheep IgG was added 50 μL/well at a 10 μg/mL dilution in assay buffer. R-Phycoerythrin F(ab′)₂ fragment donkey anti-sheep IgG Ab (Jackson ImmunoResearch, Cat.-No. 713-116-147) is sold freeze-dried and was reconstituted with 1 mL deionized water according to the manufacturer to result in a 500 μg/mL solution. After Ab addition, the plate was sealed with a foil plate seal and incubated for 30±5 min at room temperature on a plate shaker at 600 rpm.

Finally, the plate was washed with wash buffer as described above. After decanting of residual wash buffer, 100 μL/well of assay buffer were added to the plate. The plate was covered with foil plate seal and shaken at 600 rpm for 5 min in order to resuspend the microspheres prior to read. At this point, the plate may also be stored at 2-8° C. overnight for analysis on the following day. If refrigerated overnight, the plate was shaken at room temperature for at least 30 min at 600 rpm.

The plate was analyzed in a MAGPIX Luminex plate reader with the xPONENT 4.2 software (Luminex Corp., Cat.-No. MAGPIX-XPONENT). The Luminex acquisition mode was set to 50 μL sample volume and 50 microspheres of each DENV serotype per well. The number of microspheres determined by the acquisition mode specifies that the Luminex reader needs to acquire at least 50 microspheres of each DENV serotype per well in order to determine the representative (statistically) mean signal of all microspheres of each DENV serotype per well.

For data analysis, each DENV serotype antigen is independently evaluated resulting in four standalone immunoassays. Data were analyzed and plotted using GraphPad Prism 8 version 8.1.0 (GraphPad Software, Inc). Mean Fluorescent Intensity (MFI; MFI values are the raw data reported by the MAGPIX® reader) values were plotted in dependence of log₁₀-transformed serum dilutions (e.g. 5-fold dilution resulting in log₁₀(5)=0.7). Sigmoidal fitting was performed according to a dose-response curve (Sigmoidal, 4PL, X=Log(concentration)). The equation used for the non-linear regression was “log(agonist) vs. response—Variable slope”. The MFI signal threshold of the reference sample equivalent to the EC25 concentration was calculated by subtracting the estimated bottom from top signals and multiplying the result by 0.25 (signal equivalent to 25% of effective concentration). The MFI signal threshold was then interpolated on the 4PL curves of both the reference sample and the control samples (as well as corresponding test samples) to determine the sample dilution equivalent to the EC25 signal of the reference (interpolated dilution). The interpolated dilutions are then divided by the interpolated dilution calculated in the reference to result in relative dilutions. Further, relative dilutions were multiplied by a constant (EC50 of the reference against each DENV serotype) for the corresponding DENV serotype (i.e. 468 for DENV-1 VLP, 345 for DENV-2 VLP, 369 for DENV-3 VLP and 257 for DENV-4 VLP) to result in the complement-fixing antibody titers in the samples in EU/mL. The EU/mL concentration is a relative arbitrary concentration based on the levels of complement-fixing antibodies found in the reference sample.

Signals Resulting from Different DENV VLPs

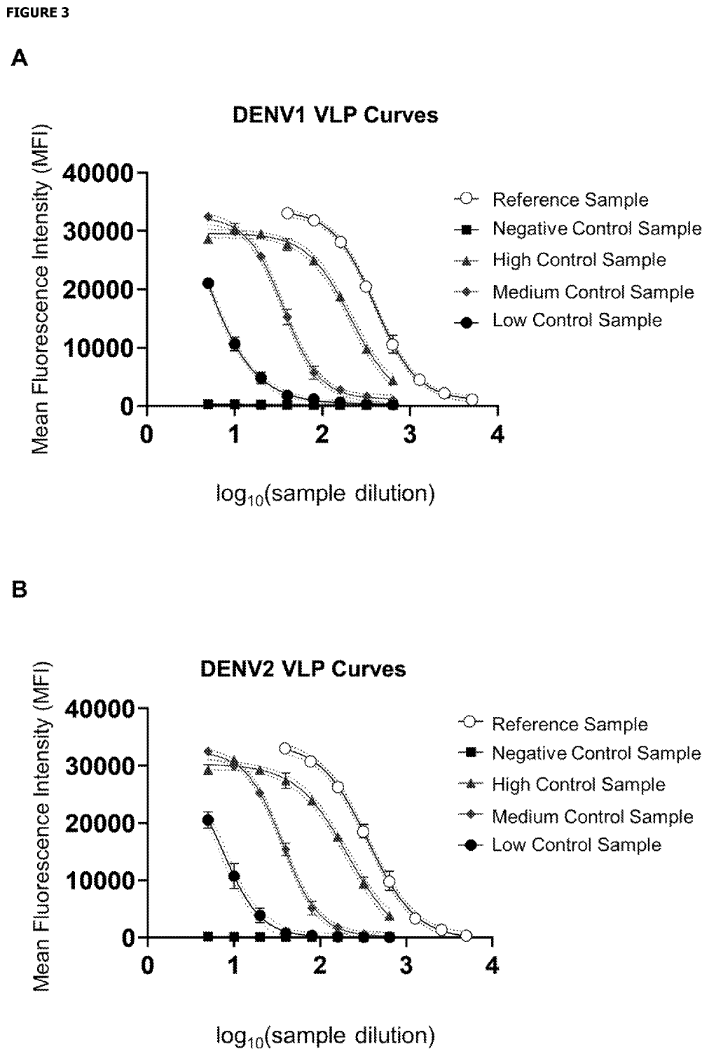

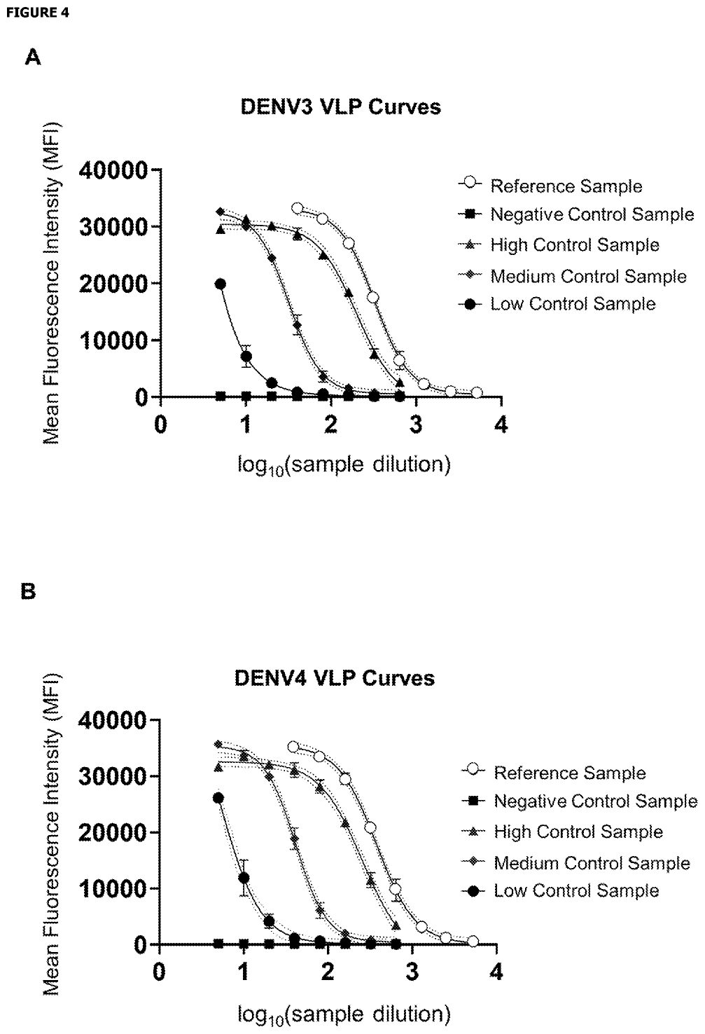

MFI values of the different sample dilutions and corresponding dose-response curve fits of reference and control samples are presented for all four DENV VLPs in FIGS. 3 and 4. Satisfyingly, for all four DENV VLPs MFI signals increased for all the samples except for the negative control with decreasing dilution factor. In addition, MFI signals increased from low to high titer control samples. Moreover, negative control serum did result in MFI signals close to zero independent of the dilution.

### Limit of Detection (LOD)

The LOD was determined by serial dilution of the reference sample in assay buffer and determination of the lowest complement-fixing antibody concentration for which the relative error (% RE=(obtained titer−expected titer)*100/obtained titer) is above 25% in one independent run using duplicates per dilution (Table 1). In summary, the LOD ranged from 0.46 EU/mL for DENV1 VLP to 0.72 EU/mL for DENV3 VLP.

TABLE 1
Complement-Fixing Antibody Titers in EU/mL dependent on the sample dilution and DENV
VLP. Presented are the expected titers, as well as the obtained titers in both replicates.
The relative error was calculated (% RE). The LOD was determined as the lowest Ab concentration
for which the relative error is above 25% (highlighted values).

| | DENV1 VLP | | | | DENV2 VLP | | | |
|---|---|---|---|---|---|---|---|---|
| | (Complement-Fixing Abs in EU/mL) | | | | (Complement-Fixing Abs in EU/mL) | | | |
| Sample | | Obtained | Obtained | | | Obtained | Obtained | |
| Dilution | Expected | Repl. 1 | Repl. 2 | % RE | Expected | Repl. 1 | Repl. 2 | % RE |
| Not dil. | 468.10 | — | — | — | 345.043 | — | — | — |
| 1:2 | 234.05 | 275.61 | 343.52 | 32% | 172.52 | 180.98 | 223.70 | 17% |
| 1:4 | 117.02 | 113.77 | 127.49 | 3% | 86.26 | 82.14 | 104.77 | 8% |
| 1:8 | 58.51 | 74.96 | 60.98 | 16% | 43.13 | 41.52 | 43.14 | −2% |
| 1:16 | 29.26 | 29.27 | 26.60 | −5% | 21.57 | 20.53 | 17.86 | −11% |
| 1:32 | 14.63 | 15.92 | 16.57 | 11% | 10.78 | 9.70 | 11.09 | −4% |
| 1:64 | 7.31 | 7.65 | 7.05 | 0% | 5.39 | 5.62 | 5.92 | 7% |
| 1:128 | 3.66 | 4.06 | 3.06 | −3% | 2.70 | 2.90 | 2.15 | −6% |
| 1:256 | 1.83 | 1.72 | 1.50 | −12% | 1.35 | 1.52 | 1.27 | 4% |
| 1:512 | 0.91 | 1.23 | 0.88 | 15% | 0.67 | 0 | 0.94 | −31% |
| 1:1024 | 0.46 | 0.66 | | 44% | 0.34 | 0 | | −100% |
| 1:2048 | 0.228 | 0 | 0.365 | −20% | 0.168 | 0 | | −100% |
| 1:4096 | 0.114 | 0 | | −100% | 0.084 | 0 | | −100% |
| | DENV3 VLP | | | | DENV4 VLP | | | |
| | (Complement-Fixing Abs in EU/mL) | | | | (Complement-Fixing Abs in EU/mL) | | | |
| Sample | | Obtained | Obtained | | | Obtained | Obtained | |
| Dilution | Expected | Repl. 1 | Repl. 2 | % RE | Expected | Repl. 1 | Repl. 2 | % RE |
| Not dil. | 368.64 | — | — | — | 257.19 | — | — | — |
| 1:2 | 184.32 | 194.14 | 227.57 | 14% | 128.59 | 128.64 | 142.64 | 5% |
| 1:4 | 92.16 | 91.76 | 103.76 | 6% | 64.30 | 63.02 | 61.29 | −3% |
| 1:8 | 46.08 | 46.82 | 45.83 | 1% | 32.15 | 29.65 | 30.91 | −6% |
| 1:16 | 23.04 | 22.20 | 21.00 | −6% | 16.07 | 15.05 | 13.35 | −12% |
| 1:32 | 11.52 | 10.86 | 11.94 | −1% | 8.04 | 7.37 | 7.80 | −6% |
| 1:64 | 5.76 | 5.09 | 5.31 | −10% | 4.02 | 4.03 | 4.03 | 0% |
| 1:128 | 2.88 | 2.92 | 2.47 | −6% | 2.01 | 1.94 | 1.66 | −10% |
| 1:256 | 1.44 | 1.33 | 1.31 | −8% | 1.00 | 1.08 | 0.98 | 2% |
| 1:512 | 0.72 | 0 | 0.76 | −47% | 0.50 | 0 | 0.62 | −38% |
| 1:1024 | 0.36 | 0 | | −100% | 0.25 | 0 | | −100% |
| 1:2048 | 0.18 | 0 | | −100% | 0.125 | 0 | | −100% |
| 1:4096 | 0.09 | 0 | | −100% | 0.063 | 0 | | −100% |

### Lower Limit of Quantification (LLOQ)

The LLOQ was determined by serial dilution of the reference sample in human IgG-depleted serum and determination of the lowest complement-fixing antibody concentration in which the relative error (% RE) is above 25% in five independent runs (Tables 2-5). The LLOQ for all DENV VLPs was determined to be 3.00 EU/mL.

TABLE 2
Expected and obtained complement-fixing Ab titers (EU/mL) for DENV1 VLP dependent
on the sample dilution. Presented are the expected titers, as well as the obtained
titers in the five independent runs. The average obtained titer of the five independent
runs was calculated, as well as the standard deviation (SD), the percent coefficient
of variation (% CV), and the relative error (% RE). As the signal threshold was not
reached, titers in rows highlighted in grey were calculated based on extrapolation
of the titration curves and, therefore, were considered not detectable.

| | Expected | Obtained Titer (EU/mL) | | | | | | | | |
|---|---|---|---|---|---|---|---|---|---|---|
| Sample | Titer | Run | Run | Run | Run | Run | | | % | % |
| Dilution | (EU/mL) | #1 | #2 | #3 | #4 | #5 | Average | SD | RE | CV |
| Not dil. | 468.10 | — | — | — | — | — | — | — | — | — |
| 1:2 | 234.05 | 257.16 | 229.55 | 184.00 | 187.70 | 214.78 | 214.64 | 30.39 | −8% | 14% |
| 1:4 | 117.02 | 126.08 | 121.01 | 112.95 | 94.45 | 94.72 | 109.84 | 14.70 | −6% | 13% |
| 1:8 | 58.51 | 64.37 | 59.34 | 50.52 | 49.12 | 50.84 | 54.84 | 6.68 | −6% | 12% |
| 1:16 | 29.26 | 28.31 | 28.39 | 23.02 | 25.29 | 26.03 | 26.21 | 2.25 | −10% | 9% |
| 1:32 | 14.63 | 12.95 | 14.87 | 12.77 | 12.46 | 12.68 | 13.15 | 0.98 | −10% | 7% |
| 1:64 | 7.31 | 7.63 | 6.95 | 8.66 | 5.99 | 7.78 | 7.40 | 1.00 | 1% | 13% |
| 1:128 | 3.66 | 3.69 | 3.36 | 4.02 | 2.90 | 3.68 | 3.53 | 0.43 | −3% | 12% |
| 1:256 | 1.83 | 1.69 | 1.68 | 1.79 | 1.09 | 1.61 | 1.57 | 0.28 | −14% | 18% |
| 1:512 | 0.91 | 0.87 | — | — | — | — | 0.87 | NC | −5% | NC |
| 1:1024 | 0.46 | — | — | — | — | — | NC | NC | NC | NC |

NC = not calculated.

TABLE 3
Expected and obtained complement-fixing Ab titers (EU/mL) for DENV2 VLP dependent
on the sample dilution. Presented are the expected titers, as well as the obtained
titers in the five independent runs. The average obtained titer of the five independent
runs was calculated, as well as the standard deviation (SD), the percent coefficient
of variation (% CV), and the relative error (% RE). As the signal threshold was not
reached, titers in rows highlighted in grey were calculated based on extrapolation
of the titration curves and, therefore, were considered not detectable.

| | Expected | Obtained Titer (EU/mL) | | | | | | | | |
|---|---|---|---|---|---|---|---|---|---|---|
| Sample | Titer | Run | Run | Run | Run | Run | | | % | % |
| Dilution | (EU/mL) | #1 | #2 | #3 | #4 | #5 | Average | SD | RE | CV |
| Not dil. | 345.043 | — | — | — | — | — | — | — | — | — |
| 1:2 | 172.52 | 216.39 | 177.48 | 133.24 | 140.42 | 161.12 | 165.73 | 33.25 | −4% | 20% |
| 1:4 | 86.26 | 99.61 | 82.69 | 81.97 | 67.33 | 64.81 | 79.28 | 14.00 | −8% | 18% |
| 1:8 | 43.13 | 49.65 | 39.87 | 35.38 | 34.54 | 34.70 | 38.83 | 6.43 | −10% | 17% |
| 1:16 | 21.57 | 22.04 | 19.70 | 17.02 | 18.20 | 19.40 | 19.27 | 1.88 | −11% | 10% |
| 1:32 | 10.78 | 9.22 | 10.95 | 9.82 | 9.23 | 9.64 | 9.77 | 0.71 | −9% | 7% |
| 1:64 | 5.39 | 5.35 | 5.21 | 6.48 | 4.62 | 6.53 | 5.64 | 0.84 | 5% | 15% |
| 1:128 | 2.70 | 2.78 | 2.55 | 2.97 | 2.22 | 3.13 | 2.73 | 0.36 | 1% | 13% |
| 1:256 | 1.35 | 1.54 | 1.33 | 1.52 | 0.76 | 1.57 | 1.34 | 0.34 | 0% | 25% |
| 1:512 | 0.67 | 0.87 | — | — | 0.51 | 0.95 | 0.77 | 0.23 | 15% | 30% |
| 1:1024 | 0.34 | — | — | — | — | — | NC | NC | NC | NC |

NC = not calculated.

TABLE 4
Expected and obtained complement-fixing Ab titers (EU/mL) for DENV3 VLP dependent
on the sample dilution. Presented are the expected titers, as well as the obtained
titers in the five independent runs. The average obtained titer of the five independent
runs was calculated, as well as the standard deviation (SD), the percent coefficient
of variation (% CV), and the relative error (% RE). As the signal threshold was not
reached, titers in rows highlighted in grey were calculated based on extrapolation
of the titration curves and, therefore, were considered not detectable.

| | Expected | Obtained Titer (EU/mL) | | | | | | | | |
|---|---|---|---|---|---|---|---|---|---|---|
| Sample | Titer | Run | Run | Run | Run | Run | | | % | % |
| Dilution | (EU/mL) | #1 | #2 | #3 | #4 | #5 | Average | SD | RE | CV |
| Not dil. | 368.64 | — | — | — | — | — | — | — | — | — |
| 1:2 | 184.32 | 211.80 | 177.43 | 157.61 | 168.53 | 174.03 | 177.88 | 20.40 | −4% | 11% |
| 1:4 | 92.16 | 104.33 | 94.66 | 93.56 | 80.86 | 76.36 | 89.96 | 11.28 | −2% | 13% |
| 1:8 | 46.08 | 53.64 | 45.65 | 40.78 | 41.95 | 41.84 | 44.77 | 5.29 | −3% | 12% |
| 1:16 | 23.04 | 23.68 | 22.19 | 20.25 | 22.14 | 20.53 | 21.76 | 1.40 | −6% | 6% |
| 1:32 | 11.52 | 10.75 | 11.90 | 10.94 | 10.86 | 10.33 | 10.96 | 0.58 | −5% | 5% |
| 1:64 | 5.76 | 6.05 | 5.41 | 6.30 | 4.70 | 5.96 | 5.68 | 0.64 | −1% | 11% |
| 1:128 | 2.88 | 3.03 | 2.64 | 2.92 | 2.30 | 2.90 | 2.76 | 0.29 | −4% | 11% |
| 1:256 | 1.44 | 1.21 | 1.12 | 1.20 | 0.63 | 1.02 | 1.04 | 0.24 | −28% | 23% |
| 1:512 | 0.72 | — | — | 0.49 | — | — | 0.49 | NC | −31% | NC |
| 1:1024 | 0.36 | — | — | — | — | — | NC | NC | NC | NC |

NC = not calculated.

TABLE 5
Expected and obtained complement-fixing Ab titers (EU/mL) for DENV4 VLP dependent
on the sample dilution. Presented are the expected titers, as well as the obtained
titers in the five independent runs. The average obtained titer of the five independent
runs was calculated, as well as the standard deviation (SD), the percent coefficient
of variation (% CV), and the relative error (% RE). As the signal threshold was not
reached, titers in rows highlighted in grey were calculated based on extrapolation
of the titration curves and, therefore, were considered not detectable.

| | Expected | Obtained Titer (EU/mL) | | | | | | | | |
|---|---|---|---|---|---|---|---|---|---|---|
| Sample | Titer | Run | Run | Run | Run | Run | | | % | % |
| Dilution | (EU/mL) | #1 | #2 | #3 | #4 | #5 | Average | SD | RE | CV |
| Not dil. | 257.19 | — | — | — | — | — | — | — | — | — |
| 1:2 | 128.19 | 151.94 | 122.52 | 101.84 | 108.87 | 121.68 | 121.37 | 19.19 | −6% | 16% |
| 1:4 | 64.30 | 72.98 | 63.56 | 62.17 | 52.93 | 52.54 | 60.84 | 8.49 | −5% | 14% |
| 1:8 | 32.15 | 37.13 | 30.94 | 26.45 | 27.23 | 27.58 | 29.86 | 4.41 | −7% | 15% |
| 1:16 | 16.07 | 16.07 | 14.53 | 13.25 | 13.92 | 14.38 | 14.43 | 10.4 | −10% | 7% |
| 1:32 | 8.04 | 7.04 | 8.04 | 7.25 | 6.90 | 7.12 | 7.27 | 0.45 | −9% | 6% |
| 1:64 | 4.02 | 4.14 | 3.82 | 4.56 | 3.38 | 4.42 | 4.06 | 0.48 | 1% | 12% |
| 1:128 | 2.01 | 2.14 | 1.86 | 2.15 | 1.68 | 2.17 | 2.00 | 0.22 | 0% | 11% |
| 1:256 | 1.00 | 1.01 | 0.89 | 0.93 | 0.56 | 0.85 | 0.85 | 0.17 | −16% | 20% |
| 1:512 | 0.50 | 0.53 | — | 0.43 | — | 0.39 | 0.45 | 0.08 | −11% | 17% |
| 1:1024 | 0.25 | — | — | — | — | — | NC | NC | NC | NC |

NC = not calculated.

### Assay Linearity

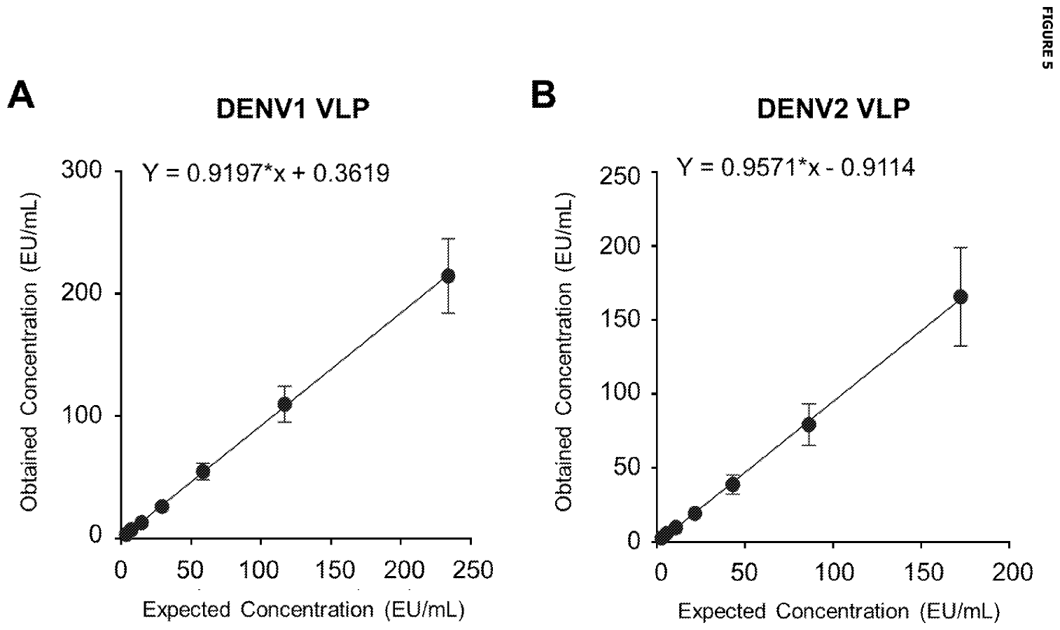

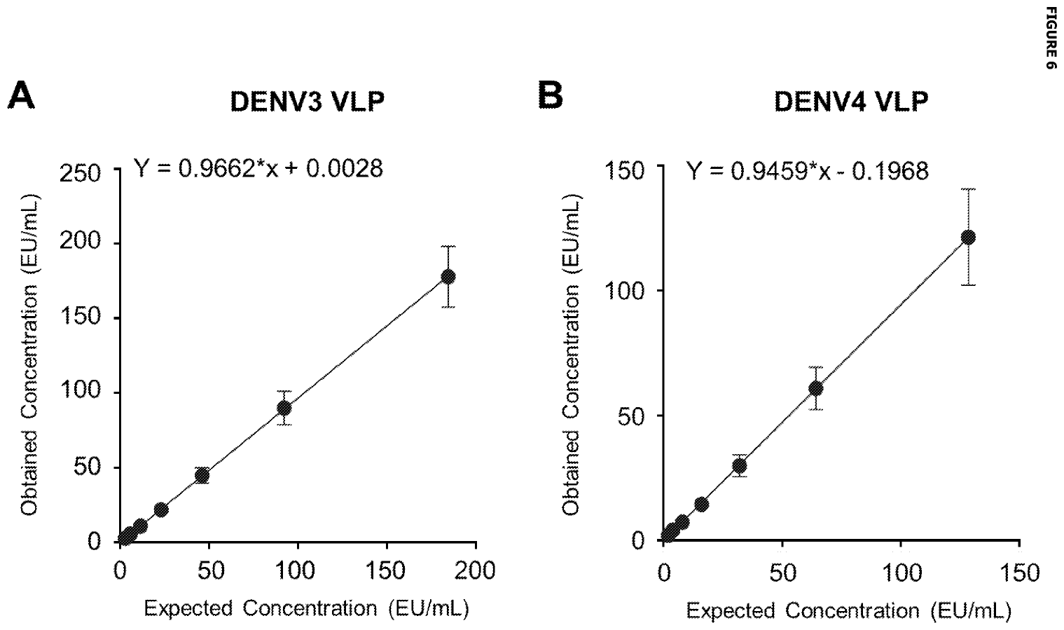

Assay linearity was evaluated by plotting the median value of the obtained concentration of the five independent runs performed for determination of the LLOQ for each reference sample dilution and DENV VLP against the expected concentration (FIGS. 5 and 6). Linear regression of the obtained and expected concentrations was performed and the slope together with the 95% confidence interval (CI) was calculated. The obtained and expected concentrations correlated well and in a linear manner with slopes close to one. The 95% Confidence Intervals (CI) were 0.8663 to 0.9731 for DENV1 VLP (slope of 0.9197), 0.8798 to 1.034 for DENV2 VLP (slope of 0.9571), 0.9193 to 1.013 for DENV3 VLP (slope of 0.9662), and 0.8856 to 1.006 for DENV4 VLP (slope of 0.9459).

### Assay Precision

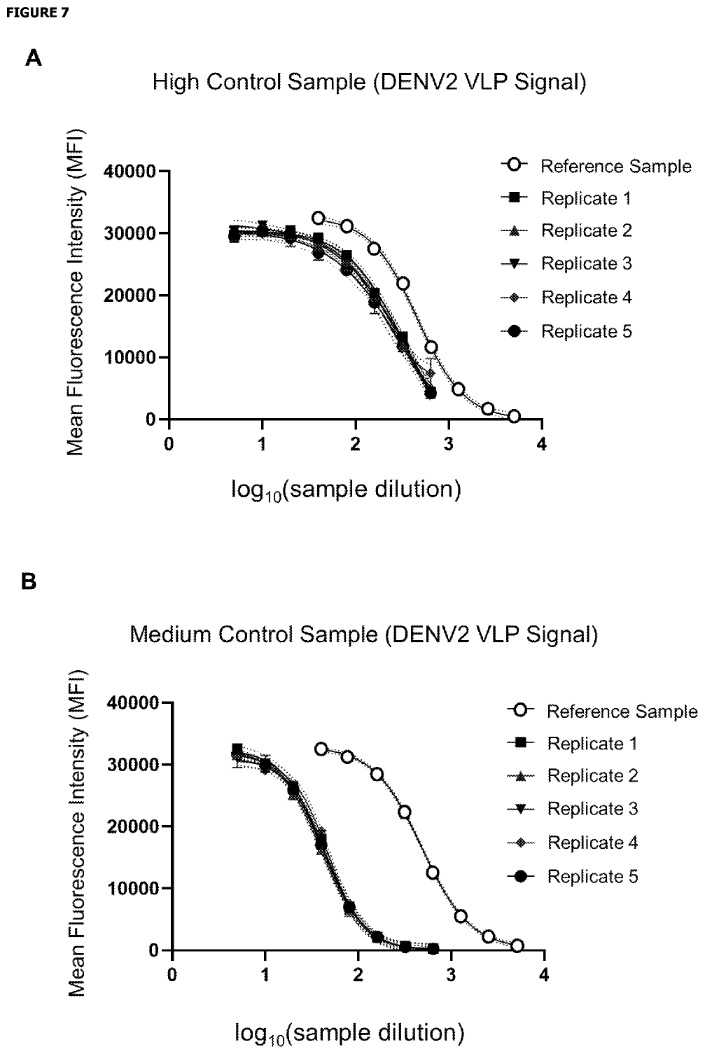

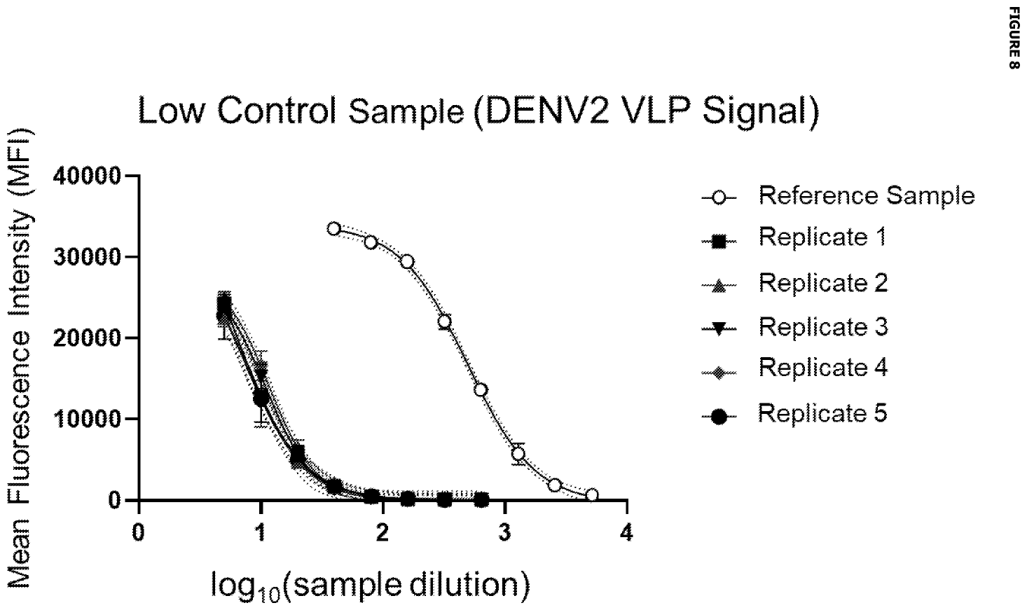

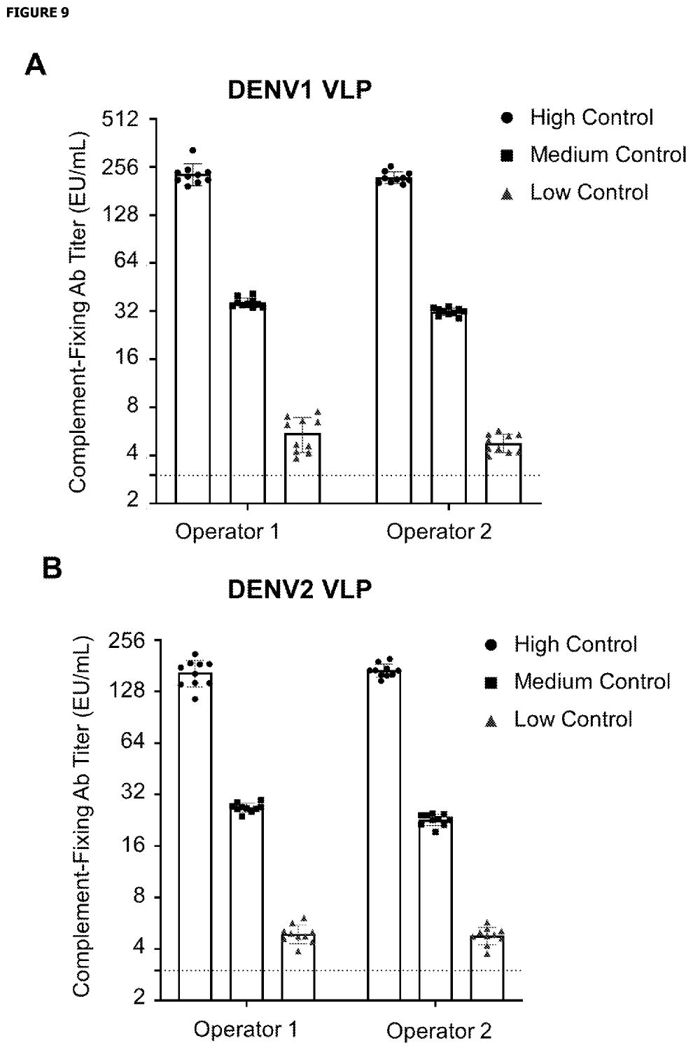

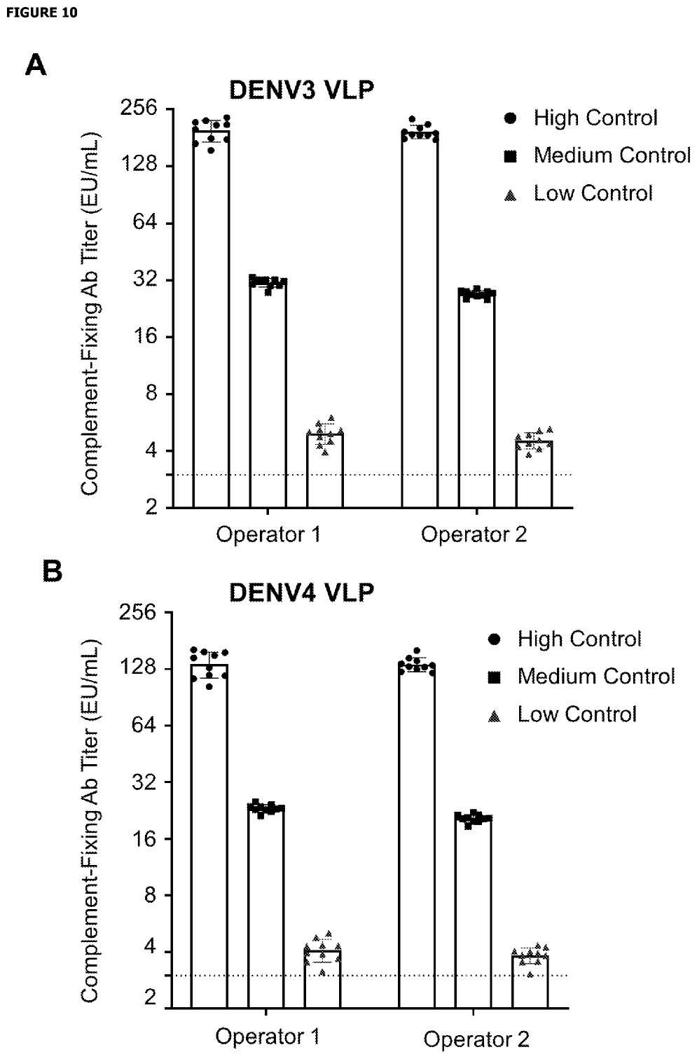

To evaluate the assay precision high, medium and low control samples were tested five times per run. In total, two different operators performed each two runs. Complete sigmoidal curves are exemplarily shown for one run and the DENV2 VLP antigen (FIGS. 7 and 8). Satisfyingly, fluorescence signals for the five replicates were highly similar independent of the signal intensity and sample type. In general, complement-fixing Ab titers were highly similar independent of the run, the control sample, the DENV VLP, and the operator (FIGS. 9 and 10).

For determination of intra-assay precision, the percent coefficient of variation (% CV=standard deviation/mean*100) of the complement-fixing Ab concentration was calculated for each sample within each run. For determination of inter-assay precision, the percent coefficient of variation (% CV) of the complement-fixing Ab concentration was calculated in between the runs.

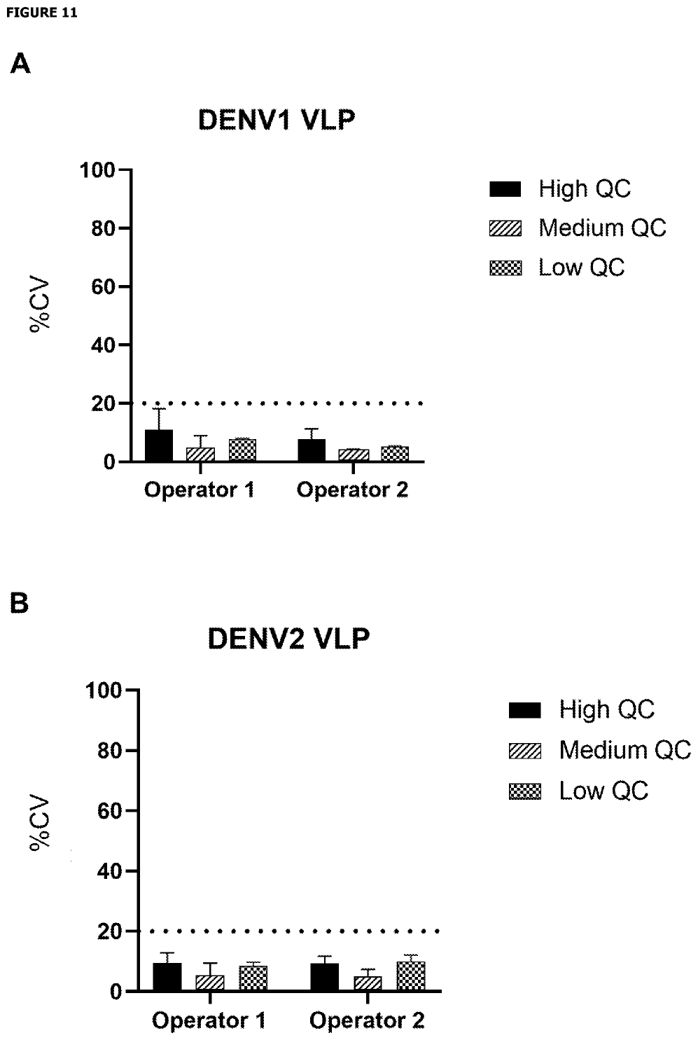

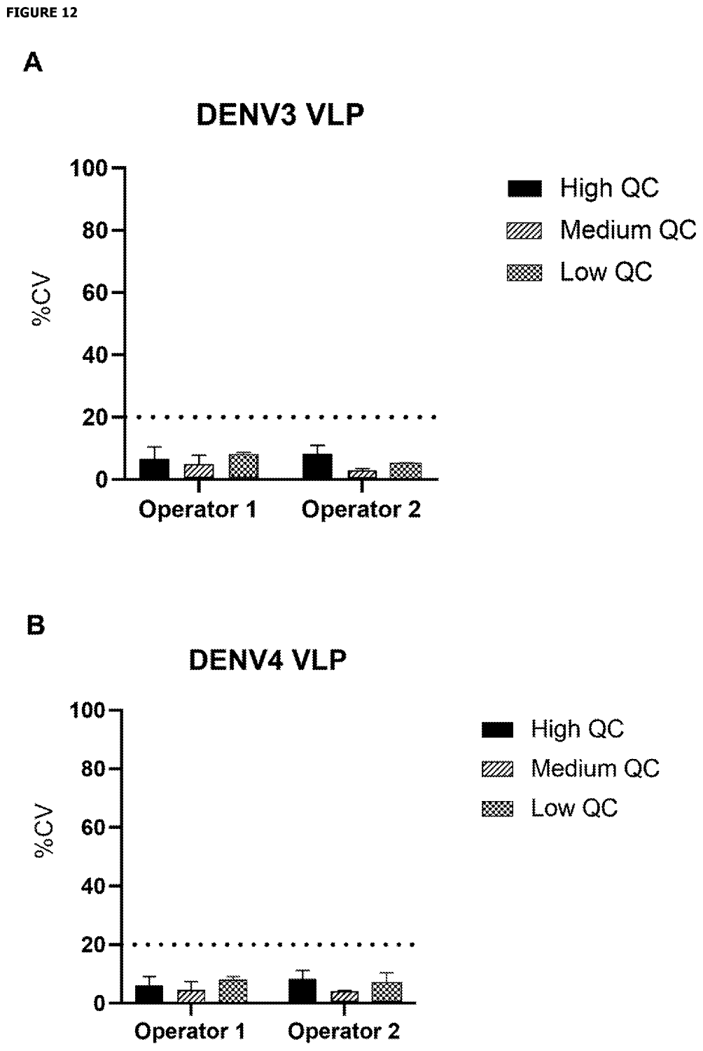

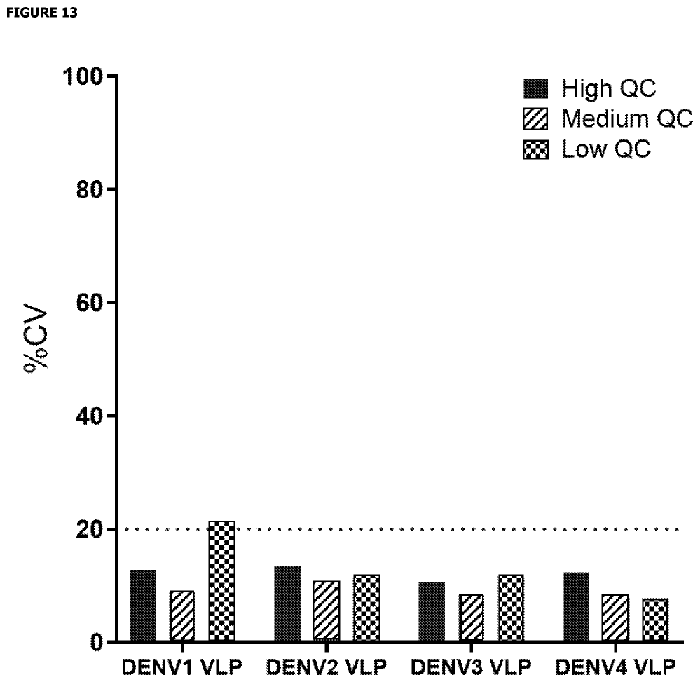

Intra-assay precision was consistently below 20% for all control samples and DENV VLPs (FIGS. 11 and 12). Inter-assay precision was below 20% for all control samples and DENV VLPs, except for the DENV1 VLP signals from the low control sample, which was solely slightly above 20% (FIG. 13).

In conclusion, the DENV-quadruplex complement-fixing assay was successfully set-up and validated.

### Example 3: Comparison of the DENV-Quadruplex Assay Set-Up with the Singleplex Assay Set-Up

In order to further evaluate the performance of the developed DENV-quadruplex complement-fixing assay set-up of Example 2, the quadruplex assay was compared to the corresponding singleplex complement-fixing assay set-ups solely comprising one DENV serotype VLP per assay. The singleplex assay was performed mutatis mutandis as described for the quadruplex assay under Example 2. The final microsphere concentration in the assay was the same for each serotype independent of singleplex or multiplex set-up. Complement fixing antibody titers were determined in the high, medium, and low titer control samples described in Example 2. In addition, a medium-high titer control sample (ABO Pharmaceuticals, Lot No. PARS_73) was included (Table 6).

TABLE 6
Complement-fixing antibody titers (EU/mL) detected in the high (H), medium-high (MH), medium (M),
and low (L) titer control samples using the quadruplex (QP) and the singleplex (SP) assay set-up
for all DENV VLPs. The titers were compared using percent coefficient of variation (% CV).

| | Complement-Fixing Antibody Titers (EU/mL) | | | | | | | | | | | |
|---|---|---|---|---|---|---|---|---|---|---|---|---|
| | DENV1 VLP | | | DENV2 VLP | | | DENV3 VLP | | | DENV4 VLP | | |
| Sample | QP | SP | % CV | QP | SP | % CV | QP | SP | % CV | QP | SP | % CV |
| H | 248 | 298 | 13% | 191 | 207 | 6% | 214 | 178 | 13% | 154 | 160 | 3% |
| MH | 144 | 157 | 6% | 82 | 88 | 5% | 81 | 101 | 16% | 60 | 61 | 1% |
| M | 27 | 29 | 4% | 16 | 20 | 16% | 19 | 18 | 6% | 17 | 19 | 8% |
| L | 5 | 7 | 24% | 4 | 6 | 34% | 4 | 4 | 1% | 3 | 4 | 15% |

In summary, the titers were consistent and showed good correlation, further validating the performance of the quadruplex complement-fixing assay.

### Example 4: Detection and Quantification of DENV-Reactive Complement-Fixing Abs in Human Serum Samples Vaccinated with a Tetravalent DENV Vaccine

Virus-reactive complement-fixing Abs can be produced upon natural virus infection or vaccination. Complement fixation improves antigen-specific B cell clone selection and affinity maturation by sequestration (via complement receptors) of antigens in germinal centers of secondary lymphoid organs and thus contributes to antibody production against the viral pathogen.

As the determination of complement-fixing Abs is an important tool to characterize the quality of the immune response after infection or vaccination, the quadruplex assay set-up developed under Example 2 was further applied to analyze human serum test samples for the presence and amount of DENV1-4-reactive complement-fixing Abs in the following. The samples were derived from subjects vaccinated with a tetravalent DENV vaccine candidate, comprising live-attenuated viruses of all four serotypes (TAK-003 vaccine from Takeda Vaccines, Inc.). The components of the tetravalent DENV vaccine are based on DENV1 strain 16007 Thailand (NCBI No. AAF59976.1), DENV2 strain 16681 Thailand (Uniprot No. P29990.1), DENV3 strain 16562 Philippines (Uniprot No. A0A173DS74), and DENV4 strain 1036 Indonesia (Uniprot No. A0A1Z1XCD1). Of note, the DENV2 strain used for tetravalent DENV vaccine production was the same as the one used for VLP production (cf. Example 1). The other strains used for tetravalent DENV vaccine production show high homology to the E protein sequences of the strains used for VLP preparation (97%, 97%, and 99% sequence identity of corresponding E protein sequences of DENV1, DENV3, and DENV4 strains).

Per analysis run, the reference sample as well as negative, high, medium, and low control samples of Example 2 were included. In addition, five human serum test samples were analyzed (samples 1 to 5). The assay and data analysis was carried out as described under Example 2, including heat inactivation of all samples. For data analysis, the human serum test samples were analyzed as described for the control samples.

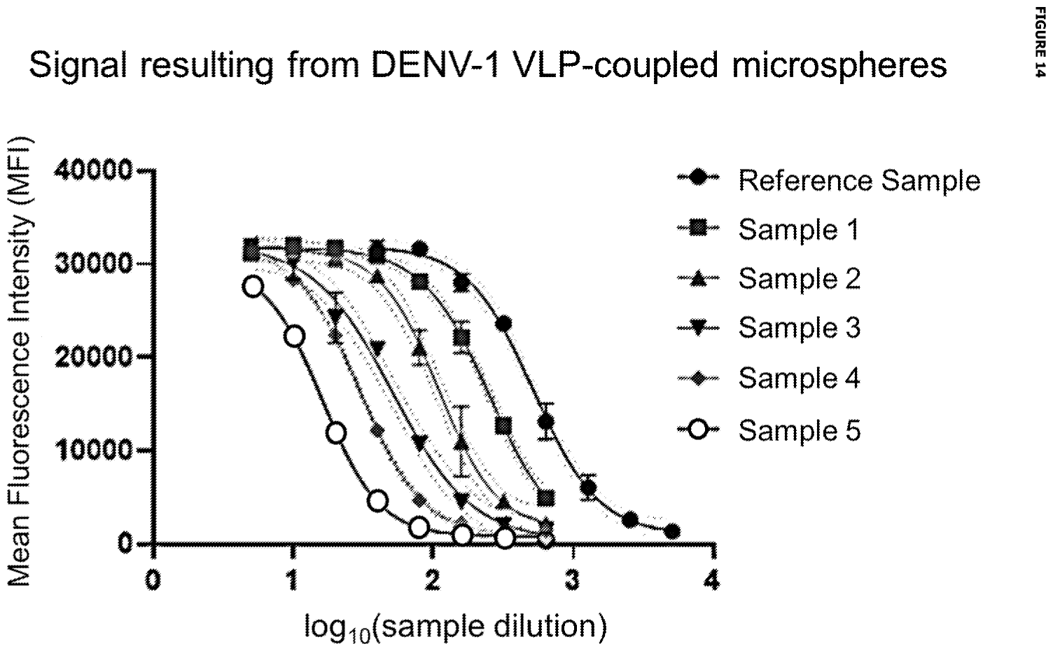

FIG. 14 shows the MFI values of the different sample dilutions of reference and human serum test samples exemplarily for DENY-1 VLP-coupled microspheres. Data analysis revealed final sample titers of 468.0 EU/mL for the reference sample, 214.8 EU/mL for sample 1, 94.7 EU/mL for sample 2, 50.8 EU/mL for sample 3, 26.0 EU/mL for sample 4, and 12.7 EU/mL for sample 5 of DENY-1-reactive complement-fixing Abs (cf. Table 7).

TABLE 7
Data analysis and evaluation of DENV-1-reactive complement-fixing Ab titers. The MFI signal
threshold from the reference sample was calculated by subtracting the estimated bottom
(MFI = 1,039) from top signals (MFI = 31,950) and multiplying the result by 0.25
(signal equivalent to 25% of effective concentration), resulting in a threshold of 7,727.75.

| | | | | | | Signal at | Signal at |
|---|---|---|---|---|---|---|---|
| | | | Complement- | | | maximum | minimum |
| | Interpolated | Relative | Fixing Abs | EC₅₀ | | dilution | dilution |
| Sample | dilution | dilution | [EU/mL] | value | R² | [MFI] | [MFI] |
| Reference | 1,037.38 | 1.000 | 468.00 | 520.10 | 0.9953 | 1,366.50 | 31,529.50 |
| Sample 1 | 476.10 | 0.459 | 214.78 | 256.90 | 0.9959 | 4,957.00 | 31,187.00 |
| Sample 2 | 209.95 | 0.202 | 94.72 | 109.20 | 0.9909 | 2,179.00 | 31,638.00 |
| Sample 3 | 112.70 | 0.109 | 50.84 | 51.27 | 0.9882 | 1,501.75 | 31,651.50 |
| Sample 4 | 57.69 | 0.056 | 26.03 | 30.08 | 0.9993 | 626.50 | 31,138.50 |
| Sample 5 | 28.11 | 0.027 | 12.68 | 16.18 | 0.9990 | 440.75 | 27,663.50 |

In conclusion, the DENV-quadruplex complement-fixing assay set-up was well suitable for evaluation of samples from subjects vaccinated with the tetravalent DENV vaccine and resulted in a robust determination of complement fixing Abs.

### Example 5: Comparison Between Seropositive and Seronegative Subjects

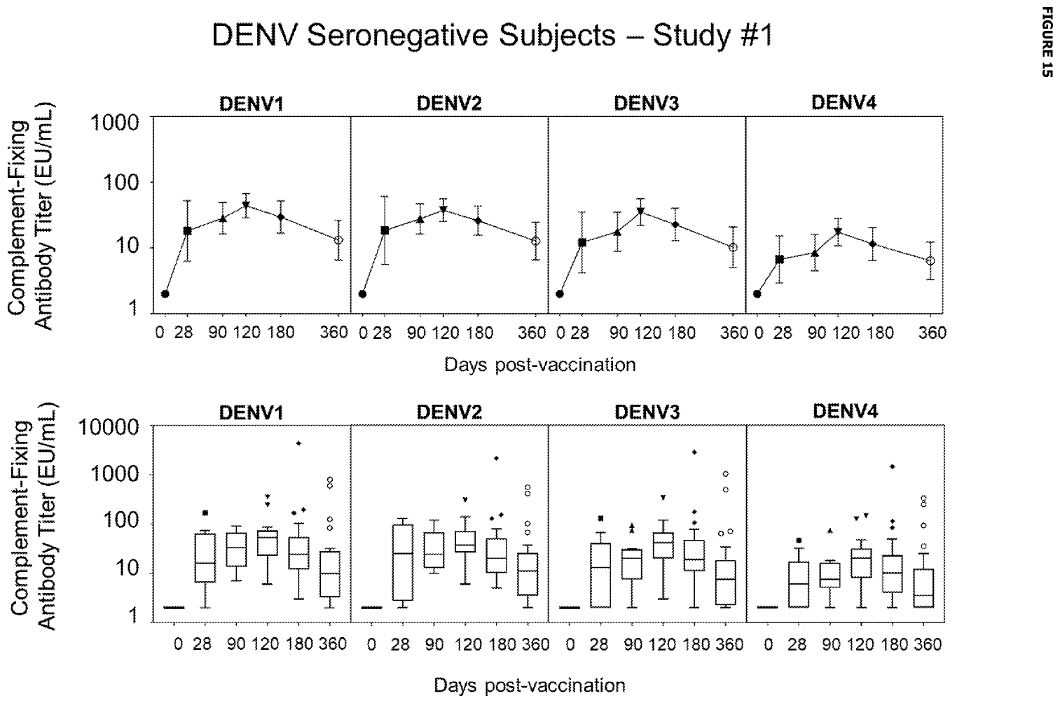

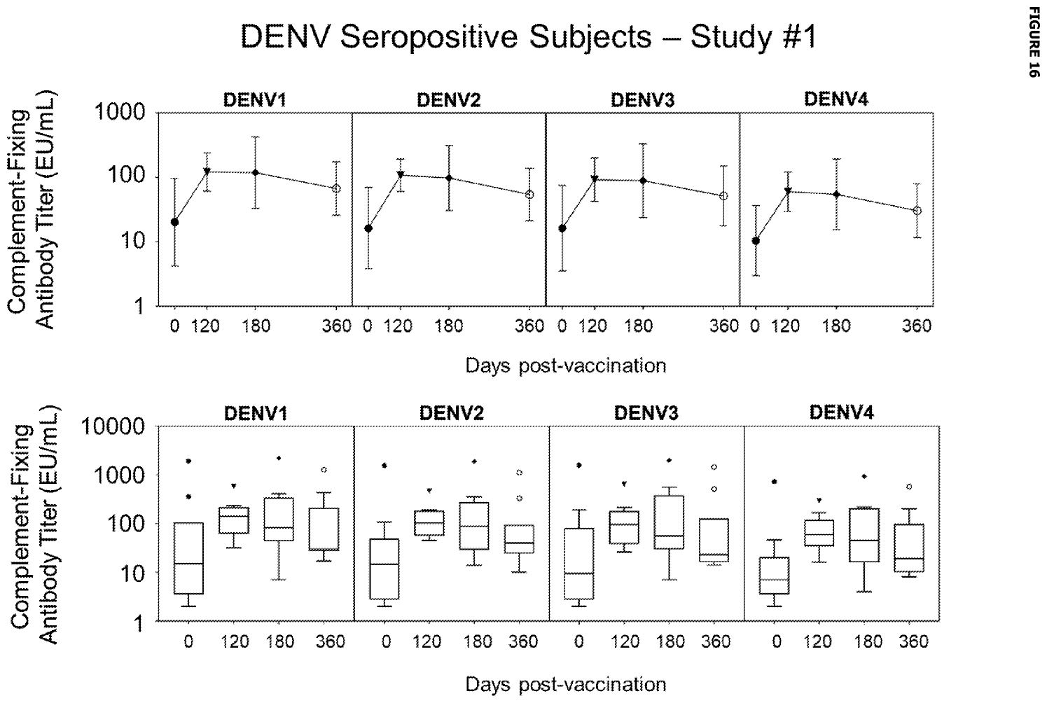

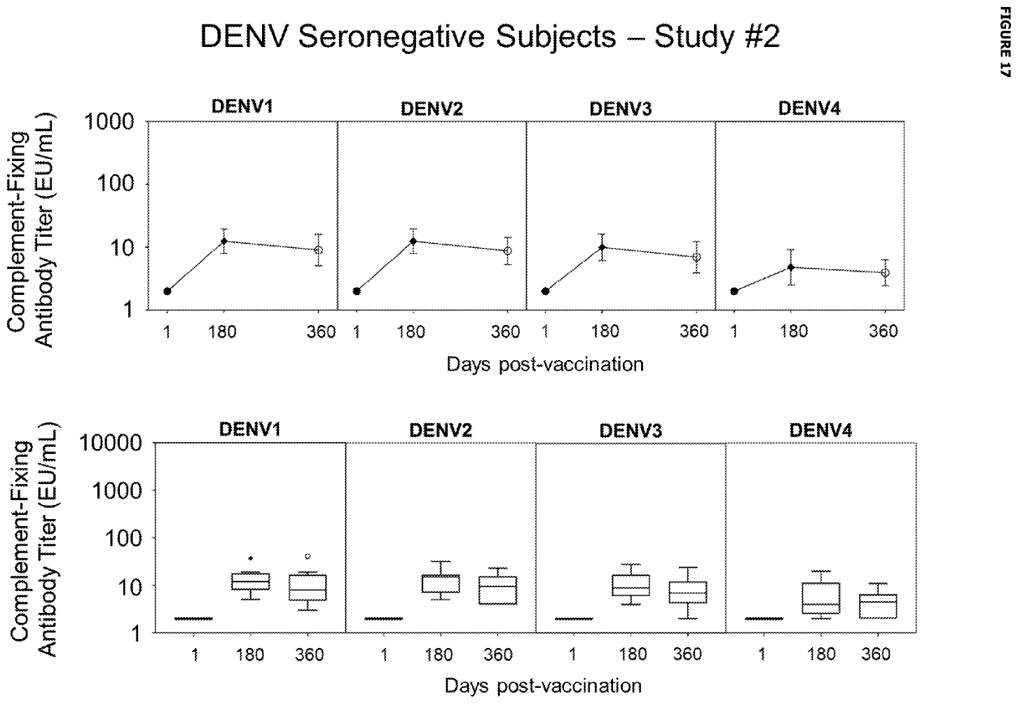

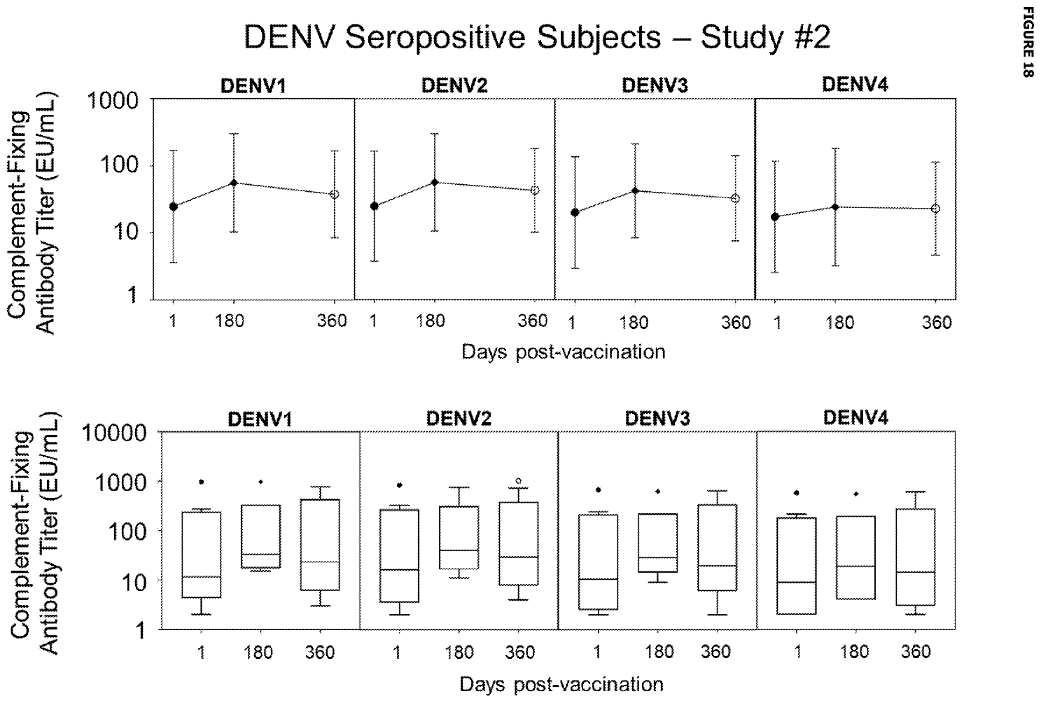

With a validated DENV-quadruplex complement-fixing assay set-up in hand, serum samples from both, seronegative and seropositive subjects from two different studies before and after vaccination with the tetravalent DENV vaccine from Example 4 were evaluated (FIGS. 15-18). 28 seronegative and 11 seropositive subjects were tested in study #1 (FIGS. 15 and 16) and 11 seronegative and 10 seropositive subjects were tested in study #2 (FIGS. 17 and 18). The titers of complement-fixing Abs against all DENV serotypes increased upon vaccination, indicating that the vaccine induced the production of Abs directed against all serotypes that can fix complement system.

Complement-fixing Ab titers prior to vaccination (samples at day 0 in FIGS. 15 and 16 and samples at day 1 in FIGS. 17 and 18, respectively) are in line with the serostatus of the subjects, i.e. close to zero for the seronegative cohort and around 10 for the seropositive cohort indicating that the assay can be applied to distinguish whether a subject has been infected with DENV or not.

Altogether, this data further validate a reliable assay performance and suitability of the assay for analysis of samples derived from subjects prior and post vaccination, independent of the serostatus of the subjects prior to vaccination.

### Example 6: Comparison of Microneutralization Test (MNT) and Complement-Fixing Assay

The DENV-quadruplex complement-fixing antibody assay of Example 2 was compared to the microneutralization test (MNT), which is a gold standard technique. Although the MNT is widely applied, the assay is time-consuming as for instance one step requires infection of and incubation with cells in culture.

To compare both assays, 53 samples derived from DENV seropositive and seronegative non-vaccinated subjects (aged 1.5 to 45 years living in dengue endemic areas) were evaluated. Out of the 53 samples, 14 samples were derived from subjects aged 1-5 years, 20 samples were derived from subjects aged 6-11 years, 12 samples were derived from subjects aged 12-20 years, and 7 samples were derived from subjects older than 20 years. The DENV-quadruplex complement-fixing antibody assay was carried out as described under Example 2, the MNT was carried out by heat-inactivating control and testing samples at 56° C. for 30 minutes. For each of the four serotype-specific MNT assays, a 30 μL heat-inactivated sample was serially diluted two-fold from 1:5 to 1:10240 and mixed with equal amounts of diluted dengue virus control for each serotype to give final dilution ratios of 1:10 to 1:20480. The virus control diluted in an equal amount of 10% (v/v) Dulbecco's Modified Eagle Medium (DMEM) provided the baseline foci count used to measure 50% neutralization. Neutralization occurred overnight at 2-8° C. This serum/virus mixture was then inoculated in triplicate on Vero cells that had been seeded onto 96-well plates at 2.5×10⁵ cells/mL approximately 24 hours previously. Absorbance was allowed to proceed for 90 minutes at 37° C. and ended with the addition of an overlay of 1% (w/v) methyl cellulose in DMEM. The infected cells were then incubated at 34° C.±2° C., 5±3% CO₂ for different time periods according to dengue serotype (DENV1 and DENV3: 66±2 hours; DENV2: 70±2 hours; DENV4: 46±2 hours). Thereafter, the cells were fixed with methanol for 60 minutes at ≤−20° C. or acetone for 20 minutes at room temperature. Infectious centers (immunofoci) were developed using a primary anti-dengue antibody (incubated at 2-8° C. for 18±4 hours), peroxidase-conjugated secondary antibody (incubated at 37° C.±2° C. for 90-120 minutes), and a precipitant substrate (2-amino-9-ethyl carbazole, Sigma). The peroxidase substrate formed a colored precipitate upon oxidation, allowing the immunofoci to be visualized. The antibody titer of each sample was determined by photographing the plates and counting the immunofoci in individual wells using a ViruSpot Plaque Counter (Autoimmun Diagnostika GmbH). Short viral amplification periods and microscopic visualization allowed for robust counting up to 136 foci. The percentage neutralization reduction was determined by comparing the number of immunofoci in averaged virus control-containing wells to the immunofoci number in serum sample wells. The MNT50 was calculated through linear interpolation using the equation MNT50=10\hat{ }[(50−c)/m], where c is the slope and m is the intercept. The linear interpolation utilized foci counts for the wells above and below the 50% virus control foci average. As described under Example 2, the LLOQ for the complement-fixing assay was 3.00 EU/mL, which was applied as a cut-off value for seropositivity. For the MNT, an MNT₅₀ titer of equal to or greater than 10 was applied as a cut-off for seropositivity.

In general, a good concordance in serostatus classification between the results from the MNT and the complement-fixing assay was observed (Table 8).

TABLE 8
Evaluation of samples from subjects that were seropositive
or seronegative for a specific DENV serotype by the MNT and
complement-fixing assay set-up. The cut-off titer for the MNT was
determined as a MNT₅₀ of equal to or greater than 10, the
cut-off titer for the complement-fixing assay as a titer
of equal to or greater than 3.00 EU/mL. In summary, samples
of 53 subjects were analyzed. The numbers show the percentage
of seropositive and seronegative subjects as predicted by
either the MNT or the complement-fixing assay.

| | | | | Complement-fixing | |
|---|---|---|---|---|---|
| | | MNT | | antibodies | |
| | | Titers ≥10 | Titers <10 | Titers ≥3.00 | Titers <3.00 |
| Viruses | Total | (%) | (%) | EU/mL (%) | EU/mL (%) |
| DENV1 | 53 | 17 (32) | 36 (68) | 15 (28) | 38 (72) |
| DENV2 | | 14 (26) | 39 (74) | 14 (26) | 39 (74) |
| DENV3 | | 16 (30) | 37 (70) | 13 (25) | 40 (75) |
| DENV4 | | 12 (23) | 41 (77) | 13 (25) | 40 (75) |

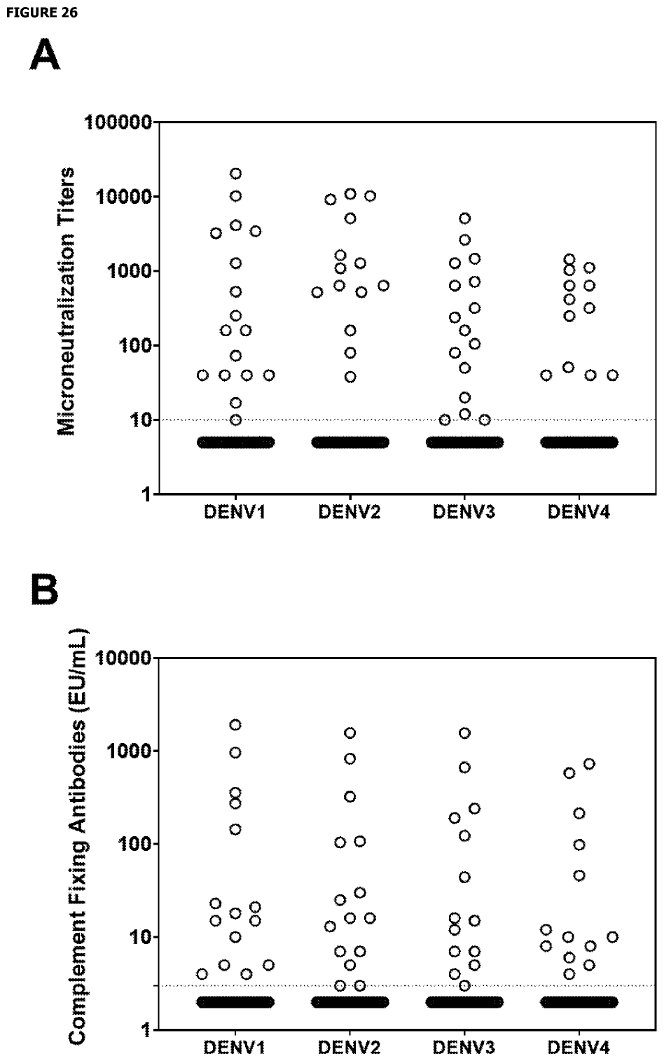

FIG. 26 and Table 9 depict the overall distribution of antibody titers in the samples, as well as geometric mean antibody titers, respectively, against all DENV serotypes by both assays. Geometric mean MNT50 titers ranged from 12 (DENV4) to 20 (DENV2) and the complement-fixing antibody geometric mean titers ranged from 4 EU/mL (DENV2-4) to 5 EU/mL (DENV1, Table 9).

TABLE 9
Neutralizing (MNT50) and complement-fixing antibody titers against
all four dengue virus serotypes from the 53 samples analyzed.
Minimum, maximum, as well as geometric mean (GeoMean) titers,
together with 95% confidence interval (CI) were determined for
both, the neutralizing and complement-fixing antibodies.

| | | | | | Complement-fixing antibody | | |
|---|---|---|---|---|---|---|---|
| | MNT50 Titers | | | | titers (EU/mL) | | |
| | Min- | Max- | GeoMean | | Min- | Max- | GeoMean |
| Virus | imum | imum | (95% CI) | | imum | imum | (95% CI) |
| DENV1 | 5 | 20480 | 19 | (10, 35) | 2 | 1915 | 5 (3, 7) |
| DENV2 | 5 | 10919 | 20 | (10, 40) | 2 | 1565 | 4 (3, 7) |
| DENV3 | 5 | 5120 | 15 | (9, 26) | 2 | 1564 | 4 (3, 7) |
| DENV4 | 5 | 1442 | 12 | (7, 20) | 2 | 730 | 4 (3, 6) |

MNT—dengue microneutralization assay;
MNT50 LLOQ is 10;
GeoMean—geometric mean;
CI—confidence interval

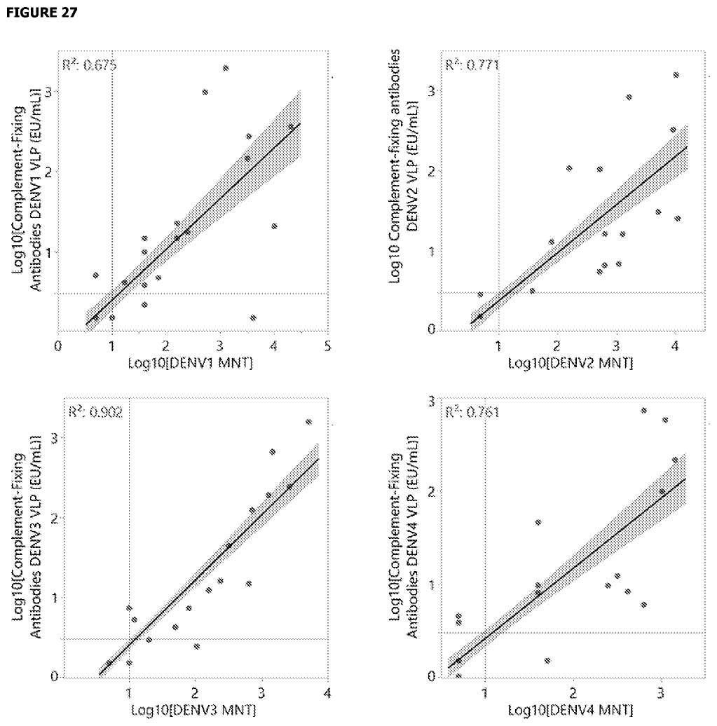

Correlation analysis between complement-fixing antibody titers and microneutralization titers was carried out using Log₁₀-transformed values using JMP version 13.1.0 software (SAS Institute, Inc.). When the relationship between the MNT50 and the complement-fixing antibodies was investigated, moderate (R²=0.675 for DENV1) to high (R²=0.902 for DENV3) correlations were observed (FIG. 27).

When compared to the MNT, specificity and sensitivity of the complement-fixing assay was above 80% for each serotype (Table 10), indicating that the performance of the complement-fixing assay is similar to the gold standard dengue MNT.

TABLE 10
Sensitivity and specificity of the complement-fixing assay relative to the DENV
MNT. The numbers indicate the true positives (samples with MNT50 ≥10) and true
negatives (samples with MNT50 <10) of the complement-fixing assay. Sensitivity
of the complement-fixing assay was calculated based on the percentage of samples
with an MNT50 ≥10 (seropositive) that were equal to or above the threshold
titer for positivity of 3.00 EU/mL. Specificity of the complement-fixing assay
was calculated based on the percentage of samples with an MNT50 <10 (seronegative)
that were below the threshold titer for positivity of 3.00 EU/mL. For instance,
all 39 DENV2-positive samples according to the MNT were also predicted to be positive
by the complement-fixing assay resulting in 100% sensitivity and specificity.

| | Number of Samples | | | | | |
|---|---|---|---|---|---|---|
| | | Comple- | | | Comple- | |
| | | ment >3.00 | Sensitivity | | ment <3.00 | Specificity |
| Viruses | MNT50 ≥10 | EU/mL | (%) | MNT50 <10 | EU/mL | (%) |
| DENV1 | 17 | 14 | 82% | 36 | 35 | 97% |
| DENV2 | 14 | 14 | 100% | 39 | 39 | 100% |
| DENV3 | 16 | 13 | 81% | 37 | 37 | 100% |
| DENV4 | 12 | 11 | 92% | 41 | 39 | 95% |

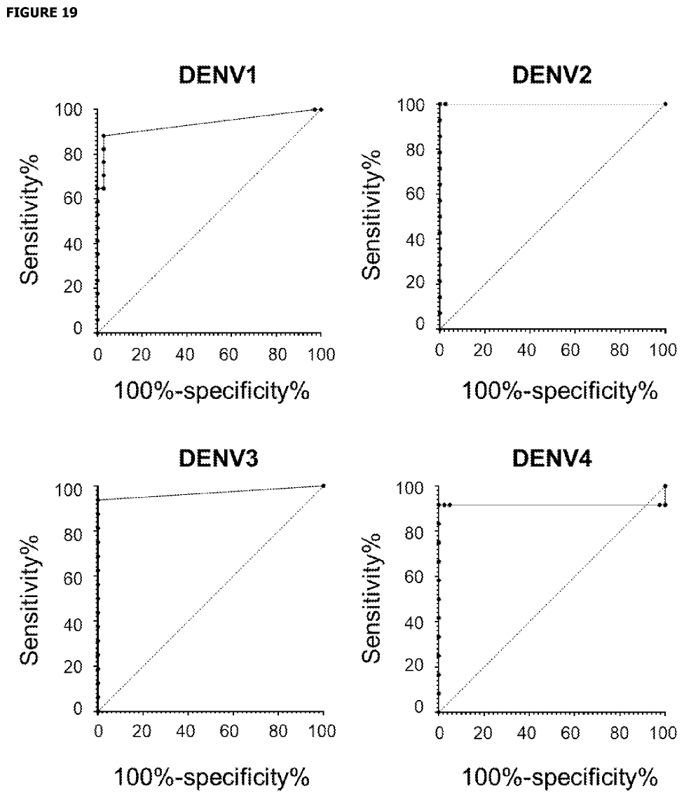

To illustrate the accuracy of the DENV-quadruplex complement-fixing assay set-up for diagnosis of a DENV infection, we evaluated the data in terms of a Receiver operating characteristic (ROC) curve. Therefore, the sensitivity, meaning the percentage of true positive samples i.e. samples from seropositive subjects with an MNT50 titer of ≥10 being also classified as positive in the complement assay is plotted on the y-axis and 1-specificity, meaning the percentage of true negative samples i.e. samples from seronegative subjects with an MNT50 titer of <10 being also classified as negative in the complement assay is plotted on the x-axis for several complement-fixing antibody titer thresholds (from <3 EU/mL to approximately 1000 EU/mL; FIG. 19). Then, the area under the curve (AUC) is calculated, which represents the accuracy of a diagnostic candidate test relative to the gold standard, i.e. the MNT. The AUC (95% confidence interval) was 0.9346 (0.8440 to 1.000) for DENV1, 1.000 (1.000 to 1.000) for DENV2, 0.9688 (0.8990 to 1.000) for DENV3, and 0.9167 (0.7603 to 1.000) for DENV4. An AUC over 0.9 represents an excellent diagnostic test, indicating that the DENV-quadruplex complement-fixing assay set-up is well suitable for diagnosing DENV infection.

### Example 7: Complement-Fixing Antibodies Evaluated in a 10-Plex Assay Set-Up

In a next step a 10-plex assay set-up including DENV1-4 VLPs, DENV1-4 NS1 proteins, as well as Zika virus (ZIKV) VLP, and ZIKV NS1 protein was developed. The goal was to determine complement-fixing Abs directed against structural and non-structural proteins of DENV and ZIKV in one single experiment. Antigens were coupled to the microspheres as described under Example 1. The assay was carried out mutatis mutandis as described under Example 2, with the exception that no titer in EU/mL was calculated (the samples were evaluated looking at MFI signal only).

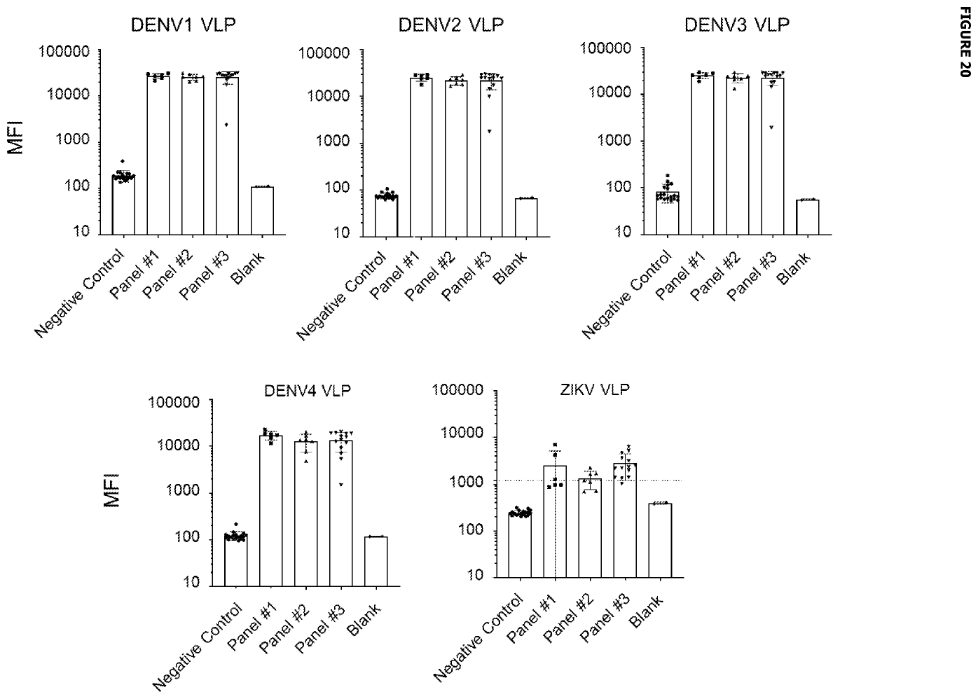

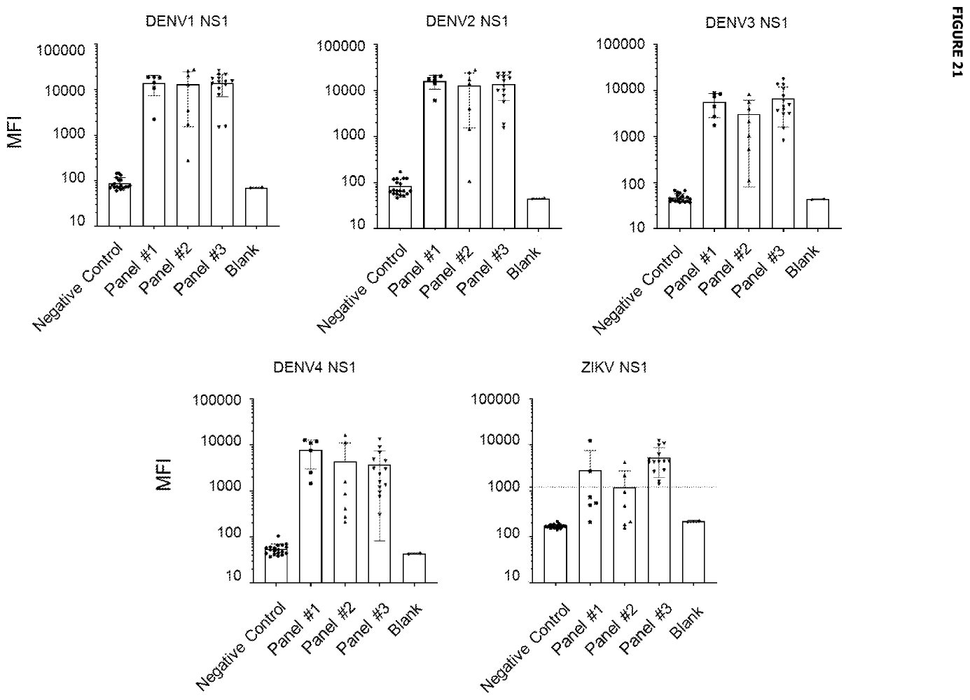

Complement-fixing Abs were determined in samples collected in the Dominican Republic in 2016 to 2017 from subjects suffering from febrile illness consistent with DENV and ZIKV infections (panel #1), as well as in samples collected in Colombia either before or after 2016 from subjects 8-40 days post febrile illness consistent with DENV and ZIKV infection (panel #2 and #3). In addition, samples, which did not contain flavivirus Abs were evaluated as negative controls (FIGS. 20-21).

Negative controls resulted in fluorescence values similar to the blank, validating the overall 10-plex assay set-up. High signals were observed for samples #1 to 3 for DENV1-4 VLPs and DENV1-4 NS1 proteins. Lower signals were detected for the ZIKV VLP and NS1 protein. In general, the complement-fixing assay was successfully transferred to a set-up comprising 10 different antigens.

Moreover, the DENV1-4 VLP signals from the 10-plex assay were similar to the signals recorded from the quadruplex assay set-up using DENV1-4 VLPs. Consequently, the number of antigens in the complement-fixing assay set-up of the present invention can be increased to any desired number as the results of the assay for specific antigens are not altered dependent on the number of antigens included.

### Example 8: Application of the DENV-Quadruplex Complement-Fixing Assay Set-Up to Distinguish Between DENV Serotype Infections

In a next step, it was evaluated if the DENV-quadruplex complement-fixing assay set-up developed under Example 2 is suitable for distinguishing between infections with different DENV serotypes. Therefore, 16 non-human primates (adult male cynomolgus macaques 5-7 years, weighing 6-9 kg of Mauritius origin and naïve to DENV) were infected subcutaneously with each one of the four DENV serotypes. For infection, 2.47e7 PFU/mL DENV1 strain Western Pacific, 3.11e6 PFU/mL DENV2 strain New Guinea C, 2.94e6 PFU/mL DENV3 strain Sleman/78, and 5.30e6 PFU/mL DENV4 strain 1228 were used per animal. All monkeys were negative for Hepatitis B virus, Simian Retrovirus, Simian Immunodeficiency virus, Simian T Lymphotropic virus, Mycobacterium Tuberculosis, Simian Varicella virus, Malaria, Salmonella, Shigella, Yersinia and internal parasites. Of note, human C1q cross-reacts with monkey complement-fixing Abs and can therefore be used for evaluation of monkey samples.

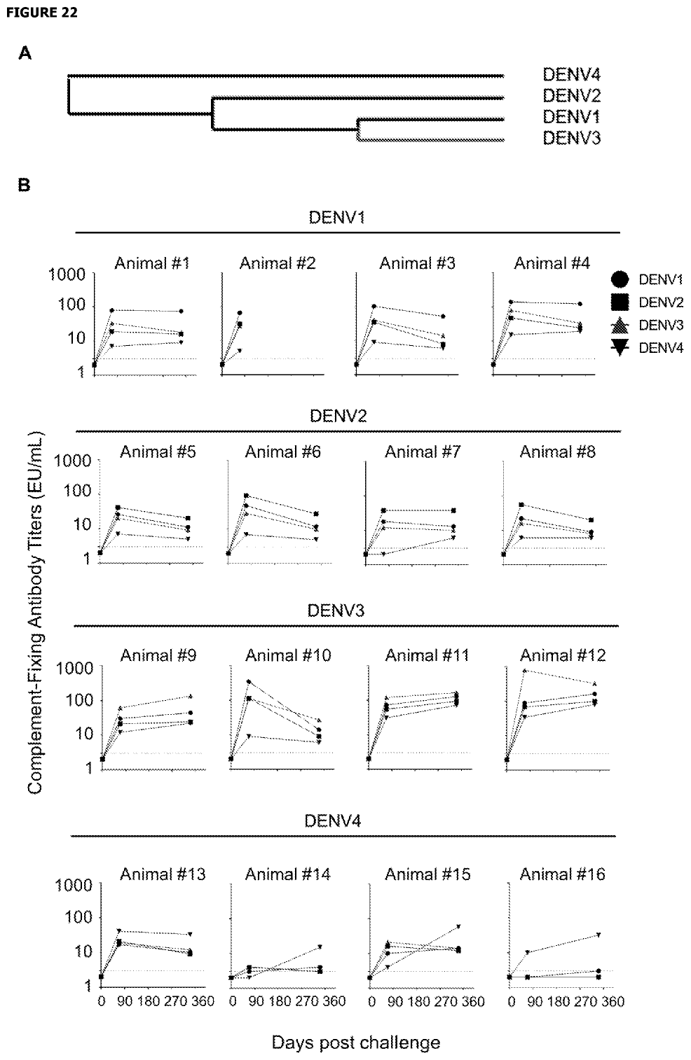

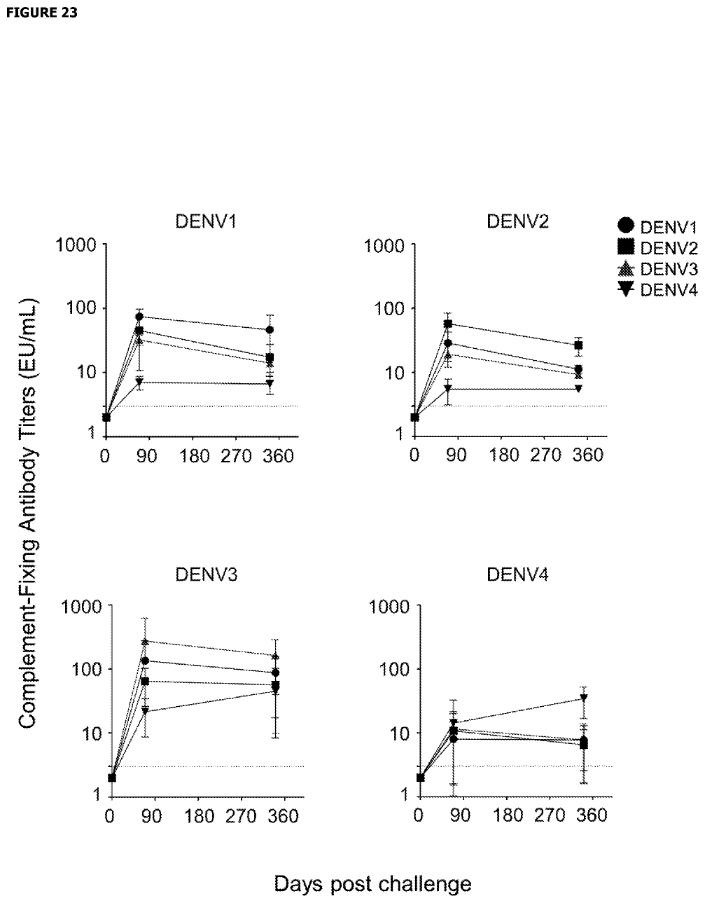

Complement-fixing Ab titers for each animal and DENV serotype were determined (FIG. 22B) and averaged for animals infected with the same serotype (FIG. 23). The titers of complement-fixing Abs for the infecting serotype were higher for each animal. Complement-fixing Abs were also cross-reactive amongst the different serotypes. The amount of cross-reactive complement-fixing Abs observed was consistent with the inter-serotype homology of the envelope protein (FIG. 22A).

Both serotype-specific and cross-reactive Abs are involved in complement fixation. The less similar the envelope amino acid sequence among the DENV serotypes, the less cross-reactive Abs are produced (e.g. the amounts of DENV4 cross-reactive Abs produced in DENV1-3 infected animals were the lowest). However, the data show that the DENV quadruplex complement-fixing assay is able to distinguish between infections caused by different DENV serotypes.

### Example 9: Application of the 10-Plex Assay Set-Up to Distinguish Between Flavivirus Infections

As Abs are often cross-reactive amongst different flaviviruses, the capacity of the complement-fixing assay of the present invention to distinguish between infections by different flaviviruses was evaluated. Therefore, the 10-plex assay set-up developed under Example 7 was applied to detect complement-fixing Abs directed against ZIKV NS1.

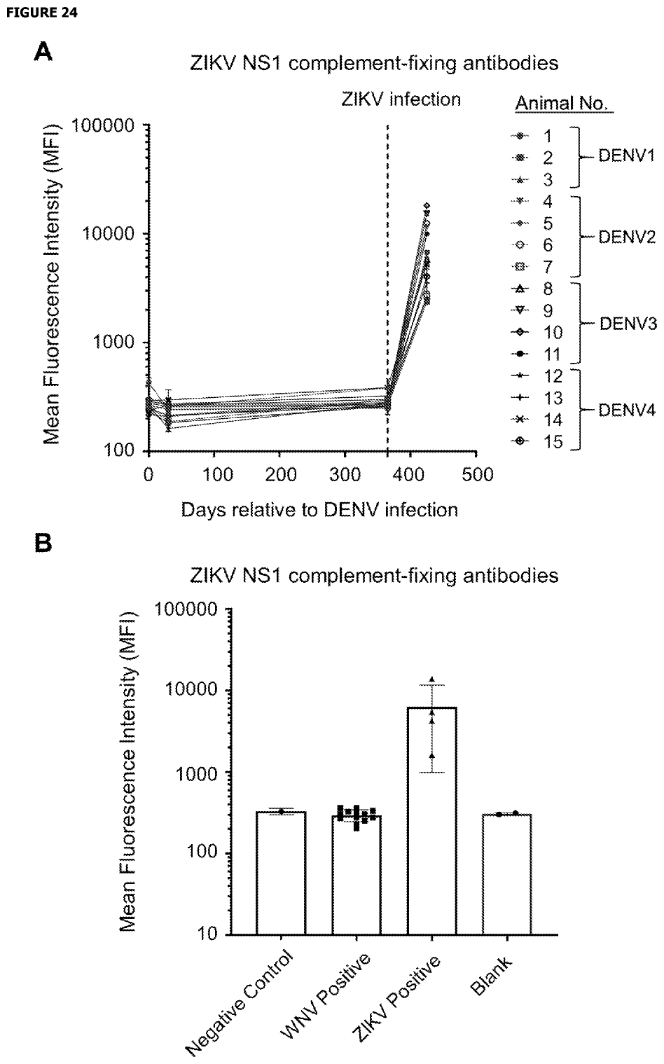

Non-human primates were infected with each one DENV serotype as described under Example 8 and complement fixing Abs directed against ZIKV NS1 were monitored for one year after infection. Then, non-human primates were infected with ZIKV and complement-fixing Abs directed against NS1 were further evaluated (FIG. 24A). Interestingly, none of the DENV infections, independent of the serotype, resulted in increasing ZIKV NS1-reactive complement-fixing Abs. However, after ZIKV infection, signal intensity significantly raised for all animals, indicating that the complement fixing assay is very specific and can also be used to reliably distinguish between ZIKV and DENV infection.

Further, the complement-fixing Abs in human samples with either Abs directed against West Nile Virus (WNV) or against ZIKV were evaluated in the 10-plex assay set-up to detect complement-fixing Abs directed against ZIKV NS1. In addition, a negative control sample lacking flavivirus-directed Abs was included. Satisfyingly, the assay was also able to distinguish between complement-fixing Abs binding to ZIKV and WNV (FIG. 24B) in line with the non-human primate data.

Altogether, this data indicate that samples can be evaluated for complement-fixing Abs directed against one specific flavivirus without interference from Abs generated upon infection with another flavivirus using NS1 as the antigen coupled to the microspheres in the complement-fixing assay set-up. This can be very useful in practice as multiple infections with different flavivirus are commonly observed as flaviviruses are co-circulating in several areas. The complement-fixing assay described in the present application is therefore also suitable for diagnosing by which flavivirus a subject was infected.

### Example 10: Application of the DENV-Quadruplex Complement-Fixing Assay Set-Up to Distinguish Between Flavivirus Infections

In a next step, it was evaluated if the DENV-quadruplex complement-fixing assay set-up developed under Example 2 is selective complement-fixing Abs generated upon DENV infection.

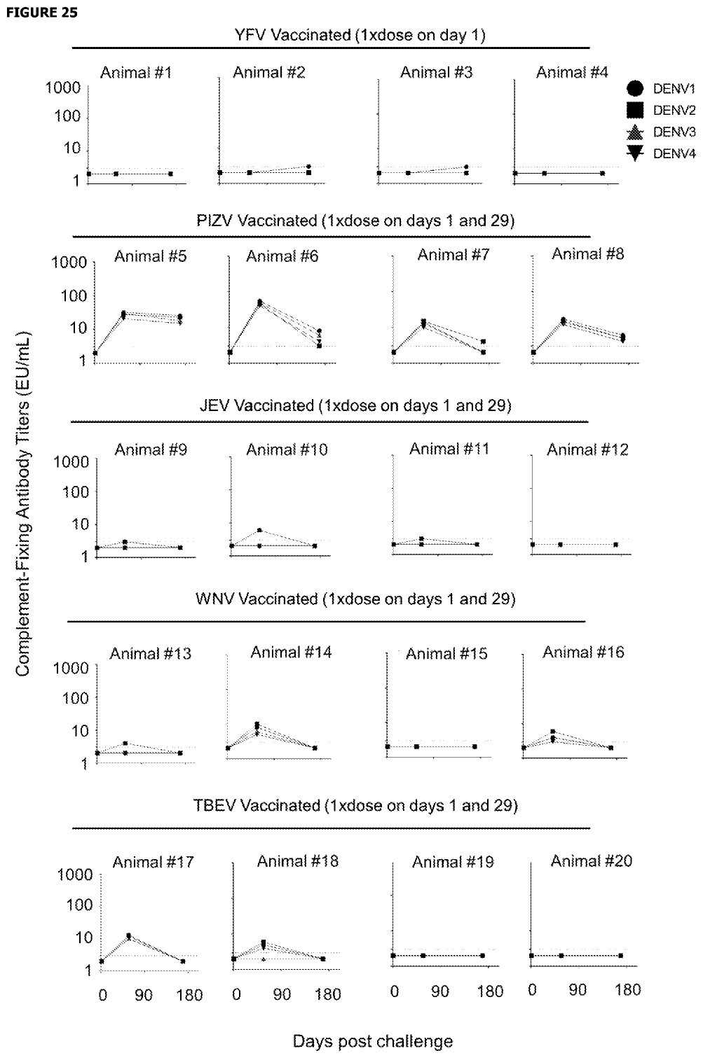

Therefore, four rhesus macaques per group (2-3 year old male flavivirus naïve Indian Rhesus macaques, weighing 3-6 kg) were vaccinated subcutaneously with one of a YFV (Stamaril®, Sanofi Pasteur), ZIKV (purified inactivated zika vaccine, PIZV, Takeda; see, for instance, WO 2019/090228), JEV (Ixiaro®, Valenva), WNV (Innovator®, Fort Dodge), or TBEV (Encepur®, GlaxoSmithKline) vaccine either in one dose (YFV vaccine at 1000 international units) or in two doses administered four weeks apart (ZIKV vaccine at 10 μg per dose; JEV vaccine at 6 AU (≤460 ng) per dose; 0.5 mL of WNV vaccine per dose; TBEV vaccine at 1.5 μg per dose). All monkeys were negative for Hepatitis B virus, Simian Retrovirus, Simian Immunodeficiency virus, Simian T Lymphotropic virus, Mycobacterium tuberculosis, Simian Varicella virus, Malaria, Salmonella, Shigella, Yersinia and internal parasites. Serum was collected for all animals on day 1 (prior to vaccination on the day of vaccination), day 57, and day 169 and analyzed by the DENV-quadruplex complement-fixing assay set-up. Except for ZIKV vaccinated animals, no or only low titers against all four DENV VLPs were observed, indicating a good specificity of the assay even in the presence of complement-fixing antibodies and other non-complement-fixing antibodies directed against various flaviviruses (FIG. 25). However, also for the ZIKV vaccinated animals signal decreased over time for most animals. This data further indicate, that vaccination against other flaviviruses except ZIKV (and probably also natural infection by other flaviviruses except ZIKV) does not induce complement-fixing antibodies that are capable of fixing and activating complement on DENV. However, also the cross-reactive complement-fixing Abs induced by vaccination against ZIKV wane overtime.

In summary, this data underline the suitability of the DENV-quadruplex complement-fixing assay set-up to reliable distinguish between flavivirus infections independent of the infection being acute or convalescent.

### Example 11: Comparison of Total IgG ELISA and Complement-Fixing Assay

The complement-fixing assay set-up was further compared to a dengue total binding IgG enzyme linked immunosorbent assay (ELISA). Therefore, the 53 samples from children and adults analyzed under Example 6 were further analyzed with the DENV-quadruplex complement-fixing assay set-up as described above under Example 2 or with a total IgG ELISA set-up as described below.

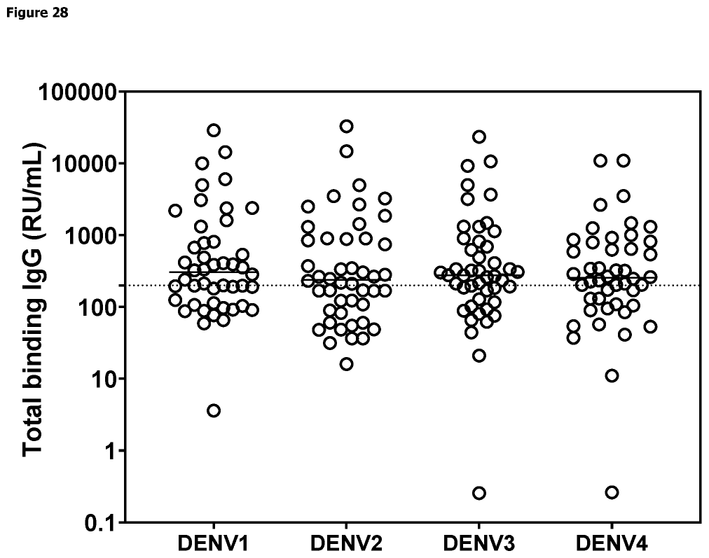

To quantitate DENV1, DENV2, DENV3 and DENV4 total binding IgG using an antigen capture ELISA, 96-well MaxiSorp microplates (Nunc) were coated with monoclonal antibody clone 4G2 (Absolute Antibody) prepared in 0.1M carbonate/bicarbonate buffer (Sigma) by overnight incubation at 4° C. Afterwards, microplates were blocked with SuperBlock T20 (Thermo Scientific) for 1 hour at 37° C. and washed with 1×PBS 0.1% (v/v) Tween 20 (PBS-T). Microplates were incubated with either serotype present in a tetravalent dengue vaccine (TAK-003 or DENvax, Takeda; for review, see, for instance, Osorio et al., 2011, Vaccine 29: 7251-7260) for 1.5 hours at 37° C. and washed with PBS-T. Serially diluted serum samples were then added to the microplate and incubated for 1 hr at 37° C. Following washes with PBS-T, horseradish peroxidase (HRP)-conjugated anti-IgG secondary antibody (Abcam) was added to the microplate and incubated for 1 hr at 37° C. After a final washing cycle with PBS-T, microplates were developed with ABTS (2,2′-Azinobis [3-ethylbenzothiazoline-6-sulfonic acid]-diammonium salt) peroxidase substrate (Seracare) for 15 minutes at room temperature. The reaction was stopped with 1×ABTS stop solution (Seracare) and microplates were read at 405 nm using a SpectraMax Plus 384 microplate reader (Molecular Devices). DENV-specific antibody levels were calculated relative to a reference standard using GraphPad Prism version 7 (Graph Pad Software). Serotype-specific total IgG levels are shown in FIG. 28.

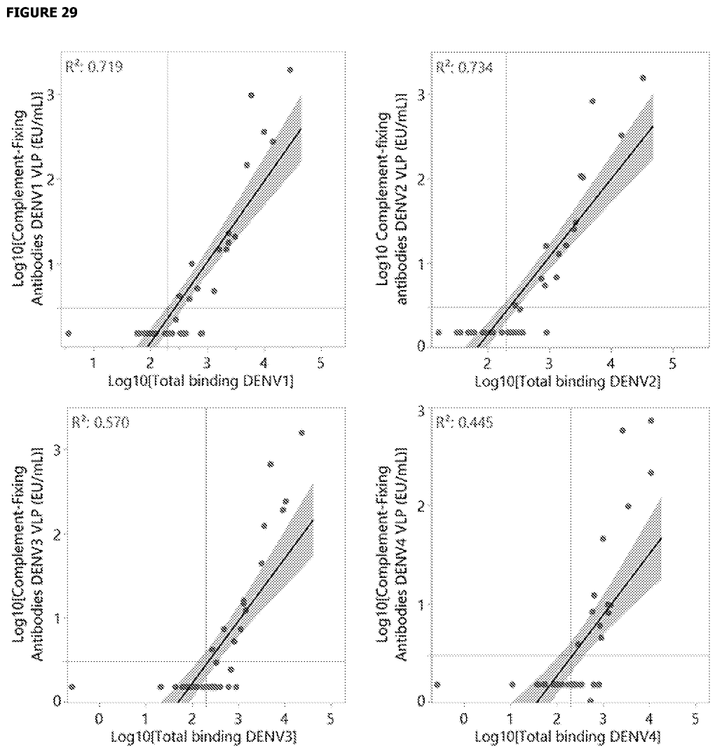

Correlation analysis between complement-fixing antibody titers and total IgG titers was carried out using Log₁₀-transformed values using JMP version 13.1.0 software (SAS Institute, Inc.). Moderate correlation was observed for the total IgG ELISA and the complement-fixing assay set-up (FIG. 29), indicating that total binding IgG produced in response to natural virus exposure are functional to activate complement.

### Example 12: The Complement-Fixing Assay Set-Up Based on C1q Fixation Translates to C3d Deposition

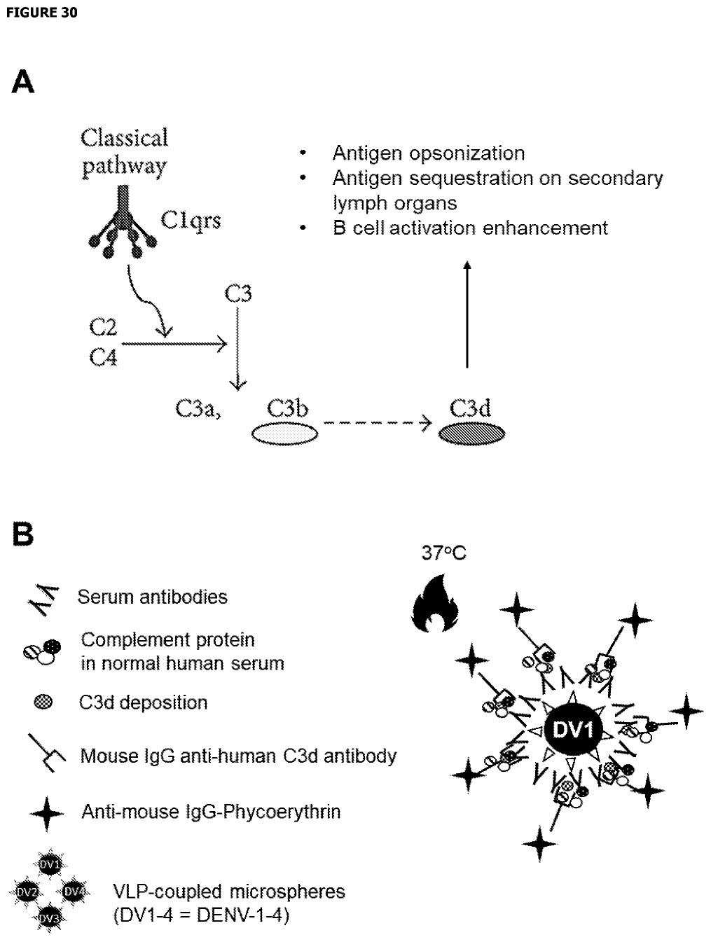

In a next step, it was evaluated whether the complement-fixing assay set-up based on the fixation of C1q as described above translates to complement C3d deposition (see also FIG. 30A), an early marker of complement system activation known to be involved in increased B cell responses.

The C3d deposition assay set-up (see also FIG. 30B) was carried out as described above for the C1q fixation set-up outlined under Example 2, unless otherwise noted. In particular, incubation temperatures and shaking, as well as microsphere counting was carried out as described above. In addition, also all samples evaluated in the C3d deposition assay were heat-inactivated as described above for the C1q fixation set-up. For the C3d deposition assay, 50 μL of VLP-coupled microspheres were combined with 50 μL of diluted samples and incubated for 1 hour as described under Example 2. Afterwards, the microspheres were washed with wash buffer (PBS supplemented with 0.05% (v/v) Tween-20) using the plate washer (BioTek, model ELx405). The antigen-antibody immunocomplex formed was detected by adding 50 μL/well of complement-competent human serum (Complement Technology) diluted 160-fold in 1×PBS and incubating for 15 min at 37° C. Following washing with wash buffer, the C3d deposition on the VLP-coupled microspheres by antigen-specific sample antibodies was detected by adding 50 μL/well of a pre-reporter antibody, i.e. mouse anti-human C3d IgG1 monoclonal antibody (Quidel) at 1.25 μg/mL and incubating for 30 min. After washing with wash buffer, microspheres were incubated with 50 μL/well of an anti-mouse IgG reporter antibody conjugated to phycoerythrin (Jackson Immunoresearch) at 10 μg/mL for 30 min. Afterwards, the microspheres were washed twice with PBS-T, reconstituted with 100 μL/well of assay buffer and read on the Magpix plate reader (Luminex Corporation) as described above under Example 2. C3d deposition antibody titers (or also shortly referred to as C3d deposition) were determined mutatis mutandis as described above for the complement-fixing antibody titers derived from the C1q-based assay. Correlation analysis between complement-fixing antibody titers based on C1q-fixation and C3d deposition antibody titers was carried out in GraphPad Prism version 8.1.0 (GraphPad Software, Inc.) using log₁₀-transformed values.

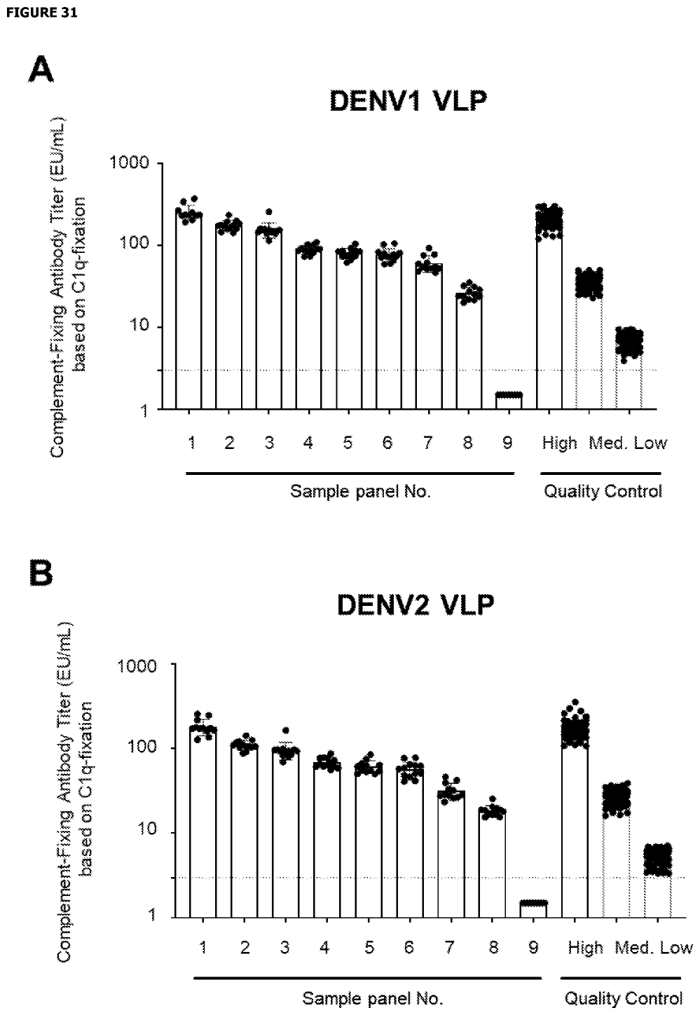

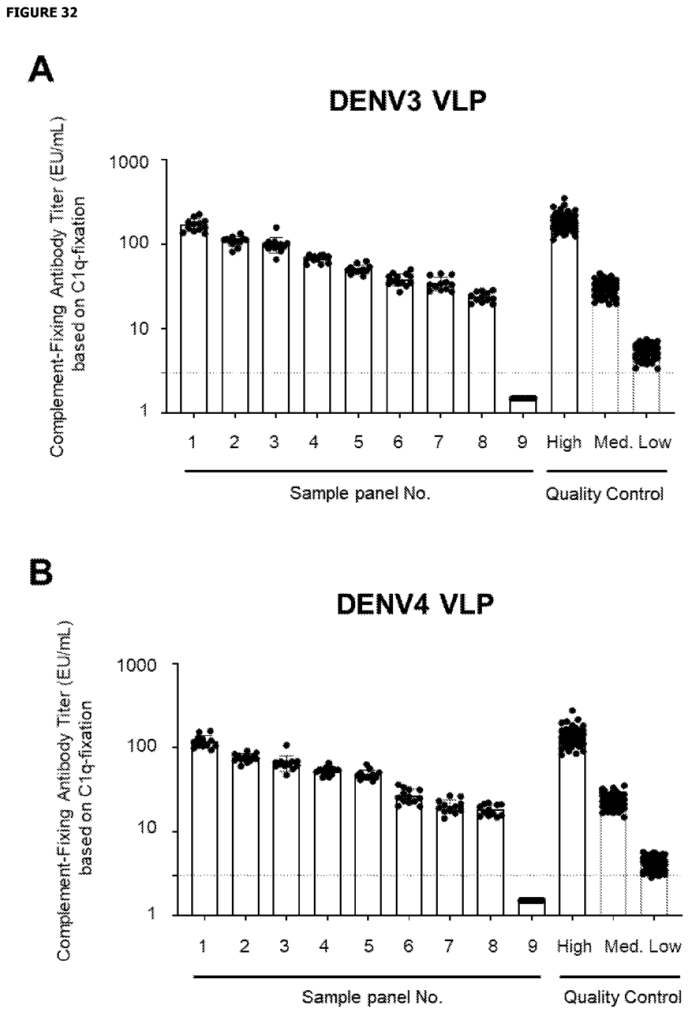

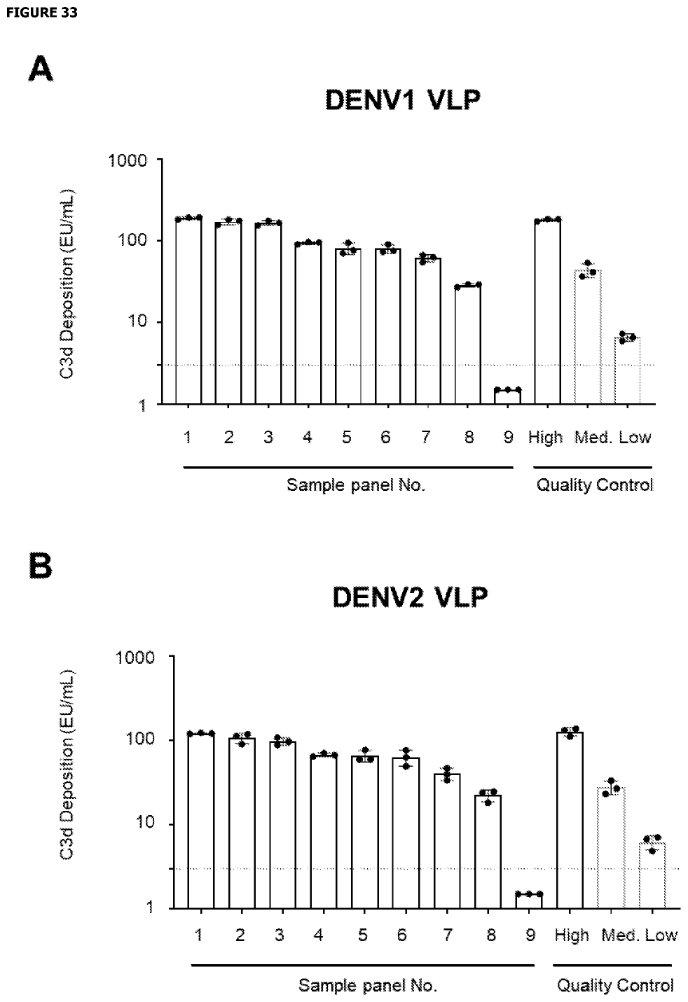

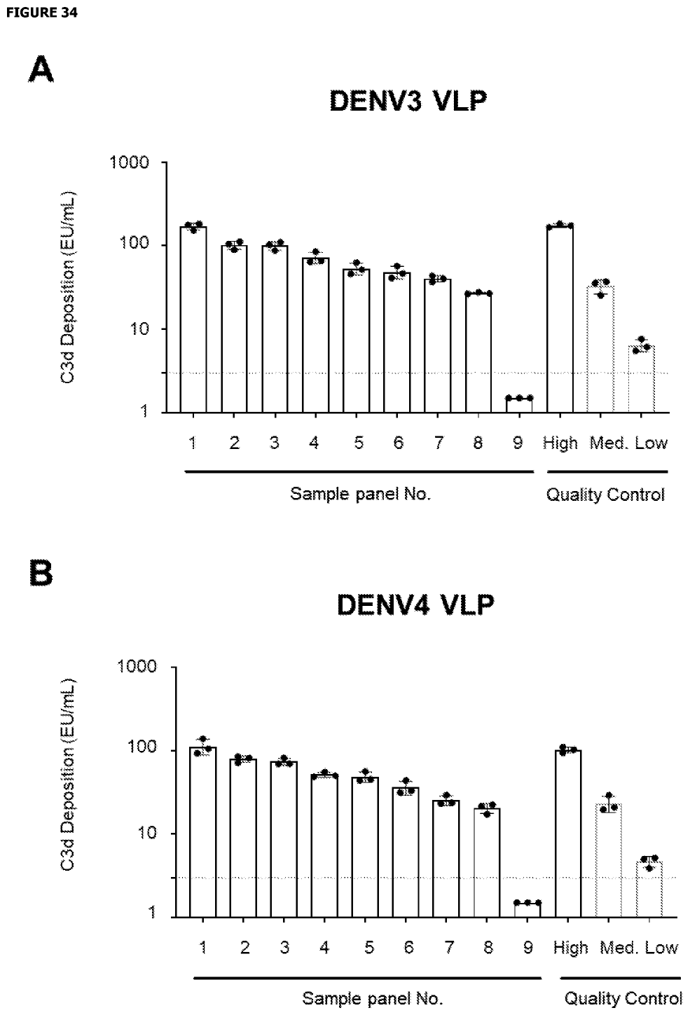

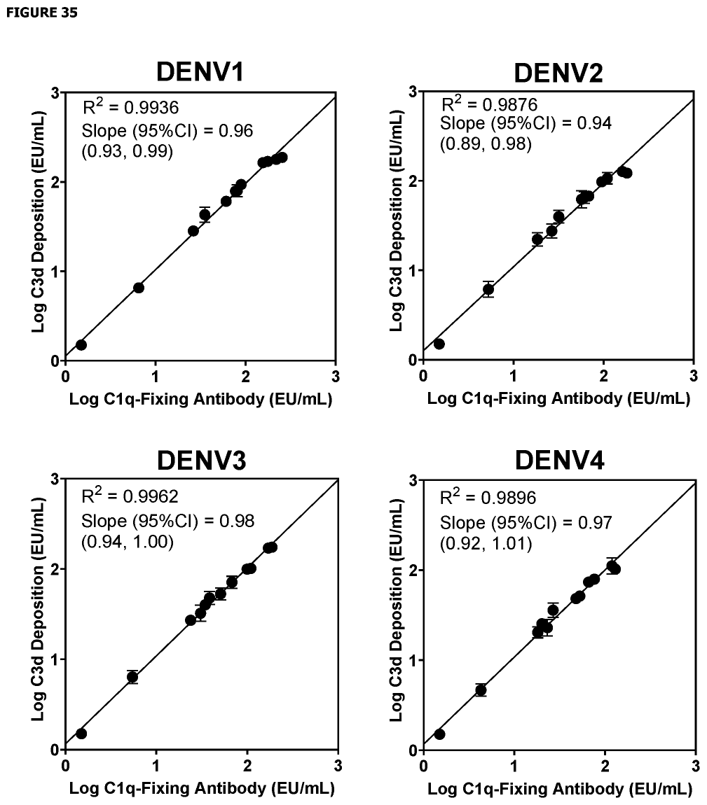

For comparison of the two set-ups (i.e. the C1q-based and the C3d-deposition DENV-quadruplex complement-fixing assay set-ups), a panel of 9 samples from healthy subjects who were seropositive to DENY, with a wide range of complement-fixing antibody levels, was used to measure C1q-based complement-fixing antibody titers (as described above under Example 2), as well as C3d deposition (FIGS. 31-34). The 9 serum samples collected from adults living in DENY-endemic areas in Colombia from 2015-2016 were purchased from ABO Pharmaceuticals. In addition, high, medium, and low control samples, comprising high, medium, and low titers of DENY-reactive complement-fixing Abs were examined (see, for instance, also Example 2). The overall pattern of C3d deposition levels was similar to complement-fixing antibody titers. Both biomarkers were highly correlated, with a correlation coefficient (R²) and slope close to 1.000 irrespective of the DENV serotype (FIG. 35), validating that the C1q-based complement-fixing assay set-up can be used as a surrogate marker for complement system activation by antigen-specific antibodies.

### SEQUENCE LISTING

Genomic sequences of flaviviruses are presented in the form of DNA in the sequence listing. As flaviviruses are RNA viruses it is clear to the person skilled in the art that the thymidine residues need to be replaced by uridine. Table 11 gives an overview of sequences of the present application.

Table 11 Overview of sequences of the present application

| SEQ ID | | | Sequence |
|---|---|---|---|
| NO: | Organism | Strain | Type |
| 1 | Dengue virus | Puerto Rico/US/BID-V853/1998 | Amino Acid |
| | serotype 1 | | |
| 2 | Dengue virus | Thailand/16681/84 | Amino Acid |
| | serotype 2 | | |
| 3 | Dengue virus | Sri Lanka D3/H/IMTSSA- | Amino Acid |
| | serotype 3 | SRI/2000/1266 | |
| 4 | Dengue virus | Dominica/814669/1981 | Amino Acid |
| | serotype 4 | | |
| 5 | Dengue virus | Puerto Rico/US/BID-V853/1998 | Genomic |
| | serotype 1 | | |
| 6 | Dengue virus | Thailand/16681/84 | Genomic |
| | serotype 2 | | |
| 7 | Dengue virus | Sri Lanka D3/H/IMTSSA- | Genomic |
| | serotype 3 | SRI/2000/1266 | |
| 8 | Dengue virus | Dominica/814669/1981 | Genomic |
| | serotype 4 | | |
| 9 | Zika virus | Suriname Z1106033 | Amino Acid |
| 10 | Zika virus | Suriname Z1106033 | Genomic |
| 11 | Dengue virus | Nauru/Western Pacific/1974 | Amino Acid |
| | serotype 1 | | |
| 12 | Dengue virus | Nauru/Western Pacific/1974 | Genomic |
| | serotype 1 | | |
| 13 | Dengue virus | NS1 | Amino Acid |
| | serotype 1 | | |
| 14 | Dengue virus | NS1 | Amino Acid |
| | serotype 2 | | |
| 15 | Dengue virus | NS1 | Amino Acid |
| | serotype 3 | | |
| 16 | Dengue virus | NS1 | Amino Acid |
| | serotype 4 | | |

### Items of the Invention

### Microsphere Complex

1. A microsphere complex comprising a microsphere coupled to a flavivirus antigen.
2. The microsphere complex according to item 1, wherein the flavivirus is selected from the group consisting of dengue virus, zika virus, West Nile virus, Japanese encephalitis virus, Tick-Borne encephalitis virus, Yellow Fever virus, Murray Valley encephalitis virus, and St. Louis encephalitis virus.
3. The microsphere complex according to any one of item 1 or 2, wherein the antigen is selected from the group consisting of virus like particle (VLP), non-structural protein 1, envelope protein, pre-membrane protein, membrane protein, capsid protein, non-structural protein 2A, non-structural protein 2B, non-structural protein 3, non-structural protein 4A, non-structural protein 4B, and non-structural protein 5 and any derivative thereof.
4. The microsphere complex according to item 3, wherein the antigen is a virus like particle.
5. The microsphere complex according to item 3, wherein the antigen is a non-structural protein 1.
6. The microsphere complex according to any one of items 1 to 5, wherein the flavivirus is a zika virus.
7. The microsphere complex according to any one of items 1 to 5, wherein the flavivirus is a West Nile virus.
8. The microsphere complex according to any one of items 1 to 5, wherein the flavivirus is a dengue virus.
9. The microsphere complex according to item 8, wherein the dengue virus is a dengue-1 virus.
10. The microsphere complex according to item 8, wherein the dengue virus is a dengue-2 virus.
11. The microsphere complex according to item 8, wherein the dengue virus is a dengue-3 virus.
12. The microsphere complex according to item 8, wherein the dengue virus is a dengue-4 virus.

### VLP

13. The microsphere complex of item 1, wherein the microsphere is coupled to a dengue-1 virus VLP.
14. The microsphere complex of item 13, wherein the dengue-1 virus VLP is derived from dengue-1 virus strain Puerto Rico/US/BID-V853/1998 characterized by SEQ ID NO: 1.
15. The microsphere complex of item 13, wherein the dengue-1 virus VLP comprises the envelope protein, the membrane protein, and the pre-membrane protein which are at least 80%, or at least 85%, or at least 90%, or at least 95% or at least 100% identical to corresponding parts of SEQ ID NO: 1.
16. The microsphere complex of item 1, wherein the microsphere is coupled to a dengue-2 virus VLP.
17. The microsphere complex of item 16, wherein the dengue-2 virus VLP is derived from dengue-2 virus strain Thailand/16681/84 characterized by SEQ ID NO: 2.
18. The microsphere complex of item 16, wherein the dengue-2 virus VLP comprises the envelope protein, the membrane protein, and the pre-membrane protein which are at least 80%, or at least 85%, or at least 90%, or at least 95% or at least 100% identical to corresponding parts of SEQ ID NO: 2.
19. The microsphere complex of item 1, wherein the microsphere is coupled to a dengue-3 virus VLP.
20. The microsphere complex of item 19, wherein the dengue-3 virus VLP is derived from dengue-3 virus strain Sri Lanka D3/H/IMTSSA-SRI/2000/1266 characterized by SEQ ID NO: 3.
21. The microsphere complex of item 19, wherein the dengue-3 virus VLP comprises the envelope protein, the membrane protein, and the pre-membrane protein which are at least 80%, or at least 85%, or at least 90%, or at least 95% or at least 100% identical to corresponding parts of SEQ ID NO: 3.
22. The microsphere complex of item 1, wherein the microsphere is coupled to a dengue-4 virus VLP.
23. The microsphere complex of item 22, wherein the dengue-4 virus VLP is derived from dengue-4 virus strain Dominica/814669/1981 characterized by SEQ ID NO: 4.
24. The microsphere complex of item 22, wherein the dengue-4 virus VLP comprises the envelope protein, the membrane protein, and the pre-membrane protein which are at least 80%, or at least 85%, or at least 90%, or at least 95% or at least 100% identical to corresponding parts of SEQ ID NO: 4.
25. The microsphere complex of item 1, wherein the microsphere is coupled to a zika virus VLP.
26. The microsphere complex of item 25, wherein the zika virus VLP is derived from zika virus strain Suriname Z1106033 characterized by SEQ ID NO: 9.
27. The microsphere complex of item 25, wherein the zika virus VLP comprises the envelope protein, the membrane protein, and the pre-membrane protein which are at least 80%, or at least 85%, or at least 90%, or at least 95% or at least 100% identical to corresponding parts of SEQ ID NO: 9.
28. The microsphere complex of any one of items 13 to 27, wherein the VLP is produced in human cells, in particular human embryonic kidney (HEK293) cells.

### NS1

29. The microsphere complex of item 1, wherein the microsphere is coupled to a dengue-1 virus non-structural protein 1.
30. The microsphere complex of item 29, wherein the dengue-1 virus non-structural protein 1 is derived from dengue-1 virus strain Nauru/Western Pacific/1974 characterized by SEQ ID NO: 11.
31. The microsphere complex of item 29, wherein the dengue-1 virus non-structural protein 1 is at least 80%, or at least 85%, or at least 90%, or at least 95% or at least 100% identical to the corresponding part of SEQ ID NO: 11, in particular the dengue-1 virus non-structural protein 1 is shown in SEQ ID NO: 13.
32. The microsphere complex of item 1, wherein the microsphere is coupled to a dengue-2 virus non-structural protein 1.
33. The microsphere complex of item 32, wherein the dengue-2 virus non-structural protein 1 is derived from dengue-2 virus strain Thailand/16681/84 characterized by SEQ ID NO: 2.
34. The microsphere complex of item 32, wherein the dengue-2 virus non-structural protein 1 is at least 80%, or at least 85%, or at least 90%, or at least 95% or at least 100% identical to the corresponding part of SEQ ID NO: 2, in particular the dengue-2 virus non-structural protein 1 is shown in SEQ ID NO: 14.
35. The microsphere complex of item 1, wherein the microsphere is coupled to a dengue-3 virus non-structural protein 1.
36. The microsphere complex of item 35, wherein the dengue-3 virus non-structural protein 1 is derived from dengue-3 virus strain Sri Lanka D3/H/IMTSSA-SRI/2000/1266 characterized by SEQ ID NO: 3.
37. The microsphere complex of item 35, wherein the dengue-3 virus non-structural protein 1 is at least 80%, or at least 85%, or at least 90%, or at least 95% or at least 100% identical to the corresponding part of SEQ ID NO: 3, in particular the dengue-3 virus non-structural protein 1 is shown in SEQ ID NO: 15.
38. The microsphere complex of item 1, wherein the microsphere is coupled to a dengue-4 virus non-structural protein 1.
39. The microsphere complex of item 38, wherein the dengue-4 virus non-structural protein 1 is derived from dengue-4 virus strain Dominica/814669/1981 characterized by SEQ ID NO: 4.
40. The microsphere complex of item 38, wherein the dengue-4 virus non-structural protein 1 is at least 80%, or at least 85%, or at least 90%, or at least 95% or at least 100% identical to the corresponding part of SEQ ID NO: 4, in particular the dengue-4 virus non-structural protein 1 is shown in SEQ ID NO: 16.
41. The microsphere complex of item 1, wherein the microsphere is coupled to a zika virus non-structural protein 1.
42. The microsphere complex of item 41, wherein the zika virus non-structural protein 1 is derived from zika virus strain Suriname 21106033 characterized by SEQ ID NO: 9.
43. The microsphere complex of item 41, wherein the zika virus non-structural protein 1 is at least 80%, or at least 85%, or at least 90%, or at least 95% or at least 100% identical to the corresponding part of SEQ ID NO: 9.

### Microsphere

44. The microsphere complex of any one of items 1 to 43, wherein the microsphere is a polystyrene microsphere.
45. The microsphere complex of any one of items 1 to 44, wherein the microsphere is magnetic.
46. The microsphere complex of any one of items 1 to 45, wherein the microsphere has a diameter in the range from about 0.01 to about 100 μm, preferably in the range from about 1 to about 20 μm, more preferably from about 5 to about 7 μm, most preferably the microsphere has a diameter of about 6.5 μm.
47. The microsphere complex of any one of items 1 to 46, wherein the microsphere contains carboxylate groups at the microsphere surface.
48. The microsphere complex of item 47, wherein coupling of the microsphere to the flavivirus antigen occurs by formation of an amide bond between a carboxylate group of the microsphere and an amine group of the flavivirus antigen.
49. The microsphere complex of any one of items 1 to 48, wherein the microsphere comprises a detectable label.
50. The microsphere complex of item 49, wherein the detectable label is at least one fluorescent dye.
51. The microsphere complex of item 50, wherein the microsphere can be identified by the emission signal of the at least one fluorescent dye.
52. The microsphere complex of any one of item 50 or 51, wherein the at least one fluorescent dye is selected from the group consisting of squaraine, phthalocyanine, naphthalocyanine, and any derivative thereof.
53. The microsphere complex of any one of items 1 to 48, wherein the microsphere can be identified by its diameter.
54. The microsphere complex of any one of items 1 to 48, wherein the microsphere can be identified by its shape.

### Method for Determining the Presence and/or Amount of Flavivirus-Reactive Complement-Fixing Antibodies

55. A method for determining the presence and/or amount of flavivirus-reactive complement-fixing antibodies in a sample from a subject comprising the steps of:
   Step 1: contacting an amount of a microsphere complex comprising a microsphere coupled to a flavivirus antigen according to any one of items 1 to 54 with the sample to allow binding of the flavivirus-reactive complement-fixing antibodies in the sample to the flavivirus antigen;
   Step 2: contacting an amount of complement component 1q (C1q) with the complement-fixing antibodies bound to the flavivirus antigen in step 1 to allow binding of the C1q to the heavy chain constant region of the complement-fixing antibodies;
   Step 3: contacting an amount of a reporter antibody with the C1q bound to the complement-fixing antibodies in step 2 to allow binding of the reporter antibody to the C1q, wherein the reporter antibody binds to the C1q with the variable region of the reporter antibody and wherein the reporter antibody is attached to a detectable label; and
   Step 4: detecting a signal from the reporter antibody bound to the C1q in step 3, wherein the signal is indicative for the presence and/or amount of the reporter antibody and wherein the presence and/or amount of the reporter antibody is indicative for the presence and/or amount of flavivirus-reactive complement-fixing antibodies in the sample.
56. The method according to item 55, comprising the further steps of:
   Step 5: determining the presence and/or amount of the reporter antibody from the signal of step 4; and
   Step 6: determining the presence and/or amount of flavivirus-reactive complement-fixing antibodies in the sample from the presence and/or amount of the reporter antibody determined in step 5.
57. The method according to item 55, comprising the steps of:
   Step 1: contacting an amount of a microsphere complex comprising a microsphere coupled to a flavivirus antigen according to any one of items 1 to 54 with the sample to allow binding of the flavivirus-reactive complement-fixing antibodies in the sample to the flavivirus antigen;
   Step 2: contacting an amount of C1q with the complement-fixing antibodies bound to the flavivirus antigen in step 1 to allow binding of the C1q to the heavy chain constant region of the complement-fixing antibodies;
   Step 3.1: contacting an amount of a pre-reporter antibody with the C1q bound to the complement-fixing antibodies in step 2 to allow binding of the pre-reporter antibody to the C1q, wherein the pre-reporter antibody binds to the C1q with the variable region of the pre-reporter antibody;
   Step 3.2: contacting an amount of a reporter antibody with the pre-reporter antibody bound to the C1q in step 3.1 to allow binding of the reporter antibody to the pre-reporter antibody, wherein the reporter antibody binds to the heavy chain constant region of the pre-reporter antibody with the variable region of the reporter antibody and wherein the reporter antibody is attached to a detectable label; and
   Step 4: detecting a signal from the reporter antibody bound to the pre-reporter antibody in step 3.2, wherein the signal is indicative for the presence and/or amount of the reporter antibody and wherein the presence and/or amount of the reporter antibody is indicative for the presence and/or amount of flavivirus-reactive complement-fixing antibodies in the sample.
58. The method according to item 57, wherein the method further comprises the steps of:
   Step 5: determining the presence and/or amount of the reporter antibody from the signal of step 4; and
   Step 6: determining the presence and/or amount of flavivirus-reactive complement-fixing antibodies in the sample from the presence and/or amount of the reporter antibody determined in step 5.
59. The method according to any one of item 57 or 58, wherein
   contacting in step 1 is carried out for about 30 to 90 minutes,
   contacting in step 2 is carried out for about 10 to 50 minutes,
   contacting in step 3.1 is carried out for about 10 to 50 minutes, and
   contacting in step 3.2 is carried out for about 10 to 50 minutes.
60. The method according to item 59, wherein
   contacting in step 1 is carried out for about 60 minutes,
   contacting in step 2 is carried out for about 30 minutes,
   contacting in step 3.1 is carried out for about 30 minutes, and
   contacting in step 3.2 is carried out for about 30 minutes.
61. The method according to any one of items 55 to 60, wherein the signal from the reporter antibody in step 4 is resulting from the detectable label to which the reporter antibody is attached.
62. The method according to any one of items 55 to 61, wherein the detectable label to which the reporter antibody is attached to is a fluorescence label selected from the group consisting of xanthene, fluorescein isothiocyanate, rhodamine, phycoerythrin, cyanine, coumarin, and any derivative thereof.
63. The method according to item 62, wherein the detectable label is phycoerythrin.

### Method for the Concomitant Determination of the Presence and/or Amount of Complement-Fixing Antibodies Reactive to Different Flaviviruses

64. A method for the concomitant determination of the presence and/or amount of complement-fixing antibodies reactive to different flaviviruses in a sample from a subject comprising the steps of:
   Step 1: contacting an amount of at least two microsphere complexes with the sample,
      wherein the first microsphere complex comprises a first microsphere coupled to a first flavivirus antigen according to any one of items 1 to 54 and the second microsphere complex comprises a second microsphere coupled to a second flavivirus antigen according to any one of items 1 to 54
   to allow binding of the complement-fixing antibodies reactive to the first and/or the second flavivirus to the first flavivirus antigen and/or the second flavivirus antigen,
      wherein the first microsphere comprises a first detectable label and the second microsphere comprises a second detectable label, and wherein the emission signal of the first detectable label differs from the emission signal of the second detectable label,
   Step 2: contacting an amount of C1q with the complement-fixing antibodies bound to the first and/or the second flavivirus antigen in step 1 to allow binding of the C1q to the heavy chain constant region of the complement-fixing antibodies,
   Step 3: contacting an amount of a reporter antibody with the C1q bound to the complement-fixing antibodies in step 2 to allow binding of the reporter antibody to the C1q, wherein the reporter antibody binds to the C1q with the variable region of the reporter antibody and wherein the reporter antibody is attached to a third detectable label,
   Step 4: detecting the emission signal of the detectable label of at least one microsphere upon irradiation with a first light source and comparing the emission signal with the emission signal of the first detectable label and with the emission signal of the second detectable label thereby identifying the at least one microsphere and the flavivirus antigen the at least one microsphere is coupled to, and
   simultaneously detecting a signal from the reporter antibody bound to the C1q in step 3 of the at least one microsphere upon irradiation with a second light source,
   Step 5: repeating step 4 until at least 30 microspheres coupled to the same flavivirus antigen are identified, and
   Step 6: summarizing the detected signal from the reporter antibody in step 4 for all identified microspheres coupled to the same flavivirus antigen, wherein the summarized signal is indicative for the presence and/or amount of the reporter antibody and wherein the presence and/or amount of the reporter antibody is indicative for the presence and/or amount of flavivirus-reactive complement-fixing antibodies in the sample.
65. The method according to item 64, wherein the method further comprises the steps of:
   Step 7: determining the presence and/or amount of the reporter antibody from the summarized signal in step 6, and
   Step 8: determining the presence and/or amount of flavivirus-reactive complement-fixing antibodies in the sample from the presence and/or amount of the reporter antibody determined in step 7.
66. The method according to item 64, comprising the steps of:
   Step 1: contacting an amount of at least two microsphere complexes with the sample,
      wherein the first microsphere complex comprises a first microsphere coupled to a first flavivirus antigen according to any one of items 1 to 54 and the second microsphere complex comprises a second microsphere coupled to a second flavivirus antigen according to any one of items 1 to 54
   to allow binding of the complement-fixing antibodies reactive to the first and/or the second flavivirus to the first flavivirus antigen and/or the second flavivirus antigen,
      wherein the first microsphere comprises a first detectable label and the second microsphere comprises a second detectable label, and wherein the emission signal of the first detectable label differs from the emission signal of the second detectable label,
   Step 2: contacting an amount of C1q with the complement-fixing antibodies bound to the first and/or the second flavivirus antigen in step 1 to allow binding of the C1q to the heavy chain constant region of the complement-fixing antibodies,
   Step 3.1: contacting an amount of a pre-reporter antibody with the C1q bound to the complement-fixing antibodies in step 2 to allow binding of the pre-reporter antibody to the C1q, wherein the pre-reporter antibody binds to the C1q with the variable region of the pre-reporter antibody,
   Step 3.2: contacting an amount of a reporter antibody with the pre-reporter antibody bound to the C1q in step 3.1 to allow binding of the reporter antibody to the pre-reporter antibody, wherein the reporter antibody binds to the heavy chain constant region of the pre-reporter antibody with the variable region of the reporter antibody and wherein the reporter antibody is attached to a third detectable label,
   Step 4: detecting the emission signal of the detectable label of at least one microsphere upon irradiation with a first light source and comparing the emission signal with the emission signal of the first detectable label and with the emission signal of the second detectable label thereby identifying the at least one microsphere and the flavivirus antigen the at least one microsphere is coupled to, and
   simultaneously detecting a signal from the reporter antibody bound to the C1q in step 3 of the at least one microsphere upon irradiation with a second light source,
   Step 5: repeating step 4 until at least 30 microspheres coupled to the same flavivirus antigen are identified, and
   Step 6: summarizing the detected signal from the reporter antibody in step 4 for all identified microspheres coupled to the same flavivirus antigen, wherein the summarized signal is indicative for the presence and/or amount of the reporter antibody and wherein the presence and/or amount of the reporter antibody is indicative for the presence and/or amount of flavivirus-reactive complement-fixing antibodies in the sample.
67. The method according to item 66, wherein the method further comprises the steps of:
   Step 7: determining the presence and/or amount of reporter antibody from the summarized signal in step 6, and
   Step 8: determining the presence and/or amount of flavivirus-reactive complement-fixing antibodies in the sample from the presence and/or amount of reporter antibody determined in step 7.
68. The method according to any one of item 66 or 67, wherein
   contacting in step 1 is carried out for about 30 to 90 minutes,
   contacting in step 2 is carried out for about 10 to 50 minutes,
   contacting in step 3.1 is carried out for about 10 to 50 minutes, and
   contacting in step 3.2 is carried out for about 10 to 50 minutes.
69. The method according to item 68, wherein
   contacting in step 1 is carried out for about 60 minutes,
   contacting in step 2 is carried out for about 30 minutes,
   contacting in step 3.1 is carried out for about 30 minutes, and
   contacting in step 3.2 is carried out for about 30 minutes.
70. The method according to any one of items 64 to 69, wherein the signal from the reporter antibody in step 4 is resulting from the third detectable label.
71. The method according to any one of items 64 to 70, wherein the third detectable label is a fluorescence label selected from the group consisting of xanthene, fluorescein isothiocyanate, rhodamine, phycoerythrin, cyanine, coumarin, and any derivative thereof.
72. The method according to item 71, wherein the fluorescence label is phycoerythrin.
73. The method according to any one of items 64 to 72, wherein the first detectable label is at least one fluorescent dye.
74. The method according to any one of items 64 to 73, wherein the second detectable label is at least one fluorescent dye.
75. The method according to item 73 or 74, wherein the at least one fluorescent dye is selected from the group consisting of squaraine, phthalocyanine, naphthalocyanine, and any derivative thereof.
76. The method according to any one of items 64 to 75, wherein in step 1, an amount of a first microsphere complex comprising a first microsphere coupled to a dengue 1 virus VLP, an amount of a second microsphere complex comprising a second microsphere coupled to a dengue 2 virus VLP, an amount of a third microsphere complex comprising a third microsphere coupled to a dengue 3 virus VLP, and an amount of a fourth microsphere complex comprising a fourth microsphere coupled to a dengue 4 virus VLP is contacted with the sample.
77. The method according to any one of items 64 to 75, wherein in step 1, an amount of a first microsphere complex comprising a first microsphere coupled to a dengue 1 virus VLP, an amount of a second microsphere complex comprising a second microsphere coupled to a dengue 2 virus VLP, an amount of a third microsphere complex comprising a third microsphere coupled to a dengue 3 virus VLP, an amount of a fourth microsphere complex comprising a fourth microsphere coupled to a dengue 4 virus VLP, an amount of a fifth microsphere complex comprising a fifth microsphere coupled to a zika virus VLP, an amount of a sixth microsphere complex comprising a sixth microsphere coupled to a dengue 1 virus non-structural protein 1, an amount of a seventh microsphere complex comprising a seventh microsphere coupled to a dengue 2 virus non-structural protein 1, an amount of an eighth microsphere complex comprising an eighth microsphere coupled to a dengue 3 virus non-structural protein 1, an amount of a ninth microsphere complex comprising a ninth microsphere coupled to a dengue 4 virus non-structural protein 1, and an amount of a tenth microsphere complex comprising a tenth microsphere coupled to a zika virus non-structural protein 1 is contacted with the sample.
78. The method according to any one of items 55 to 77, wherein the C1q is human C1q.
79. The method according to any one of claims 55 to 78, wherein the C1q is purified.
80. The method according to any one of items 55 to 79, wherein the subject is seropositive for the flavivirus.
81. The method according to any one of items 55 to 79, wherein the subject is seronegative for the flavivirus.
82. The method according to any one of items 55 to 80, wherein the subject is vaccinated with a flavivirus vaccine.

In Vitro Method for Diagnosing a Flavivirus Infection
83. An in vitro method for diagnosing a flavivirus infection in a subject comprising the steps of:
   Step 1: providing a sample obtained from the subject,
   Step 2: determining the amount of flavivirus-reactive complement-fixing antibodies in the sample according to any one of items 55 to 82, wherein the presence of flavivirus-reactive complement-fixing antibodies in the sample is indicative for a flavivirus infection.
84. The method according to item 83, wherein the flavivirus infection is acute.
85. The method according to item 83, wherein the flavivirus infection is convalescent.
86. The method according to any one of items 83 to 85, wherein the flavivirus is selected from the group consisting of dengue virus, zika virus, West Nile virus, Japanese encephalitis virus, Tick-Borne encephalitis virus, Yellow Fever virus, Murray Valley encephalitis virus, and St. Louis encephalitis virus.
87. The method according to any one of items 83 or 86, wherein the subject is infected by a dengue virus serotype selected from the group of dengue virus serotype 1, dengue virus serotype 2, dengue virus serotype 3, and dengue virus serotype 4.
88. The method according to any one of items 83 to 86, wherein the subject is infected by at least two different flaviviruses, such as zika virus and dengue virus.
89. The method according to any one of items 83 to 86, wherein the subject is infected by at least two different dengue virus serotypes selected from the group of dengue virus serotype 1, dengue virus serotype 2, dengue virus serotype 3, and dengue virus serotype 4.
90. The method according to any one of items 55 to 89, wherein the sample is heat-inactivated.
91. The method according to any one of items 55 to 90, wherein the sample is a serum sample or a plasma sample.
92. The method according to any one of items 55 to 91, wherein the subject is selected from the list consisting of mouse, primate, non-human primate, human, rabbit, cat, rat, horse, and sheep.
93. The method according to item 92, wherein the subject is human.

Kit for Detecting Flavivirus-Reactive Complement-Fixing Antibodies
94. A kit for detecting flavivirus-reactive complement-fixing antibodies in a sample comprising:
   an amount of at least one microsphere complex comprising a microsphere coupled to a flavivirus antigen according to any one of items 1 to 54,
   an amount of C1q, and
   an amount of a reporter antibody, wherein the reporter antibody is attached to a detectable label.
95. The kit according to item 94, comprising an amount of at least two microspheres complexes,
   wherein the first microsphere complex comprises a first microsphere coupled to a first flavivirus antigen according to any one of items 1 to 54 and the second microsphere complex comprises a second microsphere coupled to a second flavivirus antigen according to any one of items 1 to 54,
   wherein the first microsphere comprises a first detectable label and the second microsphere comprises a second detectable label, and
   wherein the emission signal of the first detectable label differs from the emission signal of the second detectable label.
96. The kit according to any one of item 94 or 95, comprising an amount of a first microsphere complex comprising a first microsphere coupled to a dengue 1 virus VLP, an amount of a second microsphere complex comprising a second microsphere coupled to a dengue 2 virus VLP, an amount of a third microsphere complex comprising a third microsphere coupled to a dengue 3 virus VLP, and an amount of a fourth microsphere complex comprising a fourth microsphere coupled to a dengue 4 virus VLP.
97. The kit according to any one of item 94 or 95, comprising an amount of a first microsphere complex comprising a first microsphere coupled to a dengue 1 virus VLP, an amount of a second microsphere complex comprising a second microsphere coupled to a dengue 2 virus VLP, an amount of a third microsphere complex comprising a third microsphere coupled to a dengue 3 virus VLP, an amount of a fourth microsphere complex comprising a fourth microsphere coupled to a dengue 4 virus VLP, an amount of a fifth microsphere complex comprising a fifth microsphere coupled to a zika virus VLP, an amount of a sixth microsphere complex comprising a sixth microsphere coupled to a dengue 1 virus non-structural protein 1, an amount of a seventh microsphere complex comprising a seventh microsphere coupled to a dengue 2 virus non-structural protein 1, an amount of an eighth microsphere complex comprising an eighth microsphere coupled to a dengue 3 virus non-structural protein 1, an amount of a ninth microsphere complex comprising a ninth microsphere coupled to a dengue 4 virus non-structural protein 1, and an amount of a tenth microsphere complex comprising a tenth microsphere coupled to a zika virus non-structural protein 1.
98. The kit according to any one of items 94 to 97, wherein the C1q is human C1q.
99. The kit according to any one of items 94 to 98, wherein the C1q is purified.
100. The kit according to any one of items 94 to 99, wherein the reporter antibody binds to the C1q with the variable region of the reporter antibody.
101. The kit according to any one of items 94 to 99, wherein the kit further comprises an amount of a pre-reporter antibody, and wherein the pre-reporter antibody binds to the C1q with the variable region of the pre-reporter antibody and the reporter antibody binds to the heavy chain constant region of the pre-reporter antibody with the variable region of the reporter antibody.
102. The kit according to any one of items 94 to 101, wherein the detectable label to which the reporter antibody is attached to is a fluorescence label selected from the group consisting of xanthene, fluorescein isothiocyanate, rhodamine, phycoerythrin, cyanine, coumarin, and any derivative thereof.
103. The kit according to item 102, wherein the fluorescence label is phycoerythrin.
104. The kit according to any one of items 95 to 103, wherein the first detectable label the first microsphere comprises is at least one fluorescent dye.
105. The method according to any one of items 95 to 104, wherein the second detectable label the second microsphere comprises is at least one fluorescent dye.
106. The method according to items 104 or 105, wherein the at least one fluorescent dye is selected from the group consisting of squaraine, phthalocyanine, naphthalocyanine, and any derivative thereof.

Additional Embodiments are Outlined Below:
1. A method for determining the presence and/or amount of flavivirus-reactive complement-fixing antibodies in a sample from a subject comprising the steps of:
   Step 1: contacting an amount of a microsphere complex comprising a microsphere coupled to a flavivirus antigen with the sample to allow binding of the flavivirus-reactive complement-fixing antibodies in the sample to the flavivirus antigen;
   Step 2: contacting an amount of a complement competent serum with the complement-fixing antibodies bound to the flavivirus antigen in step 1 to allow the formation of complement component fragment C3d (C3d);
   Step 3: contacting an amount of a reporter antibody with the C3d formed in step 2 to allow binding of the reporter antibody to the C3d, wherein the reporter antibody binds to the C3d with the variable region of the reporter antibody and wherein the reporter antibody is attached to a detectable label; and
   Step 4: detecting a signal from the reporter antibody bound to the C3d in step 3, wherein the signal is indicative for the presence and/or amount of the reporter antibody and wherein the presence and/or amount of the reporter antibody is indicative for the presence and/or amount of flavivirus-reactive complement-fixing antibodies in the sample.
2. The method according to item 1, comprising the steps of:
   Step 1: contacting an amount of a microsphere complex comprising a microsphere coupled to a flavivirus antigen with the sample to allow binding of the flavivirus-reactive complement-fixing antibodies in the sample to the flavivirus antigen;
   Step 2: contacting an amount of a complement competent serum with the complement-fixing antibodies bound to the flavivirus antigen in step 1 to allow the formation of C3d;
   Step 3.1: contacting an amount of a pre-reporter antibody with the C3d formed in step 2 to allow binding of the pre-reporter antibody to the C3d, wherein the pre-reporter antibody binds to the C3d with the variable region of the pre-reporter antibody;
   Step 3.2: contacting an amount of a reporter antibody with the pre-reporter antibody bound to the C3d in step 3.1 to allow binding of the reporter antibody to the pre-reporter antibody, wherein the reporter antibody binds to the heavy chain constant region of the pre-reporter antibody with the variable region of the reporter antibody and wherein the reporter antibody is attached to a detectable label; and
   Step 4: detecting a signal from the reporter antibody bound to the pre-reporter antibody in step 3.2, wherein the signal is indicative for the presence and/or amount of the reporter antibody and wherein the presence and/or amount of the reporter antibody is indicative for the presence and/or amount of flavivirus-reactive complement-fixing antibodies in the sample.
3. The method of item 1 or 2, comprising the further steps of:
   Step 5: determining the presence and/or amount of the reporter antibody from the signal of step 4; and
   Step 6: determining the presence and/or amount of flavivirus-reactive complement-fixing antibodies in the sample from the presence and/or amount of the reporter antibody determined in step 5.
4. The method of any one of items 1 to 3, wherein the antigen is selected from the group consisting of virus like particle (VLP), non-structural protein 1, envelope protein, pre-membrane protein, membrane protein, capsid protein, non-structural protein 2A, non-structural protein 2B, non-structural protein 3, non-structural protein 4A, non-structural protein 4B, and non-structural protein 5 and any derivative thereof, preferably the antigen is VLP.
5. The method according to any one of items 1 to 4, wherein the detectable label is a fluorescence label selected from the group consisting of xanthene, fluorescein isothiocyanate, rhodamine, phycoerythrin, cyanine, coumarin, and any derivative thereof.
6. A method for the concomitant determination of the presence and/or amount of complement-fixing antibodies reactive to different flaviviruses in a sample from a subject comprising the steps of:
   Step 1: contacting an amount of at least two microsphere complexes with the sample,
      wherein the first microsphere complex comprises a first microsphere coupled to a first flavivirus antigen and the second microsphere complex comprises a second microsphere coupled to a second flavivirus antigen
   to allow binding of the complement-fixing antibodies reactive to the first and/or the second flavivirus to the first flavivirus antigen and/or the second flavivirus antigen;
      wherein the first microsphere comprises a first detectable label and the second microsphere comprises a second detectable label, and wherein the emission signal of the first detectable label differs from the emission signal of the second detectable label,
   Step 2: contacting an amount of a complement competent serum with the complement-fixing antibodies bound to the flavivirus antigen in step 1 to allow the formation of C3d,
   Step 3: contacting an amount of a reporter antibody with the C3d formed in step 2 to allow binding of the reporter antibody to the C3d, wherein the reporter antibody binds to the C3d with the variable region of the reporter antibody and wherein the reporter antibody is attached to a third detectable label,
   Step 4: detecting the emission signal of the detectable label of at least one microsphere upon irradiation with a first light source and comparing the emission signal with the emission signal of the first detectable label and with the emission signal of the second detectable label thereby identifying the at least one microsphere and the flavivirus antigen the at least one microsphere is coupled to, and
   simultaneously detecting a signal from the reporter antibody bound to the C3d in step 3 of the at least one microsphere upon irradiation with a second light source,
   Step 5: repeating step 4 until at least 30 microspheres coupled to the same flavivirus antigen are identified, and
   Step 6: summarizing the detected signal from the reporter antibody in step 4 for all identified microspheres coupled to the same flavivirus antigen, wherein the summarized signal is indicative for the presence and/or amount of the reporter antibody and wherein the presence and/or amount of the reporter antibody is indicative for the presence and/or amount of flavivirus-reactive complement-fixing antibodies in the sample.
7. The method according to item 6, comprising the steps of:
   Step 1: contacting an amount of at least two microsphere complexes with the sample,
      wherein the first microsphere complex comprises a first microsphere coupled to a first flavivirus antigen and the second microsphere complex comprises a second microsphere coupled to a second flavivirus antigen
   to allow binding of the complement-fixing antibodies reactive to the first and/or the second flavivirus to the first flavivirus antigen and/or the second flavivirus antigen,
      wherein the first microsphere comprises a first detectable label and the second microsphere comprises a second detectable label, and wherein the emission signal of the first detectable label differs from the emission signal of the second detectable label,
   Step 2: contacting an amount of a complement competent serum with the complement-fixing antibodies bound to the first and/or the second flavivirus antigen in step 1 to allow the formation of C3d,
   Step 3.1: contacting an amount of a pre-reporter antibody with the C3d formed in step 2 to allow the formation of C3d and to allow binding of the pre-reporter antibody to the C3d, wherein the pre-reporter antibody binds to the C3d with the variable region of the pre-reporter antibody,
   Step 3.2: contacting an amount of a reporter antibody with the pre-reporter antibody bound to the C3d in step 3.1 to allow binding of the reporter antibody to the pre-reporter antibody, wherein the reporter antibody binds to the heavy chain constant region of the pre-reporter antibody with the variable region of the reporter antibody and wherein the reporter antibody is attached to a third detectable label,
   Step 4: detecting the emission signal of the detectable label of at least one microsphere upon irradiation with a first light source and comparing the emission signal with the emission signal of the first detectable label and with the emission signal of the second detectable label thereby identifying the at least one microsphere and the flavivirus antigen the at least one microsphere is coupled to, and
   simultaneously detecting a signal from the reporter antibody bound to the C3d in step 3 of the at least one microsphere upon irradiation with a second light source,
   Step 5: repeating step 4 until at least 30 microspheres coupled to the same flavivirus antigen are identified, and
   Step 6: summarizing the detected signal from the reporter antibody in step 4 for all identified microspheres coupled to the same flavivirus antigen, wherein the summarized signal is indicative for the presence and/or amount of the reporter antibody and wherein the presence and/or amount of the reporter antibody is indicative for the presence and/or amount of flavivirus-reactive complement-fixing antibodies in the sample.
8. The method according to item 6 or 7, wherein the method further comprises the steps of:
   Step 7: determining the presence and/or amount of the reporter antibody from the summarized signal in step 6, and
   Step 8: determining the presence and/or amount of flavivirus-reactive complement-fixing antibodies in the sample from the presence and/or amount of the reporter antibody determined in step 7.
9. The method according to any one of items 6 to 8, wherein the antigen is selected from the group consisting of virus like particle (VLP), non-structural protein 1, envelope protein, pre-membrane protein, membrane protein, capsid protein, non-structural protein 2A, non-structural protein 2B, non-structural protein 3, non-structural protein 4A, non-structural protein 4B, and non-structural protein 5 and any derivative thereof, preferably the antigen is VLP.
10. The method according to any one of items 6 to 9, wherein the third detectable label is a fluorescence label selected from the group consisting of xanthene, fluorescein isothiocyanate, rhodamine, phycoerythrin, cyanine, coumarin, and any derivative thereof.
11. The method of any one of items 6 to 10, wherein the flavivirus is selected from the group consisting of dengue virus, zika virus, West Nile virus, Japanese encephalitis virus, Tick-Borne encephalitis virus, Yellow Fever virus, Murray Valley encephalitis virus, and St. Louis encephalitis virus, preferably dengue virus
12. The method of any one of items 6 to 10, wherein the antigen is selected from the group consisting of virus like particle (VLP), non-structural protein 1, envelope protein, pre-membrane protein, membrane protein, capsid protein, non-structural protein 2A, non-structural protein 2B, non-structural protein 3, non-structural protein 4A, non-structural protein 4B, and non-structural protein 5 and any derivative thereof, preferably the antigen is VLP
13. The method according to any one of items 6 to 12, wherein in step 1, an amount of a first microsphere complex comprising a first microsphere coupled to a dengue 1 virus VLP, an amount of a second microsphere complex comprising a second microsphere coupled to a dengue 2 virus VLP, an amount of a third microsphere complex comprising a third microsphere coupled to a dengue 3 virus VLP, and an amount of a fourth microsphere complex comprising a fourth microsphere coupled to a dengue 4 virus VLP is contacted with the sample.
14. An in vitro method for diagnosing a flavivirus infection in a subject comprising the steps of:
   Step 1: providing a sample obtained from the subject,
   Step 2: determining the amount of flavivirus-reactive complement-fixing antibodies in the sample according to any one of items 1 to 13, wherein the presence of flavivirus-reactive complement-fixing antibodies in the sample is indicative for a flavivirus infection.
15. The method according to item 14, wherein the flavivirus is selected from the group consisting of dengue virus, zika virus, West Nile virus, Japanese encephalitis virus, Tick-Borne encephalitis virus, Yellow Fever virus, Murray Valley encephalitis virus, and St. Louis encephalitis virus, preferably the flavirus is dengue virus.
16. The method according to item 14 or 15, wherein the subject is infected by at least two different flaviviruses.
17. The method according to item 14 or 15, wherein the subject is infected by at least two different dengue virus serotypes selected from the group of dengue virus serotype 1, dengue virus serotype 2, dengue virus serotype 3, and dengue virus serotype 4.
18. The method according to any one of items 1 to 13, wherein the sample is heat-inactivated.
19. The method according to any one of items 1 to 13, wherein the subject is selected from the group consisting of mouse, primate, non-human primate, human, rabbit, cat, rat, horse, and sheep, preferably the subject is human.
20. A kit for detecting flavivirus-reactive complement-fixing antibodies in a sample comprising:
   an amount of at least one microsphere complex comprising a microsphere coupled to a flavivirus antigen,
   an amount of a complement competent serum, and
   an amount of a reporter antibody, wherein the reporter antibody is attached to a detectable label.
21. The kit according to item 20, comprising an amount of at least two microspheres complexes,
   wherein the first microsphere complex comprises a first microsphere coupled to a first flavivirus antigen and the second microsphere complex comprises a second microsphere coupled to a second flavivirus antigen,
   wherein the first microsphere comprises a first detectable label and the second microsphere comprises a second detectable label, and
   wherein the emission signal of the first detectable label differs from the emission signal of the second detectable label.
22. The kit of any one of item 20 or 21, wherein the antigen is selected from the group consisting of virus like particle (VLP), non-structural protein 1, envelope protein, pre-membrane protein, membrane protein, capsid protein, non-structural protein 2A, non-structural protein 2B, non-structural protein 3, non-structural protein 4A, non-structural protein 4B, and non-structural protein 5 and any derivative thereof.
23. The kit according to any one of items 20 to 22, wherein the flavivirus is selected from the group consisting of dengue virus, zika virus, West Nile virus, Japanese encephalitis virus, Tick-Borne encephalitis virus, Yellow Fever virus, Murray Valley encephalitis virus, and Saint Louis encephalitis virus, preferably the flavivirus is dengue virus.
24. The kit according to any one of item 20 or 23, comprising an amount of a first microsphere complex comprising a first microsphere coupled to a dengue 1 virus VLP, an amount of a second microsphere complex comprising a second microsphere coupled to a dengue 2 virus VLP, an amount of a third microsphere complex comprising a third microsphere coupled to a dengue 3 virus VLP, and an amount of a fourth microsphere complex comprising a fourth microsphere coupled to a dengue 4 virus VLP.
25. The kit according to any one of items 20 to 24, wherein the reporter antibody binds to the C3d with the variable region of the reporter antibody.
26. The kit according to any one of items 20 to 25, wherein the kit further comprises an amount of a pre-reporter antibody, and wherein the pre-reporter antibody binds to the C3d with the variable region of the pre-reporter antibody and the reporter antibody binds to the heavy chain constant region of the pre-reporter antibody with the variable region of the reporter antibody.
27. The kit according to any one of items 20 to 26, wherein the detectable label to which the reporter antibody is attached to is a fluorescence label selected from the group consisting of xanthene, fluorescein isothiocyanate, rhodamine, phycoerythrin, cyanine, coumarin, and any derivative thereof.

1. A method for determining the presence and/or amount of flavivirus-reactive complement-fixing antibodies in a sample from a subject comprising the steps of:
Step 1: contacting an amount of a microsphere complex comprising a microsphere coupled to a flavivirus antigen with the sample to allow binding of the flavivirus-reactive complement-fixing antibodies in the sample to the flavivirus antigen;
Step 2: contacting an amount of complement component 1q (C1q) with the complement-fixing antibodies bound to the flavivirus antigen in step 1 to allow binding of the C1q to a heavy chain constant region of the complement-fixing antibodies;
Step 3: contacting an amount of a reporter antibody with the C1q bound to the complement-fixing antibodies in step 2 to allow binding of the reporter antibody to the C1q, wherein the reporter antibody binds to the C1q with a variable region of the reporter antibody and wherein the reporter antibody is attached to a detectable label; and
Step 4: detecting a signal from the reporter antibody bound to the C1q in step 3, wherein the signal is indicative for the presence and/or amount of the reporter antibody and wherein the presence and/or amount of the reporter antibody is indicative for the presence and/or amount of flavivirus-reactive complement-fixing antibodies in the sample.

2. The method according to claim 1, comprising the further steps of:
Step 5: determining the presence and/or amount of the reporter antibody from the signal of step 4; and
Step 6: determining the presence and/or amount of flavivirus-reactive complement-fixing antibodies in the sample from the presence and/or amount of the reporter antibody determined in step 5.

3. The method according to claim 1, comprising the steps of:
Step 1: contacting an amount of a microsphere complex comprising a microsphere coupled to a flavivirus antigen with the sample to allow binding of the flavivirus-reactive complement-fixing antibodies in the sample to the flavivirus antigen;
Step 2: contacting an amount of C1q with the complement-fixing antibodies bound to the flavivirus antigen in step 1 to allow binding of the C1q to the heavy chain constant region of the complement-fixing antibodies;
Step 3.1: contacting an amount of a pre-reporter antibody with the C1q bound to the complement-fixing antibodies in step 2 to allow binding of the pre-reporter antibody to the C1q, wherein the pre-reporter antibody binds to the C1q with a variable region of the pre-reporter antibody;
Step 3.2: contacting an amount of a reporter antibody with the pre-reporter antibody bound to the C1q in step 3.1 to allow binding of the reporter antibody to the pre-reporter antibody, wherein the reporter antibody binds to a heavy chain constant region of the pre-reporter antibody with the variable region of the reporter antibody and wherein the reporter antibody is attached to a detectable label; and
Step 4: detecting a signal from the reporter antibody bound to the pre-reporter antibody in step 3.2, wherein the signal is indicative for the presence and/or amount of the reporter antibody and wherein the presence and/or amount of the reporter antibody is indicative for the presence and/or amount of flavivirus-reactive complement-fixing antibodies in the sample.

4. The method according to claim 3, wherein the method further comprises the steps of:
Step 5: determining the presence and/or amount of the reporter antibody from the signal of step 4; and
Step 6: determining the presence and/or amount of flavivirus-reactive complement-fixing antibodies in the sample from the presence and/or amount of the reporter antibody determined in step 5.

5. The method according to claim 1, to wherein the antigen is selected from the group consisting of virus like particle (VLP), non-structural protein 1, envelope protein, pre-membrane protein, membrane protein, capsid protein, non-structural protein 2A, non-structural protein 2B, non-structural protein 3, non-structural protein 4A, non-structural protein 4B, and non-structural protein 5 and any derivative thereof.

6. The method according to claim 1, wherein the detectable label is a fluorescence label selected from the group consisting of xanthene, fluorescein isothiocyanate, rhodamine, phycoerythrin, cyanine, coumarin, and any derivative thereof.

7. A method for the concomitant determination of the presence and/or amount of complement-fixing antibodies reactive to different flaviviruses in a sample from a subject comprising the steps of:
Step 1: contacting an amount of at least two microsphere complexes with the sample,
wherein a first microsphere complex comprises a first microsphere coupled to a first flavivirus antigen and a second microsphere complex comprises a second microsphere coupled to a second flavivirus antigen
to allow binding of the complement-fixing antibodies reactive to the first and/or the second flavivirus to the first flavivirus antigen and/or the second flavivirus antigen,
wherein the first microsphere comprises a first detectable label and the second microsphere comprises a second detectable label, and wherein an emission signal of the first detectable label differs from an emission signal of the second detectable label,
Step 2: contacting an amount of C1q with the complement-fixing antibodies bound to the first and/or the second flavivirus antigen in step 1 to allow binding of the C1q to a heavy chain constant region of the complement-fixing antibodies,
Step 3: contacting an amount of a reporter antibody with the C1q bound to the complement-fixing antibodies in step 2 to allow binding of the reporter antibody to the C1q, wherein the reporter antibody binds to the C1q with the variable region of the reporter antibody and wherein the reporter antibody is attached to a third detectable label,
Step 4: detecting the emission signal of the detectable label of at least one microsphere upon irradiation with a first light source and comparing the emission signal with the emission signal of the first detectable label and with the emission signal of the second detectable label thereby identifying the at least one microsphere and the flavivirus antigen the at least one microsphere is coupled to, and
simultaneously detecting a signal from the reporter antibody bound to the C1q in step 3 of the at least one microsphere upon irradiation with a second light source,
Step 5: repeating step 4 until at least 30 microspheres coupled to the same flavivirus antigen are identified, and
Step 6: summarizing the detected signal from the reporter antibody in step 4 for all identified microspheres coupled to the same flavivirus antigen, wherein the summarized signal is indicative for the presence and/or amount of the reporter antibody and wherein the presence and/or amount of the reporter antibody is indicative of the presence and/or amount of flavivirus-reactive complement-fixing antibodies in the sample.

8. The method according to claim 7, wherein the method further comprises the steps of:
Step 7: determining the presence and/or amount of the reporter antibody from the summarized signal in step 6, and
Step 8: determining the presence and/or amount of flavivirus-reactive complement-fixing antibodies in the sample from the presence and/or amount of the reporter antibody determined in step 7.

9. The method according to claim 7, comprising the steps of:
Step 1: contacting an amount of at least two microsphere complexes with the sample,
wherein the first microsphere complex comprises a first microsphere coupled to a first flavivirus antigen and the second microsphere complex comprises a second microsphere coupled to a second flavivirus antigen
to allow binding of the complement-fixing antibodies reactive to the first and/or the second flavivirus to the first flavivirus antigen and/or the second flavivirus antigen,
wherein the first microsphere comprises a first detectable label and the second microsphere comprises a second detectable label, and wherein the emission signal of the first detectable label differs from the emission signal of the second detectable label,
Step 2: contacting an amount of C1q with the complement-fixing antibodies bound to the first and/or the second flavivirus antigen in step 1 to allow binding of the C1q to a heavy chain constant region of the complement-fixing antibodies,
Step 3.1: contacting an amount of a pre-reporter antibody with the C1q bound to the complement-fixing antibodies in step 2 to allow binding of the pre-reporter antibody to the C1q, wherein the pre-reporter antibody binds to the C1q with a variable region of the pre-reporter antibody,
Step 3.2: contacting an amount of a reporter antibody with the pre-reporter antibody bound to the C1q in step 3.1 to allow binding of the reporter antibody to the pre-reporter antibody, wherein the reporter antibody binds to a heavy chain constant region of the pre-reporter antibody with a variable region of the reporter antibody and wherein the reporter antibody is attached to a third detectable label,
Step 4: detecting the emission signal of the detectable label of at least one microsphere upon irradiation with a first light source and comparing the emission signal with the emission signal of the first detectable label and with the emission signal of the second detectable label thereby identifying the at least one microsphere and the flavivirus antigen the at least one microsphere is coupled to, and
simultaneously detecting a signal from the reporter antibody bound to the C1q in step 3 of the at least one microsphere upon irradiation with a second light source,
Step 5: repeating step 4 until at least 30 microspheres coupled to the same flavivirus antigen are identified, and
Step 6: summarizing the detected signal from the reporter antibody in step 4 for all identified microspheres coupled to the same flavivirus antigen, wherein the summarized signal is indicative for the presence and/or amount of the reporter antibody and wherein the presence and/or amount of the reporter antibody is indicative for the presence and/or amount of flavivirus-reactive complement-fixing antibodies in the sample.

10. The method according to claim 9, wherein the method further comprises the steps of:
Step 7: determining the presence and/or amount of reporter antibody from the summarized signal in step 6, and
Step 8: determining the presence and/or amount of flavivirus-reactive complement-fixing antibodies in the sample from the presence and/or amount of reporter antibody determined in step 7.

11. The method according to claim 7, wherein the antigen is selected from the group consisting of virus like particle (VLP), non-structural protein 1, envelope protein, pre-membrane protein, membrane protein, capsid protein, non-structural protein 2A, non-structural protein 2B, non-structural protein 3, non-structural protein 4A, non-structural protein 4B, and non-structural protein 5 and any derivative thereof, preferably the antigen is VLP.

12. The method according to claim 7, wherein the third detectable label is a fluorescence label selected from the group consisting of xanthene, fluorescein isothiocyanate, rhodamine, phycoerythrin, cyanine, coumarin, and any derivative thereof.

13. The method of claim 1, wherein the flavivirus is selected from the group consisting of dengue virus, zika virus, West Nile virus, Japanese encephalitis virus, Tick-Borne encephalitis virus, Yellow Fever virus, Murray Valley encephalitis virus, and St. Louis encephalitis virus, preferably dengue virus

14. The method of claim 1, wherein the antigen is selected from the group consisting of virus like particle (VLP), non-structural protein 1, envelope protein, pre-membrane protein, membrane protein, capsid protein, non-structural protein 2A, non-structural protein 2B, non-structural protein 3, non-structural protein 4A, non-structural protein 4B, and non-structural protein 5 and any derivative thereof, preferably the antigen is VLP

15. The method of claim 1, wherein in step 1, an amount of a first microsphere complex comprising a first microsphere coupled to a dengue 1 virus VLP, an amount of a second microsphere complex comprising a second microsphere coupled to a dengue 2 virus VLP, an amount of a third microsphere complex comprising a third microsphere coupled to a dengue 3 virus VLP, and an amount of a fourth microsphere complex comprising a fourth microsphere coupled to a dengue 4 virus VLP is contacted with the sample.

16. The method of claim 1, wherein the C1q is purified.

17.-21. (canceled)

22. The method according to claim 1, wherein the sample is heat-inactivated.

23. The method according to claim 1, wherein the subject is selected from the group consisting of mouse, primate, non-human primate, human, rabbit, cat, rat, horse, and sheep, preferably the subject is human.

24. A kit for detecting flavivirus-reactive complement-fixing antibodies in a sample comprising:
an amount of at least one microsphere complex comprising a microsphere coupled to a flavivirus antigen,
an amount of C1q,
an amount of a reporter antibody, and
optionally, an amount of a pre-reporter antibody,
wherein the reporter antibody binds to the C1q with a variable region of the reporter antibody, and wherein the reporter antibody is attached to a detectable label and
wherein the pre-reporter antibody binds to the C1q with a variable region of the pre-reporter antibody and the reporter antibody binds to a heavy chain constant region of the pre-reporter antibody with the variable region of the reporter antibody.

25. The kit according to claim 24, comprising an amount of at least two microspheres complexes,
wherein a first microsphere complex comprises a first microsphere coupled to a first flavivirus antigen and a second microsphere complex comprises a second microsphere coupled to a second flavivirus antigen,
wherein the first microsphere comprises a first detectable label and the second microsphere comprises a second detectable label, and
wherein the emission signal of the first detectable label differs from the emission signal of the second detectable label.

26.-32. (canceled)

33. A method for determining the presence and/or amount of flavivirus-reactive complement-fixing antibodies in a sample from a subject comprising the steps of:
Step 1: contacting an amount of a microsphere complex comprising a microsphere coupled to a flavivirus antigen with the sample to allow binding of the flavivirus-reactive complement-fixing antibodies in the sample to the flavivirus antigen;
Step 2: contacting an amount of a complement competent serum with the complement-fixing antibodies bound to the flavivirus antigen in step 1 to allow the formation of a complement component fragment C3d (C3d);
Step 3: contacting an amount of a reporter antibody with the C3d formed in step 2 to allow binding of the reporter antibody to the C3d, wherein the reporter antibody binds to the C3d with a variable region of the reporter antibody and wherein the reporter antibody is attached to a detectable label; and
Step 4: detecting a signal from the reporter antibody bound to the C3d in step 3, wherein the signal is indicative for the presence and/or amount of the reporter antibody and wherein the presence and/or amount of the reporter antibody is indicative for the presence and/or amount of flavivirus-reactive complement-fixing antibodies in the sample.

34. The method according to claim 33, comprising the steps of:
Step 1: contacting an amount of a microsphere complex comprising a microsphere coupled to a flavivirus antigen with the sample to allow binding of the flavivirus-reactive complement-fixing antibodies in the sample to the flavivirus antigen;
Step 2: contacting an amount of a complement competent serum with the complement-fixing antibodies bound to the flavivirus antigen in step 1 to allow the formation of C3d;
Step 3.1: contacting an amount of a pre-reporter antibody with the C3d formed in step 2 to allow binding of the pre-reporter antibody to the C3d, wherein the pre-reporter antibody binds to the C3d with a variable region of the pre-reporter antibody;
Step 3.2: contacting an amount of a reporter antibody with the pre-reporter antibody bound to the C3d in step 3.1 to allow binding of the reporter antibody to the pre-reporter antibody, wherein the reporter antibody binds to a heavy chain constant region of the pre-reporter antibody with a variable region of the reporter antibody and wherein the reporter antibody is attached to a detectable label; and
Step 4: detecting a signal from the reporter antibody bound to the pre-reporter antibody in step 3.2, wherein the signal is indicative for the presence and/or amount of the reporter antibody and wherein the presence and/or amount of the reporter antibody is indicative for the presence and/or amount of flavivirus-reactive complement-fixing antibodies in the sample.

35. The method of claim 33, comprising the further steps of:
Step 5: determining the presence and/or amount of the reporter antibody from the signal of step 4; and
Step 6: determining the presence and/or amount of flavivirus-reactive complement-fixing antibodies in the sample from the presence and/or amount of the reporter antibody determined in step 5.

36.-37. (canceled)

38. A method for the concomitant determination of the presence and/or amount of complement-fixing antibodies reactive to different flaviviruses in a sample from a subject comprising the steps of:
Step 1: contacting an amount of at least two microsphere complexes with the sample,
wherein the first microsphere complex comprises a first microsphere coupled to a first flavivirus antigen and the second microsphere complex comprises a second microsphere coupled to a second flavivirus antigen
to allow binding of the complement-fixing antibodies reactive to the first and/or the second flavivirus to the first flavivirus antigen and/or the second flavivirus antigen,
wherein the first microsphere comprises a first detectable label and the second microsphere comprises a second detectable label, and wherein the emission signal of the first detectable label differs from the emission signal of the second detectable label,
Step 2: contacting an amount of a complement competent serum with the complement-fixing antibodies bound to the flavivirus antigen in step 1 to allow the formation of C3d,
Step 3: contacting an amount of a reporter antibody with the C3d formed in step 2 to allow binding of the reporter antibody to the C3d, wherein the reporter antibody binds to the C3d with a variable region of the reporter antibody and wherein the reporter antibody is attached to a third detectable label,
Step 4: detecting the emission signal of the detectable label of at least one microsphere upon irradiation with a first light source and comparing the emission signal with the emission signal of the first detectable label and with the emission signal of the second detectable label thereby identifying the at least one microsphere and the flavivirus antigen the at least one microsphere is coupled to, and
simultaneously detecting a signal from the reporter antibody bound to the C3d in step 3 of the at least one microsphere upon irradiation with a second light source,
Step 5: repeating step 4 until at least 30 microspheres coupled to the same flavivirus antigen are identified, and
Step 6: summarizing the detected signal from the reporter antibody in step 4 for all identified microspheres coupled to the same flavivirus antigen, wherein the summarized signal is indicative for the presence and/or amount of the reporter antibody and wherein the presence and/or amount of the reporter antibody is indicative for the presence and/or amount of flavivirus-reactive complement-fixing antibodies in the sample.

39. The method according to claim 38, comprising the steps of:
Step 1: contacting an amount of at least two microsphere complexes with the sample,
wherein a first microsphere complex comprises a first microsphere coupled to a first flavivirus antigen and a second microsphere complex comprises a second microsphere coupled to a second flavivirus antigen
to allow binding of the complement-fixing antibodies reactive to the first and/or the second flavivirus to the first flavivirus antigen and/or the second flavivirus antigen,
wherein the first microsphere comprises a first detectable label and the second microsphere comprises a second detectable label, and wherein the emission signal of the first detectable label differs from the emission signal of the second detectable label,
Step 2: contacting an amount of a complement competent serum with the complement-fixing antibodies bound to the first and/or the second flavivirus antigen in step 1 to allow the formation of C3d,
Step 3.1: contacting an amount of a pre-reporter antibody with the C3d formed in step 2 to allow the formation of C3d and to allow binding of the pre-reporter antibody to the C3d, wherein the pre-reporter antibody binds to the C3d with a variable region of the pre-reporter antibody,
Step 3.2: contacting an amount of a reporter antibody with the pre-reporter antibody bound to the C3d in step 3.1 to allow binding of the reporter antibody to the pre-reporter antibody, wherein the reporter antibody binds to a heavy chain constant region of the pre-reporter antibody with a variable region of the reporter antibody and wherein the reporter antibody is attached to a third detectable label,
Step 4: detecting the emission signal of the detectable label of at least one microsphere upon irradiation with a first light source and comparing the emission signal with the emission signal of the first detectable label and with the emission signal of the second detectable label thereby identifying the at least one microsphere and the flavivirus antigen the at least one microsphere is coupled to, and
simultaneously detecting a signal from the reporter antibody bound to the C3d in step 3 of the at least one microsphere upon irradiation with a second light source,
Step 5: repeating step 4 until at least 30 microspheres coupled to the same flavivirus antigen are identified, and
Step 6: summarizing the detected signal from the reporter antibody in step 4 for all identified microspheres coupled to the same flavivirus antigen, wherein the summarized detected signal is indicative for the presence and/or amount of the reporter antibody and wherein the presence and/or amount of the reporter antibody is indicative for the presence and/or amount of flavivirus-reactive complement-fixing antibodies in the sample.

40. The method according to claim 38, wherein the method further comprises the steps of:
Step 7: determining the presence and/or amount of the reporter antibody from the summarized signal in step 6, and
Step 8: determining the presence and/or amount of flavivirus-reactive complement-fixing antibodies in the sample from the presence and/or amount of the reporter antibody determined in step 7.

41.-51. (canceled)

52. A kit for detecting flavivirus-reactive complement-fixing antibodies in a sample comprising:
an amount of at least one microsphere complex comprising a microsphere coupled to a flavivirus antigen,
an amount of a complement competent serum,
an amount of a reporter antibody, and
optionally, an amount of a pre-reporter antibody,
wherein the reporter antibody is attached to a detectable label and wherein the reporter antibody binds to the C3d with a variable region of the reporter antibody,
wherein the pre-reporter antibody binds to the C3d with a variable region of the pre-reporter antibody and the reporter antibody binds to a heavy chain constant region of the pre-reporter antibody with the variable region of the reporter antibody.

53. The kit according to claim 52, comprising an amount of at least two microspheres complexes including a first microsphere complex and a second microsphere complex,
wherein the first microsphere complex comprises a first microsphere coupled to a first flavivirus antigen and the second microsphere complex comprises a second microsphere coupled to a second flavivirus antigen,
wherein the first microsphere comprises a first detectable label and the second microsphere comprises a second detectable label, and
wherein the emission signal of the first detectable label differs from the emission signal of the second detectable label.

54.-59. (canceled)
